(19) United States
(12) Patent Application Publication
(10) Pub. No.: US 2019/0125745 A1
(43) Pub. Date: May 2, 2019

(54) TREATMENT AND DIAGNOSIS OF CANCER

(71) Applicant: Rgenix, Inc., New York, NY (US)

(72) Inventors: Eduardo J. MARTINEZ, Bryn Mawr, PA (US); Masoud Fakhr Tavazoie, New York, NY (US)

(21) Appl. No.: 15/536,921

(22) PCT Filed: Dec. 17, 2015
(86) PCT No.: PCT/US2015/066289

§ 371 Date: Jun. 16, 2017

Related U.S. Application Data
(60) Provisional application No. 62/093,323, filed on Dec. 17, 2014.

(51) Int. Cl. A61K 31/506; A61K 45/06; A61P 35/00; A61K 31/4245; A61K 31/4155; A61K 31/41; A61K 31/575; A61K 31/585

(52) U.S. Cl. CPC A61K 31/506; A61K 45/06; A61P 35/00; A61K 31/585; A61K 31/4155; A61K 31/41; A61K 31/575; A61K 31/4245

(57) ABSTRACT

This invention features compounds that modulate the activity of liver X receptors, pharmaceutical compositions including the compounds of the invention, and methods of utilizing those compositions for modulating the activity of liver X receptors for the treatment of cancer.

### BACKGROUND OF THE INVENTION

Liver X receptors (LXRs) belong to a family of nuclear hormone receptors that are endogenously activated by cholesterol and its oxidized derivatives to mediate transcription of genes involved in maintaining glucose, cholesterol, and fatty acid metabolism. LXRα is found predominantly in the liver, with low levels found in kidney, intestine, spleen, and adrenal tissue. LXRβ is ubiquitous in mammals and was found in nearly all tissues examined. Given the intricate link between lipid metabolism and cancer cell growth, the ubiquitous expression of LXRβ in some types of cancer is unlikely to be coincidental, allowing cancer cells to synthesize lipids and lipoprotein particles to sustain their growth. At the same time, however, such stable basal expression levels make LXRβ an ideal therapeutic target.

### SUMMARY OF INVENTION

This invention features compounds that modulate the activity of liver X receptors, pharmaceutical compositions including the compounds of the invention, and methods of utilizing those compositions for modulating the activity of liver X receptors for the treatment of cancer.

In a first aspect, the invention features a method of treating cancer. This method includes: administering an effective amount (e.g., an amount sufficient to increase the expression level or activity level of ApoE to a level sufficient to slow the spread of metastasis of the cancer) an LXR agonist (e.g., an LXRβ agonist) or a pharmaceutically acceptable salt thereof to a subject in need thereof.

In another aspect, the invention features another method of treating cancer in a subject in need thereof. This method includes contacting cells (e.g., cancer cells and/or healthy cells) in the subject with an LXR agonist (e.g. an LXRβ agonist) or a pharmaceutically acceptable salt thereof, In another aspect, the invention features a method of slowing the spread of a migrating cancer.

This method includes administering an effective amount of an LXR agonist (e.g., an LXRβ agonist) to a subject in need thereof.

In another aspect, the invention features a method for inhibiting proliferation or growth of cancer stem cells or cancer initiating cells. This method includes contacting a cell with an effective amount of an LXR agonist (e.g., an LXRβ agonist).

In another aspect, the invention features a method for reducing the rate of tumor seeding of a cancer. This method includes administering an effective amount of an LXR agonist (e.g., an LXRβ agonist) to a subject in need thereof.

In another aspect, the invention features a method of reducing or treating metastatic nodule-forming of cancer. This method includes administering an effective amount of an LXR agonist (e.g., an LXRβ agonist) to a subject in need thereof.

In some embodiments of any of the foregoing methods, the cancer is a drug resistant cancer or has failed to respond to a prior therapy (e.g., a cancer resistant to, or a cancer that has failed to respond to prior treatment with, vemurafenib, dacarbazine, a CTLA4 inhibitor, a PD1 inhibitor, interferon therapy, a BRAF inhibitor, a MEK inhibitor, radiotherapy, temozolimide, irinotecan, a CAR-T therapy, herceptin, perjeta, tamoxifen, xeloda, docetaxol, platinum agents such as carboplatin, taxanes such as paclitaxel and docetaxel, ALK inhibitors, MET inihibitors, alimta, abraxane, adriamycin, gemcitabine, avastin, halaven, neratinib, a PARP inhibitor, ARN810, an mTOR inhibitor, topotecan, gemzar, a VEGFR2 inhibitor, a folate receptor antagonist, demcizumab, fosbretabulin, or a PDL1 inhibitor).

In other embodiments of any of the foregoing methods, the cancer is metastatic. The cancer can include cells exhibiting migration and/or invasion of migrating cells and/or include cells exhibiting endothelial recruitment and/or angiogenesis. In other embodiments, the cancer is a cell migration cancer. In still other embodiments, the cell migration cancer is a non-metastatic cell migration cancer.

The cancer can be a cancer spread via seeding the surface of the peritoneal, pleural, pericardial, or subarachnoid spaces. Alternatively, the cancer can be a cancer spread via the lymphatic system, or a cancer spread hematogenously.

In particular embodiments, the cancer is a cell migration cancer that is a non-metastatic cell migration cancer, such as ovarian cancer, mesothelioma, or primary lung cancer.

In certain embodiments, the LXR agonist increases the expression level of ApoE at least 2.5-fold in vitro. In certain embodiments, the LXRβ agonist is selective for LXRβ over LXRα. In other embodiments, the LXRβ agonist has activity for LXRβ that is at least 2.5-fold greater than the activity of said agonist for LXRα. In some embodiments, the LXRβ agonist has activity for LXRβ that is at least 10-fold greater than the activity of said agonist for LXRα. In further embodiments, the LXRβ agonist has activity for LXRβ that is at least 100-fold greater than the activity of said agonist for LXRα. In certain embodiments, the LXR agonist has activity for LXRβ that is at least within 2.5-fold of the activity of said agonist for LXRα.

In other embodiments of any of the foregoing methods, the cancer is breast cancer, colon cancer, renal cell cancer, non-small cell lung cancer, hepatocellular carcinoma, gastric cancer, ovarian cancer, pancreatic cancer, esophageal cancer, prostate cancer, sarcoma, glioblastoma, diffuse large B-cell lymphoma, leukemia (e.g., acute myeloid leukemia), or melanoma. In some embodiments of any of the foregoing methods, the cancer is melanoma. In other embodiments of any of the foregoing methods, the cancer is breast cancer. In certain embodiments of any of the foregoing methods, the cancer is renal cell cancer. In further embodiments of any of the foregoing methods, the cancer is pancreatic cancer. In other embodiments of any of the foregoing methods, the cancer is non-small cell lung cancer. In some embodiments of any of the foregoing methods, the cancer is colon cancer. In further embodiments of any of the foregoing methods, the cancer is ovarian cancer. In other embodiments of any of the foregoing methods, the cancer is glioblastoma. In some embodiments, the cancer is breast cancer. In other embodiments, the cancer is prostate cancer. In certain embodiments, the cancer is diffuse large B-cell lymphoma. In some embodiments, the cancer is leukemia (e.g., acute myeloid leukemia).

In some embodiments, the cancer is melanoma that is resistant to, or has failed to respond to prior treatment with, vemurafenib, dacarbazine, interferon therapy, a CTLA-4 inhibitor, a BRAF inhibitor, a MEK inhibitor, a PD1 inhibitor, a PDL-1 inhibitor, or a CAR-T therapy. In other embodiments, the cancer is glioblastoma that is resistant to, or has failed to respond to prior treatment with, temozolimide, radiotherapy, avastin, irinotecan, a VEGFR2 inhibitor, a CAR-T therapy, or an mTOR inhibitor. In certain embodiments, the cancer is non-small cell lung cancer that is resistant to, or has failed to respond to prior treatment with, an EGFR inhibitor, platinum agents (e.g., carboplatin), avastin, an ALK inhibitor, a MET inhibitor, a taxane (e.g., paclitaxel or doceltaxel), gemzar, alimta, radiotherapy, a PD1 inhibitor, a PDL1 ihibitor, or a CAR-T therapy. In some embodiments, the cancer is a breast cancer that is resistant to, or has failed to respond to prior treatment with, herceptin, perjeta, tamoxifen, xeloda, docetaxel, carboplatin, paclitaxel, abraxane, adriamycin, gemcitabine, avastin, halaven, neratinib, a PARP inhibitor, a PD1 inhibitor, a PDL1 inhibitor, a CAR-T therapy, ARN810, or an mTOR inhibitor. In other embodiments, the cancer is ovarian cancer that is resistant to, or has failed to respond to prior treatment with, a PARP inhibitor, avastin, carboplatin, paclitaxel, docetaxel, topotecan, gemzar, a VEGR2 inhibitor, a folate receptor antagonist, a PD1 inhibitor, a PDL1 inhibitor, a CAR-T therapy, demcizumab, or fosbretabulin.

In some embodiments of any of the foregoing methods, the LXR agonist is a compound of Formula I:

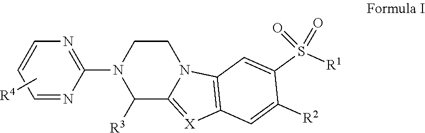

or a pharmaceutically acceptable salt thereof;

X is N or CR;

R¹ is alkyl or —NR^{a}R^{b}b

R² is H; halogen; —CN; —NRC(O)R; —C(O)OR; —C(O)NR^{a}R^{b}; monocyclic heteroaromatic optionally substituted with one or more groups selected from akyl, —CN, —NRC(O)R, —C(O)OR, —C(O)NR^{a}R^{b} and halogen; monocyclic non-aromatic heterocycle optionally substituted with one or more groups selected from alkyl, halogen, —CN and ═O; or alkyl optionally substituted by one or more groups selected from halogen, hydroxyl, alkoxy, —NR^{a}R^{b}, —NRC(O)R, —NRC(O)O(alkyl), —NRC(O)N(R)₂, —C(O)OR, thiol, alkyfthiol, nitro, —CN, ═O, —OC(O)H, —OC(O)(alkyl), —OC(O)O(alkyl), —OC(O)N(R)₂, and —C(O)NR^{a}R;

R³ is alkyl, haloalkyl, hydroxyalkyl, alkoxyalkyl, cycloalkyl, monocyclic nonaromatic heterocycle, monocyclic heteroaromatic or phenyl, wherein the phenyl, monocyclic non-aromatic heterocycle and monocyclic heteroaromatic group represented by R³ are optionally substituted with one or more groups selected from alkyl, halogen, haloalkyl, alkoxy, haloalkoxy, nitro and —CN;

R⁴ is halogen, —CN, —OR, —SR, —N(R)₂, —C(O)R, —C(O)OR, —OC(O)O(alkyl), —C(O)O(haloalkyl)-OC(O)R, —C(O)N(R)₂, —OC(O)N(R)₂, —NRC(O)R, —NRC(O)O(alkyl), —S(O)R—SO₂R, —SO₂N(R)₂, —NRS(O)R, —NRSO₂R, —NRC(O)N(R)₂, —NRSO₂N(R)₂, haloalkyl, haloalkoxy, cycloalkoxy, cycloalkyl, monocyclic non-aromatic heterocycle, monocyclic heteroaromatic or alkyl, wherein the monocyclic non-aromatic heterocycle, monocyclic heteroaromatic and alkyl group represented by R⁴ are optionally substituted with one or more groups selected from —CN, —OR, —SR, —N(R)₂, ═O, —C(O)R, —C(O)OR, —C(O)O(haloalkyl), —OC(O)R, —OC(O)O(alkyl), —C(O)N(R)₂, —OC(O)N(R)₂, —NRC(O)R, —NRC(O)O(alkyl), —S(O)R, —SO₂R, —SO₂N(R)₂, —NRS(O)R, —NRSO₂R, —NRC(O)N(R)₂ and —NRSO₂N(R)₂;

each R is, independently, H or alkyl;

R^{a} and R^{b} are, independently, H, alkyl or R^{a} and R^{b} can be taken together with the nitrogen to which they are attached to form a monocyclic non-aromatic heterocycle; and

R^{c} is H, alkyl, or halogen.

In some embodiments, the compound of Formula I has the structure of any one of Formulae II-VI:

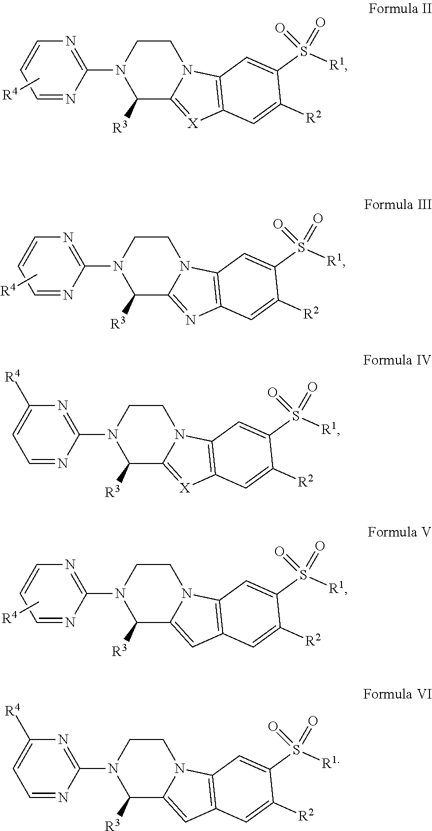

In some embodiments of the compounds of Formula I-VI, R³ is alkyl, haloalkyl, hydroxyalkyl, alkoxyalkyl, cycloalkyl or phenyl, wherein the phenyl represented by R³ is optionally substituted with one or more groups selected from alkyl, halogen, haloalkyl, alkoxy, haloalkoxy, nitro and —CN; and R⁴ is halogen, —CN, —OR, SR, —N(R)₂, —C(O)R, —C(O)OR, —OC(O)O(akyl), —C(O)O(haloalkyl), —OC(O)R, —C(O)N(R)₂, —OC(O)N(R)₂, —NRC(O)R, —NRC(O)O(alkyl), —S(O)R, —SO₂R, —SO₂N(R)₂, —NRS(O)R, —NRSO₂R, —NRC(O)N(R)₂, —NRSO₂N(R)₂, haloalkyl, haloalkoxy, cycloalkoxy, cycloalkyl, or alkyl, wherein the alkyl group represented by R⁴ is optionally substituted with one or more groups selected from —CN, —OR, —SR, —N(R)₂, ═O, —C(O)R, —C(O)OR, —C(O)O(haloalkyl), —OC(O)R, —OC(O)O(alkyl), —C(O)N(R)₂, —OC(O)N(R)₂, —NRC(O)R, —NRC(O)O(alkyl), S(O)R, —SO₂R, —SO₂N(R)₂, —NRS(O)R, —NRSO₂R, —NRC(O)N(R)₂, and —NRSO₂N(R)₂.

In other embodiments of the compounds of Formula I-VI, R¹ is methyl or —NH₂; R² is H or methyl, wherein the methyl group represented by R² is optionally substituted with one or more groups selected from halogen hydroxyl, alkoxy, —NR^{a}R^{b}, —NRC(O)R, —NRC(O)O(alkyl), —NRC(O)N(R)₂, —C(O)OR, thiol, alkyfthiol, nitro, —CN, ═O, —OC(O)H, —OC(O)(alkyl), —OC(O)O(alkyl), —C(O)NR^{a}R^{b}, and —OC(O)N(R)₂, preferably, R² is H or —CH₂OH; R³ is methyl, ethyl, propyl, isopropyl, tert-butyl, sec-butyl, iso-butyl, —CH₂CF₃, —CH(CH₂F)₂, —CH(CHF₂)₂, —CH(CF₃)₂, —CF(CH₃)₂, —CF₃, cyclopropyl, cyclobutyl, cyclopentyl, cyclohexyl, —C(OH)(CH₃)₂, —CH(OH)(CH₃), or phenyl, wherein the phenyl group represented by R³ is optionally substituted with one or more groups selected from alkyl, halogen, haloalkyl, alkoxy, haloalkoxy, nitro, and —CN; and R^{c}, where present, is H.

In certain embodiments of the compounds of Formula I-VI, R¹ is methyl; R² is —CH₂OH; and R³ is isopropyl.

In some embodiments of the compounds of Formula I-VI, R⁴ is halogen, hydroxy, alkyl, cycloalkyl, cycloalkoxy, alkoxy, haloalkoxy, haloalkyl, —N(R)₂, —C(O)OH, —C(O)O(alkyl), —C(O)O(haloalkyl), —C(O)(alkyl), —C(O)N(R)₂, —NRC(O)R, —SO₂N(R)₂, —OC(O)N(R)₂, —CN, hydroxyalkyl, or dihydroxyalkyl.

In other embodiments of the compounds of Formula I-VI, R⁴ is alkyl, haloalkyl, cycloalkyl, alkoxy, or haloalkoxy.

In certain embodiments of the compounds of Formula I-VI, R⁴ is methyl, ethyl, hydroxy, —CF₃, isopropyl, cyclopropyl, —CH₂OH, —CH(OH)(CH₂)(OH), —C(OH)(CH₃)₂, —CH(OH)(CH₃), —CH(OH)(CH₂)(CH₃), —CH(OH)(CH₂)₂(CH₃), —C(O)NH₂, —C(O)N(CH₃)₂, —C(O)OH, —C(O)NH(CH₃), —C(O)CH₃, —C(O)CH₂CH₃, —C(O)O(CH₂)(CH₃), —C(O)O(tert-butyl), —C(O)O(C)(CH₃)₂(CF₃), —NHC(O)CH₃, —OCHF₂, —OCF₃, —OCH₂CH₃, —OCH(CH₃)₂, or —OCH₃, preferably, R⁵ is —C(CH₃)₂OH.

In some embodiments of the compounds of Formula I-VI, R⁴ is methyl, halogenated methyl, cyclopropyl, —OCHF₂, or —OCH₃, preferably, R⁴ is CF₃.

In other embodiments of any of the foregoing methods, the LXR agonist is any one of compounds 1-7:

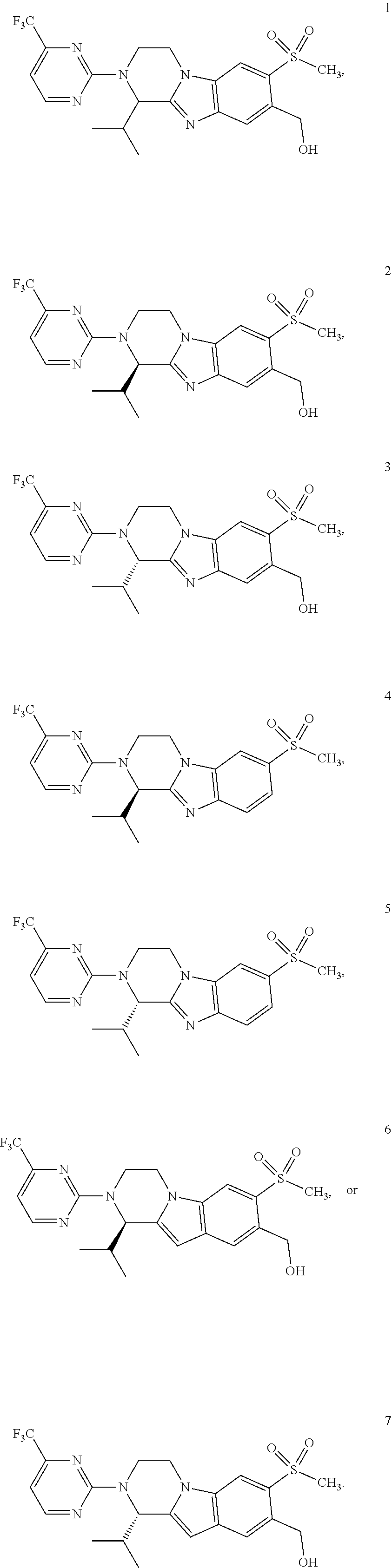

Compounds of Formula I-VI may be synthesized by methods known in the art, e.g., those described in International Patent Publication No. WO2013/138565. In some embodiments, the LXR agonist is a compound disclosed in U.S. Publication No. 2015/0246924, U.S. Publication No. 2015/0051214, U.S. Publication No. 2015/0065515, U.S. Publication No. 2015/0080406, or U.S. Publication No. 2015/033693, the compounds of which are herein incorporated by reference.

In some embodiments of any of the foregoing methods, the LXR agonist is a compound of Formula VII:

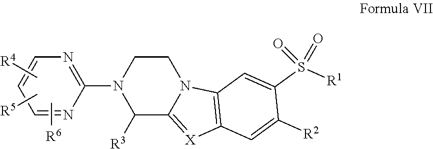

or a pharmaceutically acceptable salt thereof;

X is N or CRC;

R¹ is alkyl or —NR^{a}R^{b}

R² is H; halogen; —CN; —NRC(O)R; —C(O)OR; —C(O)NR^{a}R^{b}; monocyclic heteroaromatic optionally substituted with one or more groups selected from alkyl, —CN, —NRC(O)R, —C(O)OR, —C(O)NR^{a}R^{b}, and halogen; monocyclic non-aromatic heterocycle optionally substituted with one or more groups selected from alkyl, halogen, —CN, and ═O; or alkyl optionally substituted by one or more groups selected from halogen, hydroxy, alkoxy, —NR^{a}R^{b}, —NRC(O)R, —NRC(O)O(alkyl), —NRC(O)N(R)₂, —C(O)OR, thiol, alkytthiol, nitro, CN, ═O, —OC(O)H, —OC(O)(alkyl), —OC(O)O(alkyl), —OC(O)N(R)₂, and —C(O)NR^{a}R^{b};

R³ is alkyl, haloalkyl, hydroxyalkyl, alkoxyalkyl, cycloalkyl, monocyclic nonaromatic heterocycle, monocyclic heteroaromatic, or phenyl, wherein the phenyl, monocyclic non-aromatic heterocycle and monocyclic heteroaromatic group represented by R³ are optionally substituted with one or more groups selected from alkyl, halogen, haloalkyl, alkoxy, haloalkoxy, nitro, and —CN;

R⁴ and R⁵ are, independently, is halogen, —CN, —OR, —SR, —N(R)₂, —C(O)R, —C(O)OR, —OC(O)O(alkyl), —C(O)O(haloalkyl), —OC(O)R, —C(O)N(R)₂, —OC(O)N(R)₂, —NRC(O)R, —NRC(O)O(alkyl), —S(O)R, —SO₂R, —SO₂N(R)₂, —NRS(O)R, —NRSO₂R, —NRC(O)N(R)₂, —NRSO₂N(R)₂, haloalkyl, haloalkoxy, cycloalkoxy, cycloalkyl, monocyclic non-aromatic heterocycle, monocyclic heteroaromatic, or alkyl, wherein the alkyl, monocyclic non-aromatic heterocycle, and monocyclic heteroaromatic group represented by R⁴ or R⁵ are optionally substituted with one or more groups selected from —CN, —OR, —SR, —N(R)₂, ═O, —C(O)R, —C(O)OR, —C(O)O(haloalkyl), —OC(O)R, —OC(O)O(alkyl), —C(O)N(R)₂, —OC(O)N(R)₂, —NRC(O)R, —NRC(O)O(alkyl), —S(O)R, —SO₂R, —SO₂N(R)₂, —NRS(O)R, —NRSO₂R, —NRC(O)N(R)₂, and —NRSO₂N(R)₂;

R⁶ is H, halogen, —CN, —OR, —SR, —N(R)₂, —C(O)R, —C(O)OR, —OC(O)O(alkyl), —C(O)O(haloalkyl), —OC(O)R, —C(O)N(R)₂, —OC(O)N(R)₂, —NRC(O)R, —NRC(O)O(alkyl), —S(O)R, —SO₂R, —SO₂N(R)₂, —NRS(O)R, —NRSO₂R, —NRC(O)N(R)₂, —NRSO₂N(R)₂, haloalkyl, haloalkoxy, cycloalkoxy, cycloalkyl, or alkyl, wherein the alkyl group represented by R⁶ is optionally substituted with one or more groups selected from —CN, —OR, —SR, —N(R)₂, ═O, —C(O)R, —C(O)OR, —C(O)O(haloalkyl), —OC(O)R, —OC(O)O(alkyl), —C(O)N(R)₂, —OC(O)N(R)₂, —NRC(O)R, —NRC(O)O(alkyl), —S(O)R, —SO₂R, —SO₂N(R)₂, —NRS(O)R, —NRSO₂R, —NRC(O)N(R)₂, and —NRSO₂N(R)₂; or R⁵ and R⁶, taken together with the carbon atoms to which they are bonded, form a moncyclic non-aromatic heterocycle optionally substituted with one or more groups selected from alkyl, halogen, hydroxyalkyl, alkoxyakyl, haloalkyl, and =0;

each R is, independently, H or alkyl;

R^{a} and R^{b} are, independently, H, alkyl, or R^{a} and R^{b} can be taken together with the nitrogen to which they are attached to form a monocyclic non-aromatic heterocycle; and

R^{c} is H, alkyl, or halogen.

In some embodiments, the compound of Formula VII has the structure of Formula VIII-XIII:

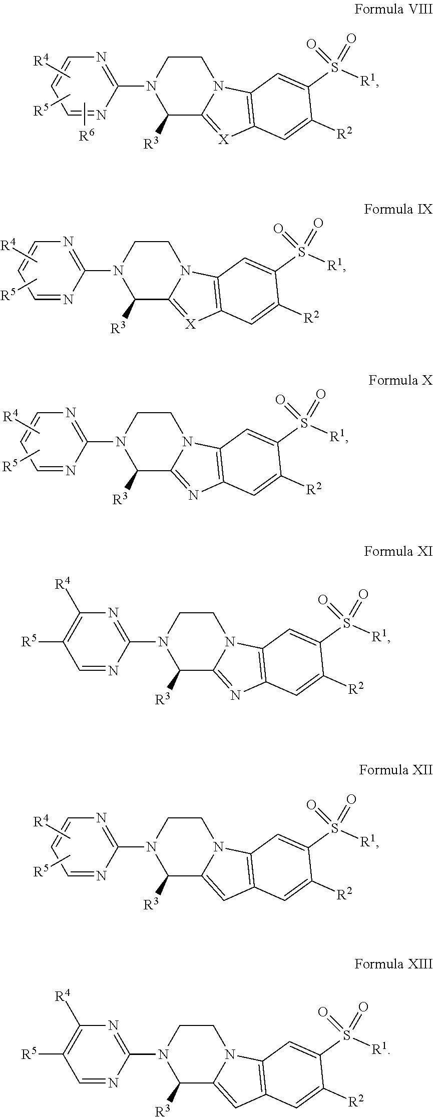

In some embodiments of the compounds of Formula VII-XIII, R³ is alkyl, haloalkyl, hydroxyalkyl, alkoxyalkyl, cycloalkyl, or phenyl, wherein the phenyl group represented by R³ is optionally substituted with one or more groups selected from alkyl, halogen, halo alkyl, alkoxy, haloalkoxy, nitro, and —CN; R⁴ and R⁵ independently are halogen, —CN, —OR, —SR, —N(R)₂, —C(O)R, —C(O)OR, —OC(O)O(alkyl), —C(O)O(haloalkyl), —OC(O)R, —C(O)N(R)₂, —OC(O)N(R)₂, —NRC(O)R, —NRC(O)O(alkyl), —S(O)R, —SO₂R, —SO₂N(R)₂, —NRS(O)R, —NRSO₂R, —NRC(O)N(R)₂, —NRSO₂N(R)₂, haloalkyl, haloalkoxy, cycloalkoxy, cycloalkyl, or alkyl, wherein the alkyl represented by R⁴ or R⁵ is optionally substituted with one or more groups selected from —CN, —OR, —SR, —N(R)₂, ═O, —C(O)R, —C(O)OR, —C(O)O(haloalkyl), —OC(O)R, —OC(O)O(alkyl), —C(O)N(R)₂, —OC(O)N(R)₂, —NRC(O)R, —NRC(O)O(alkyl), —S(O)R, —SO₂R, —SO₂N(R)₂, —NRS(O)R, —NRSO₂R, —NRC(O)N(R)₂, and —NRSO₂N(R)₂; R⁶ is H, halogen, —CN, —OR, —SR, —N(R)₂, —C(O)R, —C(O)OR, —OC(O)O(alkyl), —C(O)O(haloalkyl), —OC(O)R, —C(O)N(R)₂, —OC(O)N(R)₂, —NRC(O)R, —NRC(O)O(alkyl), —S(O)R, —SO₂R, —SO₂N(R)₂, —NRS(O)R, —NRSO₂R, —NRC(O)N(R)₂, —NRSO₂N(R)₂, haloalkyl, haloalkoxy, cycloalkoxy, cycloalkyl, or alkyl, wherein the alkyl group represented by R⁶ is optionally substituted with one or more groups selected from —CN, —OR, —SR, —N(R)₂, ═O, —C(O)R, —C(O)OR, —C(O)O(haloalkyl), —OC(O)R, —OC(O)O(akyl), —C(O)N(R)₂, —OC(O)N(R)₂, —NRC(O)R, —NRC(O)O(alkyl), —S(O)R, —SO₂R, —SO₂N(R)₂, —NRS(O)R, —NRSO₂R, —NRC(O)N(R)₂, and —NRSO₂N(R)₂.

In other embodiments of the compounds of Formula VII-XIII, R¹ is methyl or —NH₂; R² is H or methyl, wherein the methyl group represented by R² is optionally substituted with one or more groups selected from halogen, hydroxy, alkoxy, —NR^{a}R^{b}, —NRC(O)R, —NRC(O)O(alkyl), —NRC(O)N(R)₂, —C(O)OR, thiol, alkyfthiol, nitro, —CN, ═O, —OC(O)H, —OC(O)(akyl), —OC(O)O(akyl), —C(O)NR^{a}R^{b}, and —OC(O)N(R)₂, preferably, R² is H or —CH₂OH; R³ is methyl, ethyl, propyl, isopropyl, tert-butyl, sec-butyl, iso-butyl, —CH₂CF₃, —CH(CH₂F)₂, —CH(CHF₂)₂, —CH(CF₃)₂, —CF(CH₃)₂, —CF₃, cyclopropyl, cyclobutyl, cyclopentyl, cyclohexyl, —C(OH)(CH₃)₂, —CH(OH)(CH₃), or phenyl, wherein the phenyl group represented by R³ is optionally substituted with one or more groups selected from alkyl, halogen, haloalkyl, alkoxy, haloalkoxy, nitro, and —CN; and R^{c}, where present, is H.

In certain embodiments of the compounds of Formula VII-XIII, R¹ is methyl; R₂ is —CH₂OH; and R3 is isopropyl.

In some embodiments of the compounds of Formula VII-XIII, R⁴ and R⁵ independently are halogen, hydroxy, alkyl, cycloalkyl, cycloalkoxy, alkoxy, haloalkoxy, haloalkyl, —N(R)₂, —C(O)OH, —C(O)O(alkyl), —C(O)O(haloalkyl), —C(O)(alkyl), —C(O)N(R)₂, —NRC(O)R, —SO₂N(R)₂, —OC(O)N(R)₂, —CN, hydroxyalkyl, or dihydroxyalkyl.

In other embodiments of the compounds of Formula VII-XIII, R⁴ is alkyl, halo alkyl, cycloalkyl, alkoxy, or haloalkoxy.

In certain embodiments of the compounds of Formula VII-XIII, R⁴ and R⁵ independently are methyl, ethyl, hydroxy, —CF₃, isopropyl, cyclopropyl, —CH₂OH, —CH(OH)(CH₂)(OH), —C(OH)(CH₃)₂, —CH(OH)(CH₃), —CH(OH)(CH₂)(CH₃), —CH(OH)(CH₂)₂(CH₃), —C(O)NH₂, —C(O)N(CH₃)₂, —C(O)OH, —C(O)NH(CH₃), —C(O)CH₃, —C(O)CH₂CH₃, —C(O)O(CH₂)(CH₃), —C(O)O(tert-butyl), —C(O)O(C)(CH₃)₂(CF₃), —NHC(O)CH₃, —OCHF₂, —OCF₃, —OCH₂CH₃, —OCH(CH₃)₂, or —OCH₃, preferably, R⁴ is as just described and R⁵ is —C(OH)(CH₃)₂.

In some embodiments of the compounds of Formula VII-XIII, R⁴ is methyl, halogenated methyl, cyclopropyl, —OCHF₂, or —OCH₃, preferably, R⁴ is CF₃.

In other embodiments of any of the foregoing methods, the LXR agonist is any one of compounds 8-54:

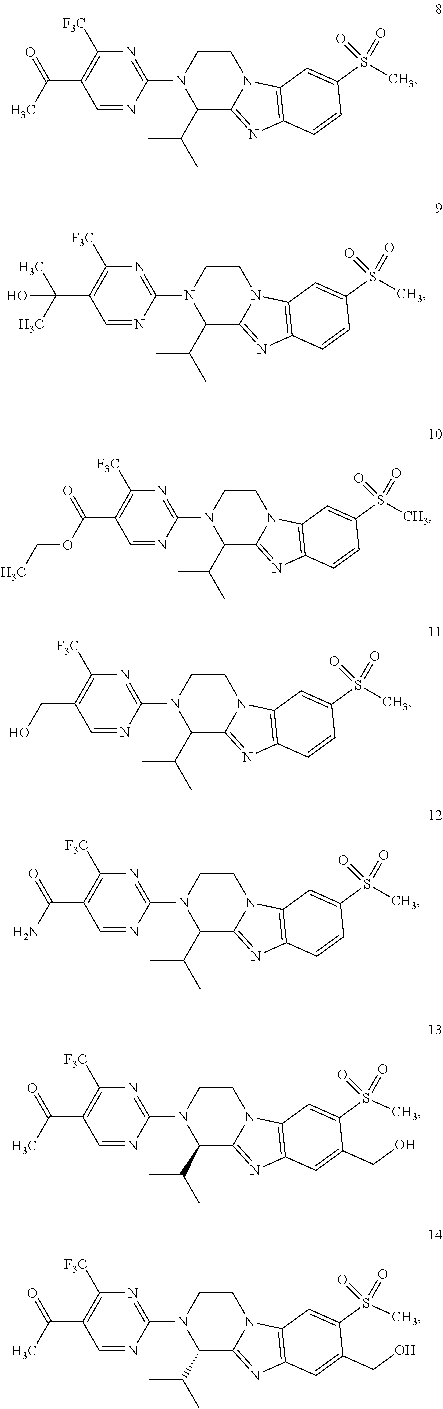

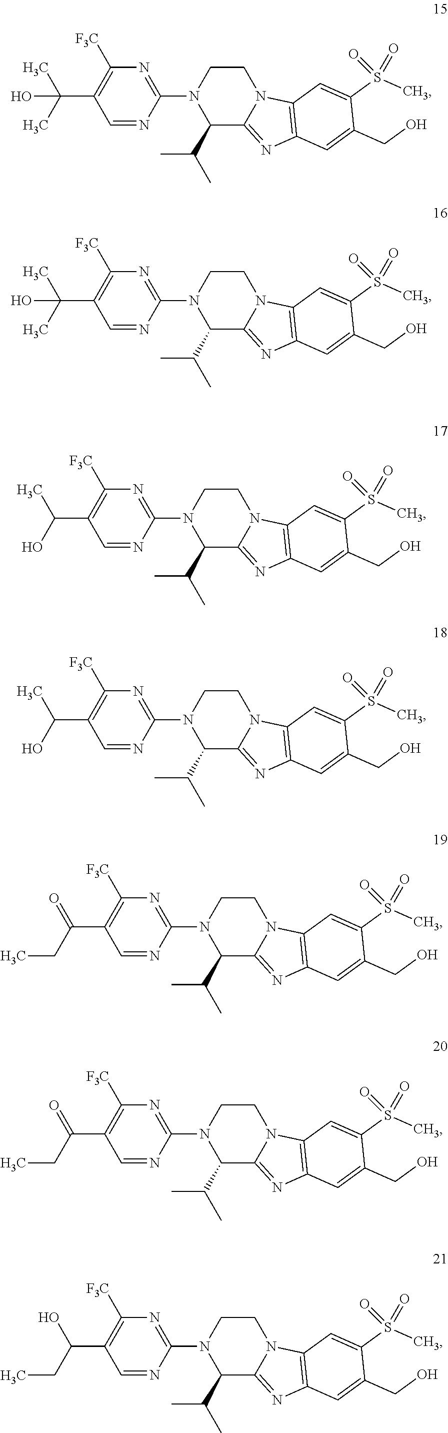

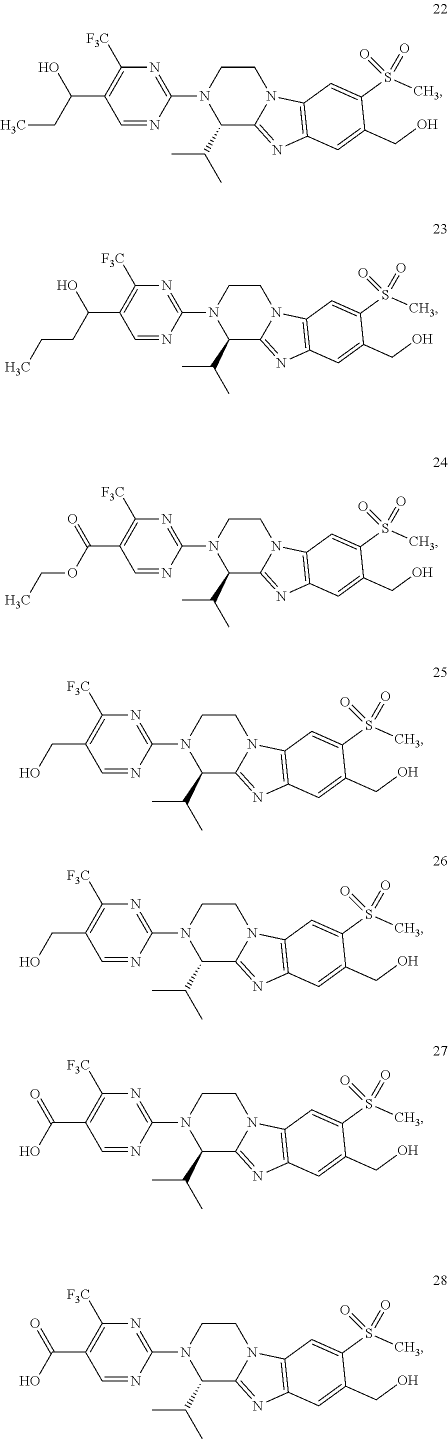

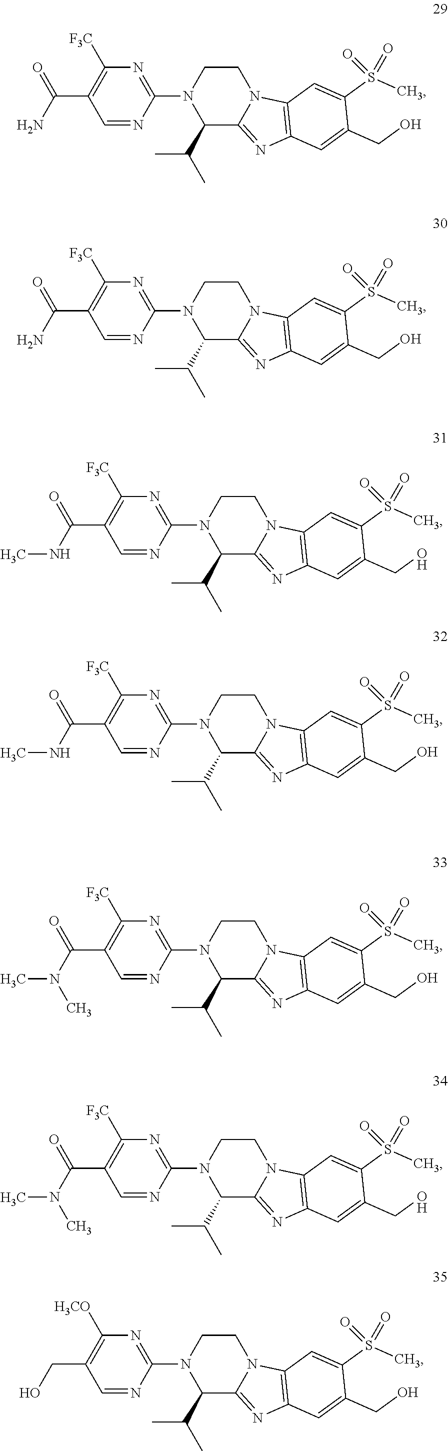

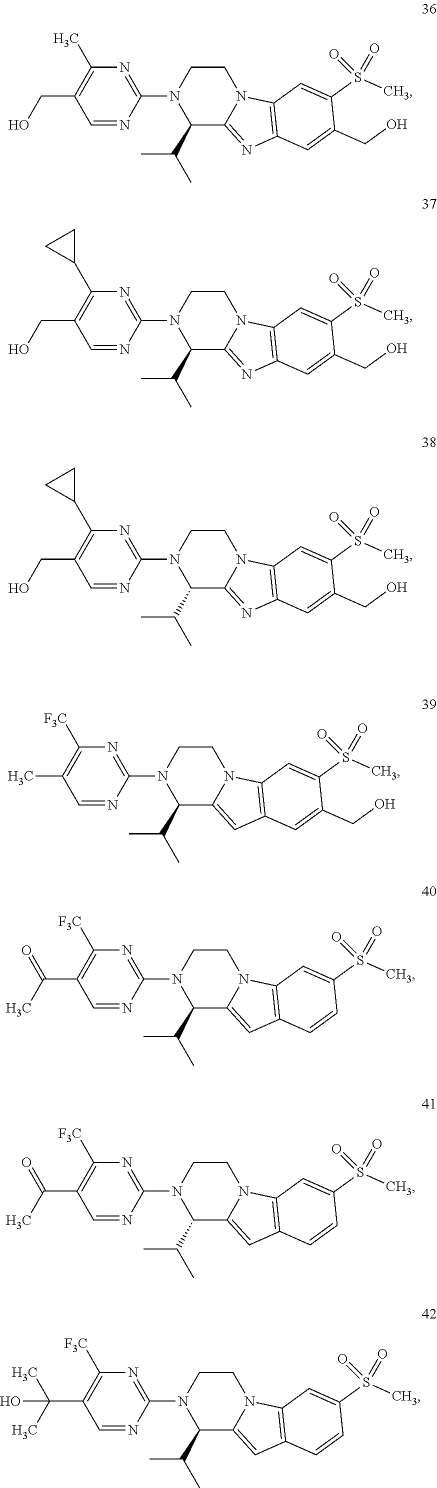

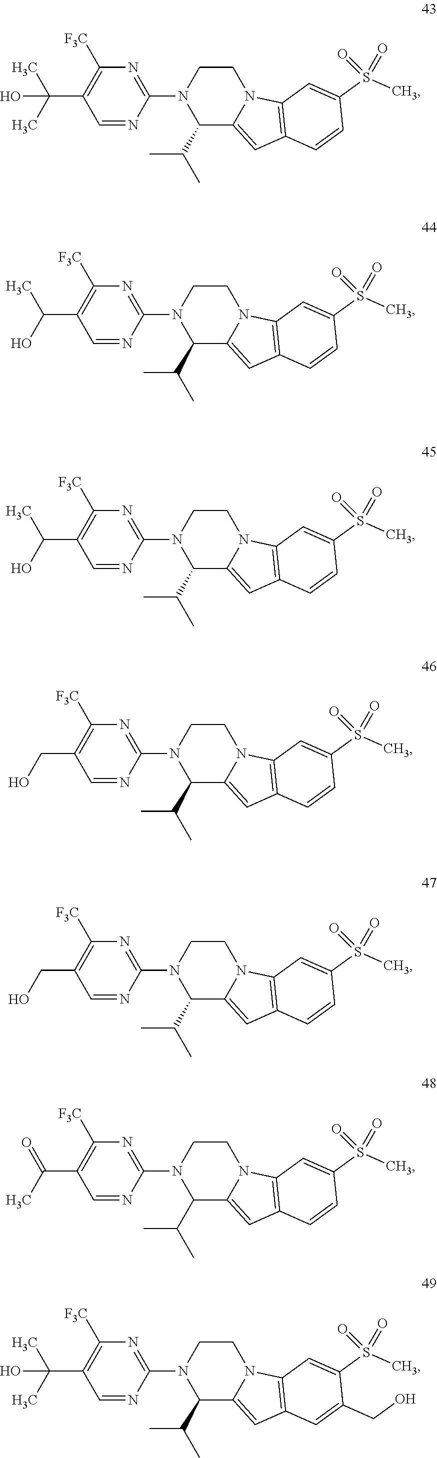

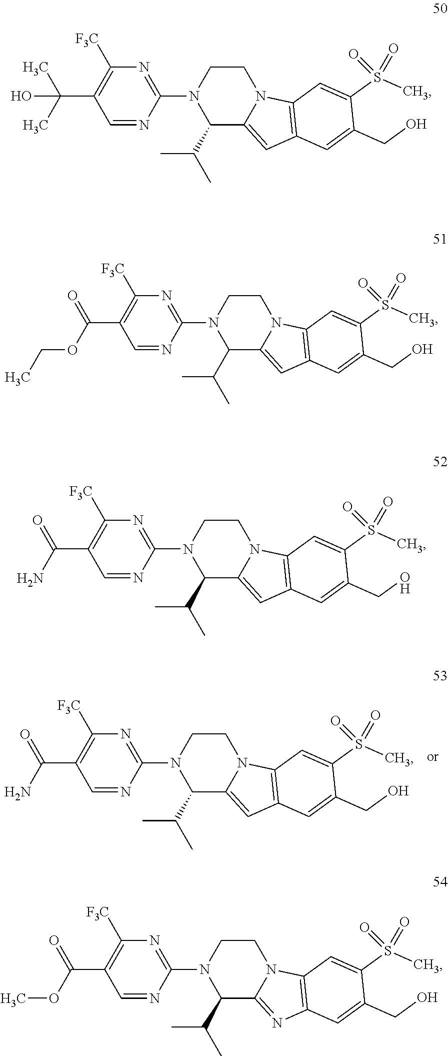 [SMILES: CC(C)[C@H]1c2cc3cc(CO)c(S(C)(=O)=O)cc3n2CCN1c1ncc(C(C)(C)O)c(C(F)(F)F)n1 ; CCOC(=O)c1cnc(N2CCn3c(cc4cc(CO)c(S(C)(=O)=O)cc43)C2C(C)C)nc1C(F)(F)F ; CC(C)[C@@H]1c2cc3cc(CO)c(S(C)(=O)=O)cc3n2CCN1c1ncc(C(N)=O)c(C(F)(F)F)n1 ; CC(C)[C@H]1c2cc3cc(CO)c(S(C)(=O)=O)cc3n2CCN1c1ncc(C(N)=O)c(C(F)(F)F)n1 ; "or" ; COC(=O)c1cnc(N2CCn3c(nc4cc(CO)c(S(C)(=O)=O)cc43)[C@H]2C(C)C)nc1C(F)(F)F]

Compounds of Formula VII may be synthesized by methods known in the art, e.g., methods described in International Patent Publication No. WO2013/138568.

In certain embodiments of any of the foregoing methods, the LXR agonist is a compound of Formula XIV:

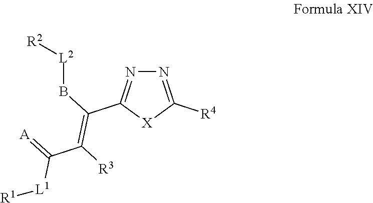

or a pharmaceutically acceptable salt thereof;

wherein X is —O— or —S—;

A and B are each nitrogen, wherein A and B are bonded together to form a five-membered heteroaryl ring;

L¹ and L² are each independently a bond, C₁-C₆ alkyl, or C₁-C₆ heteroalkyl;

R¹ is hydrogen, halogen, —CF₃, —OR⁸, —N(Re)₂, —C(═O)R⁸, —C(═O)OR⁸, —C(═O)N(R⁸)₂, —C(═NOH)R⁸, —C(═S)N(R⁸)₂, or —C(═O)OCH₂SCH₃;

R² is —OR⁹, —N(R⁹)₂, —C(═O)R⁹, —C(═O)OR⁹, —C(═O)N(R⁹)₂, —NR¹⁰C(═O)R⁹, —C(═N—OH)R⁹, C(═S)N(R⁹)₂, —C(═O)OCH₂SCH₃, C₁-C₆alkyl, C₃-C₈cycloalkyl, C₁-C₆ haloalkyl, C₁-C₆ heteroalkyl, optionally substituted heterocycloalkyl, optionally substituted aryl, or optionally substituted heteroaryl;

R³ is hydrogen, halogen, C₁-C₆ alkyl, or C₁-C₆haloalkyl;

R⁴ is aryl or heteroaryl; wherein aryl or heteroaryl is substituted with at least one R¹¹;

each R⁸, each R⁹, and each R¹⁰ are each, independently, hydrogen, C₁-C₆ alkyl, C₁-C₆ heteroalkyl, C₁-C₆alkyl-aryl, aryl, or heteroaryl; and

R¹¹ is, independently, halogen, nitro, —OR¹⁰, —N(R¹⁰)₂, —CN, —C(═O)R¹⁰, —C(═O)OR¹⁰, C(═O)N(R¹⁰)₂, —NR¹⁰C(═O)R¹⁰, NR¹⁰SO₂R¹⁰, —SOR¹⁰, —SO₂R¹⁰, —SO₂N(R¹⁰)₂, —C(═O)OCH₂SCH₃, C₁-C₆ alkyl, C₁-C₆ cycloalkyl, C₁-C₆ haloalkyl, C₁-C₆ heteroalkyl, C₁-C₆ alkyl-aryl, optionally substituted aryl, or optionally substituted heteroaryl.

In some embodiments of any of the foregoing methods, the LXR agonist is a compound of Formula XV:

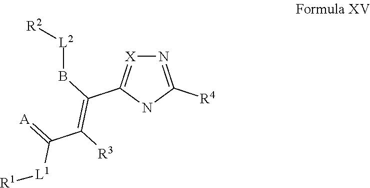

or a pharmaceutically acceptable salt thereof;

wherein X is —O— or —S—;

A and B are each nitrogen, wherein A and B are bonded together to form a five-membered heteroaryl ring;

L¹ and L² are each independently a bond, C₁-C₆ alkyl, or C₁-C₆ heteroalkyl;

R¹ is hydrogen, halogen, —CF₃, —OR⁸, —N(Re)₂, —C(═O)R⁸, —C(═O)OR⁸, —C(═O)N(R⁸)₂, —C(═NOH)R⁸, —C(═S)N(R⁸)₂, or —C(═O)OCH₂SCH₃;

R² is —OR⁹, —N(R⁹)₂, —C(═O)R⁹, —C(═O)OR⁹, —C(═O)N(R⁹)₂, —NR¹⁰C(═O)R⁹, —C(═N—OH)R⁹, C(═S)N(R⁹)₂, —C(═O)OCH₂SCH₃, C₁-C₆ alkyl, C₃-C₈ cycloalkyl, C₁-C₆ haloalkyl, C₁-C₆ heteroalkyl, optionally substituted heterocycloalkyl, optionally substituted aryl, or optionally substituted heteroaryl;

R³ is hydrogen, halogen, C₁-C₆ alkyl, or C₁-C₆ haloalkyl;

R⁴ is aryl or heteroaryl; wherein aryl or heteroaryl is substituted with at least one R¹¹;

each R⁸, each R⁹, and each R¹⁰ are each, independently, hydrogen, C₁-C₆ alkyl, C₁-C₆ heteroalkyl, C₁-C₆ alkyl-aryl, aryl, or heteroaryl; and

R¹¹ is, independently, halogen, nitro, —OR¹⁰, —N(R¹⁰)₂, —CN, —C(═O)R¹⁰, —C(═O)OR¹⁰, C(═O)N(R¹⁰)₂, —NR¹⁰C(═O)R¹⁰, NR¹⁰SO₂R¹⁰, —SOR¹, —SO₂R¹⁰, —SO₂N(R¹⁰)₂, —C(═O)OCH₂SCH₃, C₁-C₆ alkyl, C₃-C₈ cycloalkyl, C₁-C₆ haloalkyl, C₁-C₆ heteroalkyl, C₁-C₆ alkyl-aryl, optionally substituted aryl, or optionally substituted heteroaryl.

In other embodiments of any of the foregoing methods, the LXR agonist is a compound of Formula XVI:

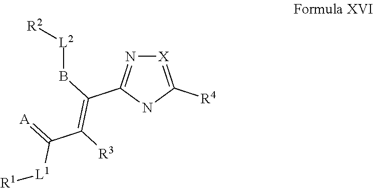

or a pharmaceutically acceptable salt thereof;

wherein X is —O— or —S—;

A and B are each nitrogen, wherein A and B are bonded together to form a five-membered heteroaryl ring;

L¹ and L² are each independently a bond, C₁-C₆ alkyl, or C₁-C₆ heteroalkyl;

R¹ is hydrogen, halogen, —CF₃, —OR⁸, —N(Re)₂, —C(═O)R⁸, —C(═O)OR⁸, —C(═O)N(R⁸)₂, —C(═NOH)R⁸, —C(═S)N(R⁸)₂, or —C(═O)OCH₂SCH₃;

R² is —OR⁹, —N(R⁹)₂, —C(═O)R⁹, —C(═O)OR⁹, —C(═O)N(R⁹)₂, —NR¹⁰C(═O)R⁹, —C(═N—OH)R⁹, C(═S)N(R⁹)₂, —C(═O)OCH₂SCH₃, C₁-C₆ alkyl, C₃-C₈ cycloalkyl, C₁-C₆ haloalkyl, C₁-C₆ heteroalkyl, optionally substituted heterocycloalkyl, optionally substituted aryl, or optionally substituted heteroaryl;

R³ is hydrogen, halogen, C₁-C₆ alkyl, or C₁-C₆ haloalkyl;

R⁴ is aryl or heteroaryl; wherein aryl or heteroaryl is substituted with at least one R¹¹;

each R⁸, each R⁹, and each R¹⁰ are each, independently, hydrogen, C₁-C₆ alkyl, C₁-C₆ heteroalkyl, C₁-C₆alkyl-aryl, aryl, or heteroaryl; and

R¹¹ is, independently, halogen, nitro, —OR¹⁰, —N(R¹⁰)₂, —CN, —C(═O)R¹⁰, —C(═O)OR¹⁰, C(═O)N(R¹⁰)₂, —NR¹⁰C(═O)R¹⁰, NR¹⁰SO₂R¹⁰, —SOR¹⁰, —SO₂R¹⁰, —SO₂N(R¹⁰)₂, —C(═O)OCH₂SCH₃, C₁-C₆ alkyl, C₃-C₈ cycloalkyl, C₁-C₆ haloalkyl, C₁-C₆ heteroalkyl, C₁-C₆ alkyl-aryl, optionally substituted aryl, or optionally substituted heteroaryl.

In certain embodiments of any of the foregoing methods, the LXR agonist is a compound of Formula XVII:

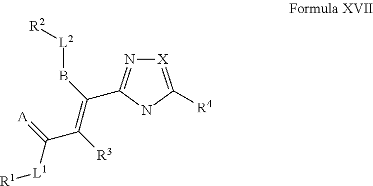

or a pharmaceutically acceptable salt thereof;

wherein X is —N(R¹²)—, or —O—;

A and B are each nitrogen, wherein A and B are bonded together to form a five-membered heteroaryl ring;

L¹ is a bond, C₁-C₆ alkyl, or C₁-C₆ heteroalkyl;

L² is C₁-C₆ alkyl or C₁-C₆ heteroalkyl;

R¹ is hydrogen, halogen, —CF₃, —OR⁸, —N(Re)₂, —C(═O)R⁸, —C(═O)OR⁸, —C(═O)N(R⁸)₂, —C(═NOH)R¹⁰, —C(═S)N(R⁸)₂, —C(═CH₂)CH₃, or —C(═O)OCH₂SCH₃;

R² is —C(═O)OR⁹, —C(═O)N(R⁹)₂, —NR¹⁰C(═O)R⁹, —C(═N—OH)R⁹, —C(═S)N(R⁹)₂, or —C(═O)OCH₂SCH₃;

R³ is hydrogen, halogen, C₁-C₆ alkyl, or C₁-C₆ haloalkyl;

R⁴ is aryl or heteroaryl; wherein aryl or heteroaryl is substituted with at least one R¹¹;

each R⁸, each R⁹, and each R¹⁰ are each, independently, hydrogen, C₁-C₆ alkyl, C₁-C₆ heteroalkyl, —C₁-C₆ alkyl-aryl, aryl, or heteroaryl;

R¹¹ is, independently, halogen, nitro, —OR¹⁰, —N(R¹⁰)₂, —CN, —C(═O)R¹⁰, —C(═O)OR¹⁰, C(═O)N(R¹⁰)₂, —NR¹⁰C(═O)R¹⁰, NR¹⁰SO₂R¹⁰, —SOR¹⁰, —SO₂R¹⁰, —SO₂N(R¹⁰)₂, —C(═O)OCH₂SCH₃, C₁-C₆ alkyl, C₃-cycloalkyl, C₁-C₆ haloalkyl, C₁-C₆ heteroalkyl, —C₁-C_{6b}alkyl-aryl, optionally substituted aryl, or optionally substituted heteroaryl; and

R¹² is hydrogen or C₁-C₆ alkyl.

In some embodiments of any of the foregoing methods, the LXR agonist is a compound of Formula XVIII:

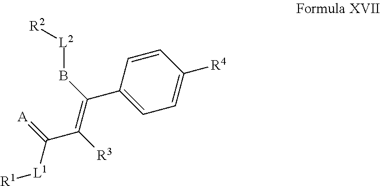

or a pharmaceutically acceptable salt thereof;

wherein A and B are each nitrogen, wherein A and B are bonded together to form a five-membered

heteroaryl ring;

L¹ and L² are each independently a bond, C₁-C₆ alkyl, or C₁-C₆ heteroalkyl;

R¹ is hydrogen, halogen, —CF₃, —OR⁸, —N(R⁸)₂, —C(═O)R⁸, —C(═O)OR⁸, —C(═O)N(R⁸)₂, —C(═NOH)R⁸, —C(═S)N(R⁸)₂, or —C(═O)OCH₂SCH₃;

R² is —OR⁹, —N(R⁹)₂, —C(═O)R⁹, —C(═O)OR⁹, —C(═O)N(R⁹)₂, —NR¹⁰C(═O)R⁹, —C(═N—OH)R⁹, C(═S)N(R⁹)₂, —C(═O)OCH₂SCH₃, C₁-C₆ alkyl, C₃-C₈ cycloalkyl, C₁-C₆ haloalkyl, C₁-C₆ heteroalkyl, optionally substituted heterocycloalkyl, optionally substituted aryl, or optionally substituted heteroaryl;

R³ is hydrogen, halogen, C₁-C₆ alkyl, or C₁-C₆ haloalkyl;

R⁴ is aryl or heteroaryl; wherein aryl or heteroaryl is substituted with at least one R¹¹;

each R⁸, each R⁹, and each R¹⁰ are each, independently, hydrogen, C₁-C₆ alkyl, C₁-C₆ heteroalkyl, C₁—C alkyl-aryl, aryl, or heteroaryl; and

R¹¹ is, independently, halogen, nitro, —OR¹⁰, —N(R¹⁰)₂, —CN, —C(═O)R¹⁰, —C(═O)OR¹⁰, C(═O)N(R¹⁰)₂, —NR¹⁰C(═O)R¹⁰, NR¹⁰SO₂R¹⁰, —SOR¹⁰, —SO₂R¹⁰, —SO₂N(R¹⁰)₂, —C(═O)OCH₂SCH₃, C₁-C₆ alkyl, C₃-C₈ cycloalkyl, C₁-C₆ haloalkyl, C₁-C₆ heteroalkyl, C₁-C₆ alkyl-aryl, optionally substituted aryl, or optionally substituted heteroaryl.

In other embodiments of any of the foregoing methods, the LXR agonist is a compound of Formula XIX:

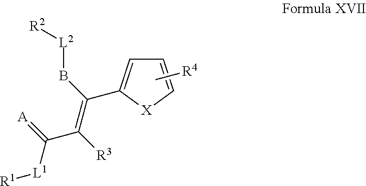

or a pharmaceutically acceptable salt thereof;

wherein X is —S—;

A and B are each nitrogen, wherein A and B are bonded together to form a five-membered heteroaryl ring;

L¹ is a bond, C₁-C₆ alkyl, or C₁-C₆ heteroalkyl;

L² is C₁-C₆ alkyl or C₁-C₆ heteroalkyl;

R¹ is hydrogen, halogen, C₁-C₆ alkyl, C₂-C₆ alkenyl, C₂-C₆ alkynyl, —CF₃, —OR⁸, —N(R⁸)₂, C(═O)R⁸, —C(═O)OR⁸, —C(═O)N(Re)₂, —C(═N—OH)R⁸, —C(═S)N(Re)₂, —C(═CH₂)CH₃, or C(═O)OCH₂SCH₃;

R² is —C(═O)OR¹³, —NR¹⁰C(═O)R⁹, —C(═N—OH)R⁹, —C(═S)N(R⁹)₂, or —C(═O)OCH₂SR¹⁵;

R³ is hydrogen, halogen, C₁-C₆ alkyl, or C₁-C₆ haloalkyl;

R⁴ is aryl or heteroaryl; wherein aryl or heteroaryl is substituted with at least one R¹¹;

each R⁸, each R⁹, and each R¹⁰ are each, independently, hydrogen, C₁-C₆ alkyl, C₁-C₆ heteroalkyl, —C₁-C₆ alkyl-aryl, aryl, or heteroaryl;

R¹¹ is, independently, halogen, nitro, —OR¹⁰, —N(R¹⁰)₂, —CN, —C(═O)R¹⁰, —C(═O)OR¹⁰, C(═O)N(R¹⁰)₂, —NR¹⁰C(═O)R¹⁰, —NR¹⁰SO₂R¹⁰, —SOR¹⁰, —SO₂R¹⁴, —SO₂N(R¹⁰)₂, —C(═O)OCH₂SCH₃, optionally substituted C₁-C₆ alkyl, optionally substituted C₃-C₈cycloalkyl, C₁-C₆ haloalkyl, optionally substituted C₁-C₆ heteroalkyl, optionally substituted —C₁-C₆ alkyl-aryl, optionally substituted aryl, or optionally substituted heteroaryl;

R¹³ is hydrogen, C₁-C₆ alkyl, C₁-C₆ heteroalkyl, —C₁-C₆ alkyl-aryl, aryl, or heteroaryl;

R¹⁴ is C₁-C₆ alkyl, C₁-C₆ heteroalkyl, —C₁-C₆ alkyl-aryl, aryl, or heteroaryl; and

R¹⁵ is C₁-C₆ alkyl.

In certain embodiments of any of the foregoing methods, the LXR agonist is any one of compounds 55-61:

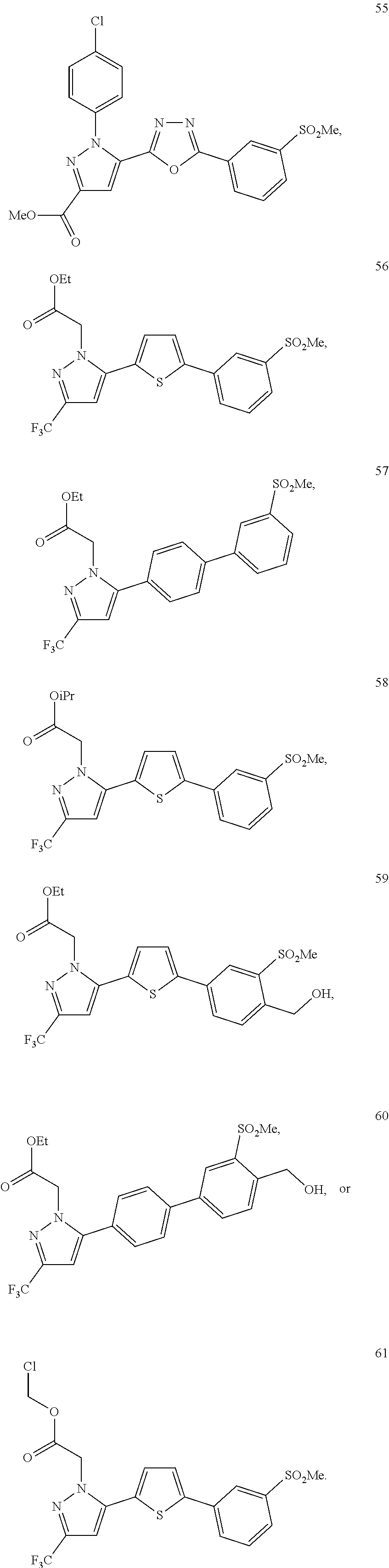

Compounds of Formula VII may be synthesized by methods known in the art, e.g., methods described in International Patent Publication No. WO2013/130892.

In some embodiments, the LXR agonist is a compound disclosed in U.S. Publication No. 2015/0152094 or U.S. Publication No. 2015/0045399, the compounds of which are herein incorporated by reference.

In some embodiments of any of the foregoing methods, the LXR agonist is a compound of Formula XVIII:

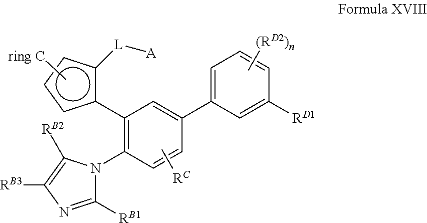

or a pharmaceutically acceptable salt thereof;

L is a bond, —[C(R¹)₂]ₘ—, -cyclopropyl-, or —CO—;

m is 1 or 2;

n is 0, 1, 2, 3, or 4;

R¹ is independently selected from H, C₁₋₃alkyl, —OH, or halo;

A is phenyl, cyclohexyl, a 5 or 6 membered heterocyclyl, or a 5 or 6 membered heteroaryl, wherein the phenyl is optionally fused to a 5 or 6 membered heterocyclyl or 5 or 6 membered heteroaryl, wherein A is optionally substituted with 1, 2, or 3 R^{A} groups, wherein each R^{A} is independently R^{A1}, —C₁-C₆ alkyl-R^{A1}, C₁-C₆ alkyl, C₁-C₆ haloalkyl, C₂-C₆ alkenyl, C₂-C₆ alkynyl, C₃-C₈cycloalkyl, or heterocyclyl, wherein the cycloalkyl or heterocyclyl are each optionally substituted with 1, 2, 3, or 4 groups that are independently R^{A1}, C₁-C₆ alkyl, or —C₁-C₆ alkyl-R^{A1}, wherein each R^{A1} is independently halogen, cyano, nitro, —OR, —NR₂, —SR, —C(O)R, or —C(O)OR; alternatively, 2 R^{A} on adjacent carbons can join to form a —O—CH₂—O—, —O—CH₂—CH₂—, —O—CH₂—CH₂—O—, —O—CF₂—O—, or —CH₂—CH₂—CH₂—;

ring C is a 5 membered heterocyclic ring selected from triazolyl, imidazolyl, pyrrazolyl, oxazolyl; wherein when ring C is pyrrazolyl, imidazolyl, or oxazolyl, then ring C is optionally substituted with C₁-C₄ alkyl, C₂-C₃ alkenyl, C₁-C₃ haloalkyl, C₃-C₆ cycloalkyl, CF₃, C₁-C₄ alkyl-OH, C₁-C₄ alkyl-O—C₁-C₄ alkyl, C₁-C₃ alkyl-NR₂; C₁-C₃ alkyl-CO₂H, C₁-C₃ alkyl-NHSO₂—C₁-C₃ alkyl, —NH—C₁-C₃alkyl-OR, C₁-C₃ alkyl-pyrrolidinyl;

R^{B1} is hydrogen, C₁-C₃alkyl, halo, or C₁-C₃haloalkyl;

R^{B2} is hydrogen, C₁-C₃ alkyl, halo, or C₁-C₃ haloalkyl;

R^{B3} is hydrogen, C₁-C₄ alkyl, halo, CN, C₁-C₄ haloalkyl, —C(O)—C₁-C₃ alkyl, —CO—NH₂, —CO—NR₂, or —C₁-C₃ alkyl-OH;

each R^{D1} and R^{D2} are independently R^{D3}, C₁-C₆ alkyl, —C₁-C₆ alkyl-R^{D3}, C₁-C₆ haloalkyl, C₂-C₆ alkenyl, C₂-C₆ alkynyl, C₃-C₈ cycloalkyl, or heterocyclyl, wherein the cycloalkyl and heterocyclyl are each optionally substituted with 1, 2, 3, or 4 groups that are independently R^{D3}, C₁-C₆ alkyl, C₃-C₈ cycloalkyl, or —C₁-C₆ alkyl-R^{D3}, wherein each R^{D3} is independently halogen, cyano, —OR, —NR₂, —SR, —C(O)R, —C(O)OR, —C(O)NR₂, —S(O)R, —S(O)₂R, —S(O)NR₂, —S(O)₂NR₂, —OC(O)R, —OC(O)OR, —OC(O)NR₂, —N(R)C(O)R, —N(R)C(O)OR, —N(R)C(O)NR₂, —N(R)S(O)₂R, —N(R)S(O)₂OR, —N(R)S(O)₂NR₂, or —S(O)₂N(R)C(O)NR₂; and

R^{C} is hydrogen, halogen, cyano, or C₁-C₆ alkyl;

each R group is independently hydrogen, C₁-C₆ alkyl, —C₁-C₆ alkyl-R², C₁-C₆ haloalkyl, —C₁-C₆ haloalkyl-R², C₂-C₆ alkenyl, C₂-C₆ alkynyl, or C₃-C₈cycloalkyl, wherein each R² is independently cyano, —OR³, —N(R³)₂, —N(R³)S(O)₂R³, —N(R³)S(O)₂OR³, or —N(R³)S(O)₂N(R³)₂, wherein each R³ is independently hydrogen, C₁-C₆ alkyl, or C₁-C₆ haloalkyl.

In other embodiments of any of the foregoing methods, the LXR agonist is a compound of Formula XIX:

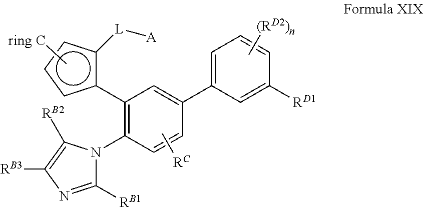

or a pharmaceutically acceptable salt thereof;

L is a bond, —[C(R¹)₂]ₘ—, -cyclopropyl-, or —CO—;

m is 1 or 2;

R¹ is independently selected from H, C₁-C₃alkyl, —OH, or halo;

A is phenyl, cyclohexyl, benzofuranyl, 2,3-dihydro-1H-indenyl, pyridyl, pyrazinyl, pyrimidinyl, dihydrobenzofuranyl, pyridin-2(1H)-one, imidazo[1,2-a]pyridinyl, or piperidinyl, wherein A is optionally substituted with 1, 2, or 3 R^{A} groups, wherein each R^{A} is independently halo, CN, C₁-C₆alkyl, C₁-C₆ haloalkyl, —O—R, NR₂, —O—C₁-C₆ alkyl, —O—C₁-C₆alkyl-C₃-C₆cycloalkyl, —S—R, —CO—R, —C(O)OR, —C₁-C₆ alkyl-CO—NR₂, pyrrolidinone, or pyrrolidinyl, alternatively, 2 R^{A} on adjacent carbons can join to form a —O—CH₂—O—, —O—CH₂CH₂—, —O—CH₂—CH₂—O—, or —O—CF₂—O—;

ring C is a 5 membered heterocyclic ring selected from triazolyl, imidazolyl, pyrrazolyl, oxazolyl; wherein when ring C is pyrrazolyl, imidazolyl, or oxazolyl, then ring C is optionally substituted with C₁-C₄ alkyl, C₂-C₃ alkenyl, C₁-C₃ haloalkyl, C₃-C₆ cycloalkyl, —CF₃, —C₁-C₄ alkyl-OH, —C₁-C₄ alkyl-O—C₁-C₃ alkyl, —C₁-C₃ alkyl-NR₂, —C₁-C₃ alkyl-CO₂H, —C₁-C₃ alkyl-NHSO₂—C₁-C₃ alkyl, —NH—C₁-C₃ alkyl-OR, or —C₁-C₃ alkyl-pyrrolidinyl;

R^{B1} is hydrogen, C₁-C₃ alkyl, halo, or C₁-C₃ haloalkyl;

R^{B2} is hydrogen, methyl or halo;

R^{B3} is hydrogen, C₁-C₄ alkyl, halo, CN, C₁-C₄ haloalkyl, cyclopropyl, —CO—NH₂, —CONR₂, or —C₁-C₃ alkyl-OH,

R^{C} is hydrogen, halogen, or cyano;

n is 0, 1, 2, 3, or 4;

R^{D1} is —SO₂—C₁-C₆ alkyl, —SO₂—C₁-C₆ haloalkyl, —SO₂—C₃-C₆ cycloalkyl, —SO₂—C₁-C₆ alkyl-OH, —SO₂—C₁-C₆ alkyl-O—C₁-C₆ alkyl, —C(Me)₂—COOH, C(Me)₂—CONR₂, cyclopropyl-CONR₂, —SO₂NR₂, —SO₂NR—C₁-C₆ alkyl-OH, —SO₂-pyrrolidinyl, or CONR₂;

R^{D2} is independently C₁-C₆ haloalkyl, —C₁-C₆ alkyl-OH, halo, —C₁-C₆ alkyl-O—C₁-C₆ alkyl, —C₁-C₆ alkyl-NHSO₂—C₁-C₆ alkyl, C₁-C₆ haloalkyl, or —O—C₁-C₆ alkyl-O—C₁-C₆ haloalkyl,

each R group is independently hydrogen, C₁-C₆ alkyl, —C₁-C₆ alkyl-R², C₁-C₆ haloalkyl, —C₁-C₆ haloalkyl-R², C₂-C₆ alkenyl, C₂-C₆ alkynyl, C₁-C₆ cycloalkyl;

each R² is independently —OR³, wherein each R³ is independently hydrogen; and C₁-C₆ alkyl, or C₁-C₆ haloalkyl.

In certain embodiments of any of the foregoing methods, the LXR agonist is a compound of Formula XX:

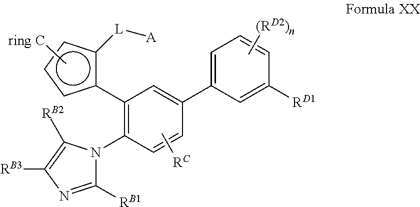

or a pharmaceutically acceptable salt thereof;

L is a bond, —[C(R¹)₂]ₘ—, -cyclopropyl-, or —CO—;

m is 1 or 2;

n is 0, 1, 2, 3, or 4;

R¹ is independently selected from H, C₁-C₃ alkyl, C₁-C₃ haloalkyl, —OH, and halo; A is phenyl, cyclohexyl, a 5 or 6 membered heterocycle, or a 5 or 6 membered heteroaryl, wherein the phenyl is optionally fused to a 5 or 6 membered heterocycle or 5 or 6 membered heteroaryl, wherein A is optionally substituted with 1, 2, or 3 R^{A} groups, wherein each R^{A} is independently R^{A1}, —C₁-C₆ alkyl-R^{A1}, C₁-C₆ alkyl, C₁-C₆ haloalkyl, C₂-C₆ alkenyl, C₂-C₆ alkynyl, C₃-C₈cycloalkyl, or heterocyclyl, wherein the cycloalkyl or heterocyclyl are each optionally substituted with 1, 2, 3, or 4 groups that are independently R^{A1}, C₁-C₆ alkyl, or —C₁-C₆ alkyl-R^{A1}, wherein each R^{A1} is independently halogen, cyano, nitro, —OR, —NR₂, —SR, —C(O)R, or —C(O)OR, alternatively, 2 R^{A} on adjacent carbons can join to form a —O—CH₂—O—, —O—CH₂—CH₂—, —O—CH₂—CH₂—O—, —CH₂—CH₂—CH₂—, or —O—CF₂—O—;

ring C is a 5 membered heterocyclic ring selected from triazolyl, imidazolyl, pyrrazolyl, and oxazolyl; wherein when ring C is pyrrazolyl, imidazolyl, or oxazolyl, then ring C is optionally substituted with C₁-C₄ alkyl, C₂-C₃ alkenyl, C₁-C₃ haloalkyl, C₃-C₆ cycloalkyl, —CF₃, —C₁-C₄ alkyl-OH, —C₁-C₄ alkyl-O—C₁-C₃ alkyl, —C₁—C alkyl-NR₂, —C₁-C₃ alkyl-CO₂H, —C₁—C alkyl-NHSO₂—CO₃alkyl, —NH—C₁-C₃ alkyl-OR, or —C₁-C₃ alkyl-pyrrolidinyl;

R^{B1} is hydrogen, C₁-C₃ alkyl, halo, or C₁-C₃ haloalkyl;

R^{B2} is hydrogen or halo;

R^{B3} is hydrogen, C₁-C₃ alkyl, halo, CN, C₁-C₃ haloalkyl, —C(O)—C₁-C₃ alkyl, —CO—NH₂, —CO—N(R)₂, or —C₁-C₃ alkyl-OH;

R^{D1} and R² are each independently R^{D3}, C₁-C₆ alkyl, —C₁-C₆ alkyl-R^{D}, C₁-C₆ haloalkyl, C₂-C₆ alkenyl, C₂-C₆ alkynyl, or C₃-C₈ cycloalkyl, or heterocyclyl, wherein the cycloalkyl or heterocyclyl are each optionally substituted with 1, 2, 3, or 4 groups that are independently R^{D3}, C₁—C alkyl, C₃-C₆ cycloalkyl, or —C₁-C₆ alkyl-R^{D3}, wherein each R^{D3} is independently halogen, cyano, —OR, —NR₂, —SR, —C(O)R, —C(O)OR, —C(O)NR₂, —S(O)R, —S(O)₂R, —S(O)NR₂, —S(O)₂NR₂, —OC(O)R, —OC(O)OR, —OC(O)NR₂, —N(R)C(O)R, —N(R)C(O)OR, —N(R)C(O)NR₂, —N(R)S(O)₂R, —N(R)S(O)₂OR, —N(R)S(O)₂NR₂, or-S(O)₂N(R)C(O)NR₂;

R^{C} is hydrogen, halogen, C₁—C alkyl, cyano, or nitro; and

each R group is independently hydrogen, C₁-C₆ alkyl, —C₁-C₆ alkyl-R², C₁-C₆ haloalkyl, —C₁-C₆ haloalkyl-R², C₂-C₆ alkenyl, C₂-C₆ alkynyl, C₃-C₈ cycloalkyl, or —C₁-C₆ alkyl-C₃-C₈ cycloalkyl, wherein each R² is independently cyano, —OR³, —N(R³)₂, —N(R³)S(O)₂R³, —N(R³)S(O)₂OR³, or —N(R³)S(O)₂N(R³)₂, wherein each R³ is independently hydrogen, C₁-C₆ alkyl, or C₁-C₆ haloalkyl.

In some embodiments of any of the foregoing methods, the LXR agonist is a compound of Formula XXI:

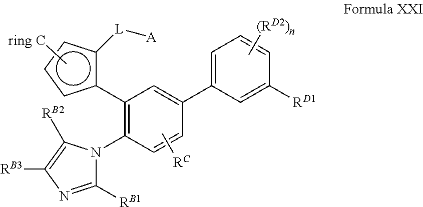

or a pharmaceutically acceptable salt thereof;

L is a bond, —[C(R¹)₂]ₘ—, -cyclopropyl-, or —CO—;

m is 1 or 2;

R¹ is independently selected from H, C₁-C₃ alkyl, C₁-C₃ haloalkyl, —OH, and halo; A is phenyl, cyclohexyl, naphthalenyl, benzofuranyl, 2,3-dihydro-1H-indenyl, 1H-indolyl, pyridyl, pyrazinyl, pyrimidinyl, dihydrobenzofuranyl, pyridin-2(1H)-one, imidazo[1,2-a]pyridinyl, or piperidinyl, wherein A is optionally substituted with 1, 2, or 3 R^{A} groups; wherein each R^{A} is independently halo, CN, C₁-C₆ alkyl, C₁-C₆ haloalkyl, —O—R, —NR₂, —O—C₁-C₆ alkyl, —O—C₁-C₆ alkyl-C₃-C₆ cycloalkyl, —S—R, —CO—R, —C(O)O—R, —C₁-C₆ alkyl-CO—NR₂, pyrrolidinone, or pyrrolidinyl, alternatively, 2 R^{A} on adjacent carbons can join to form a —O—CH₂—O—, —O—CH₂—CH₂—, —O—CH₂—CH₂—O—, or —O—CF₂—O—;

ring C is a 5 membered heterocyclic ring selected from triazolyl, imidazolyl, pyrrazolyl, and oxazolyl; wherein when ring C is pyrrazolyl, imidazolyl, or oxazolyl, then ring C is optionally substituted with C₁-C₄ alkyl, C₂-C₃ alkenyl, C₁₋₃ haloalkyl, C₃-C₆ cycloalkyl, —CF₃, —C₁-C₄ alkyl-OH, —C₁-C₄ alkyl-O—C₁-C₃ alkyl, —C₁-C₃ alkyl-NR₂; —C₁-C₃ alkyl-CO₂H, —C₁-C₃ alkyl-NHSO₂—C₁-C₃ alkyl, —NH—C₁-C₃ alkyl-OR, or —C₁-C₃ alkyl-pyrrolidinyl;

R^{B1} is hydrogen, C₁—C alkyl, halo, or C₁-C₃ haloalkyl;

R^{B2} is hydrogen or halo;

R^{B3} is hydrogen, C₁—C alkyl, halo, CN, C₁-C₄ haloalkyl, cyclopropyl, —CO—NH₂, —CONR₂, or —C₃—C alkyl-OH;

R^{C} is hydrogen, halogen, or cyano;

n is 0, 1, 2, 3, or 4;

R^{D1} is —SO₂—C₁-C₆ alkyl, —SO₂—C₁-C₆ haloalkyl, —SO₂—C₃-C₆ cycloalkyl, —SO₂—C₁-C₆ alkylOH, —SO₂—C₁-C₆ alkyl-O—C₁-C₆ alkyl, —C(Me)₂—COOH, —C(Me)₂—CONR₂, -cyclopropyl-CONR₂—, —SO₂NR₂, —SO₂NR—C₁-C₆ alkyl-OH, —SO₂-pyrrolidinyl, or —CONR₂;

R^{D2} is independently —C₁-C₆ haloalkyl—C₁-C₆ alkyl-OH, halo, —C₁-C₆ alkyl-O—C, —C₆ alkyl, —C₁-C₆alkyl-NHSO₂—C₁-C₆ alkyl, C₁-C₆ haloalkyl, or —O—C₁-C₆ alkyl-O—C₁-C₆ haloalkyl, each R group is independently hydrogen, C₁-C₆ alkyl, —C, —C₆ alkyl-R², C, —C₆ haloalkyl, —C₁-C₆ haloalkyl-R², C₂-C₆ alkenyl, C₂-C₆ alkynyl, or C₃-C₈cycloalkyl; and

each R² is independently —OR³, wherein each R³ is independently hydrogen, C₁-C₆ alkyl, or C₁-C₆ haloalkyl.

Compounds of Formula XVIII-XXI may be synthesized by methods known in the art, e.g., methods described in International Patent Publication No. WO2014/144037.

In other embodiments of any of the foregoing methods, the LXR agonist is a compound of Formula XXII:

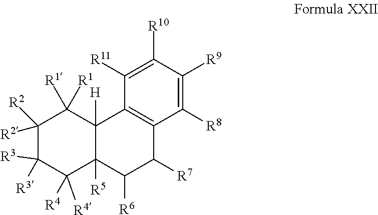

or a pharmaceutically acceptable salt thereof;
wherein R¹ and R^{1′} are independently selected from —H, OR, or —OR^{a}, wherein R^{a} is a hydroxyl protecting group or COR^{b}, or R¹ and R^{1′} taken together form a keto function;

R² and R^{r} are independently selected from the group consisting of —H, —C₁-C₆ alkyl, phenyl, or substituted phenyl, —OH, —OR^{a}, or R² and R^{2′} taken together to form a keto function;

R³ and R^{3′} are independently selected from the group consisting of —H, —C₁-C₆ alkyl, phenyl, or substituted phenyl, —OH, —OR^{a}; or R³ and R^{3′} taken together form a keto function;

R^{b} is selected from the group consisting of —C₁-C₆ alkyl, —C₃-C₇ cycloalkyl, phenyl, aryl, alkylaryl, and alkylheterocyclic;

R⁴ is selected from the group consisting of —H, —OH, —OR^{a}, —C₁-C₆ alkyl, phenyl, or substituted phenyl; R^{4′} is —H;

R⁵ is a group selected from hydrogen, —C₁-C₆ alkyl, phenyl, or substituted phenyl;

R⁶ and R⁷ are each independently selected from the group consisting of —H, —C₁-C₆ alkyl, —C₂-C₈ alkenyl, phenyl, or substituted phenyl;

R⁸ and R⁹ are each independently selected from —H, —C₁-C₆ alkyl, phenyl, or substituted phenyl, halo, —NO₂, —NR¹²R¹³, —CONR¹⁴R¹⁵, and —COOR¹⁶;

R¹⁰ is —H, OH, OR^{a}, COR^{a}, —C₁-C₆ alkyl, —C₂—C alkenyl, —C₂-C₆ alkynyl, phenyl, or substituted phenyl, CH₂OR′, —CHO, —CONR⁴R¹⁵, or —COOR¹⁶;

R¹¹ is —H, —C₁-C₆ alkyl, —C₂-C₈ alkenyl, phenyl or substituted phenyl, aryl, alkylaryl, or alkylheterocycle;

R¹² and R¹³ are independently selected from —H, —C₁-C₆ alkyl, —C₃-C₇ cycloalkyl, phenyl, aryl, alkylaryl, or R¹² taken together with R¹³ forms a 4, 5, 6, or 7-membered heterocyclic ring containing a nitrogen atom;

R¹⁴ and R¹⁵ are each independently selected from H, —C₁-C₆ alkyl, —C₃-C₇ cycloalkyl, phenyl, aryl, alkylaryl, or taken together form a 4, 5, 6, or 7-membered heterocyclic ring containing a nitrogen atom; and

R¹⁶ is —H, —C₁-C₆ alkyl, phenyl, substituted phenyl, or benzyl;

In some embodiments of the compounds of Formula XXII, when R⁹ is pyrolidine, R⁵ is methyl, and R¹⁰ is carboxyethyl ester group, and R¹ is in a trans relationship to R^{5′} then R¹ is not —OH; and if R¹ and R^{1′} are —OH and H respectively, or taken together to form a ketone, then R⁹ is not pyrolidinyl and R¹⁰ is not methyl, or hydroxylmethyl.

In certain embodiments of any of the foregoing methods, the LXR agonist is: trans-8-Hydroxy-9-hydro-1,2-[a,b] [(1-carboxyethyl-2-Npyrolidinyl)benzo-4,5-yl]-cis-10-methyldecalin; 8-keto-1,2-[a,b] [(1-carboxyethyl-1-N-pyrolidinyl)benzo-4,5-yl]-10-methyldecalin; 8-hydroxy-1,2-[a,b] [(1-hydroxymethyl-1-N-pyrolidinyl)benzo-4,5-yl]-10-methyldecalin; or 8-hydroxy-1,2-[a,b] [(1-methyl-1-N-pyrolidinyl)benzo-4,5-yl]-10-methyldecalin.

Compounds of Formula XXII may be synthesized by methods known in the art, e.g., methods described in International Patent Publication No. WO03/031408.

In some embodiments of any of the foregoing methods, the LXR agonist is a compound of Formula XXIII:

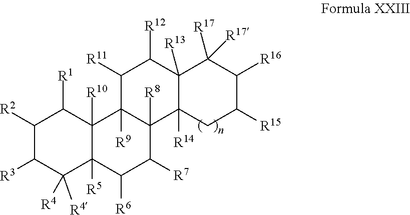

wherein R³ is hydrogen, amino, carboxyl, oxo, halo, sulfonic acid, —O-sulfonic acid, or. alkyl that is optionally inserted with —NH—, —N(alkyl)-, —O—, —S—, —SO—, —SO₂—, —OSO₂—, —SO₂—O—, —O—SO₃—, —SO₃—O—, —CO—, —CO—O—, —O—CO—, —CO—NH—, —CON(alkyl)-, —NH—CO—, or —N(alkyl)-CO—, and further optionally substituted with hydroxy, halo, amino, carboxyl, sulfonic acid, or —O-sulfonic acid. Each of R¹, R², R⁴, R^{4′}, R⁶, R⁷, R¹¹, R¹², R¹⁵, R¹⁶, and R^{17′} is, independently, hydrogen, hydroxy, amino, carboxyl, oxo, halo, sulfonic acid, —O-sulfonic acid, or alkyl that is optionally inserted with —NH—, —N(alkyl)-, —O—, —S—, —SO—, —SO₂-—O—SO₂—, —SO₂O—, —O—SO₃—, —SO₃—O—, —CO—, —CO—O—, —O—CO—, —CO—NH—, —CO—N(alkyl)-, —NH—CO—, or —N(alkyl)-CO—, and further optionally substituted with hydroxy, halo, amino, carboxyl, sulfonic acid, or —O-sulfonic acid. Each of R⁵, R⁸, R⁹, R¹⁰, R¹³, and R¹⁴, independently, is hydrogen, alkyl, haloalkyl, hydroxyalkyl, alkoxy, hydroxy, or amino. R¹¹ is —X—Y—Z. X is a bond, or alkyl or alkenyl, optionally inserted with —NH—, —N(alkyl)-, —O—, or —S—, and further optionally forming a cyclic moiety with R¹⁶ and the 2 ring carbon atoms to which R¹⁶ and R¹⁷ are bonded. Y is —CO—, —SO—, —SO₂—, —O—SO₂—, —SO₂—O—, —O—SO₃—, —SO₃—O—, —CO—O—, —O—CO—, —CONH—, —CO—N(alkyl)-, —NH—CO—, —N(alkyl)-CO—, or a bond. Z is alkyl, alkenyl, alkynyl, cycloalkyl, heterocycloalkyl, cycloalkenyl, heterocycloalkenyl, aryl, heteroaryl, aralkyl, or heteroaralkyl, and is optionally substituted with hydroxy, alkoxy, amino, halo, sulfonic acid, —O-sulfonic acid, carboxyl, oxo, alkyloxycarbonyl, alkylcarbonyloxy, alkylaminocarbonyl, alkylcarbonylamino, alkylcarbonyl, alkylsulfinyl, alkylsulfonyl, or alkylthio; or is —CH(A)-B. A being a side chain of an amino acid, and B is hydrogen, —NR^{a}R^{b}, or —COOR^{c} wherein each of R^{a}, R^{b}, and R^{c}, independently, is hydrogen or alkyl. n is 0, 1, or 2. Note that when Z is substituted with carboxyl or alkyloxycarbonyl, Y is a bond and either X or Z contains at least one double bond, and that when Y is a bond, either X is —NH-alkyl-, —NH— alkenyl-, —N(alkyl)-alkyl-, —N(alkyl)-alkenyl-, —O-alkyl-, —O-alkenyl-, —S-alkyl-, or —S-alkenyl-; or Z is substituted with halo, sulfonic acid, —O-sulfonic acid, alkylsulfinyl, or alkylsulfonyl, or is alkenyl, or a pharmaceutically acceptable salt thereof.

In other embodiments of any of the foregoing methods, the LXR agonist is any one of compounds 62-87:

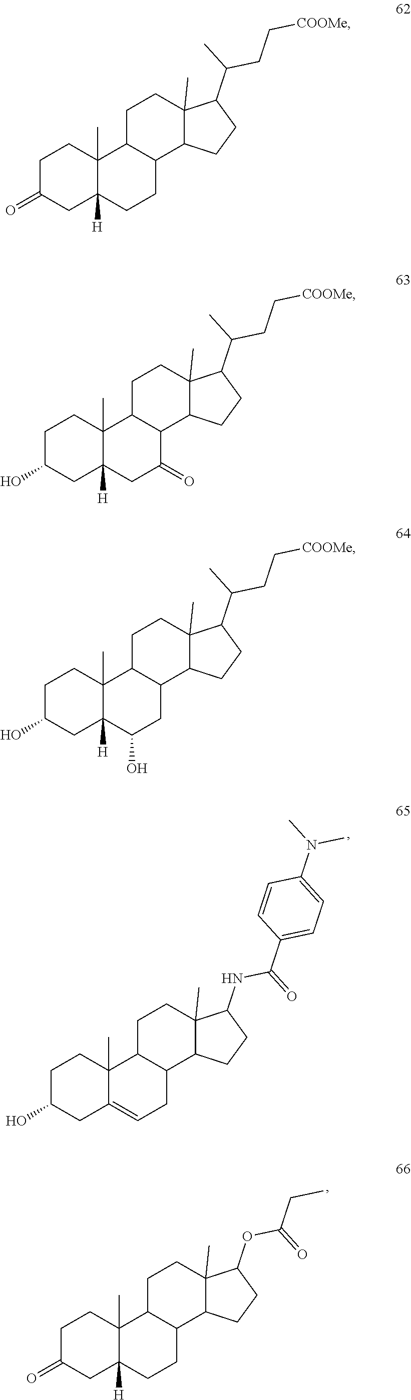 [SMILES: COC(=O)CCC(C)C1CCC2C3CC[C@@H]4CC(=O)CCC4(C)C3CCC12C ; COC(=O)CCC(C)C1CCC2C3C(=O)C[C@@H]4C[C@H](O)CCC4(C)C3CCC12C ; COC(=O)CCC(C)C1CCC2C3C[C@H](O)[C@@H]4C[C@H](O)CCC4(C)C3CCC12C ; CN(C)c1ccc(C(=O)NC2CCC3C4CC=C5C[C@H](O)CCC5(C)C4CCC23C)cc1 ; CCC(=O)OC1CCC2C3CC[C@@H]4CC(=O)CCC4(C)C3CCC12C]

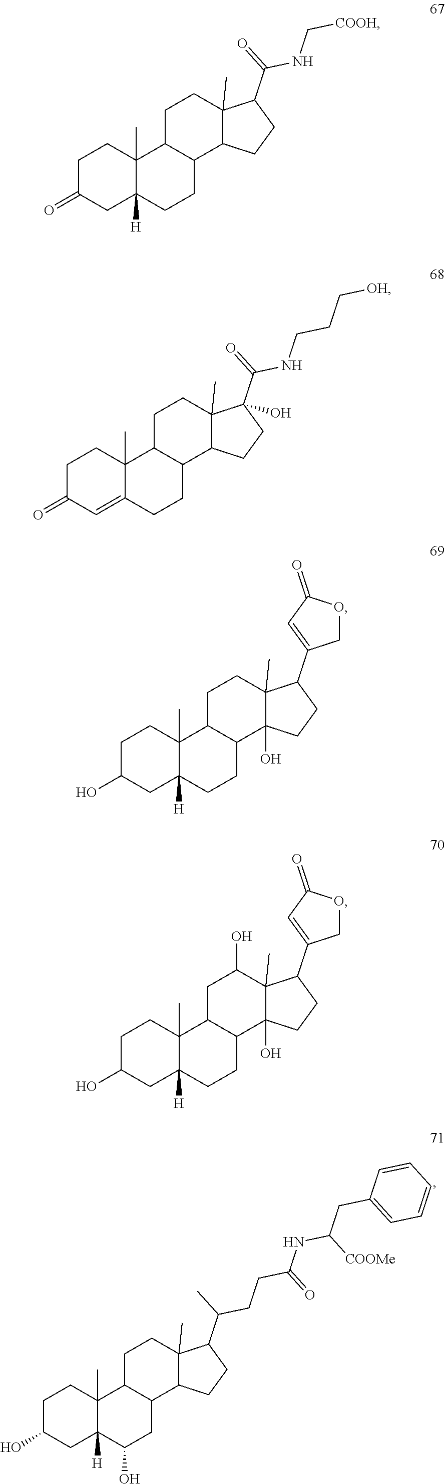 [SMILES: CC12CCC3C(CC[C@@H]4CC(=O)CCC34C)C1CCC2C(=O)NCC(=O)O ; CC12CCC(=O)C=C1CCC1C2CCC2(C)C1CC[C@]2(O)C(=O)NCCCO ; CC12CCC(O)C[C@H]1CCC1C2CCC2(C)C(C3=CC(=O)OC3)CCC12O ; CC12CCC(O)C[C@H]1CCC1C2CC(O)C2(C)C(C3=CC(=O)OC3)CCC12O ; COC(=O)C(Cc1ccccc1)NC(=O)CCC(C)C1CCC2C3C[C@H](O)[C@@H]4C[C@H](O)CCC4(C)C3CCC12C]

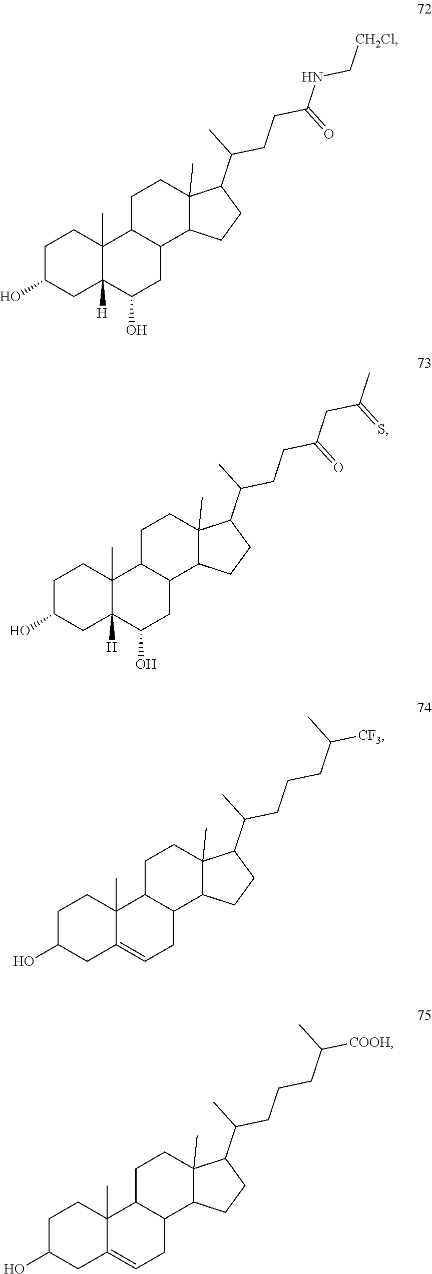

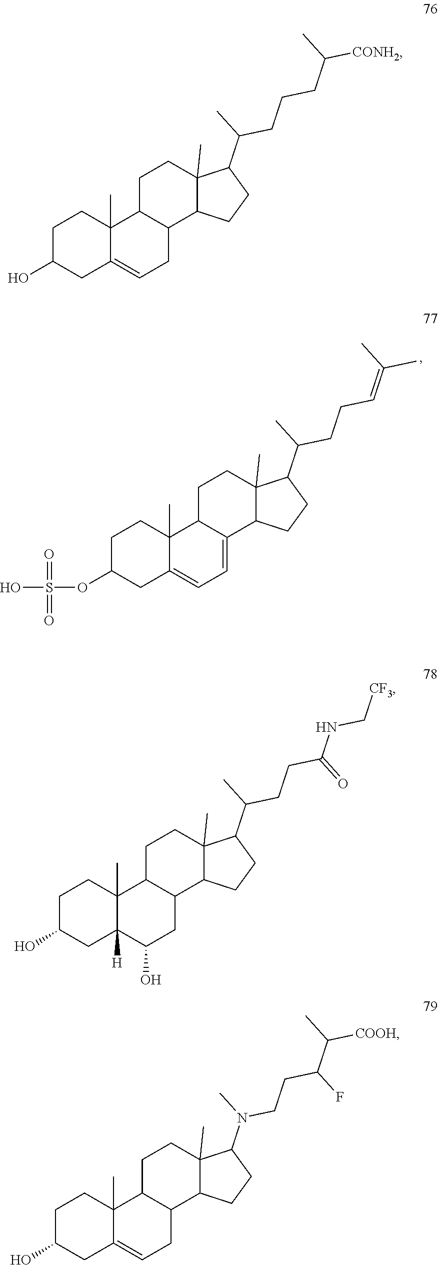 [SMILES: CC(CCCC(C)C1CCC2C3CC=C4CC(O)CCC4(C)C3CCC12C)C(N)=O ; CC(C)=CCCC(C)C1CCC2C3=CC=C4CC(OS(=O)(=O)O)CCC4(C)C3CCC21C ; CC(CCC(=O)NCC(F)(F)F)C1CCC2C3C[C@H](O)[C@@H]4C[C@H](O)CCC4(C)C3CCC12C ; CC(C(=O)O)C(F)CCN(C)C1CCC2C3CC=C4C[C@H](O)CCC4(C)C3CCC21C]

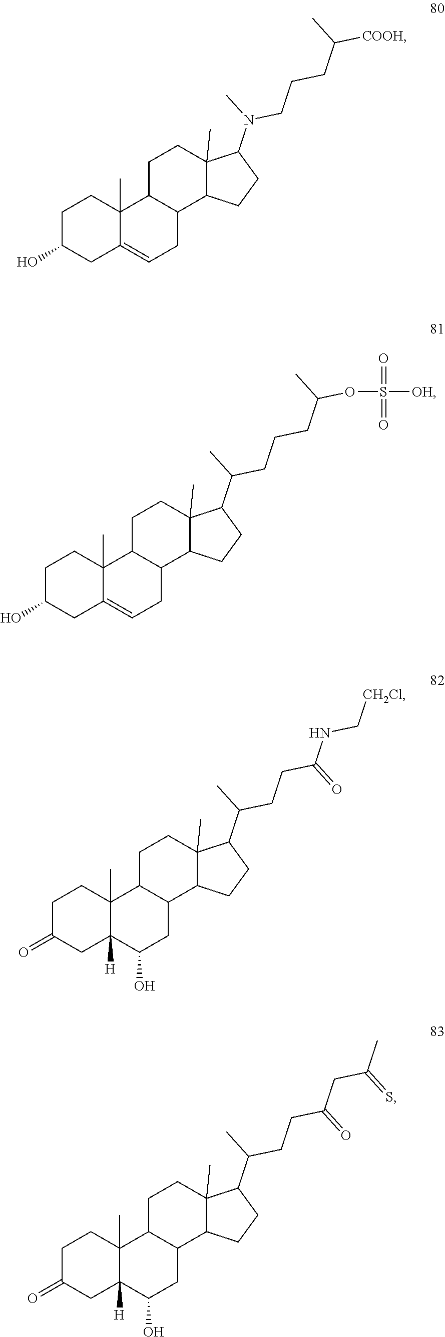

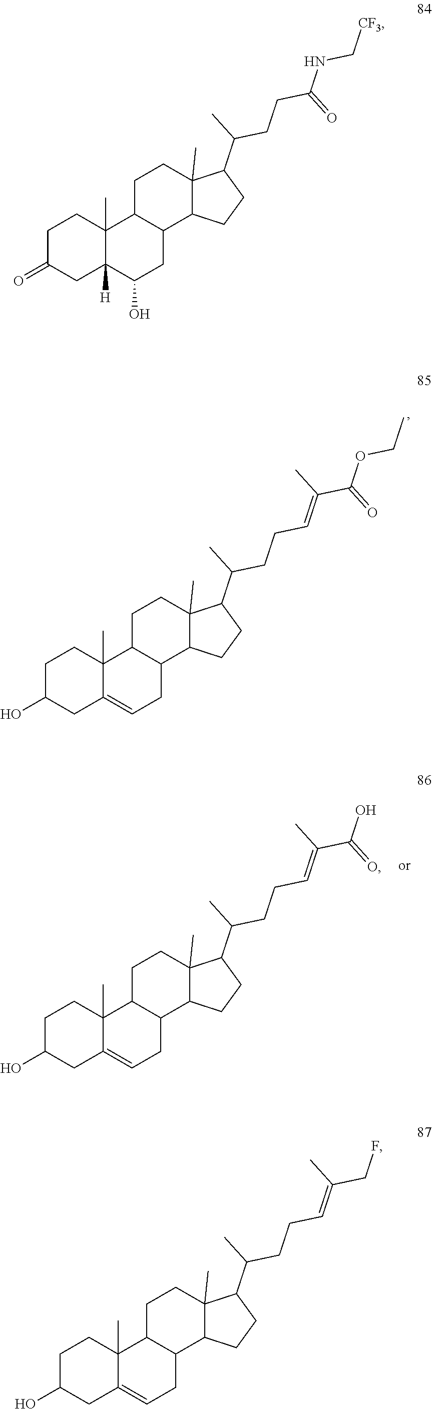 [SMILES: CC(CCC(=O)NCC(F)(F)F)C1CCC2C3C[C@H](O)[C@@H]4CC(=O)CCC4(C)C3CCC12C ; CCOC(=O)/C(C)=C/CCC(C)C1CCC2C3CC=C4CC(O)CCC4(C)C3CCC12C ; C/C(=C\CCC(C)C1CCC2C3CC=C4CC(O)CCC4(C)C3CCC12C)C(=O)O ; "or" ; C/C(=C\CCC(C)C1CCC2C3CC=C4CC(O)CCC4(C)C3CCC12C)CF]

or pharmaceutically acceptable salts thereof.

Compounds of Formula XXIII may be synthesized by methods known in the art, e.g., methods described in International Patent Publication No. WO00/66611.

In some embodiments of any of the foregoing methods, the LXR agonist is a compound of Formula XXIV:

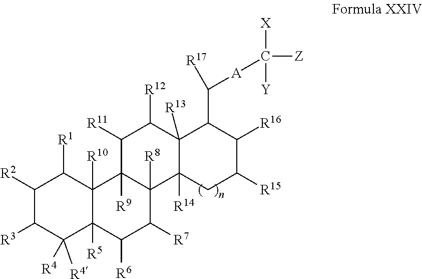

wherein each of R¹, R², R³, R⁴, R⁴, R⁵, R⁶, R⁷, R¹¹, R¹², R¹⁵, R¹⁶, and R¹⁷, independently, is hydrogen, halo, alkyl, halo alkyl, hydroxy, amino, carboxyl, oxo, sulfonic acid, or alkyl that is optionally inserted with —NH—, —N(alkyl)-, —O—, —S—, SO—, —SO₂—, —O—SO₂—, —SO₂—O—, —SO₃—O—, —CO—, —CO—O—, —O—CO—, —CO—NR′—, —NR′—CO—; or R³ and R⁴, together, R⁴, and R⁵, together, R⁵, and R⁶ together, or R⁶ and R⁷ together are eliminated so that a C═C bond is formed between the carbons to which they are attached;

each of R⁸, R⁹, R¹⁰, R¹³, and R¹⁴, independently, is hydrogen, halo, alkyl, haloalkyl, hydroxyalkyl, alkoxy, hydroxy, or amino;

n is 0, 1, or 2;

A is alkylene, alkenylene, or alkynylene; and

each of X, Y, and Z, independently, is alkyl, haloakyl, —OR′, —SR′, —NR′R″, —N(OR′)R″, or —N(SR′)R″; or X and Y together are =0, ═S, or ═NR′;

wherein each of R′ and R″, independently, is hydrogen, alkyl, or halo alkyl,

or a pharmaceutically acceptable salt thereof.

In other embodiments of any of the foregoing methods, the LXR agonist is any one of compounds 88-93:

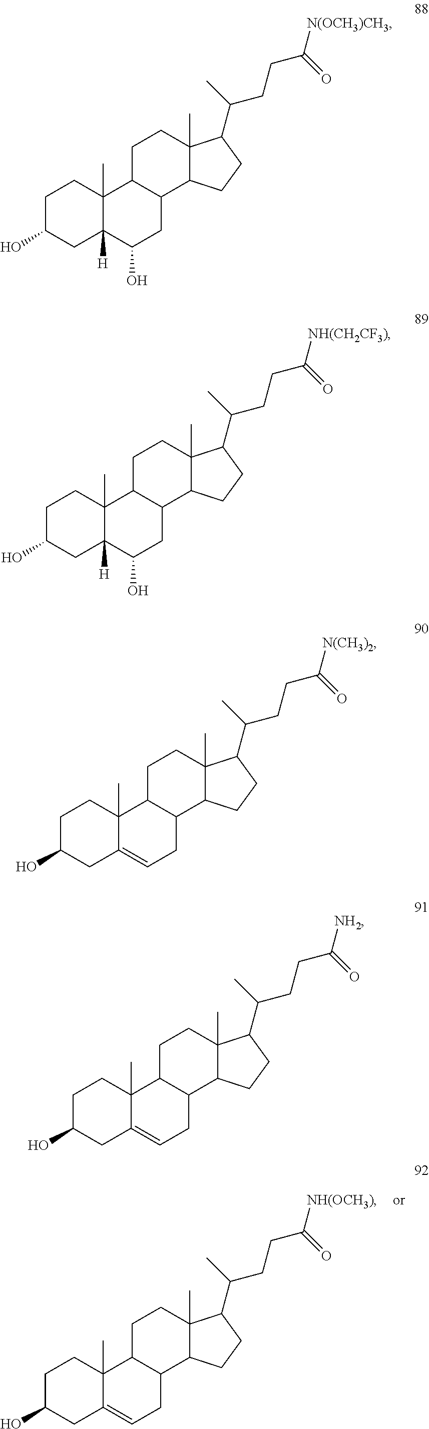

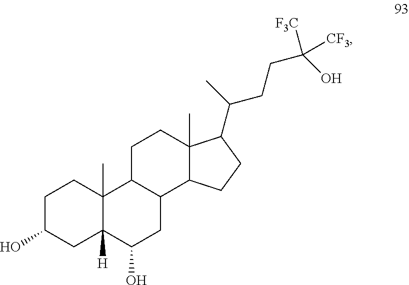 [SMILES: CC(CCC(O)(C(F)(F)F)C(F)(F)F)C1CCC2C3C[C@H](O)[C@@H]4C[C@H](O)CCC4(C)C3CCC12C]

or pharmaceutically acceptable salts thereof.

Compounds of Formula XXIV may be synthesized by methods known in the art, e.g., methods described in International Patent Publication No. WO02/13594.

In certain embodiments of any of the foregoin methods, the LXR agonist is a compound of Formula XXV:

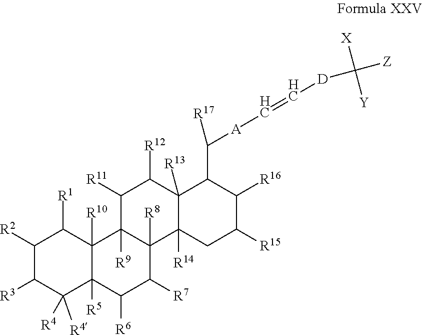

wherein in which each of R¹, R², R⁴, R⁵, R⁷, R¹¹, R¹⁵, R¹⁶, and R¹⁷, independently, is hydrogen, halo, alkyl, hydroxyl, amino, carboxyl, or sulfonic acid; each of R³, R^{3′}, R⁶, and R^{6′}, independently, is hydrogen, halo, alkyl, hydroxyl, amino, carboxyl, or sulfonic acid, or R³ and R^{3′}, together or R⁶ and R^{6′}, together are ═O; each of R⁸, R⁹, R¹⁰, R¹³, and R¹⁴, independently, is hydrogen, halo, alkyl, hydroxyalkyl, alkoxy, hydroxyl, or amino;

each of A and D, independently, is deleted or alkylene; X and Y, independently, is alkyl;

and Z is hydroxyl or alkoxy,

or a pharmaceutically acceptable salt thereof.

In some embodiments of any of the foregoing methods, the LXR agonist is any one of compounds 94-97:

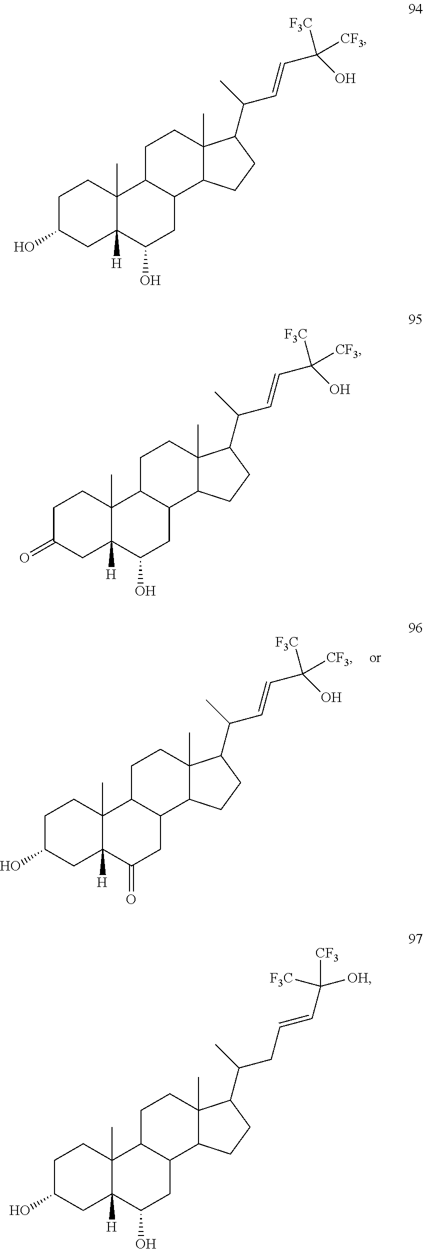 [SMILES: CC(/C=C/C(O)(C(F)(F)F)C(F)(F)F)C1CCC2C3C[C@H](O)[C@@H]4C[C@H](O)CCC4(C)C3CCC12C ; CC(/C=C/C(O)(C(F)(F)F)C(F)(F)F)C1CCC2C3C[C@H](O)[C@@H]4CC(=O)CCC4(C)C3CCC12C ; CC(/C=C/C(O)(C(F)(F)F)C(F)(F)F)C1CCC2C3CC(=O)[C@@H]4C[C@H](O)CCC4(C)C3CCC12C ; CC(C/C=C/C(O)(C(F)(F)F)C(F)(F)F)C1CCC2C3C[C@H](O)[C@@H]4C[C@H](O)CCC4(C)C3CCC12C]

or pharmaceutically acceptable salts thereof.

Compounds of Formula XXV may be synthesized by methods known in the art, e.g., methods described in International Patent Publication No. WO2011/014661.

In other embodiments of any of the foregoing methods, the LXR agonist is a compound of Formula XXVI:

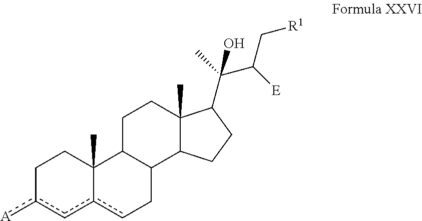

wherein A is selected from the group consisting of hydrogen, hydroxy, or oxygen,

wherein the dashed lines are optional double bonds, wherein there are no consecutive double bonds,

wherein E is hydrogen or hydroxy,

wherein RI is selected from the group consisting of:

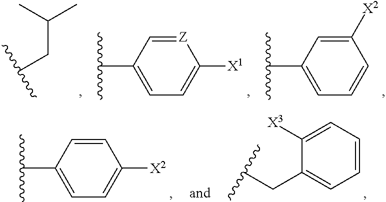

wherein Z is nitrogen that can be anywhere in the ring,

wherein X¹ can be bonded to any position on the ring and is selected from the group consisting of hydrogen, fluorine, chlorine, bromine, and iodine, and

wherein X² is selected from the group consisting of fluorine, chlorine, bromine, and iodine,

wherein X³ can be bonded to any position on the ring and is selected from the group consisting of hydrogen, fluorine, chlorine, bromine, and iodine,

or a pharmaceutically acceptable salt thereof.

In certain embodiments of any of the foregoing methods, the LXR agonist is any one of compounds 98-107:

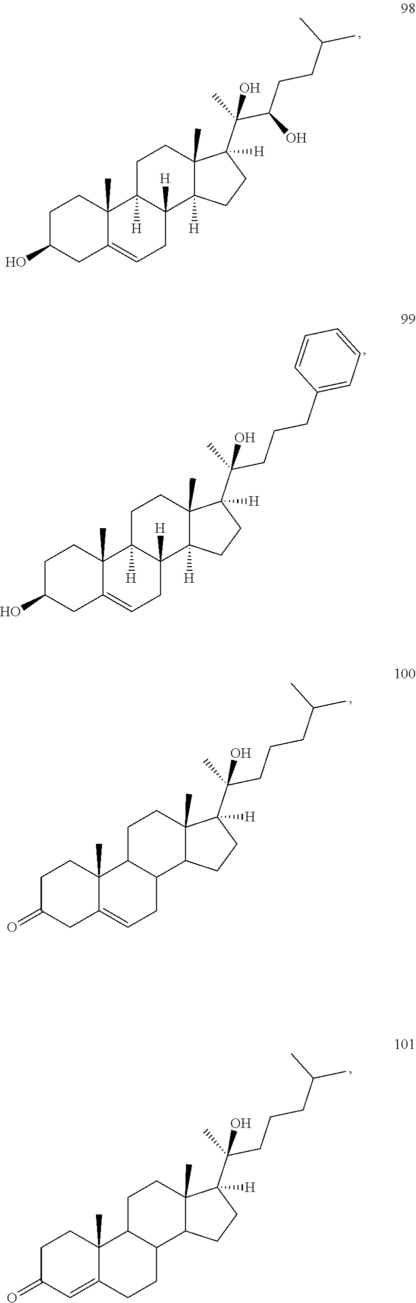

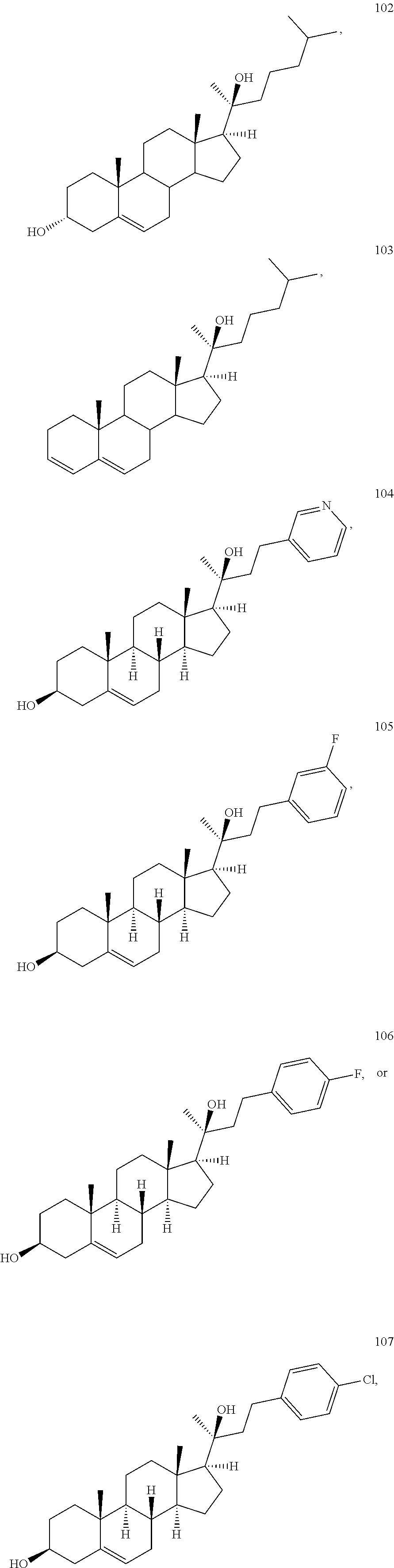

or pharmaceutically acceptable salts thereof.

Compounds of Formula XXVI may be synthesized by methods known in the art, e.g., methods described in International Patent Publication No. WO2011/103175.

In further embodiments of any of the foregoing methods, the LXR agonist is hyodeoxycholic acid (also known as 4-[(5R,8S,10R,13R,17R)-3,6-dihydroxy-10,13-dimethyl-2,3,4,5,6,7,8,9,11,12,14,15,16,17-tetradecahydro-1H-cyclopenta[a]phenanthren-17-yl]pentanoic acid) or a pharmaceutically acceptable salt thereof.

In further embodiments of any of the foregoing methods, the LXR agonist is a compound described in International Patent Publication No. WO2006/046593, e.g., any one of (4′-{[2-(tert-butoxycarbonyl)-3-hydroxy-4-(trifluoromethyl)benzyl]oxy}-1,1′-biphenyl-4-yl)acetic acid; 2-(4′-{[2-(tert-butoxycarbonyl)-3-hydroxy-4-(trifluoromethyl)benzyl]oxy}-1,1′-biphenyl-4-yl)propanoic acid; 1-(4′-{[2-(tert-butoxycarbonyl)-3-hydroxy-4-(trifluoromethyl)benzyl]oxy}-1,1′-biphenyl-4-yl)cyclopropanecarboxylic acid; 2-(4′-{[2-(tert-butoxycarbonyl)-3-hydroxy-4-(trifluoromethyl)benzyl]oxy}-1,1′-biphenyl-4-yl)-3-hydroxypropanoic acid; 2-[4′-{[2-(tert-butoxycarbonyl)-3-hydroxy-4-(trifluoromethyl)benzyl]oxy}-1,1′-biphenyl-4-yl]butanoic acid; (4′-{[2-(tert-butoxycarbonyl)-3-hydroxy-4-(trifluoromethyl)benzyl]oxy}-2-methyl-1,1′-biphenyl-3-yl)acetic acid; (4′-{[2-(tert-butoxycarbonyl)-3-hydroxy-4-(trifluoromethyl)benzyl]oxy}-2-methyl-1,1′-biphenyl-4-yl)acetic acid; (4′-{[2-(tert-butoxycarbonyl)-3-hydroxy-4-(trifluoromethyl)benzyl]oxy}-2-chloro-1,1-biphenyl-4-yl)acetic acid; (4′-{[2-(tert-butoxycarbonyl)-3-hydroxy-4-(trifluoromethyl)benzyl]oxy}-3-fluoro-1,1-biphenyl-4-yl)acetic acid; (4′-{[2-(tert-butoxycarbonyl)-3-hydroxy-4-(trifluoromethyl)benzyl]oxy}-3-chloro-1,1-biphenyl-4-yl)acetic acid; 2-(4′-{[2-(tert-butoxycarbonyl)-3-hydroxy-4-(trifluoromethyl)benzyl]oxy}-2-methoxy-1,1-biphenyl-3-yl)propanoic acid; 2-(4′-{[2-(tert-butoxycarbonyl)-3-hydroxy-4-(trifluoromethyl)benzyl]oxy}-3-fluoro-1,1-biphenyl-4-yl)propanoic acid; 1-(4′-{[2-(tert-butoxycarbonyl)-3-hydroxy-4-(trifluoromethyl)benzyl]oxy}-3-fluoro-1,1′-biphenyl-4-yl)cyclopropanecarboxylic acid; (4′-{[2-(tert-butoxycarbonyl)-3-hydroxy-4-(trifluoromethyl)benzyl]oxy}-3-methoxy-1,1-biphenyl-4-yl)acetic acid; (4′-{[2-(tert-butoxycarbonyl)-3-hydroxy-4-(trifluoromethyl)benzyl]oxy}-2-trifluoromethyl-1,1-biphenyl-4-yl)acetic acid; (4′-{[2-(tert-butoxycarbonyl)-3-hydroxy-4-(trifluoromehyl)benzyl]oxy}-2-ethyl-1,1-biphenyl-4-yl)acetic acid; tert-butyl 6-[({2-ethyl-4′-[(methoxycarbonyl)methyl]-1,1-biphenyl-4-yl}oxy)methyl]--2-hydroxy-3-(trifluoromethyl)benzoate; (4′-{[2-(tert-butoxycarbonyl)-3-hydroxy-4-(trifluoromethyl)benzyl]oxy}-2-nitro-1,1-biphenyl-4-yl)acetic acid; (2-amino-4′-{[2-(tert-butoxycarbonyl)-3-hydroxy-4-(trifluoromethyl)benzyl-]oxy}-1,1′-biphenyl-4-yl)acetic acid; (4′-{[2-(tert-butoxycarbonyl)-3-hydroxy-4-(trifluoromethyl)benzyl]oxy}-2-isopropyl-1,1′-biphenyl-4-yl)acetic acid; (4′-{[2-(tert-butoxycarbonyl)-3-hydroxy-4-(trifluoromethyl)benzyl]oxy}-2-formyl-1,1′-biphenyl-4-yl)acetic acid; (4′-{[2-(tert-butoxycarbonyl)-3-hydroxy-4-(trifluoromethyl)benzyl]oxy}-2-(hydroxymethyl)-1,1′-biphenyl-4-yl)acetic acid; (4′-{[2-(tert-butoxycarbonyl)-3-hydroxy-4-(trifluoromethyl)benzyl]oxy}-2-cyano-1,1′-biphenyl-4-yl)acetic acid; (4′-{[2-(tert-butoxycarbonyl)-3-hydroxy-4-(trifluoromethyl)benzyl]oxy}-2-cyclopropyl-1,1-biphenyl-4-yl)acetic acid; (4′-{[2-(tert-butoxycarbonyl)-3-hydroxy-4-(trifluoromehyl)benzyl]oxy}-3-ethyl-1,1′-biphenyl-4-yl)acetic acid; (4′-{[2-(tert-butoxycarbonyl)-3-hydroxy-4-(trifluoromehyl)benzyl]oxy}-2-ethyl-1,1′-biphenyl-3-yl)acetic acid; 2-(4′-{[2-(tert-butoxycarbonyl)-3-hydroxy-4-(trifluoromethyl)benzyl]oxy}-3-fluoro-1,1′-biphenyl-4-yl)-3-(dimethylamino)propanoic acid; 2-(4′-{[2-(tert-butoxycarbonyl)-3-hydroxy-4-(trifluoromethyl)benzyl]oxy}-2-ethyl-1,1′-biphenyl-4-yl)propanoic acid; 2-(4′-{[2-(tert-butoxycarbonyl)-3-hydroxy-4-(trifluoromethyl)benzyl]oxy}-2-nitro-1,1′-biphenyl-4-yl)propanoic acid; 2-(4′-{[2-(tert-butoxycarbonyl)-3-hydroxy-4-(trifluoromethyl)benzyl]oxy}-2-isopropyl-1,1′-biphenyl-4-yl)propanoic acid; 2-(4′-{[2-(tert-butoxycarbonyl)-3-hydroxy-4-(trifluoromethyl)benzyl]oxy}-2,3-dimethyl-1,1′-biphenyl-4-yl)propanoic acid; or 2-(4′-{[2-(tert-butoxycarbonyl)-3-hydroxy-4-(trifluoromethyl)benzyl]oxy}-2-cyclopropyl-1,1′-biphenyl-4-yl)propanoic acid; or pharmaceutically acceptable salts thereof.

In other embodiments of any of the foregoing methods, the LXR agonist is a compound described in International Patent Publication No. WO2006/073366, e.g., any one of: 2-tert-butyl-4-({3-[3-(hydroxymethyl)phenoxy]propyl}amino)-5-phenylisothiazol-3(2H)-one 1,1-dioxide; 2-tert-butyl-5-phenyl-4-[(4-phenylbutyl)amino]isothiazol-3(2H)-one 1,1-dioxide; 2-tert-butyl-4-{[3-(2-methoxyphenoxy)propyl]amino}-5-phenylisothiazol-3(2H)-one 1,1-dioxide; 2-tert-butyl-4-({3-[4-(hydroxymethyl)phenoxy]propyl}amino)-5-phenylisothiazol-3(2H)-one 1,1-dioxide; N-(3-{3-[(2-tert-butyl-1,1-dioxido-3-oxo-5-phenyl-2,3-dihydroisothiazol-4-yl)amino]propoxy}phenyl)acetamide; 2-tert-butyl-4-{[3-(2-fluorophenoxy)propyl] amino}-5-phenylisothiazol-3 (2H)-one 1,1-dioxide; 2-isopropyl-5-phenyl-4-[(4-phenylbutyl)amino]isothiazol-3(2H)-one 1,1-dioxide; 2-(4-{3-[(2-tert-butyl-1,1-dioxido-3-oxo-5-phenyl-2,3-dihydroisothiazol-4-yl)amino]propoxy}phenyl)-N,N-dimethylacetamide; 2-tert-butyl-4-{[3-(2-chlorophenoxy)propyl] amino}-5-phenylisothiazol-3(2H)-one 1,1-dioxide; 2-tert-butyl-4-{[3-(3-methoxyphenoxy)propyl]amino}-5-phenylisothiazol-3(2H)-one 1,1-dioxide; (3-{3-[(2-tert-butyl-1,1-dioxido-3-oxo-5-phenyl-2,3-dihydroisothiazo-4-yl)amino]propaxy}phenyl)acetic acid; 2-tert-butyl-5-phenyl-4-{[3-(pyridin-3-yloxy)propyl]amino}isothiazol-3(2H)-one 1,1-dioxide; methyl (3-{3-[(2-tert-butyl-1,1-dioxido-3-oxo-5-phenyl-2,3-dihydroisothiazol-4-yl)amino]propoxy}phenyl)acetate; 2-tert-butyl-5-phenyl-4-{[3-(pyridin-4-yloxy)propyl]amino}isothiazol-3(2H)-one 1,1-dioxide; 4-(benzylamino)-2-tert-butyl-5-phenylisothiazol-3(2H)-one 1,1-dioxide; 4-({1-[3-chloro-5-(trifluoromethyl)pyridin-2-yl]piperidin-4-yl}amino)-2-isopropyl-5-phenylisothiazol-3(2H)-one 1,1-dioxide; 2-tert-butyl-5-phenyl-4-[(2-phenylethyl)amino]isothiazol-3(2H)-one 1,1-dioxide; 2-cyclopentyl-5-phenyl-4-[(4-phenylbutyl)amino]isothiazol-3(2H)-one 1,1-dioxide; 2-tert-butyl-5-phenyl-4-{[3-(phenylthio)propyl]amino}isothiazol-3(2H)-one 1,1-dioxide 2-tert-butyl-4-[(3-phenoxypropyl)amino]-5-phenylisothiazol-3(2H)-one 1,1-dioxide; 2-tert-butyl-4-{[3-(3-chlorophenoxy)propyl]amino}-5-phenylisothiazol-3(2H)-one 1,1-dioxide; methyl 3-{3-[(2-tert-butyl-1,1-dioxido-3-oxo-5-phenyl-2,3-dihydroisothiazol-4-yl)amino]propaxy}benzoate; 2-benzyl-5-phenyl-4-[(4-phenylbutyl)amino]isothiazol-3(2H)-one 1,1-dioxide; (4-{3-[(2-tert-butyl-1,1-dioxido-3-oxo-5-phenyl-2,3-dihydroisothiazol-4-yl)amino]propoxy}phenyl)acetic acid; 2-tert-butyl-4-{[3-(3-fluorophenoxy)propyl]amino}-5-phenylisothiazol-3(2H)-one 1,1-dioxide; methyl (4-{3-[(2-tert-butyl-1,1-dioxido-3-oxo-5-phenyl-2,3-dihydroisothiazol-4-yl)amino]propoxy}phenyl)acetate; 2-tert-butyl-4-{[3-(4-fluorophenoxy)propyl]amino}-5-phenylisothiazol-3(2H)-one 1,1-dioxide; 2-isopropyl-5-phenyl-4-({1-[4-(trifluoromethyl)pyrimidin-2-yl]piperidin-4-yl}amino)isothiazol-3(2H)-one 1,1-dioxide; N-(3-{3-[(2-butyl-1,1-dioxido-3-oxo-5-phenyl-2,3-dihydroisothiazol-4-yl)amino]propaxy}phenyl)acetamide; 3-[(2-tert-butyl-1,1-dioxido-3-oxo-5-phenyl-2,3-dihydroisothiazol-4-yl)amino]propyl 4-hydroxybenzoate; 4-(benzylammo)-2-isopropyl-5-phenylisothiazol-3(2H)-one 1,1-dioxide; 2-[2-(3-fluorophenyl)ethyl]-5-phenyl-4-[(4-phenylbutyl)amino]isothiazol-3(2H)-one 1,1-dioxide; 4-[(cis-4-hydroxycyclohexyl)amino]-2-isopropyl-5-phenylisothiazol-3(2H)-one 1,1-dioxide; 2-tert-butyl-4-[(4-phenoxybutyl)amino]-5-phenylisothiazol-3(2H)-one 1,1-dioxide; 2-tert-butyl-4-({3-[(1-oxidapyridin-3-yl)oxy]propyl}amino)-5-phenylisothiazol-3(2H)-one 1,1-dioxide; 2-tert-butyl-4-[(2-phenoxyethyl)amino]-5-phenylisothiazol-3(2H)-one 1,1-dioxide; 4-(benzylamino)-2-cyclopentyl-5-phenylisothiazol-3(2H)-one 1,1-dioxide; 2-tert-butyl-4-{[3-(4-methoxyphenoxy)propyl]amino}-5-phenylisothiazol-3(2H)-one 1,1-dioxide; 4-[(4,4-difluorocyclohexyl)amino]-2-isopropyl-5-phenylisothiazol-3(2H)-one 1,1-dioxide; 2-isopropyl-4-[(2-phenoxyethyl)amino]-5-phenylisothiazol-3(2H)-one 1,1-dioxide; 5-phenyl-4-[(4-phenylbutyl)amino]-2-(tetrahydrofuran-2-ylmethyl)isothiazol-3(2H)-one 1,1-dioxide; 4-(benzylamino)-2-butyl-5-phenylisothiazol-3(2H)-one 1,1-dioxide; 2-butyl-4-(hexylamino)-5-phenylisothiazol-3(2H)-one 1,1-dioxide; 2-butyl-5-phenyl-4-[(2-phenylethyl)amino]isothiazol-3(2H)-one 1,1-dioxide; 2-butyl-4-{[4-(difluoromethoxy)benzyl]amino}-5-phenylisothiazol-3(2H)-one 1,1-dioxide; 4-[(trans-4-hydroxycyclohexyl)amino]-2-isopropyl-5-phenylisothiazol-3(2H)-one 1,1-dioxide; 2-tert-butyl-4-[(3-hydroxypropyl)amino]-5-phenylisothiazol-3(2H)-one 1,1-dioxide; 5-phenyl-4-[(4-phenylbutyl)amino]-2-(pyridin-3-ylmethyl)isothiazol-3(2H)-one 1,1-dioxide; 2-tert-butyl-4-[(2,3-dihydro-1,4-benzodioxin-2-ylmethyl)amino]-5-phenylisothiazol-3(2H)-one 1,1-dioxide; 2-butyl-4-[(4-hydroxycyclohexyl)amino]-5-phenylisothiazol-3(2H)-one 1,1-dioxide; 4-{3-[(2-tert-butyl-1,1-dioxido-3-oxo-5-phenyl-2,3-dihydroisothiazol-4-yl)amino]propaxy}benzoic acid; 3-{4-[(2-isopropyl-1,1-dioxido-3-oxo-5-phenyl-2,3-dihydroisothiazol-4-yl)amino]piperidin-1-yl}propanenitrile; 2-tert-butyl-4-{[3-(4-chlorophenoxy)propyl]amino}-5-phenylisothiazol-3 (2H)-one 1,1-dioxide; 5-phenyl-4-[(4-phenylbutyl)amino]-2-(pyridin-4-ylmethyl)isothiazol-3(2H)-one 1,1-dioxide; 4-[(1,3-benzodioxol-5-ylmethyl)amino]-2-butyl-5-phenylisothiazol-3(2H)-one 1,1-dioxide; 4-(2,3-dihydro-1H-inden-2-ylamino)-2-(2-methoxyethyl)-5-phenylisothiazol-3(2H)-one 1,1-dioxide; 2-butyl-4-[(2-morpholin-4-ylethyl)amino]-5-phenylisothiazol-3(2H)-one 1,1-dioxide; 2-tert-butyl-4-{[3-(4-isopropylphenoxy)propyl]amino}-5-phenylisothiazol-3(2H)-one 1,1-dioxide; 4-{(3-[benzyl(butyl)amino]propyl}amino)-2-tert-butyl-5-phenylisothiazol-3(2H)-one 1,1-dioxide; 2-tert-butyl-4-{[3-(3,5-dipropoxyphenoxy)propyl]amino}-5-phenylisothiazol-3(2H)-one 1,1-dioxide; 2-tert-butyl-4-[(2,2-diphenylethyl)amino]-5-phenylisothiazol-3(2H)-one 1,1-dioxide; 2-ethyl-4-{[2-(1H-imidazol-4-yl)ethyl]amino}-5-phenylisothiazol-3(2H)-one 1,1-dioxide; 2-butyl-4-[(4-morpholin-4-ylbenzyl)amino]-5-phenylisothiazol-3(2H)-one 1,1-dioxide; 2-butyl-4-{[3-(2-methoxyethoxy)propyl]amino}-5-phenylisothiazol-3(2H)-one 1,1-dioxide; 2-butyl-4-[(3-morpholin-4-ylpropyl)amino]-5-phenylisothiazol-3(2H)-one 1,1-dioxide; 2-butyl-4-[(2-methoxyethyl)amino]-5-phenylisothiazol-3(2H)-one 1,1-dioxide; 2-(2-methoxyethyl)-5-phenyl-4-(tetrahydro-2H-pyran-4-ylamino)isothiazol-3(2H)-one 1,1-dioxide; 4-(hexylamino)-2-(2-methoxyethyl)-5-phenylisothiazol-3(2H)-one 1,1-dioxide; 4-[(4-hydroxycyclohexyl)amino]-2-(2-methoxyethyl)-5-phenylisothiazol-3(2H)-one 1,1-dioxide; 4-[(1,3-benzodioxol-5-ylmethyl)amino]-2-(2-methoxyethyl)-5-phenylisothiazol-3(2H)-one 1,1-dioxide; 2-butyl-4-[(4-methoxybenzyl)amino]-5-phenylisothiazol-3(2H)-one 1,1-dioxide; 5-phenyl-4-[(4-phenylbutyl)amino]-2-(pyridin-2-ylmethyl)isothiazol-3(2H)-one 1,1-dioxide; 2-tert-butyl-4-{[3-(3-hydroxyphenoxy)propyl]amino}-5-phenylisothiazol-3(2H)-one 1,1-dioxide; 3-{3-[(2-tert-butyl-1,1-dioxido-3-oxo-5-phenyl-2,3-dihydroisothiazol-4-yl)amino]propaxy}benzoic acid; 4-{2-[(2-tert-butyl-1,1-dioxido-3-oxo-5-phenyl-2,3-dihydroisothiazol-4-yl)amino]ethyl}phenyl methanesulfonate; 4-{2-[(2-tert-butyl-1,1-dioxido-3-oxo-5-phenyl-2,3-dihydroisothiazol-4-yl)amino]ethyl}benzenesulfonamide; 2-tert-butyl-4-({1-[3-chloro-5-(trifluorome)pyridin-2-ylpiperidin-4-ylamino)-5-phenylisothiazol-3-(2H)-one 1,1-dioxide; tert-butyl 3-(2-[(2-tert-butyl-1,1-dioxido-3-oxo-5-phenyl-2,3-dihydroisothiazol-4-yl)amino]ethyl}azetidine-1-carbaxylate; 2-{2-[(2-tert-butyl-1,1-dioxido-3-oxo-5-phenyl-2,3-dihydroisothiazol-4-yl)aminoethoxy}phenyl methanesulfonate; 4-{2-[(2-tert-butyl-1,1-dioxido-3-oxo-5-phenyl-2,3-dihydroisothiazol-4-yl)amino]ethyl]benzonitrile; 4-({4-[(2-tert-butyl-1,1-dioxido-3-oxo-5-phenyl-2,3-dihydroisothiazol-4-yl)amino]piperidin-1-yl]methyl)benzonitrile; 2-tert-butyl-4-(isopropylamino)-5-phenylisothiazol-3 (2H)-one 1,1-dioxide; 4-(2-[(2-isopropyl-1,1-dioxido-3-oxo-5-phenyl-2,3-dihdroisothiazol-4-yl)aminoethyl}phenyl methanesulfonate; tert-butyl 3-((2-[(2-tert-butyl-1,1-dioxido-3-oxo-5-phenyl-2,3-dihydroisothiazol-4-yl)amino]ethyl]thio)pyrrolidine-1-carbaxylate; 2-tert-butyl-5-phenyl-4-([3-(pyridin-2-yloxy)propyl]amino}isothiazol-3(2H)-one 1,1-dioxide; 2-tert-butyl-4-[(2-{[3-chloro-5-(trifluoromethyl)pyridin-2-yl]amino}ethyl)amino]-5-phenylisothiazol-3(2H)-one 1,1-dioxide; 2-tert-butyl-5-phenyl-4-(1-[2-(trifluorome)benzoylpiperidin-4-ylamino)isothiazol-3(2H)-one 1,1-dioxide; 2-tert-butyl-4-[1-(5-methylpyridin-2-yl)piperidin-4-yl]amino]-5-phenylisothiazol-3(2H)-one 1,1-dioxide; 2-tert-butyl-4-{[1-(6-chloropyridazin-3-yl)piperidin-4-yl] amino}-5-phenylisothiazol-3-(2H)-one 1,1-dioxide; tert-butyl-4-[(2-tert-butyl-1,1-dioxido-3-oxo-5-phenyl-2,3-dihydroisothiazol-4-yl)amino]piperidine-1-carboxylate; methyl 2-{2-[(2-tert-butyl-1,1-dioxido-3-oxo-5-phenyl-2,3-dihydroisothiazol-4-yl)amino]ethoxy}benzoate; methyl 3-({4-[(2-tert-butyl-1,1-dioxido-3-oxo-5-phenyl-2,3-dihydroisothiazol-4-yl)amino]piperidin-1-yl}methyl)benzoate; 2-tert-butyl-4-{[1-(6-methoxypyridazin-3-yl)piperidin-4-yl]amino}-5-phenylisothiazol-3(2H)-one 1,1-dioxide; 2-tert-butyl-4-({1-[(2-chloropyridin-3-yl)carbonyl]piperidin-4-yl}amino)-5-phenylisothiazol-3(2H)-one 1,1-dioxide; 2-{2-[(2-isopropyl-1,1-dioxido-3-oxo-5-phenyl-2,3-dihydroisothiazol-4-yl)amino]ethoxy}phenyl methanesulfonate; 2-tert-butyl-4-{[1-(6-chloropyridin-3-yl)piperidin-4-yl] amino}-5-phenylisothiazol-3(2H)-one 1,1-dioxide; 4-[(1-benzylpiperidin-4-yl)amino]-2-tert-butyl-5-phenylisothiazol-3(2H)-one 1,1-dioxide; 4-{2-[(2-isopropyl-1,1-dioxido-3-oxo-5-phenyl-2,3-dmydroisothiazol-4-yl)amino]ethyl}benzenesulfonamide; 4-{2-[(2-isopropyl-1,1 dioxido-3-oxo-5-phenyl-2,3-dihydroisothiazol-4-yl)amino]ethyl}benzonitrile; 2-tert-butyl-4-(ethylamino)-5-phenylisothiazol-3(2H)-one 1,1-dioxide; 2-tert-butyl-4-({1-[(5-methylisoxazol-3-yl)methyl]piperidin-4-yl}amino)-5-phenylisothiazol-3(2H)-one 1,1-dioxide; 4-[(1-benzoylpiperidin-4-yl)amino]-2-tert-butyl-5-phenylisothiazol-3(2H)-one 1,1-dioxide; 2-tert-butyl-5-phenyl-4-{[1-(phenylacetyl)piperidin-4-yl]amino}isothiazol-3(2H)-one 1,1-dioxide; 2-tert-butyl-5-phenyl-4-[(1-pyridin-2-ylpiperidin-4-yl)amino]isothiazol-3(2H)-one 1,1-dioxide; 2-tert-butyl-5-phenyl-4-[(1-pyridazin-3-ylpiperidin-4-yl)amino]isothiazol-3(2H)-one 1,1-dioxide; 2-tert-butyl-5-phenyl-4-{[2-(pyridin-3-yloxy)ethyl]amino}isothiazol-3(2H)-one 1,1-dioxide; 2-tert-butyl-4-{[1-(5-fluoropyridin-2-yl)piperidin-4-yl]amino}-5-phenylisothiazol-3(2H)-one 1,1-dioxide; 2-tert-butyl-4-{[3-(3,5-dimethyl-1H-pyrazol-1-yl)propyl]amino}-5-phenylisothiazol-3(2H)-one 1,1-dioxide; 2-tert-butyl-4-{[1-(2-chloro-6-methylisonictinoyl)piperidin-4-yl] amino}-5-phenylisothiazol-3(2H)-one 1,1-dioxide; 2-tert-butyl-4-{[1-(5-chloropyridin-2-yl)piperidin-4-yl]amino}-5-phenylisothiazol-3(2H)-one 1,1-dioxide; 2-tert-butyl-5-phenyl-4-({1-[4-(trifluoromethyl)pyridin-2-yl]piperidin-4-yl}amino)isothiazol-3(2H)-one 1,1-dioxide; 4-({4-[(2-tert-butyl-1,1-dioxido-3-oxo-5-phenyl-2,3-dihydroisothiazol-4-yl)amino]piperidin-1-yl}carbonyl)benzonitrile; 2-tert-butyl-4-{[1-(3,4-difluorobenzoyl)piperidin-4-yl] amino}-5-phenylisothiazol-3 (2H)-one 1,1-dioxide; 4-[(1-acetylpiperidin-4-yl)amino]-2-tert-butyl-5-phenylisothiazol-3(2H)-one 1,1-dioxide; 3-{2-[(2-isopropyl 1,1-dioxido-3-oxo-5-phenyl-2,3-dihydroisothiazol-4-yl)amino]ethyl}benzonitrile; 2-tert-butyl-5-phenyl-4-({2-[2-(trifluoromethoxy)phenyl]ethyl}amino)isothiazol-3(2H)-one 1,1-dioxide; 4-{2-[(2-tert-butyl-1,1-dioxido-3-oxo-5-phenyl-2,3-dihydroisothiazol-4-yl)amino]ethoxy}phenyl methanesulfonate; 4-[(1-benzylpyrrolidin-3-yl)amino]-2-tert-butyl-5-phenylisothiazol-3(2H)-one 1,1-dioxide; 4-({1-[3-chloro-5-(trifluoromethyl)pyridin-2-yl]azetidin-3-yl}amino)-2-isopropyl-5-phenylisothiazol-3 (2H)-one 1,1-dioxide; N-benzyl-N-(2-tert-butyl-1,1-dioxido-3-oxo-5-phenyl-2,3-dihydroisothiazol-4-yl-glycinamide; 2-tert-butyl-4-[(1-isobutyrylpiperidin-4-yl)amino]-5-phenylisothiazol-3(2H)-one 1,1-dioxide; 2-tert-butyl-5-phenyl-4-[(2-pyridin-2-ylethyl)amino]isothiazol-3(2H)-one 1,1-dioxide; 2-tert-butyl-4-{[2-(2-chlorophenyl)ethyl]amino}-5-phenylisothiazol-3(2H)-one 1,1-dioxide; 2-tert-butyl-5-phenyl-4-{[1-(2-phenylethyl)piperidin-4-yl]amino}isothiazol-3(2H)-one 1,1-dioxide; 2-tert-butyl-5-phenyl-4-({2-[3-(trifluorome)phenyl]ethyl}amino)isothiazol-3(2H)-one 1,1-dioxide; 2-tert-butyl-5-phenyl-4-[(4-{[4-(trifluoromethyl)phenyl]thio}cyclohexyl)amino]isothiazol-3(2H)-one 1,1-dioxide; 2-tert-butyl-5-phenyl-4-[(2-{[3-(trifluoromethoxy)phenyl]thio}ethyl)amino]isothiazol-3(2H)-one 1,1-dioxide; 2-tert-butyl-4-{[2-(4-chlorophenoxy)ethyl]amino}-5-phenylisothiazol-3(2H)-one 1,1-dioxide; 2-tert-butyl-5-phenyl-4-({1-[5-(trifluoromethyl)pyridin-2-yl]piperidin-4-yl}amino)isothiazol-3(2H)-one 1,1-dioxide; 2-tert-butyl-5-phenyl-4-({2-[3-(trifluoromethoxy)phenoxy]ethyl}amino)isothiazol-3(2H)-one 1,1-dioxide; tert-butyl 3-{2-[(2-tert-butyl-1,1-dioxido-3-oxo-5-phenyl-2,3-dihydroisothiazol-4-yl)amino]ethoxy}azetidine-1-carboxylate; 2-tert-butyl-4-[(2,2-dimethylpropyl)amino]-5-phenylisothiazol-3(2H)-one 1,1-dioxide; 2-tert-butyl-4-(tert-butylamino)-5-phenylisothiazol-3(2H)-one 1,1-dioxide; methyl ({[(2-tert-butyl-1,1-dioxido-3-oxo-5-phenyl-2,3-dihydroisothiazol-4-yl)amino] acetyl}amino)acetate; 2-tert-butyl-5-phenyl-4-(piperidin-4-ylamino)isothiazol-3 (2H)-one 1,1-dioxide; 2-tert-butyl-4-[(1-methylpiperidin-4-yl)amino]-5-phenylisothiazol-3(2H)-one 1,1-dioxide; 2-tert-butyl-4-[(2-hydroxyethyl)amino]-5-phenylisothiazol-3(2H)-one 1,1-dioxide; 4-{[2-(biphenyl-2-ylthio)ethyl]amino}-2-tert-butyl-5-phenylisothiazol-3(2H)-one 1,1-dioxide; 2-tert-butyl-5-phenyl-4-{[2-(pyrrolidin-3-ylthio)ethyl]amino}isothiazol-3(2H)-one 1,1-dioxide; 2-tert-butyl-4-{[(5-methyl-3-phenylisoxazol-4-yl)methyl]amino}-5-phenylisothiazol-3(2H)-one 1,1-dioxide; 2-tert-butyl-5-phenyl-4-{[(1,3,5-trimethyl-1H-pyrazol-4-yl)methyl]amino}isothiazol-3(2H)-one 1,1-dioxide; 2-tert-butyl-5-phenyl-4-[(2-{[5-(trifluoromethyl)pyridin-2-yl]oxy}ethyl)amino]isothiazol-3(2H)-one 1,1-dioxide; 2-tert-butyl-5-phenyl-4-(2-[4-(trifluoromethoxy)phenyl]ethylamino)isothiazol-3(2H)-one 1,1-dioxide; 2-tert-butyl-5-phenyl-4-[(2,2,2-trifluoroethyl)amino]isothiazol-3(2H)-one 1,1-dioxide; 2-tert-butyl-4-[(2,3-dihydroxypropyl)amino]-5-phenylisothiazol-3(2H)-one 1,1-dioxide; 3-[(2-tert-butyl-1,1-dioxido-3-oxo-5-phenyl-2,3-dihydroisothiazol-4-yl)amino]propanenitrile; 4-{[2-(3,4-dimethoxyphenyl)ethyl]amino}-2-isopropyl-5-phenylisothiazol-3(2H)-one 1,1-dioxide; 4-{[2-(3-chloro-4-methoxyphenyl)ethyl] amino}-2-isopropyl-5-phenylisothiazol-3(2H)-one 1,1-dioxide; 4-{2-[(2-isobutyl-1,1-dioxido-3-oxo-5-phenyl-2,3-dihydroisothiazol-4-yl)amino]ethyl}phenyl methanesulfonate; 2-isopropyl-5-phenyl-4-[(1-pyridin-2-ylpiperidin-4-yl)amino]isothiazol-3(2H)-one 1,1-dioxide; 4-(2-([2-(4-fluorobenzyl)-1,1-dioxido-3-oxo-5-phenyl-2,3-dihydroisothiazol-4-yl] amino)ethyl)phenyl methanesulfonate; 2-isopropyl-4-(isopropylamino)-5-phenylisothiazol-3(2H)-one 1,1-dioxide; 2-isopropyl-5-phenyl-4-[(1-pyridin-2-yl-azetidin-3-yl)amino]isothiazol-3(2H)-one 1,1-dioxide; 2-tert-butyl-4-{[(5-methylisoxazol-3-yl)methyl]amino}-5-phenylisothiazol-3(2H)-one 1,1-dioxide; 4-{[2-(4-hydroxy-3,5-dimethoxyphenyl)ethyl]amino}2-isopropyl-5-phenylisothiazol-3(2H)-one 1,1-dioxide; 4-([2-(2-aminopyridin-4-yl)ethyl]amino)-2-isopropyl-5-phenylisothiazol-3(2H)-one 1,1-dioxide; 2-isopropyl-5-phenyl-4-[(2-pyridin-4-ylethyl)amino]isothiazol-3(2H)-one 1,1-dioxide; 2-isopropyl-5-phenyl-4-[(2-pyridin-3-ylethyl)amino]isothiazol-3(2H)-one 1,1-dioxide; 4-([2-(3,5-dimethylisoxazol-4-yl)ethyl] amino)-2-isopropyl-5-phenylisothiazol-3(2H)-one 1,1-dioxide; 4-[2-({2-[(5-methylisoxazol-3-yl)methyl]-1,1-dioxido-3-oxo-5-phenyl-2,3-dihydroisothiazol-4-yl}amino)ethyl]phenyl methanesulfonate; 4-([2-(3,5-dimethyl-1H-pyrazol-4-yl)ethyl]amino)-2-isopropyl-5-phenylisothiazol-3(2H)-one 1,1-dioxide; 4-[2-({2-[(methylthio)methyl]-1,1-dioxido-3-oxo-5-phenyl-2,3-dihydroisothiazol-4-yl}amino)ethyl]phenyl methanesulfonate; 2,6-dimethylphenyl 4-[(2-tert-butyl-1,1-dioxido-3-oxo-5-phenyl-2,3-dihydroisothiazol-4-yl)amino]butanoate; 2-mesitylethyl N-(2-tert-butyl-1,1-dioxido-3-oxo-5-phenyl-2,3-dihydroisothiazol-4-yl)glycinate; 2-[(2-tert-butyl-1,1-dioxido-3-oxo-5-phenyl-2,3-dihydroisothiazol-4-yl)amino]ethyl (2,6-dimethylphenyl)acetate; phenyl N-(2-tert-butyl-1,1-dioxido-3-oxo-5-phenyl-2,3-dihydroisothiazol-4-yl)-beta-alaninate; 4-(trifluoromethoxy)phenyl N-(2-tert-butyl-1,1-dioxido-3-oxo-5-phenyl-2,3-dihydroisothiazol-4-yl)-beta-alaninate; 1-methylpiperidin-4-yl N-(2-tert-butyl-1,1-dioxido-3-oxo-5-phenyl-2,3-dihydroisothiazol-4-yl)-beta-alaninate; 2-mesityl-1-methylethyl[(2-tert-butyl-1,1-dioxido-3-oxo-5-phenyl-2,3-dihydroisothiazol-4-yl)amino]acetate; 4-methoxybenzyl N-(2-tert-butyl-1,1-dioxido-3-oxo-5-phenyl-2,3-dihydroisothiazol-4-yl)glycinate; or 4-methoxyphenyl N-(2-tert-butyl-1,1-dioxido-3-oxo-5-phenyl-2,3-dihydroisothiazol-4-yl)glycinate; or pharmaceutically acceptable salts thereof.

In other embodiments of any of the foregoing methods, the LXR agonist is a compound described in U.S. Patent Publication No. US2009/0247587, e.g., any one of: 5-(2H-1,3-benzodioxol-5-yl)-5-methyl-3-(4-{[7-propyl-3,3-bis(trifluoromethyl)-2,3-dihydro-1-benzofuran-6-yl]oxy}butyl)imidazolidine-2,4-dione; 5-(3-methoxyphenyl)-5-methyl-3-(4-{[7-propyl-3,3-bis(trifluoromethyl)-2,3-dihydro-1-benzofuran-6-yl]oxy}butyl)imidazolidine-2,4-dione; 5-(3-bromo-4-fluorophenyl)-5-methyl-3-(4-{[7-propyl-3,3-bis(trifluoromethyl)-2,3-dihydro-1-benzofuran-6-yl]oxy}butyl)imidazolidine-2,4-dione; 5,5-dimethyl-3-(5-{[7-propyl-3,3-bis(trifluoromethyl)-2,3-dihydro-1-benzofuran-6-yl]oxy}pentyl)imidazolidine-2,4-dione; 5,5-dimethyl-3-(7-([7-propyl-3,3-bis(trifluoromethyl)-2,3-dihydro-1-benzofuran-6-yl]oxy)heptyl)imidazolidine-2,4-dione; 3-(4-{[5,7-dipropyl-3,3-bis(trifluoromethyl)-2,3-dihydro-1-benzofuran-6-yloxy}butyl)-5-methyl-5-phenylimidazolidine-2,4-dione; 3-(4-{[5,7-dipropyl-3,3-bis(trifluoromethyl)-2,3-dihydro-1-benzofuran-6-yl]oxy}butyl)-5-(4-ethoxyphenyl)-5-methylimidazolidine-2,4-dione; 3-(4-([5,7-dipropyl-3,3-bis(trifluoromethyl)-2,3-dihydro-1-benzofuran-6-yl]oxy]butyl)-5-methyl-5-(4-propoxyphenyl)imidazolidine-2,4-dione; or 3-(4-{[5,7-dipropyl-3,3-bis(trifluoromethyl)-2,3-dihydro-1-benzofuran-6-yl]oxy}butyl)-5-methyl-5-[4-(propan-2-yloxy)phenyl]imidazolidine-2,4-dione; or pharmaceutically acceptable salts thereof.

In further embodiments of any of the foregoing methods, the LXR agonist is a compound described in International Patent Publication No. WO2009/133692, e.g., any one of: 5-(2H-1,3-benzodioxol-5-yl)-3-(6-([3-benzyl-8-(trifluoromethyl)quinolin-4-yl]oxy)hexyl)-5-methylimidazolidine-2,4-dione; 3-(6-([3-benzyl-8-(trifluoromethylquinolin-4-yl]oxy)hexyl)-5-methyl-5-(quinoxalin-6-yl)imidazolidine-2,4-dione; 5-(2H-1,3-benzodioxol-5-yl)-3-(3-(3-[3-benzyl-8-(trifluoromethyl)quinolin-4-yl]phenoxy)propy)-5-methylimidazolidine-2,4-dione; 3-(3-{3-[3-benzyl-8-(trifluoromethyl)quinolin-4-yl]phenoxy}propyl)-5-methyl-5-[4-(propan-2-yloxy)phenyl]imidazolidine-2,4-dione; 5-(2H-1,3-benzodioxol-5-yl)-3-(4-{3-[3-benzyl-8-(trifluoromethyl)quinolin-4-yl]phenoxy}butyl)-5-methylimidazolidine-2,4-dione; 3-(4-{3-[3-benzyl-8-(trifluoromethylquinolin-4-yl]phenoxy}butyl)-5-methyl-5-[4-(propan-2-yloxy)phenyl]imidazolidine-2,4-dione; or 5-methyl-5-[3-(propan-2-yloxy)phenyl]-3-(6-{[3-propyl-8-(trifluoromethyl)quinolin-4-yl]oxy}hexyl)imidazolidine-2,4-dione; or pharmaceutically acceptable salts thereof.

In some embodiments of any of the foregoing methods, the LXR agonist is a compound described in International Patent Publication No. WO2010/125811, e.g., any one of: 3-(2-{4-[4-(1,1,1,3,3,3-hexafluoro-2-hydroxypropan-2-yl)-2-propylphenyl]piperazin-1-yl}-2-oxoethyl)-5-methyl-5-[5-(propan-2-yloxy)pyridin-2-yl]imidazolidine-2,4-dione; 3-(2-{4-[4-(1,1,1,3,3,3-hexafluoro-2-hydroxypropan-2-yl)-2-propylphenyl]piperazin-1-yl}-2-oxoethyl)-5-methy-5-[4-(propan-2-yloxy)phenyl]imidazolidine-2,4-dione; 5-(2H-1,3-benzodioxol-5-yl)-3-(2-{4-[4-(1,1,1,3,3,3-hexafluoro-2-hydroxypropan-2-yl)-2-propylphenyl]piperazin-1-yl}-2-oxoethyl)-5-methylimidazolidine-2,4-dione; 3-{2-[(3R)-4-[4-(1,1,1,3,3,3-hexafluoro-2-hydroxypropan-2-yl)-2-propylphenyl]-3-methylpiperazin-1-yl]-2-oxoethyl}-5-methyl-5-[4-(propan-2-yloxy)phenyl]imidazolidine-2,4-dione; 3-{2-[(3S)-4-[4-(1,1,1,3,3,3-hexafluoro-2-hydroxypropan-2-yl)-2-propylphenyl]-3-methylpiperazin-1-yl]-2-oxoethyl}-5-methyl-5-[4-(propan-2-yloxy)phenyl]imidazolidine-2,4-dione; 3-{2-[(2R,5S)-4-[4-(1,1,1,3,3,3-hexafluoro-2-hydroxypropan-2-yl)-2-[(1 Z)-prop-1-en-1-yl]phenyl]-2,5-dimethylpiperazin-1-yl]-2-oxoethyl}-5-methyl-5-[4-(propan-2-yloxy)phenyl]imidazolidine-2,4-dione; 3-{2-[(2R,5S)-4-[4-(1,1,1,3,3,3-hexafluoro-2-hydroxypropan-2-yl)-2-propylphenyl]-2,5-dimethylpiperazin-1-yl]-2-oxoethyl}-5-methyl-5-[4-(propan-2-yloxy)phenyl]imidazolidine-2,4-dione; 5-(2,3-dihydro-1-benzofuran-5-yl)-3-{2-[(2R,5S)-4-[4-(1,1,1,3,3,3-hexafluoro-2-hydroxypropan-2-yl)-2-[(1 Z)-prop-1-en-1-yl]phenyl]-2,5-dimethylpiperazin-1-yl]-2-oxoethyl}-5-methylimidazolidine-2,4-dione; 5-(2,3-dihydro-1-benzofuran-5-yl)-3-{2-[(2R,5S)-4-[4-(1,1,1,3,3,3-hexafluoro-2-hydroxypropan-2-yl)-2-propylphenyl]-2,5-dimethylpiperazin-1-yl]-2-oxoethyl}-5-methylimidazolidine-2,4-dione; 5-(2,2-dimethyl-2,3-dihydro-1-benzofuran-5-yl)-3-{2-[(3S)-4-[4-(1,1,1,3,3,3-hexafluoro-2-hydroxypropan-2-yl)-2-propylphenyl]-3-methylpiperazin-1-yl]-2-oxoethyl}-5-methylimidazolidine-2,4-dione; 3-{2-[(2R,5S)-4-[4-(1,1,1,3,3,3-hexafluoro-2-hydroxypropan-2-yl)-2-[(1 Z)-prop-1-en-1-yl]phenyl]-2,5-dimethylpiperazin-1-yl]-2-oxoethyl}-5-methyl-5-[5-(propan-2-yloxy)pyridin-2-yl]imidazolidine-2,4-dione; 3-{2-[(2R,5S)-4-[4-(1,1,1,3,3,3-hexafluoro-2-hydroxypropan-2-yl)-2-propylphenyl]-2,5-dimethylpiperazin-1-yl]-2-oxoethyl}-5-methyl-5-[5-(propan-2-yloxy)pyridin-2-yl]imidazolidine-2,4-dione; 5-[4-(cyclopropylsulfanyl)phenyl]-3-{2-[(2R,5S)-4-[4-(1,1,1,3,3,3-hexafluoro-2-hydroxypropan-2-yl)-2-[(1 Z)-prop-1-en-1-yl]phenyl]-2,5-dimethylpiperazin-1-yl]-2-oxoethyl}-5-methylimidazolidine-2,4-dione; 5-(2,3-dihydro-1-benzofuran-5-yl)-3-(2-{4-[4-(1,1,1,3,3,3-hexafluoro-2-hydroxypropan-2-yl)-2-propylphenyl]-1,4-diazepan-1-yl}-2-oxoethyl)-5-methylimidazolidine-2,4-dione; 5-{2H,3H-[1,4]dioxino[2,3-b]pyridin-7-yl}-3-{2-[(3S)-4-[4-(1,1,1,3,3,3-hexafluoro-2-hydroxypropan-2-yl)-2-propylphenyl]-3-methylpiperazin-1-yl]-2-oxoethyl}-5-methylimidazolidine-2,4-dione; 5-(1-benzofuran-6-yl)-3-(2-{4-[4-(1,1,1,3,3,3-hexafluoro-2-hydroxypropan-2-yl)-2-propylphenyl]piperazin-1-yl}-2-oxoethyl)-5-methylimidazolidine-2,4-dione; or 5-(1-benzofuran-5-yl)-3-(2-[(3S)-4-[4-(1,1,1,3,3,3-hexafluoro-2-hydroxypropan-2-yl)-2-propylphenyl]-3-methylpiperazin-1-yl]-2-oxoethyl-5-methylimidazolidine-2,4-dione; or pharmaceutically acceptable salts thereof.

In other embodiments of any one of the foregoing methods, the LXR agonist is a compound described in International Patent Publication No. WO2009/138438, e.g., any one of: 1-(cyclopropylmethyl)-3-(4-(4-(4-(1,1,1,3,3,3-hexafluoro-2-hydroxypropan-2-yl)-benzyl)piperazine-1-carbonyl)phenyl)urea; 1-butyl-3-(4-(4-(4-(1,1,1,3,3,3-hexafluoro-2-hydroxypropan-2-yl)benzyl)piperazine-1-carbonyl)phenyl)urea; 1-(4-(4-(4-(1,1,1,3,3,3-hexafluoro-2-hydroxypropan-2-yl)benzyl)piperazine-1-carbonyl)phenyl)-3-isobutylurea; 1-cyclobutyl-3-(4-(4-(4-(1,1,1,3,3,3-hexafluoro-2-hydroxypropan-2-yl)benzyl)-piperazine-1-carbonyl)phenyl)urea; 1-(2-fluoro-4-(4-(4-(1,1,1,3,3,3-hexafluoro-2-hydroxypropan-2-yl)benzyl)piperazine-1-carbonyl)phenyl)-3-(2-hydroxy-2-methylpropyl)urea; 1-(2-fluoro-4-(4-(4-(1,1,1,3,3,3-hexafluoro-2-hydroxypropan-2-yl)benzyl)piperazine-1-carbonyl)phenyl)-3-(3-hydroxy-3-methylbutyl)urea; 1-(cyclopropylmethyl)-3-(2-fluoro-4-(4-(4-(1,1,1,3,3,3-hexafluoro-2-hydroxypropan-2-yl)benzyl)piperazine-1-carbonyl)phenyl)urea; (S)-1-(2-fluoro-4-(4-(4-(1,1,1,3,3,3-hexafluoro-2-hydroxypropan-2-yl)benzyl)-piperazine-1-carbonyl)phenyl)-3-(2-hydroxypropyl)urea; (R)-1-(2-fluoro-4-(4-(4-(1,1,1,3,3,3-hexafluoro-2-hydroxypropan-2-yl)benzyl)-piperazine-1-carbonyl)phenyl)-3-(2-hydroxypropyl)urea; 1-(2-fluoro-4-(4-(4-(1,1,1,3,3,3-hexafluoro-2-hydroxypropan-2-yl)benzyl)piperazine-1-carbonyl)phenyl)-3-((1-hydroxycyclopropyl)methyl)urea; (S)-1-(2-fluoro-4-(4-(4-(1,1,1,3,3,3-hexafluoro-2-hydroxypropan-2-yl)benzyl)-piperazine-1-carbonyl)phenyl)-3-(1-hydroxy-3-methylbutan-2-yl)urea; trans-1-(2-fluoro-4-(4-(4-(1,1,1,3,3,3-hexafluoro-2-hydroxypropan-2-yl)benzyl)piperazine-1-carbonyl)phenyl)-3-(4-hydroxycyclohexyl)urea; (S)-1-(2-fluoro-4-(4-(4-(1,1,1,3,3,3-hexafluoro-2-hydroxypropan-2-yl)benzyl)-piperazine-1-carbonyl)phenyl)-3-(1-hydroxypentan-2-yl)urea; 1-(2-chloro-4-(4-(4-(1,1,1,3,3,3-hexafluoro-2-hydroxypropan-2-yl)benzyl)piperazine-1-carbonyl)phenyl)-3-(2-hydroxy-2-methylpropyl)urea; (S)-1-(2-chloro-4-(4-(4-(1,1,1,3,3,3-hexafluoro-2-hydroxypropan-2-yl)benzyl)-piperazine-1-carbonyl)phenyl)-3-(2-hydroxypropyl)urea; (R)-1-(2-chloro-4-(4-(4-(1,1,1,3,3,3-hexafluoro-2-hydroxypropan-2-yl)benzyl)-piperazine-1-carbonyl)phenyl)-3-(2-hydroxypropyl)urea; 1-(2-chloro-4-(4-(4-(1,1,1,3,3,3-hexafluoro-2-hydroxypropan-2-yl)benzyl)piperazine-1-carbonyl)phenyl)-3-(3-hydroxy-3-methylbutyl)urea; 1-(2-chloro-4-(4-(4-(1,1,1,3,3,3-hexafluoro-2-hydroxypropan-2-yl)benzyl)piperazine-1-carbonyl)phenyl)-3-((1-hydroxycyclopropyl)methyl)urea; trans-1-(2-chloro-4-(4-(4-(1,1,1,3,3,3-hexafluoro-2-hydroxypropan-2-yl)benzyl)piperazine-1-carbonyl)phenyl)-3-(4-hydroxycyclohexyl)urea; 1-(2-amino-2-methylpropyl)-3-(2-fluoro-4-(4-(4-(1,1,1,3,3,3-hexafluoro-2-hydroxypropan-2-yl)benzyl)piperazine-1-carbonyl)phenyl)urea; 1-(2-fluoro-4-(4-(4-(1,1,1,3,3,3-hexafluoro-2-hydroxypropan-2-yl)benzyl)piperazine-1-carbonyl)phenyl)-3-((1 S,2R)-2-hydroxycyclopentyl)urea; 1-(2-fluoro-4-(4-(4-(1,1,1,3,3,3-hexafluoro-2-hydroxypropan-2-yl)benzyl)piperazine-1-carbonyl)phenyl)-3-(3-hydroxycyclobutyl)urea; 1-(2-fluoro-4-(4-(4-(1,1,1,3,3,3-hexafluoro-2-hydroxypropan-2-yl)benzyl)piperazine-1-carbonyl)phenyl)-3-((1-hydroxycyclobutyl)methyl)urea; 1-(2-(dimethylamino)-2-methylpropyl)-3-(2-fluoro-4-(4-(4-(1,1,1,3,3,3-hexafluoro-2-hydroxypropan-2-yl)benzyl)piperazine-1-carbonyl)phenyl)urea; 1-(2-fluoro-4-(4-(4-(1,1,1,3,3,3-hexafluoro-2-hydroxypropan-2-yl)benzyl)piperazine-1-carbonyl)phenyl)-3-(3,3,3-trifluoro-2-hydroxypropyl)urea; 1-(2-fluoro-4-(4-(4-(1,1,1,3,3,3-hexafluoro-2-hydroxypropan-2-yl)benzyl)piperazine-1-carbonyl)phenyl)-3-(tetrahydro-2H-pyran-4-yl)urea; (R)-1-(2-fluoro-4-(4-(4-(1,1,1,3,3,3-hexafluoro-2-hydroxypropan-2-yl)benzyl)piperazine-1-carbonyl)phenyl)-3-(2-hydroxy-2-phenylethyl)urea; (S)-1-(2-fluoro-4-(4-(4-(1,1,1,3,3,3-hexafluoro-2-hydroxypropan-2-yl)benzyl)piperazine-1-carbonyl)phenyl)-3-(2-hydroxy-2-phenylethyl)urea; (S)-1-(2-fluoro-4-(4-(4-(1,1,1,3,3,3-hexafluoro-2-hydroxypropan-2-yl)benzyl)-piperazine-1-carbonyl)phenyl)-3-(6-oxopiperidin-3-yl)urea; cis-1-(2-fluoro-4-{4-[4-(2,2,2-trifluoro-1-hydroxy-1-trifluoromethyl-ethyl)-benzyl]-piperazine-1-carbonyl}-phenyl)-3-(4-hydroxy-1,1-dioxo-tetrahydro-1λ6-thiophen-3-yl)-urea; 1-(1,1-dioxo-tetrahydro-1λ6-thiophen-3-yl)-3-(2-fluoro-4-{4-[4-(2,2,2-trifluoro-1-hydroxy-1-trifluoromethyl-ethyl)benzyl]piperazine-1-carbonyl}phenyl)urea; (R)-1-(2-fluoro-4-(4-(4-(1,1,1,3,3,3-hexafluoro-2-hydroxypropan-2-yl)benzyl)-piperazine-1-carbonyl)phenyl)-3-(tetrahydrofuran-3-yl)urea; 1-(1,1-dioxo-hexahydro-1λ6-thiopyran-4-yl)-3-(2-fluoro-4-{4-[4-(2,2,2-trifluoro-1-hydroxy-1-trifluoromethyl-ethyl)benzyl]piperazine-1-carbonyl}phenyl)urea; 1-(2-fluoro-4-(4-(4-(1,1,1,3,3,3-hexafluoro-2-hydroxypropan-2-yl)benzyl)piperazine-1-carbonyl)phenyl)-3-((4-hydroxytetrahydro-2H-pyran-4-yl)methyl)urea; (S)-1-(2-fluoro-4-(4-(4-(1,1,1,3,3,3-hexafluoro-2-hydroxypropan-2-yl)benzyl)-piperazine-1-carbonyl)phenyl)-3-(tetrahydrofuran-3-yl)urea; cis-1-(2-fluoro-4-(4-(4-(1,1,1,3,3,3-hexafluoro-2-hydroxypropan-2-yl)benzyl)piperazine-1-carbonyl)phenyl)-3-(4-hydroxytetrahydrofuran-3-yl)urea; 1-(2-fluoro-4-(4-(4-(1,1,1,3,3,3-hexafluoro-2-hydroxypropan-2-yl)benzyl)piperazine-1-carbonyl)phenyl)-3-((1 S,2R)-2-hydroxycyclohexyl)urea; 1-(2-fluoro-4-(4-(4-(1,1,1,3,3,3-hexafluoro-2-hydroxypropan-2-yl)benzyl)piperazine-1-carbonyl)phenyl)-3-(2-hydroxybutyl)urea; cis-1-(2-chloro-4-{4-[4-(2,2,2-trifluoro-1-hydroxy-1-trifluoromethyl-ethyl)-benzyl]-piperazine-1-carbonyl}-phenyl)-3-(4-hydroxy-1,1-dioxo-tetrahydro-1λ6-thiophen-3-yl)urea; 1-(5-tert-butylisoxazol-3-yl)-3-(2-fluoro-4-(4-(4-(1,1,1,3,3,3-hexafluoro-2-hydroxypropan-2-yl)benzyl)piperazine-1-carbonyl)phenyl)urea; 1-(2-fluoro-4-(4-(4-(1,1,1,3,3,3-hexafluoro-2-hydroxypropan-2-yl)benzyl)piperazine-1-carbonyl)phenyl)-3-(2-methylpyridin-4-yl)urea; 1-(2-fluoro-4-(4-(4-(1,1,1,3,3,3-hexafluoro-2-hydroxypropan-2-yl)benzyl)piperazine-1-carbonyl)phenyl)-3-(2-(trifluoromethyl)pyridin-4-yl)urea; 1-(2-fluoro-4-(4-(4-(1,1,1,3,3,3-hexafluoro-2-hydroxypropan-2-yl)benzyl)piperazine-1-carbonyl)phenyl)-3-(5-methylisoxazol-3-yl)urea; 1-(2-fluoro-4-(4-(4-(1,1,1,3,3,3-hexafluoro-2-hydroxypropan-2-yl)benzyl)piperazine-1-carbonyl)phenyl)-3-(3-fluoropyridin-4-yl)urea; 1-(2-fluoro-4-(4-(4-(1,1,1,3,3,3-hexafluoro-2-hydroxypropan-2-yl)benzyl)piperazine-1-carbonyl)phenyl)-3-(1,3,4-thiadiazol-2-yl)urea; 1-(2-fluoro-4-(4-(4-(1,1,1,3,3,3-hexafluoro-2-hydroxypropan-2-yl)benzyl)piperazine-1-carbonyl)phenyl)-3-(pyridin-4-yl)urea; 1-(2-fluoro-4-(4-(4-(1,1,1,3,3,3-hexafluoro-2-hydroxypropan-2-yl)benzyl)piperazine-1-carbonyl)phenyl)-3-(isoxazol-3-yl)urea; 1-(5-cyanothiazol-2-yl)-3-(2-fluoro-4-(4-(4-(1,1,1,3,3,3-hexafluoro-2-hydroxypropan-2-yl)benzyl)piperazine-1-carbonyl)phenyl)urea; 1-(2-fluoro-4-(4-(4-(1,1,1,3,3,3-hexafluoro-2-hydroxypropan-2-yl)benzyl)piperazine-1-carbonyl)phenyl)-3-(isoxazol-4-yl)urea; 1-(2-fluoro-4-(4-(4-(1,1,1,3,3,3-hexafluoro-2-hydroxypropan-2-yl)benzyl)piperazine-1-carbonyl)phenyl)-3-(pyridin-2-yl)urea; 1-(2-fluoro-4-(4-(4-(1,1,1,3,3,3-hexafluoro-2-hydroxypropan-2-yl)benzyl)piperazine-1-carbonyl)phenyl)-3-(3-methylisoxazol-5-yl)urea; 1-(2-fluoro-4-(4-(4-(1,1,1,3,3,3-hexafluoro-2-hydroxypropan-2-yl)benzyl)piperazine-1-carbonyl)phenyl)-3-(3-methyl-1,2,4-oxadiazol-5-yl)urea; 1-(2-fluoro-4-(4-(4-(1,1,1,3,3,3-hexafluoro-2-hydroxypropan-2-yl)benzyl)piperazine-1-carbonyl)phenyl)-3-(pyridin-3-yl)urea; 1-(2-fluoro-4-(4-(4-(1,1,1,3,3,3-hexafluoro-2-hydroxypropan-2-yl)benzyl)piperazine-1-carbonyl)phenyl)-3-(pyrimidin-4-yl)urea; 1-(2-fluoro-4-(4-(4-(1,1,1,3,3,3-hexafluoro-2-hydroxypropan-2-yl)benzyl)piperazine-1-carbonyl)phenyl)-3-(pyrazin-2-yl)urea; 1-(2-fluoro-4-{4-[4-(2,2,2-trifluoro-1-hydroxy-1-trifluoromethyl-ethyl)-benzyl]-piperazine-1-carbonyl}-phenyl)-3-(1-oxo-tetrahydro-thiopyran-4-yl)-urea; or 1-(2-fluoro-4-{4-[4-(2,2,2-trifluoro-1-hydroxy-1-trifluoromethyl-ethyl)-benzyl]-piperazine-1-carbonyl}-phenyl)-3-(1-oxo-tetrahydro-thiophen-3-yl)-urea; or pharmaceutically acceptable salts thereof.

In further embodiments of any of the foregoing methods, the LXR agonist is a compound described in International Patent Publication No. WO2010/025179, e.g., any one of: N-tert-butyl-5-((4-(4-(3-cyclobutylureido)-3-fluorobenzoyl)piperazin-1-yl)methyl)furan-2-carboxamide; N-tert-butyl-5-((4-(3-fluoro-4-(3-isobutylureido)benzoyl)piperazin-1-yl)methyl)furan-2-carboxamide; N-tert-butyl-5-((4-(4-(3-(cyclopropylmethyl)ureido)-3-fluorobenzoyl)piperazin-1-yl)methyl)furan-2-carboxamide; N-tert-butyl-5-((4-(3-fluoro-4-(3-neopentylureido)benzoyl)piperazin-1-yl)methyl)furan-2-carboxamide; N-tert-butyl-5-((4-(3-fluoro-4-(3-isopentylureido)benzoyl)piperazin-1-yl)methyl)furan-2-carboxamide; N-tert-butyl-5-((4-(4-(3-butylureido)-3-fluorobenzoyl)piperazin-1-yl)methyl)furan-2-carboxamide hydrochloride; N-tert-butyl-5-((4-(3-chloro-4-(3-neopentylureido)benzoyl)piperazin-1-yl)methyl)furan-2-carboxamide; N-tert-butyl-5-((4-(3-chloro-4-(3-isobutylureido)benzoyl)piperazin-1-yl)methyl)furan-2-carboxamide; N-tert-butyl-5-((4-(3-chloro-4-(3-(cyclopropylmethyl)ureido)benzoyl)piperazin-1-yl)methyl)furan-2-carboxamide; N-tert-butyl-5-((4-(3-chloro-4-(3-cyclobutylureido)benzoyl)piperazin-1-yl)methyl)furan-2-carboxamide; N-tert-butyl-5-((4-(3-chloro-4-(3-isopentylureido)benzoyl)piperazin-1-yl)methyl)furan-2-carboxamide; N-tert-butyl-5-((4-(4-(3-butylureido)-3-chlorobenzoyl)piperazin-1-yl)methyl)furan-2-carboxamide; N-tert-butyl-5-((4-(4-(3-(cyclopropylmethyl)ureido)-2,3-difluorobenzoyl)piperazin-1-yl)methyl)furan-2-carboxamide; N-tert-butyl-5-((4-(2,3-difluoro-4-(3-neopentylureido)benzoyl)piperazin-1-yl)methyl)furan-2-carboxamide; N-tert-butyl-5-((4-(2,3-difluoro-4-(3-isobutylureido)benzoyl)piperazin-1-yl)methyl)furan-2-carboxamide; N-tert-butyl-5-((4-(4-(3-cyclobutylureido)-2,3-difluorobenzoyl)piperazin-1-yl)methyl)furan-2-carboxamide; N-tert-butyl-5-((4-(4-(3-butylureido)-2,3-difluorobenzoyl)piperazin-1-yl)methyl)furan-2-carboxamide; N-tert-butyl-5-((4-(2,3-difluoro-4-(3-isopentylureido)benzoyl)piperazin-1-yl)methyl)furan-2-carboxamide; N-tert-butyl-6-((4-(4-(3-cyclobutylureido)-3-fluorobenzoyl)piperazin-1-yl)methyl)picolinamide; N-tert-butyl-6-((4-(3-fluoro-4-(3-neopentylureido)benzoyl)piperazin-1-yl)methyl)picolinamide; N-tert-butyl-6-((4-(3-fluoro-4-(3-isopentylureido)benzoyl)piperazin-1-yl)methyl)picolinamide; or N-tert-butyl-6-((4-(4-(3-(cyclopropylmethyl)ureido)-3-fluorobenzoyl)piperazin-1-yl)methyl)picolinamide; or pharmaceutically acceptable salts thereof.

In further embodiments of any of the foregoing methods, the LXR agonist is a compound described in International Patent Publication No. WO2009/144961, e.g., any one of: 5-(2H-1,3-benzodioxol-5-yl)-3-({3-[4-(1,1,1,3,3,3-hexafluoro-2-hydroxypropan-2-yl)-2-propyphenoxy]phenyl}methyl)-5-methylimidazolidine-2,4-dione; 5-(2,3-dihydro-1-benzofuran-5-yl)-3-({3-[4-(1,1,1,3,3,3-hexafluoro-2-hydroxypropan-2-yl)-2-propylphenoxy]phenyl}methyl)-5-methylimidazolidine-2,4-dione; 5-(2,3-dihydro-1-benzofuran-5-yl)-3-(1-{3-[4-(1,1,1,3,3,3-hexafluoro-2-hydroxypropan-2-yl)-2-propylphenoxy]phenyl}ethyl)-5-methylimidazolidine-2,4-dione; 5-(2H-1,3-benzodioxol-5-yl)-3-({4-[4-(1,1,1,3,3,3-hexafluoro-2-hydroxypropan-2-yl)-2-propylphenoxy]pyridin-2-yl}methyl)-5-methylimidazolidine-2,4-dione; 3-({4-[4-(1,1,1,3,3,3-hexafluoro-2-hydroxypropan-2-yl)-2-propylphenoxy]pyridin-2-yl}methyl)-5-methyl-5-[4-(propan-2-yloxy)phenyl]imidazolidine-2,4-dione; 3-({5-[4-(1,1,1,3,3,3-hexafluoro-2-hydroxypropan-2-yl)-2-propylphenoxy]pyridin-3-yl}methyl)-5-methyl-5-[4-(propan-2-yloxy)phenyl]imidazolidine-2,4-dione; 5-(2,3-dihydro-1-benzofuran-5-yl)-3-{(2-[4-(1,1,1,3,3,3-hexafluoro-2-hydroxypropan-2-yl)-2-propylphenoxy]pyridin-4-yl}methyl)-5-methylimidazolidine-2,4-dione; 3-({2-[4-(1,1,1,3,3,3-hexafluoro-2-hydroxyropan-2-yl)-2-propylphenoxy]pyridin-4-yl}methyl)-5-methyl-5-[4-(propan-2-yloxy)phenyl]imidazolidine-2,4-dione; 3-({6-[4-(1,1,1,3,3,3-hexafluoro-2-hydroxypropan-2-yl)-2-propylphenoxy]pyridin-2-yl}methyl)-5-methyl-5-[4-(propan-2-yloxy)phenyl]imidazolidine-2,4-dione; 5-(2H-1,3-benzodioxol-5-yl)-3-(2-{3-[4-(1,1,1,3,3,3-hexafluoro-2-hydroxypropan-2-yl)-2-propylphenyl]phenylethyl)-5-methylimidazolidine-2,4-dione; 3-((2-chloro-5-[4-(1,1,1,3,3,3-hexafluoro-2-hydroxyropan-2-yl)-2-propylphenoxy]phenyl}methyl)-5-methyl-5-[4-(propan-2-yloxy)phenyl]imidazolidine-2,4-dione; 3-((3-[4-(1,1,1,3,3,3-hexafluoro-2-hydroxypropan-2-yl)-2,6-dipropylphenoxy]phenylmethyl)-5-methyl-5-[4-(propan-2-yloxy)phenyl]imidazolidine-2,4-dione; 3-(2-(4-[4-(1,1,1,3,3,3-hexafluoro-2-hydroxypropan-2-yl)-2-propylphenoxyphenyl]ethyl)-5-methyl-5-[5-(propan-2-yloxy)pyridin-2-yl]imidazolidine-2,4-dione; 3-(2-{4-[4-(1,1,1,3,3,3-hexafluoro-2-hydroxypropan-2-yl)-2-propylphenoxy]-2-methylphenyl}ethyl)-5-methyl-5-[4-(propan-2-yloxy)phenyl]imidazolidine-2,4-dione; 3-(2-{4-[4-(1,1,1,3,3,3-hexafluoro-2-hydroxypropan-2-yl)-2-propylphenoxy]-2-methoxyphenyl}ethyl)-5-methyl-5-[4-(propan-2-yloxy)phenyl]imidazolidine-2,4-dione; 3-(2-{4-[4-(1,1,1,3,3,3-hexafluoro-2-hydroxypropan-2-yl)-2-propylphenoxy-2-hydroxyphenyl}ethyl)-5-methyl-5-[4-(propan-2-yloxy)phenylimidazolidine-2,4-dione; 3-(2-(4-[4-(1,1,1,3,3,3-hexafluoro-2-hydroxypropan-2-yl)-2-propylphenoxy]-2-(methoxymethyl)phenyl]ethyl)-5-methyl-5-[4-(propan-2-yloxy)phenyl]imidazolidine-2,4-dione; 5-[4-(1,1,1,3,3,3-hexafluoro-2-hydroxypropan-2-yl)-2-propylphenoxy]-2-(2-{4-methyl-2,5-dioxo-4-[4-(propan-2-yloxy)phenyl]imidazolidin 1-yl}ethyl)benzonitrile; 3-((4-[4-(1,1,1,3,3,3-hexafluoro-2-hydroxypropan-2-yl)-2-propylphenoxy]pyridin-2-yl]methyl)-5-methyl-5-[5-(propan-2-yloxy)pyridin-2-yl]imidazolidine-2,4-dione; 5-(1-benzofuran-5-yl)-3-({4-[4-(1,1,1,3,3,3-hexafluoro-2-hydroxypropan-2-yl)-2-propylphenoxy]pyridin-2-yl}methy)-5-methylimidazolidine-2,4-dione; 3-({4-[4-(1,1,1,3,3,3-hexafluoro-2-hydroxypropan-2-yl)-2,6-dipropylphenoxy]pyridin-2-yl}methyl)-5-methyl-5-[4-(propan-2-yloxy)phenyl]imidazolidine-2,4-dione; 3-[(4-{[6-(1,1,1,3,3,3-hexafluoro-2-hydroxypropan-2-yl)-2-propylpyridin-3-yl]oxy}pyridin-2-yl)methyl]-5-methyl-5-[4-(propan-2-yloxy)phenyl]imidazolidine-2,4-dione; 3-{(2-[4-(1,1,1,3,3,3-hexafluoro-2-hydroxypropan-2-yl)-2-propylphenoxy]-5-iodopyridin-4-yl}methy)-5-methyl-5-[4-(propan-2-yl)phenyl]imidazolidine-2,4-dione; 3-({2-[4-(1,1,1,3,3,3-hexafluoro-2-hydroxypropan-2-yl)-2-propylphenoxy]-5-iodopyridin-4-yl}methyl)-5-methyl-5-[4-(propan-2-yloxy)phenyl]imidazolidine-2,4-dione; 3-(2-{5-[4-(1,1,1,3,3,3-hexafluoro-2-hydroxypropan-2-yl)-2-propylphenoxy]pyridin-2-yl}ethyl)-5-methyl-5-[4-(propan-2-yloxy)phenyl]imidazolidine-2,4-dione; 5-[3-fluoro-4-(propan-2-yloxy)phenyl-3-(2-{5-[4-(1,1,1,3,3,3-hexafluoro-2-hydroxypropan-2-yl)-2-propylphenoxy]pyridin-2-yl}ethyl)-5-methylimidazolidine-2,4-dione; 3-(2-{5-[4-(1,1,1,3,3,3-hexafluoro-2-hydroxypropan-2-yl)-2-propylphenoxy]pyridin-2-yl}ethyl)-5-methyl-5-[5-(propan-2-yloxy)pyridin-2-yl]imidazolidine-2,4-dione; 3-(2-{6-[4-(1,1,1,3,3,3-hexafluoro-2-hydroxypropan-2-yl)-2-propylphenoxy]pyridin-3-yl}]ethyl)-5-methyl-5-[4-(propan-2-yloxy)phenyl]imidazolidine-2,4-dione; 3-[2-(4-{[6-(1,1,1,3,3,3-hexafluoro-2-hydroxypropan-2-yl)-2-propylpyridin-3-yl]oxy}phenyl)ethyl]-5-(6-methoxypyridin-3-yl)-5-methylimidazolidine-2,4-dione; 3-[2-(5-([6-(1,1,1,3,3,3-hexafluoro-2-yl]imidazolidine-2,4-dione; 3-(2-(4-[4-(1,1,1,3,3,3-hexafluoro-2-hydroxypropan-2-yl)-2-propylphenoxy]phenyl]-2-oxoethyl)-5-methyl-5-[4-(propan-2-yloxy)phenyl]imidazolidine-2,4-dione; 5-(2,3-dihydro-1-benzofuran-5-yl)-3-(2-{4-[4-(1,1,1,3,3,3-hexafluoro-2-hydroxypropan-2-yl)-2-propylphenoxy]-2-methoxyphenyl}-2-oxoethyl)-5-methylimidazolidine-2,4-dione; 3-(2-{4-[4-(1,1,1,3,3,3-hexafluoro-2-hydroxypropan-2-yl)-2-propylphenoxy]-2-methylphenyl}-2-oxoethyl)-5-methyl-5-[4-(propan-2-yloxy)phenyl]imidazolidine-2,4-dione; 3-(2-{4-[4-(1,1,1,3,3,3-hexafluoro-2-hydroxypropan-2-yl)-2propylphenoxy]-2-methylphenyl}-2-oxoethyl)-5-methyl-5-[5-(propan-2-yloxy)pyridin-2-yl]imidazolidine-2,4-dione; 3-(2-{4-[4-(1,1,1,3,3,3-hexafluoro-2-hydroxypropan-2-yl)-2,6-dipropylphenoxy]-2-methylphenyl}-2-oxoethyl)-5-methyl-5-[5-(propan-2-yloxy)pyridin-2-yl]imidazolidine-2,4-dione; 3-(2-{4-[4-(1,1,1,3,3,3-hexafluoro-2-hydroxyropan-2-yl)-2-propylphenoxy]-2-hydroxyphenyl}-2-oxoethyl)-5-methyl-5-[4-(propan-2-yloxy)phenyl]imidazolidine-2,4-dione; 3-(2-{4-[4-(1,1,1,3,3,3-hexafluoro-2-hydroxypropan-2-yl)-2-propylphenoxy]-2-hydroxyphenyl}-2-oxoethyl)-5-methyl-5-[5-(propan-2-yloxy)pyridin-2-yl]imidazolidine-2,4-dione; 3-(2-{5-[4-(1,1,1,3,3,3-hexafluoro-2-hydroxypropan-2-yl)-2-propylphenoxy]pyridin-2-yl}-2-oxoethyl)-5-methyl-5-[4-(propan-2-yloxy)phenyl]imidazolidine-2,4-dione; or 3-(1-{5-[4-(1,1,1,3,3,3-hexafluoro-2-hydroxypropan-2-yl)-2-propylphenoxy]pyridin-2-yl}-1-oxopropan-2-yl)-5-methyl-5-[4-(propan-2-yloxy)phenyl]imidazolidine-2,4-dione; or pharmaceutically acceptable salts thereof.

In further embodiments of any of the foregoing methods, the LXR agonist is a compound described in International Patent Publication No. WO2013/076257, e.g., any one of: 5α-hydroxy-6β-[2-(1H-imidazol-4-yl)ethylamino]cholestan-3β-ol (Dendrogenin A), 5α-hydroxy-6β-[2-(1H-imidazol-4-yl)ethylamino]campestan-3β-ol, 5α-hydroxy-6β-[2-(1H-imidazol-4-yl)ethylamino]sitostan-3β-ol, 3β-acetoxy-5α-hydroxy-6β-[2-(1H-imidazol-4-yl)ethylamino]cholestane, 3β-acetoxy-5α-hydroxy-6-[2-(1H-imidazol-4-yl)ethylamino]campestane, 3β-acetoxy-5α-hydroxy-6β-[2-(1H-imidazol-4-yl)ethylamino]sitostane, 5α-hydroxy-6β-[2-(1H-indol-3-yl)ethylamino]cholestan-3β-ol, 5α-hydroxy-6β-[2-(1H-indol-3-yl)ethylamino]campestan-3β-ol, 5α-hydroxy-6β-[2-(1H-indol-3-yl)ethylamino]sitostan-3β-ol, 5α-hydroxy-6β-[2-(1H-indol-3-yl)ethylamino]cholest-7-en-3β-ol, 3β-acetoxy-5α-hydroxy-6β-[2-(1H-indol-3-yl)ethylamino]cholestane, 3β-acetoxy-5α-hydroxy-6β-[2-(1H-indol-3-yl)ethylamino]campestane, 3β-acetoxy-5α-hydroxy-6β-[2-1H-indol-3-yl)ethylamino]sitostane, 3β-acetoxy-5α-hydroxy-6β-[2-(1H-indol-3-yl)ethylamino]cholest-7-ene, 5α-hydroxy-6β-[2-(1H-indol-3-yl-5-ol)ethylamino]cholestan-3β-ol, 5α-hydroxy-6β-[2-(1H-indol-3-yl-5-ol)ethylamino]cholest-7-en-3β-ol, 5α-hydroxy-6β-[2-(1H-indol-3-yl-5-ol)ethylamino]campestan-3β-ol, 5α-hydroxy-6β-[2-(2-(1H-indol-3-yl-5-ol)ethylamino]sitostan-3β-ol, 3β-acetoxy-5α-hydroxy-6-[2-(2-(1H-indol-3-yl-5-ol)ethylamino]cholestane, 3β-acetoxy-5α-hydroxy-6β-[2-(1H-indol-3-yl-5-ol)ethylamino]cholest-7-ene, 3β-acetoxy-5α-hydroxy-6β-[2-(2-(1H-indol-3-yl-5-ol)ethylamino]campestane, 3β-acetoxy-5α-hydroxy-6β-[2-(1H-indol-3-yl-5-ol)ethylamino]sitostane, 5α-hydroxy-6β-[3-(4-aminobutylamino)propylamino]cholest-7-en-3β-ol (Dendrogenin B), 5α-hydroxy-6β-[4-(3-aminopropylamino)butylamino]cholest-7-en-3β-ol, 5α-hydroxy-6β-[3-(4-aminobutylamino)propylamino]-cholestan-3β-ol, 5α-hydroxy-6β-[4-(3-aminopropylamino)butylamino]-cholestan-3β-ol, 5α-hydroxy-6β-[3-(4-aminobutylamino)propylamino]-campestan-3β-ol, 5α-hydroxy-6β-[4-(3-aminopropylamino)butylamino]-campestan-3β-ol, 5α-hydroxy-6β-[3-(4-aminobutylamino)propylamino]sitostan-3β-ol, 5α-hydroxy-6β0-[4-(3-aminopropylamino)butylamino]sitostan-3β-ol, 5α-hydroxy-6β-[(4-aminobutyl)(3-aminopropyl)amino]cholest-7-en-3β-ol, 5α-hydroxy-6β-[(4-aminobutyl)(3-aminopropyl)amino]cholestan-3β-ol, 5α-hydroxy-6β-[(4-aminobutyl)(3-aminopropyl)amino]campestan-3β-ol, 5α-hydroxy-6β-[(4-aminobutyl)(3-aminopropyl)amino]-sitostan-3β-ol, 5α-hydroxy-6β-{3-[4-(3-aminopropylamino)butylamino]propylamino}cholest-7-en-3β-ol, 5α-hydroxy-6β-{3-[4-(3-aminopropylamino)butylamino]propylamino}cholestan-3β-ol, 5α-hydroxy-6β-{3-[4-(3-aminopropylamino)butylamino]propylamino}campestan-3β-ol, 5α-hydroxy-6β-{3-[4-(3-aminopropylamino)-butylamino]propylamino}sitostan-3β-ol, 5α-hydroxy-6β-(4-aminobutylamino)cholest-7-en-3β-ol, 5α-hydroxy-6β-(4-aminobutylamino)cholestan-3β-ol, 5α-hydroxy-6β-(4-aminobutylamino)campestan-3β-ol, 5α-hydroxy-6β-(4-aminobutylamino)sitostan-3β-ol, 5α-hydroxy-6β-(3-aminopropylamino)cholest-7-en-3β-ol, 5α-hydroxy-6β-(3-aminopropylamino)cholestan-3β-ol, 5α-hydroxy-6β-(3-aminopropylamino)campestan-3β-ol, 5α-hydroxy-6β-(3-aminopropylamino)sitostan-3β-ol, 3β-acetoxy-5α-hydroxy-6β-[3-(4-aminobutylamino)-propylamino]cholest-7-ene, 3β-acetoxy-5α-hydroxy-6β-[4-(3-aminopropylamino)butylamino]cholest-7-ene, 3β-acetoxy-5α-hydroxy-6β-[3-(4-aminobutylamino)propylamino]cholestane, 3β-acetoxy-5α-hydroxy-6β-[4-(3-aminopropylamino)butylamino]cholestane, 3β-acetoxy-5α-hydroxy-6β-[3-(4-aminobutylamino)-propylamino]campestane, 3β-acetoxy-5α-hydroxy-6β-[4-(3-aminopropylamino)butylamino]campestane, 3β-acetoxy-5α-hydroxy-6β-[3-(4-aminobutylamino)propylamino]sitostane, 3β-acetoxy-5α-hydroxy-6β-[4-(3-aminopropylamino)butylamino]sitostane, 3β-acetoxy-5α-hydroxy-6β-[(4-aminobutyl)(3-aminopropyl)-amino]cholest-7-ene, 3β-acetoxy-5α-hydroxy-6β-[(4-aminobutyl)(3-aminopropyl)amino]cholestane, 3β-acetoxy-5α-hydroxy-6β-[(4-aminobutyl)(3-aminopropyl)amino]campestane, 3β-acetoxy-5α-hydroxy-6β-[(4-aminobutyl)(3-aminopropyl)amino]sitostane, 3β-acetoxy-5α-hydroxy-6β-{3-[4-(3-aminopropylamino)-butylamino]propylamino}cholest-7-ene, 3β-acetoxy-5α-hydroxy-6β-{3-[4-(3-aminopropylamino)-butylamino]propylamino}cholestane, 3β-acetoxy-5α-hydroxy-6β-{3-[4-(3-aminopropylamino)-butylamino]propylamino}campestane, 3β-acetoxy-5α-hydroxy-6β-{3-[4-(3aminopropylamino) butylamino]-propylamino}sitostane, 3β-acetoxy-5α-hydroxy-6β-(4-aminobutylamino)cholest-7-ene, 3β-acetoxy-5α-hydroxy-6β-(4-aminobutylamino)cholestane, 3β-acetoxy-5α-hydroxy-6β-(4-aminobutylamino)-campestane, 3β-acetoxy-5α-hydroxy-6β-(4-aminobutylamino)sitostane, 3β-acetoxy-5α-hydroxy-6β-(3-aminopropylamino)-cholest-7-ene, 3β-acetoxy-5α-hydroxy-6β-(3-aminopropylamino)cholestane, 3β-acetoxy-5α-hydroxy-6β-(3-aminopropylamino)campestane, or 3β-acetoxy-5α-hydroxy-6β-(3-aminopropylamino)sitostane. or pharmaceutically acceptable salts thereof.

In further embodiments of any of the foregoing methods, the LXR agonist is a compound described in International Patent Publication No. WO2013/057148, e.g., acid addition salt of 5α-hydroxy-6β-[2-(1H-imidazol-4yl)ethylamino]cholestan-3β-ol such as acid addition salts formed with benzenesulfonic acid, benzoic acid, 4methylbenzenesulfonic acid, 4,4′methylenebis-3-hydroxy-2-naphtoic acid, mesylic acid, L-tartaric acid, D-tartaric acid, L-malic acid, citric acid, 2-(S)-hydroxypropanoic acid, succinic acid, glutaric acid, malonic acid, fumaric acid, acetic acid, hydrochloride acid, or sulfuric acid.

In further embodiments of any of the foregoing methods, the LXR agonist is an LXR agonist described in International Patent Publication Nos: WO2006/046593; WO2006/073366; WO2010/125811; WO2009/144961; WO2009/133692; WO2010/025169; WO2010/125811; WO2011/051282; WO2010/023317; WO2012/135082; WO2009/150109; WO2013/130892; WO2010/059627; WO2012/004748; WO2013/138565; WO2013/138568; WO2011/014661; WO2002/090375; WO00/066611; WO2006/109633; WO2006/003923; WO2005/113499; WO2006/073365; WO2006/073364; WO2006/073363; WO2006/073367; WO2009/021868; WO2006000323; WO2010/125811; WO2009/144961; WO2009/133692; WO2010/025179; WO2009/138438; WO2003/043998, WO2003/045382, WO2003/059874, WO2003/059884, WO2003/060078, WO2003/090732, WO2003/090746, WO2003/090869, WO2003/099769, WO2003/099775, WO2003/106435, WO2004/009091, WO2004/011448, WO2004/026816, WO2004/058717, WO2004/072041, WO2004/072042, WO2004/072046, WO2005/005416, WO2005/005417, WO2005/016277, WO2005/023782, WO2005/077122, WO2005/077124, WO2006/094034, WO2007/024954, WO2007/047991, WO2007/092065, WO2008/049047, WO2009/086123, WO2009/086129, WO2009/086130, WO2009/086138, and WO2011/055391; U.S. Pat. Nos. 6,906,069 and 7,790,745, and U.S. Patent Publication Nos: US2005/0080111, US2006/0135601; US2006/0074115; US2005/0245515; US2005/0215577; US2009/0247587; US2002/0107233, US2003/0125357, US2003/0153541, US2005/0080111, US2005/0113419, US2005/0131014, US2005/0261319, US2006/0030612, US2006/0178398, US2007/0093524, US2009/0030082, and US2015/0299136, the compounds of which are herein incorporated by reference. In other embodiments of any of the foregoing methods, the LXR agonist is an LXR agonist described in Li et al, Expert Opin. Ther. Patents (2010) 20(4):535-562 and Tice et al., J. Med. Chem. (2014) 57:7182-7205, the compounds of which are herein incorporated by reference.

In some embodiments of any of the foregoing methods, the method further includes administration of an additional anticancer therapy (e.g., an antiproliferative). In other embodiments, the additional anticancer therapy is any one of the antiproliferatives listed in Table 2. In some embodiments, the additional anticancer therapy is an immunotherapy (e.g., a PD-1 inhibitor such as a PD-1 antibody, a PD-L1 inhibitor such as a PD-L1 antibody, a CTLA-4 inhibitor such as a CTLA-4 antibody, a CSF-1R inhibitor, an IDO inhibitor, an A1 adenosine inhibitor, an A2A adenosine inhibitor, an A2B adenosine inhibitor, an A3A adenosine inhibitor, an arginase inhibitor, or an HDAC inhibitor). In some embodiments, the immunotherapy is a PD-1 inhibitor (e.g., nivolumab, pembrolizumab, pidilizumab, BMS 936559, and MPDL328OA). In some embodiments, the immunotherapy is a PD-L1 inhibitor (e.g., atezolizumab and MED14736). In some embodiments, the immunotherapy is a CTLA-4 inhibitor (e.g., ipilimumab). In some embodiments, the immunotherapy is a CSF-1R inhibitor (e.g., pexidartinib and AZD6495). In some embodiments, the immunotherapy is an IDO inhibitor (e.g., norharmane, rosmarinic acid, and alpha-methyl-tryptophan). In some embodiments, the immunotherapy is an A1 adenosine inhibitor (e.g., 8-cyclopentyl-1,3-dimethylxanthine, 8-cyclopentyl-1,3-dipropylxanthine, 8-phenyl-1,3-dipropylxanthine, bamifylline, BG-9719, BG-9928, FK-453, FK-838, rolofylline, or N-0861). In some embodiments, the immunotherapy is an A2A adenosine inhibitor (e.g., ATL-4444, istradefylline, MSX-3, preladenant, SCH-58261, SCH-412,348, SCH-442,416, ST-1535, VER-6623, VER-6947, VER-7835, viadenant, or ZM-241,385). In some embodiments, the immunotherapy is an A2B adenosine inhibitor (e.g., ATL-801, CVT-6883, MRS-1706, MRS-1754, OSIP-339,391, PSB-603, PSB-0788, or PSB-1115). In some embodiments, the immunotherapy is an A3A adenosine inhibitor (e.g., KF-26777, MRS-545, MRS-1191, MRS-1220, MRS-1334, MRS-1523, MRS-3777, MRE-3005-F20, MRE-3008-F20, PSB-11, OT-7999, VUF-5574, and SSR161421). In some embodiments, the immunotherapy is an arginase inhibitor (e.g., an arginase antibody, (2s)-(+)-amino-5-iodoacetamidopentanoic acid, NG-hydroxy-L-arginine, (2S)-(+)-amino-6-iodoacetamidohexanoic acid, or (R)-2-amino-6-borono-2-(2-(piperidin-1-yl)ethyl)hexanoic acid. In some embodiments, the immunotherapy is an HDAC inhibitor (e.g., valproic acid, SAHA, or romidepsin).

In some embodiments, the antiproliferative and LXR agonist are administered within 28 days of each (e.g., within 21, 14, 10, 7, 5, 4, 3, 2, or 1 days) or within 24 hours (e.g., 12, 6, 3, 2, or 1 hours; or concomitantly) other in amounts that together are effective to treat the subject.

In certain embodiments, the antiproliferative is: a chemotherapeutic or cytotoxic agent, a differentiation-inducing agent (e.g. retinoic acid, vitamin D, cytokines), a hormonal agent, an immunological agent, or an anti-angiogenic agent. Chemotherapeutic and cytotoxic agents include, but are not limited to, alkylating agents, cytotoxic antibiotics, antimetabolites, vinca alkaloids, etoposides, and others (e.g., paclitaxel, taxol, docetaxel, taxotere, cis-platinum). A list of additional compounds having antiproliferative activity can be found in L. Brunton, B. Chabner and B. Knollman (eds). Goodman and Gilman's The Pharmacological Basis of Therapeutics, Twelfth Edition, 2011, McGraw Hill Companies, New York, N.Y.

In other embodiments, the antiproliferative is a PD1 inhibitor, a VEGF inhibitor, a VEGFR2 inhibitor, a PDL1 inhibitor, a BRAF inhibitor, a CTLA-4 inhibitor, a MEK inhibitor, an ERK inhibitor, vemurafenib, dacarbazine, trametinib, dabrafenib, MEDI-4736, an mTOR inhibitor, a CAR-T therapy, abiraterone, enzalutamine, ARN-509, 5-FU, FOLFOX, FOLFIRI, herceptin, xeloda, a PD1 antibody (e.g., pembrolizumab or nivolumab), a PDL-1 antibody, a CTLA-4 antibody (e.g, ipilimumab), ramucirumab, rindopepimut, glembatumumab, vedotin, ANG1005, and/or ANG4043.

In some embodiments, the cancer is a renal cell carcinoma and the antiproliferative is a PD1 inhibitor, a PDL-1 inhibitor, or an mTOR inhibitor. In other embodiments, the cancer is diffuse large B-cell lymphoma and the antiproliferative is a CAR-T therapy. In certain embodiments, the cancer is prostate cancer and the antiproliferative is abiraterone, enzalutamide, or ARN-509. In some embodiments, the cancer is hepatocellular carcinoma, gastric cancer, or esophageal cancer and the antiproliferative is 5-FU, FOLFOX, FOLFIRI, herceptin, or xeloda. In some embodiments, the cancer is sarcoma and the antiproliferative is gemcitabine. In other embodiments, the cancer is pancreatic cancer and the antiproliferative is irinotecan, cisplatin, abraxane, a taxane (e.g., paclitaxel or docetaxel), or capecitabine.

The method may further include administering an antiproliferative selected from the group consisting of alkylating agents, platinum agents, antimetabolites, topoisomerase inhibitors, antitumor antibiotics, antimitotic agents, aromatase inhibitors, thymidylate synthase inhibitors, DNA antagonists, farnesyttransferase inhibitors, pump inhibitors, histone acetyltransferase inhibitors, metalloproteinase inhibitors, ribonucleoside reductase inhibitors, TNF alpha agonists/antagonists, endothelin A receptor antagonist, retinoic acid receptor agonists, immuno-modulators, hormonal and antihormonal agents, photodynamic agents, tyrosine kinase inhibitors, antisense compounds, corticosteroids, HSP90 inhibitors, proteosome inhibitors (for example, NPI-0052), CD40 inhibitors, anti-CSI antibodies, FGFR3 inhibitors, VEGF inhibitors, MEK inhibitors, cyclin D1 inhibitors, NF-kB inhibitors, anthracyclines, histone deacetylases, kinesin inhibitors, phosphatase inhibitors, COX2 inhibitors, mTOR inhibitors, calcineurin antagonists, IMiDs, or other agents used to treat proliferative diseases. Examples of such compounds are provided in Tables 1.

In some embodiments of any of the foregoing methods, administering comprises contacting a cell with an effective amount of an LXR agonist (e.g., an LXRβ agonist).

### Chemical Terms

The term “alkyl” used is the present application relates a saturated branched or unbranched aliphatic univalent substituent. The alkyl substituent has 1 to 100 carbon atoms, (e.g., 1 to 22 carbon atoms, 1 to 10 carbon atoms 1 to 6 carbon atoms, 1 to 3 carbon atoms). Accordingly, examples of the alkyl substituent include methyl, ethyl, n-propyl, isopropyl, n-butyl, isobutyl, sec-butyl, tert-butyl, n-pentyl and n-hexyl.

The term “alkoxy” represents a chemical substituent of formula —OR, where R is an optionally substituted C1-C6 alkyl group, unless otherwise specified. In some embodiments, the alkyl group can be substituted, e.g., the alkoxy group can have 1, 2, 3, 4, 5 or 6 substituent groups as defined herein.

The term “alkoxyalkyl” represents a heteroalkyl group, as defined herein, that is described as an alkyl group that is substituted with an alkoxy group. Exemplary unsubstituted alkoxyalkyl groups include between 2 to 12 carbons. In some embodiments, the alkyl and the alkoxy each can be further substituted with 1, 2, 3, or 4 substituent groups as defined herein for the respective group.

As used herein, the term “cycloalkyl” refers to a monocyclic, bicyclic, or tricyclic substituent, which may be saturated or partially saturated, i.e. possesses one or more double bonds. Monocyclic substituents are exemplified by a saturated cyclic hydrocarbon group containing from 3 to 8 carbon atoms. Examples of monocyclic cycloalkyl substituents include cyclopropyl, cyclobutyl, cyclopentyl, cyclopentenyl, cyclohexyl, cyclohexenyl, cycloheptyl and cyclooctyl. Bicyclic fused cycloalkyl substituents are exemplified by a cycloalkyl ring fused to another cycloalkyl ring. Examples of bicyclic cycloalkyl substituents include, but are not limited to decalin, 1,2,3,7,8,8a-hexahydro-naphthalene, and the like. Tricyclic cycloalkyl substituents are exemplified by a cycloalkyl bicyclic fused ring fused to an additional cycloalkyl substituent.

The term “alkylene” used is the present application relates a saturated branched or unbranched aliphatic bivalent substituent (e.g. the alkylene substituent has 1 to 6 carbon atoms, 1 to 3 carbon atoms). Accordingly, examples of the alkylene substituent include methylene, ethylene, trimethylene, propylene, tetramethylene, isopropylidene, pentamethylene and hexamethylene.

The term “alkenylene or alkenyl” as used in the present application is an unsaturated branched or unbranched aliphatic bivalent substituent having a double bond between two adjacent carbon atoms (e.g. the alkenylene substituent has 2 to 6 carbon atoms, 2 to 4 carbon atoms). Accordingly, examples of the alkenylene substituent include but are not limited to vinylene, 1-propenylene, 2-propenylene, methylvinylene, 1-butenylene, 2-butenylene, 3-butenylene, 2-methyl-1-propenylene, 2-methyl-2-propenylene, 2-pentenylene, 2-hexenylene.

The term “alkynylene or alkynyl” as used is the present application is an unsaturated branched or unbranched aliphatic bivalent substituent having a triple bond between two adjacent carbon atoms (e.g. the alkynylene substituent has 2 to 6 carbon atoms 2 to 4 carbon atoms). Examples of the alkynylene substituent include but are not limited to ethynylene, 1-propynylene, 1-butynylene, 2-butynylene, 1-pentynylene, 2-pentynylene, 3-pentynylene and 2-hexynylene.

The term “alkadienylene” as used is the present application is an unsaturated branched or unbranched aliphatic bivalent substituent having two double bonds between two adjacent carbon atoms(e.g. the alkadienylene substituent has 4 to 10 carbon atoms). Accordingly, examples of the alkadienylene substituent include but are not limited to 2,4-pentadienylene, 2,4-hexadienylene, 4-methyl-2,4-pentadienylene, 2,4-heptadienylene, 2,6-heptadienylene, 3-methyl-2,4-hexadienylene, 2,6-octadienylene, 3-methyl-2,6-heptadienylene, 2-methyl-2,4-heptadienylene, 2,8-nonadienylene, 3-methyl-2,6-octadienylene, 2,6-decadienylene, 2,9-decadienylene and 3,7-dimethyl-2,6-octadienylene substituents.

The term “heteroaliphatic substituent or heteroalkyl”, as used herein, refers to a monovalent or a bivalent substituent, in which one or more carbon atoms have been substituted with a heteroatom, for instance, with an oxygen, sulfur, nitrogen, phosphorus or silicon atom, wherein the nitrogen and sulfur atoms may optionally be oxidized, and the nitrogen heteroatom may optionally be quaternized. The heteroatom(s) O, N and S may be placed at any interior position of the heteroaliphatic substituent. Examples include —CH₂—CH₂—O—CH₃, —CH₂—CH₂—NH—CH₃, —CH₂—CH₂—N(CH₃)—CH₃, —CH₂—S—CH₂—CH₃, —S(O)—CH₃, —CH₂—CH₂—S(O)₂—CH₃, —CH═CH—O—CH₃, —CH₂—CH═N—OCH₃, and —CH═CH—N(CH₃)—CH₃. A heteroaliphatic substituent may be linear or branched, and saturated or unsaturated.

In one embodiment, the heteroaliphatic substituent has 1 to 100, (e.g 1 to 42 carbon atoms). In yet another embodiment, the heteroaliphatic substituent is a polyethylene glycol residue.

As used herein, “aromatic substituent or aryl” is intended to mean any stable monocyclic, bicyclic or polycyclic carbon ring of up to 10 atoms in each ring, wherein at least one ring is aromatic, and may be unsubstituted or substituted. Examples of such aromatic substituents include phenyl, p-toluenyl (4-methylphenyl), naphthyl, tetrahydronaphthyl, indanyl, biphenyl, phenanthryl, anthryl or acenaphthyl. In cases where the aromatic substituent is bicyclic and one ring is non-aromatic, it is understood that attachment is via the aromatic ring.

The term “alkylaryl substituents or arylalkyl” refers to alkyl substituents as described above wherein one or more bonds to hydrogen contained therein are replaced by a bond to an aryl substituent as described above. It is understood that an arylalkyl substituents is connected to the carbonyl group if the compound of the invention through a bond from the alkyl substituent. Examples of arylalkyl substituents include, but are not limited to, benzyl (phenylmethyl), p-trifluoromethylbenzyl (4-trifluoromethylphenylmethyl), 1-phenylethyl, 2-phenylethyl, 3-phenylpropyl, 2-phenylpropyl and the like.

The term “heteroaromatic substituent or heteroaryl” as used herein, represents a stable monocyclic, bicyclic or polycyclic ring of up to 10 atoms in each ring, wherein at least one ring is aromatic and contains from 1 to 4 heteroatoms selected from the group consisting of O, N and S. Bicyclic heteroaromatic substituents include phenyl, pyridine, pyrimidine or pyridizine rings that are
a) fused to a 6-membered aromatic (unsaturated) heterocyclic ring having one nitrogen atom;
b) fused to a 5- or 6-membered aromatic (unsaturated) heterocyclic ring having two nitrogen atoms;
c) fused to a 5-membered aromatic (unsaturated) heterocyclic ring having one nitrogen atom together with either one oxygen or one sulfur atom; or
d) fused to a 5-membered aromatic (unsaturated) heterocyclic ring having one heteroatom selected from O, N or S.

Heteroaryl groups within the scope of this definition include but are not limited to: benzoimidazolyl, benzofuranyl, benzofurazanyl, benzopyrazolyl, benzotriazolyl, benzothiophenyl, benzoxazolyl, carbazolyl, carbolinyl, cinnolinyl, furanyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrazolyl, tetrazolopyridyl, thiadiazolyl, thiazolyl, thienyl, triazolyl, azetidinyl, aziridinyl, 1,4-dioxanyl, hexahydroazepinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, tetrahydrothienyl, acridinyl, carbazolyl, cinnolinyl, quinoxalinyl, pyrrazolyl, indolyl, benzotriazolyl, benzothiazolyl, benzoxazolyl, isoxazolyl, isothiazolyl, furanyl, thienyl, benzothienyl, benzofuranyl, quinolinyl, isoquinolinyl, oxazolyl, isoxazolyl, indolyl, pyrazinyl, pyridazinyl, pyridinyl, pyrimidinyl, pyrrolyl, tetrahydroquinoline. In cases where the heteroaryl substituent is bicyclic and one ring is non-aromatic or contains no heteroatoms, it is understood that attachment is via the aromatic ring or via the heteroatom containing ring, respectively. If the heteroaryl contains nitrogen atoms, it is understood that the corresponding N-oxides thereof are also encompassed by this definition.

The aliphatic, heteroaliphatic, aromatic and heteroaromatic substituents can be optionally substituted one or more times, the same way or differently with any one or more of the following substituents including, but not limited to: aliphatic, heteroaliphatic, aromatic and heteroaromatic substituents, aryl, heteroaryl; alkylaryl; heteroalkylaryl; alkyiheteroaryl; heteroalkyiheteroaryl; alkoxy; aryloxy; heteroalkoxy; heteroaryloxy; alkylthio; arytthio; heteroalkythio; heteroarytthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)Rₓ; —S(O)₂Rₓ; —NRₓ(CO)Rₓ wherein each occurrence of Rₓ independently includes, but is not limited to, aliphatic, alicyclic, heteroaliphatic, heterocyclic, aromatic, heteroaromatic, aryl, heteroaryl, alkylaryl, alkyiheteroaryl, heteroalkylaryl or heteroalkyiheteroaryl, wherein any of the aliphatic, alicyclic, heteroaliphatic, heterocyclic, alkylaryl, or alkyiheteroaryl substituents described above and herein may be substituted or unsubstituted, branched or unbranched, saturated or unsaturated, and wherein any of the aromatic, heteroaromatic, aryl, heteroaryl, (alkyl)aryl or (alkyl)heteroaryl substituents described above and herein may be substituted or unsubstituted. Additionally, it will be appreciated, that any two adjacent substituents taken together may represent a 4, 5, 6, or 7-membered substituted or unsubstituted alicyclic or heterocyclic substituents. Additional examples of generally applicable substituents are illustrated by the specific embodiments shown below.

The terms “halo” and “halogen” refer to a halogen atom selected from the group consisting of F, Cl, Br and I.

The term “halogenated alkyl substituent, haloalkyl” refers to an alkyl substituents as defined above which is substituted with at least one halogen atom. In an embodiment, the halogenated alkyl substituent is perhalogenated. In another embodiment, perfluoroalkyl refers to the halogenated alkyl substituent is a univalent perfluorated substituent of formula CₙF₂ₙ₊₁. For example, the halogenated alkyl substituent may have 1 to 6 carbon atoms, (e.g. 1 to 3 carbon atoms). Accordingly, examples of the alkyl group include trifluoromethyl, 2,2,2-trifluoroethyl, n-perfluoropropyl, n-perfluorobutyl and n-perfluoropentyl.

The term “amino,” as used herein, represents —N(R^{N1})₂, wherein each R^{N1} is, independently, H, OH, NO₂, N(R^{N2})₂, SO₂OR^{N2}, SO₂R^{N2}, SOR^{N2}, an N-protecting group, alkyl, alkenyl, alkynyl, alkoxy, aryl, alkaryl, cycloalkyl, alkcycloalkyl, heterocyclyl (e.g., heteroaryl), alkheterocyclyl (e.g., alkheteroaryl), or two R^{N1} combine to form a heterocyclyl or an N-protecting group, and wherein each R^{N2} is, independently, H, alkyl, or aryl. In a preferred embodiment, amino is —NH₂, or —NHR^{N1}, wherein R^{N1} is, independently, OH, NO₂, NH₂, NR^{N2}, SO₂OR^{N2}, SO₂R^{N2}, SOR^{N2}, alkyl, or aryl, and each R^{N2} can be H, alkyl, or aryl. The term “aminoalkyl,” as used herein, represents a heteroalkyl group, as defined herein, that is described as an alkyl group, as defined herein, substituted by an amino group, as defined herein. The alkyl and amino each can be further substituted with 1, 2, 3, or 4 substituent groups as described herein for the respective group. For example, the alkyl moiety may comprise an oxo (═O) substituent.

As used herein, the term “aryloxy” refers to aromatic or heteroaromatic systems which are coupled to another residue through an oxygen atom. A typical example of an O-aryl is phenoxy. Similarly, “arylalkyl” refers to aromatic and heteroaromatic systems which are coupled to another residue through a carbon chain, saturated or unsaturated, typically of C1-C8, C1-C6, or more particularly C1-C4 or C1-C3 when saturated or C2-C8, C2-C6, C2-C4, or C2-C3 when unsaturated, including the heteroforms thereof. For greater certainty, arylalkyl thus includes an aryl or heteroaryl group as defined above connected to an alkyl, heteroalkyl, alkenyl, heteroalkenyl, alkynyl or heteroalkynyl moiety also as defined above. Typical arylalkyls would be an aryl(C6-C12)alkyl(C1-C8), aryl(C6-C12)alkenyl(C2-C8), or aryl(C6-C12)alkynyl(C2-C8), plus the heteroforms. A typical example is phenylmethyl, commonly referred to as benzyl.

Typical optional substituents on aromatic or heteroaromatic groups include independently halo, CN, NO₂, CF₃, OCF₃, COOR′, CONR′₂, OR′, SR′, SOR′, SO₂R′, NR′₂, NR′(CO)R′,NR′C(O)OR′, NR′C(O)NR′₂, NR′SO₂NR′₂, or NR′SO₂R′, wherein each R′ is independently H or an optionally substituted group selected from alkyl, alkenyl, alkynyl, heteroalkyl, heteroalkenyl, heteroalkynyl, heteroaryl, and aryl (all as defined above); or the substituent may be an optionally substituted group selected from alkyl, alkenyl, alkynyl, heteroalkyl, heteroalkenyl, heteroalkynyl, aryl, heteroaryl, O-aryl, O-heteroaryl and arylalkyl.

Optional substituents on a non-aromatic group (e.g., alkyl, alkenyl, and alkynyl groups), are typically selected from the same list of substituents suitable for aromatic or heteroaromatic groups, except as noted otherwise herein. A non-aromatic group may also include a substituent selected from ═O and =NOR‘ where R’ is H or an optionally substituted group selected from alkyl, alkenyl, alkynyl, heteroalkyl, heteroalkenyl, heteralkynyl, heteroaryl, and aryl (all as defined above).

In general, a substituent group (e.g., alkyl, alkenyl, alkynyl, or aryl (including all heteroforms defined above) may itself optionally be substituted by additional substituents. The nature of these substituents is similar to those recited with regard to the substituents on the basic structures above. Thus, where an embodiment of a substituent is alkyl, this alkyl may optionally be substituted by the remaining substituents listed as substituents where this makes chemical sense, and where this does not undermine the size limit of alkyl per se; e.g., alkyl substituted by alkyl or by alkenyl would simply extend the upper limit of carbon atoms for these embodiments, and is not included. However, alkyl substituted by aryl, amino, halo and the like would be included. For example, where a group is substituted, the group may be substituted with 1, 2, 3, 4, 5, or 6 substituents. Optional substituents include, but are not limited to: C1-C6 alkyl or heteroaryl, C2-C6 alkenyl or heteroalkenyl, C2-C6 alkynyl or heteroalkynyl, halogen; aryl, heteroaryl, azido(—N₃), nitro (—NO₂), cyano (—CN), acyloxy(—OC(═O)R′), acyl (—C(═O)R′), alkoxy (—OR′), amido (—NR′C(═O)R″ or —C(═O)NRR′), amino (—NRR′), carboxylic acid (—CO₂H), carboxylic ester (—CO₂R′), carbamoyl (—OC(≡O)NR′R″ or —NRC(═O)OR′), hydroxy (—OH), isocyano (—NC), sulfonate (—S(═O)₂OR), sulfonamide (—S(═O)₂NRR′ or —NRS(═O)₂R′), or sulfonyl (—S(═O)₂R), where each R or R′ is selected, independently, from H, C1-C6 alkyl or heteroaryl, C2-C6 alkenyl or heteroalkenyl, 2C-6C alkynyl or heteroalkynyl, aryl, or heteroaryl. A substituted group may have, for example, 1, 2, 3, 4, 5, 6, 7, 8, or 9 substituents.

The term “heterocyclyl, heterocyclic, or Het” as used herein represents cyclic heteroalkyl or heteroalkenyl that is, e.g., a 3-, 4-, 5-, 6- or 7-membered ring, unless otherwise specified, containing one, two, three, or four heteroatoms independently selected from the group consisting of nitrogen, oxygen, and sulfur. The 5-membered ring has zero to two double bonds, and the 6- and 7-membered rings have zero to three double bonds. The term “heterocyclyl” also represents a heterocyclic compound having a bridged multicyclic structure in which one or more carbons and/or heteroatoms bridges two non-adjacent members of a monocyclic ring, e.g., a quinuclidinyl group. The term “heterocyclyl” includes bicyclic, tricyclic, and tetracyclic groups in which any of the above heterocyclic rings is fused to one, two, or three carbocyclic rings, e.g., an aryl ring, a cyclohexane ring, a cyclohexene ring, a cyclopentane ring, a cyclopentene ring, or another monocyclic heterocyclic ring, such as indolyl, quinolyl, isoquinolyl, tetrahydroquinolyl, benzofuryl, benzothienyl and the like.

Some of the compounds of the present invention can comprise one or more stereogenic centers, and thus can exist in various isomeric forms, e.g. stereoisomers and/or diastereomers. Thus, the compounds of the invention and pharmaceutical compositions thereof may be in the form of an individual enantiomer, diastereomer or geometric isomer, or may be in the form of a mixture of stereoisomers. In certain embodiments, the compounds of the invention are enantiopure compounds. In certain other embodiments, mixtures of stereoisomers or diastereomers are provided. Moreover, when compounds of the invention exist in tautomeric forms, each tautomer is embraced herein.

Furthermore, certain compounds, as described herein may have one or more double bonds that can exist as either the Z or E isomer, unless otherwise indicated. The invention additionally encompasses the compounds as individual isomers substantially free of other isomers and alternatively, as mixtures of various isomers, e.g., racemic mixtures of stereoisomers. In addition to the above-mentioned compounds per se, this invention also encompasses pharmaceutically acceptable derivatives of these compounds and compositions comprising one or more compounds of the invention and one or more pharmaceutically acceptable excipients or additives.

### Definitions

As used herein, “migrating cancer” refers to a cancer in which the cancer cells forming the tumor migrate and subsequently grow as malignant implants at a site other than the site of the original tumor. The cancer cells migrate via seeding the surface of the peritoneal, pleural, pericardial, or subarachnoid spaces to spread into the body cavities; via invasion of the lymphatic system through invasion of lymphatic cells and transport to regional and distant lymph nodes and then to other parts of the body; via haematogenous spread through invasion of blood cells; or via invasion of the surrounding tissue. Migrating cancers include metastatic tumors and cell migration cancers, such as ovarian cancer, mesothelioma, and primary lung cancer, each of which is characterized by cellular migration.

As used herein, “slowing the spread of migrating cancer” refers to reducing or stopping the formation of new loci; or reducing, stopping, or reversing the tumor load. In some embodiments, slowing the spread of migrating cancer comprises contacting a cell with an effective amount of an LXR agonist (e.g., LXRβ agonist).

As used herein, “metastatic tumor” refers to a tumor or cancer in which the cancer cells forming the tumor have a high potential to or have begun to, metastasize, or spread from one location to another location or locations within a subject, via the lymphatic system or via haematogenous spread, for example, creating secondary tumors within the subject. Such metastatic behavior may be indicative of malignant tumors. In some cases, metastatic behavior may be associated with an increase in cell migration and/or invasion behavior of the tumor cells.

As used herein, “slowing the spread of metastasis” refers to reducing or stopping the formation of new loci; or reducing, stopping, or reversing the tumor load. In some embodiments, slowing the spread of metastasis comprises contacting a cell with an effective amount of an LXR agonist (e.g., LXRβ agonist).

The term “cancer” refers to any cancer caused by the proliferation of malignant neoplastic cells, such as tumors, neoplasms, carcinomas, sarcomas, leukimias, lymphomas, and the like.

As used herein, “drug resistant cancer” refers to any cancer that is resistant to an antiproliferative in Table 2.

Examples of cancers that can be defined as metastatic include but are not limited to non-small cell lung cancer, breast cancer, ovarian cancer, colorectal cancer, biliary tract cancer, bladder cancer, brain cancer including glioblastomas and medullablastomas, cervical cancer, choriocarcinoma, endometrial cancer, esophageal cancer, gastric cancer, hematological neoplasms, multiple myeloma, leukemia, intraepithelial neoplasms, livercancer, lymphomas, neuroblastomas, oral cancer, pancreatic cancer, prostate cancer, sarcoma, skin cancer including melanoma, basocellular cancer, squamous cell cancer, testicular cancer, stromal tumors, germ cell tumors, thyroid cancer, and renal cancer.

“Proliferation” as used in this application involves reproduction or multiplication of similar forms (cells) due to constituting (cellular) elements.

“Cell migration” as used in this application involves the invasion by the cancer cells into the surrounding tissue and the crossing of the vessel wall to exit the vasculature in distal organs of the cancer cell.

By “cell migration cancers” is meant cancers that migrate by invasion by the cancer cells into the surrounding tissue and the crossing of the vessel wall to exit the vasculature in distal organs of the cancer cell.

“Non-metastatic cell migration cancer” as used herein refers to cancers that do not migrate via the lymphatic system or via haematogenous spread.

As used herein, “cell to cell adhesion” refers to adhesion between at least two cells through an interaction between a selectin molecule and a selectin specific ligand. Cell to cell adhesion includes cell migration.

A “cell adhesion related disorder” is defined herein as any disease or disorder which results from or is related to cell to cell adhesion or migration. A cell adhesion disorder also includes any disease or disorder resulting from inappropriate, aberrant, or abnormal activation of the immune system or the inflammatory system. Such diseases include but are not limited to, myocardial infarction, bacterial or viral infection, metastatic conditions, e.g. cancer. The invention further features methods for treating a cell adhesion disorder by administering an LXR agonist or ApoE polypeptide.

As used herein, “cancer stem cells” or “cancer initiating cells” refers to cancer cells that possess characteristics associated with normal stem cells, specifically the ability to give rise to all cell types found in a particular cancer sample. Cancer stem cells are therefore tumorgenic or tumor forming, perhaps in contrast to other non-tumorgenic cancer cells. Cancer stem cells may persist in tumors as a distinct population and cause cancer recurrence and metastasis by giving rise to new tumors.

As used herein, “tumor seeding” refers to the spillage of tumor cell clusters and their subsequent growth as malignant implants at a site other than the site of the original tumor.

As used herein, “metastatic nodule” refers to an aggregation of tumor cells in the body at a site other than the site of the original tumor.

The term “PD-1 inhibitor,” as used herein, refers to a compound such as an antibody capable of inhibiting the activity of the protein that in humans is encoded by the PDCD1 gene. Known PD-1 inhibitors include nivolumab, pembrolizumab, pidilizumab, BMS 936559, and MPDL328OA.

The term “PD-L1 inhibitor,” as used herein, refers to a compound such as an antibody capable of inhibiting the activity of the protein that in humans is encoded by the CD274 gene. Known PD-L1 inhibitors include atezolizumab and MED14736.

The term “CTLA-4 inhibitor,” as used herein, refers to a compound such as an antibody capable of inhibiting the activity of the protein that in humans is encoded by the CTLA4 gene. Known CTLA-4 inhibitors include ipilimumab.

The term “CSF-1R inhibitors,” as used herein refers to a compound such as an antibody capable of inhibiting the activity of the protein that in humans is encoded by the CSF1R gene. Known CSF-1R inhibitors include pexidartinib and AZD6495.

The term “IDO inhibitor,” as used herein, refers to a compound such as an antibody capable of inhibiting the activity of the protein that in humans is encoded by the IDO1 gene. Known IDO inhibitors include norharmane, rosmarinic acid, and alpha-methyl-tryptophan.

The term “A1 adenosine inhibitor,” as used herein, refers to a compound such as an antibody capable of inhibiting the activity of the protein that in humans is encoded by the ADORA1 gene. Known A1 adenosine inhibitors include 8-cyclopentyl-1,3-dimethylxanthine, 8-cyclopentyl-1,3-dipropylxanthine, 8-phenyl-1,3-dipropylxanthine, bamifylline, BG-9719, BG-9928, FK-453, FK-838, rolofylline, and N-0861.

The term “A2A adenosine inhibitor,” as used herein, refers to a compound such as an antibody capable of inhibiting the activity of the protein that in humans is encoded by the ADORA2A gene. Known A2A adenosine inhibitors include ATL-4444, istradefylline, MSX-3, preladenant, SCH-58261, SCH-412,348, SCH-442,416, ST-1535, VER-6623, VER-6947, VER-7835, viadenant, and ZM-241,385.

The term “A2B adenosine inhibitor,” as used herein, refers to a compound such as an antibody capable of inhibiting the activity of the protein that in humans is encoded by the ADORA2B gene. Known A2B adenosine inhibitors include ATL-801, CVT-6883, MRS-1706, MRS-1754, OSIP-339,391, PSB-603, PSB-0788, and PSB-1115.

The term “A3A adenosine inhibitor,” as used herein, refers to a compound such as an antibody capable of inhibiting the activity of the protein that in humans is encoded by the ADORA3 gene. Known A3A adenosine inhibitors include KF-26777, MRS-545, MRS-1191, MRS-1220, MRS-1334, MRS-1523, MRS-3777, MRE-3005-F20, MRE-3008-F20, PSB-11, OT-7999, VUF-5574, and SSR161421.

The term “arginase inhibitor,” as used herein, refers to a compound capable of inhibiting the activity of a protein that in humans is encoded by the ARG1 or ARG2 genes. Known arginase inhibitors include (2s)-(+)-amino-5-iodoacetamidopentanoic acid, NG-hydroxy-L-arginine, (2S)-(+)-amino-6-iodoacetamidohexanoic acid, and (R)-2-amino-6-borono-2-(2-(piperidin-1-yl)ethyl)hexanoic acid.

The term “HDAC inhibitor,” as used herein, refers to a compound such as an antibody that is capable of inhibiting the activity of the protein that is a member of the histone deacetylase class of enzymes, e.g., HDAC1, HDAC2, HDAC3, HDAC4, HDAC5, HDAC6, HDAC7, HDAC8, HDAC9, HDAC10, HDAC11, SIRT1, SIRT2, SIRT3, SIRT4, SIRT5, SIRT6, and SIRT7. Known HDAC inhibitors include valproic acid, SAHA, and romidepsin.

The details of one or more embodiments of the invention are set forth in the description below. Other features, objects, and advantages of the invention will be apparent from the description and from the claims.

### BRIEF DESCRIPTION OF THE DRAWINGS

FIG. 1. Treatment with the LXR agonist GW3965 elevates melanoma cell ApoE levels and suppresses cancer cell invasion, endothelial recruitment, and metastatic colonization. (A-B) Parental MeWo cells were incubated in the presence of DMSO or GW3965 at the indicated concentrations. After 48 hours, total RNA was extracted, and the levels of ApoE (A) and DNAJA4 (B) were determined by qRT-PCR. n=3. (C) Cell invasion by 1×10⁵ parental MeWo cells pre-treated with GW3965 or DMSO for 48 hours. n=6-7. p-values based on a one-sided student's t-test. All data are represented as mean±SEM. (D) Endothelial recruitment by 5×10⁴ parental MeWo cells pre-treated with GW3965 or DMSO for 48 hours. n=6-7. p-values based on a one-sided student's t-test. (E) Mice were fed with grain-based chow diet containing GW3965 (20 mg/kg) or a control diet. After 10 days, 4×10⁴ parental MeWo cells were tail-vein injected into mice, and the mice were continuously fed with GW3965-containing chow or a control diet throughout the experiment. Lung colonization was assessed by bioluminescence imaging. n=5-6; p-values obtained using a one-way Mann-Whitney t-test All data are represented as mean±SEM.

FIG. 2. Activation of LXRβ Signaling Suppresses Melanoma Cell Invasion and Endothelial Recruitment. (A) Heat-map depicting microarray-based expression levels of LXR and RXR isoforms in the NCI-60 melanoma cell line collection. The heat map for these genes is extracted from the larger nuclear hormone receptor family heat map (FIG. 3). Color-map key indicates the change in standard deviations for the expression value of each receptor relative to the average expression value of all microarray-profiled genes (>39,000 transcript variants) in each cell line. (B) Cell invasion by 1×10⁵ MeWo, 5×10⁴ HT-144, 5×10⁵ SK-Mel-2, and 5×10⁴ SK-Mel-334.2 human melanoma cells. Cells were treated with DMSO, GW3965, T0901317, or Bexarotene at 1 μM for 72 hours and subjected to a trans-well matrigel invasion assay. n=4-8. (C) 5×10⁴ MeWo, HT-144, SK-Mel-2, and SK-Mel-334.2 human melanoma cells were tested for their ability to recruit 1×10⁵ endothelial cells in a trans-well migration assay, following treatment of the melanoma cells with DMSO, GW3965, T0901317, or Bexarotene at 1 μM for 72 hours. n=4-8. (D-E) 1×10⁵ MeWo (D) and 1×10⁵ HT-144 (E) melanoma cells expressing a control shRNA or shRNAs targeting LXRα or LXRβ were subjected to the cell invasion assay following treatment of the cells with DMSO, GW3965, or T0901317 at 1 μM for 72 hours. n=4-12. (F-G) 5×10⁴ MeWo (F) and 5×10⁴ HT-144 (G) cells, transduced with lentiviral shRNAs targeting LXRα or LXRβ or a control shRNA, were treated with DMSO, GW3965, or T0901317 at 1 μM for 72 hours and tested for their ability to recruit 1×10⁵ endothelial cells in a trans-well migration assay. n=7-8. All data are represented as mean±SEM. Scale bar, 50 μm. *p<0.05, **p<0.01, ***p<0.001, ****p<0.0001.

FIG. 3. Analysis of Nuclear Hormone Receptor Expression in Melanoma and Effects of LXR and RXR Agonists on In Vitro Cell Growth, Related to FIG. 2(A-G). (A) Heat-map showing microarray-based expression levels of all nuclear hormone receptor family members across the NCI-60 collection of melanoma lines. The expression levels of each receptor is presented as the number of standard deviations below or above the average expression levels of all genes (>39,000 transcript variants) detected by the microarray in each respective cell line. (B) 2.5×10⁴ MeWo, HT-144, or SK-Mel-334.2 human melanoma cells were seeded in 6-well plates and cultured in the presence of DMSO, GW3965, T0901317, or Bexarotene at 1 μM. Viable cells were counted on day 5 post-seeding. n=3-6. (C) 2.5×10⁴ MeWo, HT-144, or SK-Mel-334.2 cells were plated in triplicates and incubated in media containing DMSO, GW3965, T0901317, or Bexarotene at 1 μM for 5 days, after which the number of dead cells was quantified using trypan blue dead cell stain. n=3. (D-G) Relative expression of LXRα and LXRβ, determined by qRT-PCR, in MeWo (D, E) and HT-144 (F, G) human melanoma cells expressing a control shRNA or shRNAs targeting LXRα or LXRβ. All data are represented as mean±SEM.

FIG. 4. Therapeutic LXR Activation Inhibits Melanoma Tumor Growth. (A-B) Primary tumor growth by 5×10⁴ B16F10 mouse melanoma cells subcutaneously injected into C57BL/6-WT mice. Following tumor growth to 5-10 mm³ in volume, mice were continuously fed a control chow or a chow supplemented with GW3965 (20 mg/kg/day or 100 mg/kg/day) (A) or T0901317 (20 mg/kg/day) (B). Representative tumor images shown correspond to tumors extracted at the final day (d12). n=10-18 (A), 8-10 (B). (C-E) Primary tumor growth by 1×10⁶ MeWo (C), 7.5×10s SK-Mel-334.2 (D), and 2×10⁶ SK-Mel-2 (E) human melanoma cells subcutaneously injected into immunocompromised mice. Following tumor growth to 5-10 mm³ in volume, mice were randomly assigned to a control diet or a diet supplemented with GW3965 (20 mg/kg or 100 mg/kg, as indicated). Tumor images shown correspond to last day of measurements. n=6-34 (C), 8 (D), 5 (E). (F) 5×10⁴ B16F10 cells were injected subcutaneously into C57BL/6-WT mice. Upon tumor growth to 150 mm³, mice were fed continuously with a control chow or a chow containing GW3965 (150 mg/kg), and tumor growth was measured daily. n=6-13. (G-I) Mouse overall survival following subcutaneous grafting of 5×10⁴ B16F10 (G), 1×10⁶ MeWo (H), and 7.5×10s SK-Mel-334.2 cells (I) into mice that were administered a normal chow or a chow supplemented with GW3965 (100 mg/kg) upon formation of tumors measuring 5-10 mm³ in volume. n=6-9 (F), 4-7(H), 3-6 (I). (J-L) Tumor endothelial cell density, determined by immunohistochemical staining for the mouse endothelial cell antigen MECA-32 (J), tumor cell proliferation, determined by staining for the proliferative marker Ki-67 (K), and tumor cell apoptosis, determined by staining for cleaved caspase-3 (L), in subcutaneous melanoma tumors formed by 1×10⁶ MeWo human melanoma cells in response to mouse treatment with a control diet or a GW3965-supplemented diet (20 mg/kg) for 35 days. n=5. Tumor volume was calculated as (small diameter)²×(large diameter)/2. All data are represented as mean±SEM. Scale bars, 5 mm (A-D), 50 μm (J, K), 25 μm (L).

FIG. 5. LXRβ Agonism Suppresses Melanoma Tumor Growth, Related to FIG. 4(A-E). (A) Weight measurements of mice fed a control diet or a diet supplemented with GW3965 (20 mg/kg/day or 100 mg/kg/day) or T0901317 (20 mg/kg) for 65 days. n=5-6.

FIG. 6. LXR Agonism Suppresses Melanoma Metastasis to the Lung and Brain. (A) MeWo cells were pre-treated with DMSO or GW3965 (1 μM) for 48 hours and 4×10⁴ cells were intravenously injected via the tail-vein into NOD Scid mice. Lung colonization was monitored by weekly bioluminescence imaging. Representative H&E-stained lungs correspond to the final day (d70) are shown. n=4-5. (B-C) Bioluminescence imaging of lung metastasis by 4×10⁴ MeWo cells intravenously injected into NOD Scid mice that were fed a control chow or a chow containing GW3965 (20 mg/kg) or T0901317 (20 mg/kg) starting 10 days prior to cancer cell injection. Representative H&E-stained lungs correspond to final imaging day n=5-6. (D-E) Bioluminescence imaging of lung metastasis by HT-144 cells (D) or SK-Mel-334.2 cells (E) intravenously injected into NOD Scid mice that were fed a control chow or a chow containing GW3965 or T0901317. (F) Systemic and brain photon flux following intracardiac injection of 1×10⁵ MeWo brain metastatic derivative cells into athymic nude mice that were fed a control diet or a GW3965-supplemented diet (100 mg/kg) starting on day 0 post-injection. n=7. (G) Schematic of experimental orthotopic metastasis model used to assess the ability of GW3965 treatment to suppress lung metastasis post-tumor excision. (H) Ex-vivo lung photon flux, determined by bioluminescence imaging, in NOD Scid mice that were administered a control chow or a chow containing GW3965 (100 mg/kg) for 1 month following the excision of size-matched (˜300-mm³ in volume) subcutaneous melanoma tumors formed by 1×10⁶ MeWo melanoma cells. Representative lungs stained for human vimentin are also shown. n=7-9. (I) 4×10⁴ MeWo cells were intravenously injected into NOD Scid mice. Following initiation of metastases, detected by bioluminescence imaging on d42, mice were administered a control diet or a GW3965 diet (100 mg/kg) as indicated, and lung colonization progression was measured weekly. n=6. (J) Number of macroscopic metastatic nodules in H&E-stained lungs extracted at the final day (d77) from NOD Scid mice administered a control diet or a diet supplemented with GW3965 (100 mg/kg), as indicated in (I). n=4-5. (K) Overall mouse survival following intravenous injection of 4×10⁴ MeWo cells into NOD-Scid mice that were continuously fed a control chow or a GW3965-supplemented chow (20 mg/kg) starting 10 days prior to cancer cell injection. n=5-6. All data are represented as mean±SEM.

FIG. 7. Suppression of Genetically-Driven Melanoma Progression by LXR Activation Therapy. (A) Overall survival of Tyr::CreER; Braf^{V600E/+}; Pten^{lox/+} C57BL/6 mice following general melanoma induction by intraperitoneal administration of 4-HT (25 mg/kg) on three consecutive days. After the first 4-HT injection, mice were randomly assigned to a control diet or a diet supplemented with GW3965 (100 mg/kg). n=10-11. (B) Melanoma tumor burden, expressed as the percentage of dorsal skin area, measured on day 35 in Tyr::CreER; Braf^{V600E/+}; Pten^{lox/lox} mice administered a control chow or a chow supplemented with GW3965 (100 mg/kg) upon melanoma induction as described in (A). n=4-5. (C) Number of macroscopic metastatic nodules to the salivary gland lymph nodes detected post-mortem in Tyr::CreER; Braf^{V600E/+}; Pten^{lox/lox} mice that were fed a control chow or a chow containing GW3965 (100 mg/kg) following global induction of melanoma progression as described in (A). n=7-8. (D) Tumor growth following subcutaneous injection of 1×10⁵ Braf^{V600E/+}; Pten^{-/-}; CDKN2A^{-/-} primary melanoma cells into syngeneic C57BL/6-WT mice. Upon tumor growth to 5-10 mm³ in volume, mice were fed with a control chow or a chow supplemented with GW3965 (100 mg/kg). n=16-18. (E) Overall survival of C57BL/6-WT mice subcutaneously injected with 1×10⁵ Braf^{V600E/+}; Pten^{-/-}; CDKN2A^{-/-} melanoma cells and treated with a GW3965 diet (100 mg/kg) or a control diet following tumor growth to 5-10 mm³ in volume. n=7-8. (F) Lung colonization by 1×10⁵ Braf^{V600E/+}; Pten^{-/-}; CDKN2A′ primary melanoma cells intravenously injected into C57BL/6-WT mice. Immediately following cancer cell injection, mice were randomly assigned to a control diet or a GW3965-supplemented diet (100 mg/kg) for the remainder of the experiment. n=14-15. All data are represented as mean±SEM. Scale bar, 2 mm (B), 5 mm (D).

FIG. 8. LXR-Mediated Suppression of Melanoma Progression in a Genetically-Driven Melanoma Mouse Model, Related to FIG. 7 (A-C). (A) Overall survival of Tyr::CreER; Braf^{V600E/+}; Pten^{lox/lox} C57BL/6 mice following general melanoma induction by intraperitoneal administration of 4-HT (25 mg/kg) on three consecutive days. After the first 4-HT injection, mice were randomly assigned to a control diet or a diet supplemented with GW3965 (100 mg/kg). n=7. (B) Representative images of Tyr::CreER; Braf^{V600E/+}; Pten^{lox/lox} C57BL/6 mice fed a control diet of GW3965-supplemented diet (100 mg/kg) taken 43 days following melanoma induction by intraperitoneal 4-HT administration.

FIG. 9. A List of the 50 most upregulated genes in MeWo human melanoma cells in response to GW3965 treatment.

FIG. 10. LXRβ Activation Induces ApoE Expression in Melanoma Cells; ApoE mediates LXRβ-Dependent Suppression of In Vitro Melanoma Progression Phenotypes. (A-C) MeWo (A), HT-144 (B), and WM-266-4 (C) human melanoma cells were treated with GW3965 or T0901317 at the indicated concentrations for 48 hours, and the expression levels of ApoE were analysed by qRT-PCR. n=3. (D) Extracellular ApoE protein levels, quantified by ELISA, in serum-free conditioned media collected from HT-144 human melanoma cells treated with DMSO, GW3965, or T0901317 at 1 μM for 72 hours. n=3-4. (E-F) 5×10⁴ HT-144 cells, treated with DMSO, GW3965, or T0901317 at 1 μM for 72 hours, were tested for the cell invasion (E) and endothelial recruitment phenotypes (F) in the presence of an ApoE neutralization antibody (1D7) or an IgG control antibody added at 40 μg/mL to each trans-well at the start of the assay. n=4. (G-H) Cell invasion (G) and endothelial recruitment (F) by 1×10⁵ and 5×10⁴ MeWo cells, respectively, expressing a control shRNA or an shRNA targeting ApoE and treated with DMSO or GW3965 at 1 μM for 72 hours prior to each assay. n=7-8. (I-J) Relative ApoE expression, quantified by qRT-PCR, in MeWo (I) and HT-144 (J) cells transduced with a control shRNA or shRNAs targeting LXRα or LXRβ and subsequently treated with DMSO, GW3965, or T0901317 at 1 μM for 48 hours. n=3-9. (K) Extracellular ApoE protein levels, measured by ELISA, in serum-free conditioned media harvested from HT-144 cells transduced with a control shRNA or an shRNA targeting LXRα or LXRβ and treated with DMSO or GW3965 at 1 μM for 72 hours. n=3. All data are represented as mean±SEM. Scale bar, 50 μm.

FIG. 11. LXRβ Activation Suppresses Melanoma Invasion and Endothelial Recruitment by Transcriptionally Enhancing Melanoma-Cell ApoE Expression. (A) Luciferase activity driven off the ApoE promoter fused downstream of multi-enhancer element 1 (ME.1) or multi-enhancer element 2 (ME.2) sequences and transfected into MeWo cells treated with DMSO, GW3965, or T0901317 at 1 μM for 24 hours. n=4-8. (B) Extracellular ApoE protein levels were quantified by ELISA in serum-free conditioned media harvested from MeWo cells treated with DMSO, GW3965, or T0901317 at 1 μM for 72 hours. n=3-4. (C) Cell invasion by 1×10⁵ MeWo cells pre-treated with DMSO, GW3965, or T0901317 at 1 μM for 72 hours. At the start of the assay, an ApoE neutralization antibody (1D7) or an IgG control antibody was added at 40 μg/mL to each trans-well, as indicated. n=7-8. (D) 5×10⁴ MeWo cells, pre-treated with DMSO, GW3965, or T0901317 at 1 μM for 72 hours, were tested for their ability to recruit 1×10⁵ endothelial cells in the presence of 1 D7 or IgG antibodies at 40 μg/mL. n=6-8. (E) Extracellular ApoE protein levels, quantified by ELISA, in serum-free conditioned media from SK-Mel-334.2 primary human melanoma cells treated with DMSO or GW3965 at 1 μM for 72 hours. n=4. (F-G) 5×10⁴ SK-Mel-334.2 cells, pre-treated with GW3965 at 1 μM for 72 hours, were subjected to the cell invasion (F) and endothelial recruitment (G) assays in the presence of 1 D7 or IgG antibodies at 40 μg/mL. n=7-8. (H) Activity of the ApoE promoter fused to ME.1 or ME.2 enhancer elements was determined through measuring luciferase reporter activity in MeWo cells expressing a control shRNA or shRNAs targeting LXRα or LXRβ in the presence of DMSO or GW3965 (1 μM) for 24 hours. n=3-8. (I) Extracellular ApoE protein levels, quantified by ELISA, were assessed in serum-free conditioned media collected from human MeWo melanoma cells expressing a control shRNA or shRNAs targeting LXRα or LXRβ in response to treatment with GW3965 or T0901317 (1 μM) for 72 hours. n=3-8. All data are represented as mean±SEM. Scale bar, 50 μm.

FIG. 12. Therapeutic Delivery of LXR Agonists Upregulates Melanoma-Derived and Systemic ApoE Expression. (A-B) ApoE expression levels, quantified by qRT-PCR, in subcutaneous tumors formed by B16F10 mouse melanoma cells injected into C57BL/6 mice. After 5-mm³ tumor formation, mice were fed a control diet or diet containing GW3965 (20 mg/kg) (A) or T0901317 (20 mg/kg) (B) for 7 days. n=3-4. (C-E) ApoEtranscript expression in primary tumors (C), lung metastases (D), and brain metastases (E) formed by MeWo human melanoma cells grafted onto NOD Scid mice that were administered control chow or chow supplemented with GW3965 (20 mg/kg). ApoE levels were assessed on day 35 (C), day 153 (D), and day 34 (E) post-injection of the cancer cells. n=3-5. (F) Relative expression levels of LXRα, LXRβ, and ApoE were determined by qRT-PCR in B16F10 mouse melanoma cells expressing a control hairpin or an shRNA targeting mouse LXRα (sh_mLXRα), mouse LXRβ (sh_mLXRβ), or mouse ApoE (sh_mApoE). (G-H) ApoE (G) and ABCA1(H) mRNA levels, measured by qRT-PCR, in B16F10 cells expressing a control shRNA or shRNAs targeting mouse LXRβ or mouse ApoE. The cells were treated with DMSO or GW3965 at 5 μM for 48 hours. n=3. (I) ABCA1 mRNA levels, measured by qRT-PCR, in systemic white blood cells extracted from LXRα −/− or LXRβ −/− mice fed a control diet or a GW3965-supplemented diet (20 mg/kg) for 10 days. n=3-4. (J) Relative expression of ApoE mRNA, expressed as the frequency of SAGE tags, in mouse skin and lung tissues was determined using the public mSAGE Expression Matrix database available through the NCI-funded Cancer Genome Anatomy Project (CGAP). (K) Relative expression of ApoE mRNA, determined by qRT-PCR, in MeWo melanoma cells dissociated from lung metastatic nodules (LM2) or primary tumors relative to control unselected MeWo parental cells. n=3.

FIG. 13. LXRβ Agonism Suppresses Melanoma Tumor Growth and Metastasis by Inducing Melanoma-Derived and Systemic ApoE Expression. (A) Western blot measurements of ApoE protein levels in adipose, lung, and brain tissue lysates extracted from wild-type mice fed with a control chow or a chow supplemented with GW3965 (20 mg/kg) or T0901317 (20 mg/kg) for 10 days. (B) Quantification of ApoE protein expression based on western blots shown in (A). Total tubulin was used as an endogenous control for normalization. n=3-5. (C) Expression levels of ApoE, determined by qRT-PCR, in systemic white blood cells from mice fed a control diet or a diet supplemented with GW3965 or T0901317 at 20 mg/kg for 10 days. n=3-6. (D) B16F10 control cells or B16F10 cells expressing shRNAs targeting mouse LXRα (sh_mLXRα) or mouse LXRβ (sh_mLXRβ) were subcutaneously injected into C57BL/6-WT, LXRα−/−, or LXRβ−/− mice. Once the tumors reached 5-10 mm³ in volume, mice were fed a control diet or a diet supplemented with GW3965 (20 mg/kg) for 7 days, after which final tumor volume was measured. Representative tumor images extracted at the end point are shown in the right panel. n=6-18. (E) ApoE transcript levels, quantified by qRT-PCR, in systemic white blood cells extracted from LXRα −/− or LXRβ −/− mice fed a control diet or a GW3965-supplemented diet (20 mg/kg) for 10 days. n=3-5. (F) Subcutaneous tumor growth by 5×10⁴ B16F10 control cells or B16F10 cells expressing an shRNA targeting mouse ApoE (sh_mApoE) in C57BL/6-WT or ApoE−/− mice. Following the formation of tumors measuring 5-10 mm³ in volume, mice were fed a control diet or a diet supplemented with GW3965 (20 mg/kg) for 7 days, and final tumor volume was quantified. Representative images of tumors extracted at the final day of measurement (d12) are shown on the right. n=8-18. (G) Lung colonization by 5×10⁴ B16F10 cells transduced with a control shRNA or sh_mApoE and intravenously injected into C57BL/6-WT or ApoE−/− mice. Starting 10 days prior to cancer cell injection, mice were assigned to a control diet or a GW3965-supplemented diet (20 mg/kg) treatment. Lung metastasis was quantified on d22 by bioluminescence imaging. Representative lungs extracted at the end point (d22) are shown in the right panel. n=5-10. (H) ApoE protein expression, determined by blinded immunohistochemical analysis, in non-metastatic (n=39) and metastatic (n=34) primary melanoma skin lesion samples obtained from patients at MSKCC. The fraction of ApoE-positively staining cell area was quantified as a percentage of total tumor area. (I) Kaplan-Meier curves for the MSKCC cohort (n=71) depicting the metastasis-free survival of patients as a function of ApoE protein expression in patients' primary melanoma lesions. Melanomas that had ApoE levels above the median of the population were classified as ApoE-positive (pos), whereas tumors with ApoE expression below the median were classified as ApoE-negative (neg). All data are represented as mean±SEM. Scale bar, 5 mm (D and F), 100 μm (H).

FIG. 14. Activation of LXRβ Suppresses the In Vivo Growth of Melanoma Lines Resistant to Dacarbazine and Vemurafenib. (A) In vitro cell growth by 2.5×10⁴ B16F10 parental cells and in vitro-derived B16F10 DTIC-resistant cells in response to varying doses of dacarbazine (DTIC) added to the cell media for 4 days. n=3. (B-D) Tumor growth by 5×10⁴ DTIC-sensitive B16F10 parental cells (B) or 5×10⁴ DTIC-resistant B16F10 cells (C) subcutaneously injected into C57BL/6-WT mice. Following tumor growth to 5-10 mm³ in volume, mice were treated with dacarbazine (50 mg/kg, i.p., daily) or a control vehicle and randomly assigned to regular chow or a chow supplemented with GW3965 (100 mg/kg). Final day tumor volume measurements are shown in (D). n=8-16 (B), 7-8 (C). (E-F) Tumor growth by DTIC-sensitive MeWo parental cells and in vivo-derived DTIC-resistant MeWo human melanoma cells in response to DTIC or GW3965 treatments. 5×10⁵ cells were subcutaneously injected into NOD Scid gamma mice. After formation of tumors measuring 5-10 mm³ in volume, mice were blindedly assigned to a control treatment, a DTIC treatment (50 mg/kg, i.p., administered daily in 5-day cycles with 2-day off-treatment intervals), or a GW3965-supplemented diet treatment (100 mg/kg). Final day tumor measurements are show in (F). n=6-8. (G) Tumor growth by 2×10⁶ SK-Mel-239 vemurafenib-resistant clone cells subcutaneously injected into NOD Scid gamma mice that were assigned to a control diet or a diet supplemented with GW3965 (100 mg/kg) subsequent to growth of tumors to 5-10 mm³ in volume. n=7-8. (H) Overall mouse survival post-grafting of 2×10⁶ SK-Mel-239 vemurafenib-resistant cells. Upon the growth of tumors to 5-10 mm³ in volume, mice were continuously fed a control diet or a diet supplemented with GW3965 (100 mg/kg). n=7. (I) Experimentally derived model depicting the engagement of systemic and melanoma-autonomous ApoE by LXRβ activation therapy in mediating the suppression of melanoma progression phenotypes. Extracellular ApoE suppresses melanoma metastasis by coordinately inhibiting melanoma cell invasion and non-cell-autonomous endothelial recruitment through targeting melanoma-cell LRP1 and endothelial-cell LRP8 receptors, respectively. All data are represented as mean±SEM. Scale bar, 5 mm.

FIG. 15. Dacarbazine-Induced Suppression of Tumor Growth by Human Melanoma Cells. (A) Tumor growth by 5×10⁵ DTIC-sensitive MeWo parental cells subcutaneously injected into Nod SCID gamma mice. When tumors reached 5-10 mm³ volume, mice were treated with a control vehicle or DTIC (50 mg/kg, i.p., administered daily in 5-day cycles with 2-day off-treatment intervals), and tumor volume was measured twice a week. n=6.

FIG. 16. Effects of LXR agonists LXR-623, WO-2007-002563 Ex. 19, WO-2010-0138598 Ex. 9, and SB742881 on ApoE expression in human melanoma cells. (A-D) MeWo human melanoma cells were treated with DMSO or the LXR agonists LXR-623 (A), WO-2007-002563 (B), WO-2010-0138598 (C), or SB742881 (D) at 500 nM, 1 μM, or 2 μM for 48 hours. The expression levels of ApoE were subsequently quantified by qRT-PCR. n=3. All data are represented as mean±SEM. *p<0.05, **p<0.01.

FIG. 17. Treatment with the LXR agonist GW3965 inhibits In Vitro tumor cell invasion of renal cancer, pancreatic cancer, and lung cancer. (A-C) Trans-well matrigel invasion by 5×10⁴ RCC human renal cancer cells (A), 5×10⁴ PANC1 human pancreatic cancer cells (B), and 5×10⁴H460 human lung cancer cells (C) that were treated with DMSO or GW3965 at 1 μM for 72 hours prior to the assay. n=4. All data are represented as mean±SEM. *p<0.05, **p<0.01.

FIG. 18. Treatment with the LXR agonist GW3965 inhibits breast cancer tumor growth In Vivo. Primary tumor growth by 2×10⁶ MDA-468 human breast cancer cells injected into the mammary fat pads of NOD Scid gamma mice. Two days prior to cancer cell injection, the mice were assigned to a control diet treatment or a diet supplemented with GW3965 (75 mg/kg) and maintained on the corresponding diet throughout the experiment. n=8. All data are represented as mean±SEM. ***p<0.001.

FIG. 19. Effects of LXR agonists LXR-623, WO-2007-002563 Ex. 19, WO-2010-0138598 Ex. 9, and SB742881 on in vitro melanoma progression phenotypes. (A) Cell invasion by 1×10⁵ MeWo human melanoma cells pre-treated with DMSO, LXR-623, WO-2007-002563 Ex. 19, WO-2010-0138598 Ex. 9, or SB742881 at 1 μM each for 72 hours. The number of cells invading into the basal side of matrigel-coated trans-well inserts was quantified. n=5. (B) Endothelial recruitment by 5×10⁴ MeWo cells pre-treated with DMSO, LXR-623, WO-2007-002563 Ex. 19, WO-2010-0138598 Ex.9, or SB742881 at 1 μM each for 72 hours. Cancer cells were seeded at the bottom of a 24-well plate. Endothelial cells were seeded in a trans-well insert fitted into each well and allowed to migrate towards the cancer cells. The number of endothelial cells migrating to the basal side of each trans-well insert was quantified. n=4-5. All data are represented as mean±SEM. *p<0.05, **p<0.01.

FIG. 20. Effects of LXR agonists LXR-623, WO-2007-002563 Ex. 19, WO-2010-0138598 Ex. 9, and SB742881 on in vivo tumor growth. (A-D) Tumor growth by 5×10⁴ B16F10 mouse melanoma cells subcutaneously injected into 7-week-old C57BL/6 mice. After tumors reached 5-10 mm³ in volume, the mice were randomly assigned to a control diet treatment, an LXR-623-supplemented diet treatment at 20 mg/kg/day (A) a WO-2007-002563 Ex. 19-supplemented diet treatment at 100 mg/kg/day (B), a WO-2010-0138598 Ex. 19-supplemented diet treatment at 10 mg/kg/day or 100 mg/kg/day (C), or an SB742881-supplemented diet treatment at 100 mg/kg/day (D). n=8-10. All data are represented as mean±SEM.

### DETAILED DESCRIPTION OF THE INVENTION

The present invention features methods for preventing or reducing aberrant proliferation, differentiation, or survival of cells. For example, the methods of the invention may be useful in reducing the risk of, or preventing, tumors from increasing in size or from reaching a metastatic state. The subject compounds may be administered to halt the progression or advancement of cancer. In addition, the instant invention includes use of the subject compounds to reduce the risk of, or prevent, a recurrence of cancer.

Metastatic progression requires that sets of effector proteins involved in common cellular phenotypes be coherently expressed (Gupta and Massagué, 2006 Cell 127, 679-695; Hanahan and Weinberg, 2011 Cell 144, 646-674; Talmadge and Fidler, 2010 Cancer Res. 70, 5649-5669; Hynes, 2003 Cell 113, 821-823). Such concerted expression states are apparent in gene expression profiles of primary breast cancers that metastasize (Wang et al., 2005 Lancet 365, 671-679), as well as profiles of human cancer cell clones that display enhanced metastatic activity (Kang et al., 2003 Cancer Cell 3, 537-549; Minn et al., 2005 Nature 436, 518-524). In recent years, post-transcriptional regulation has emerged as a pervasive and robust mode of concerted expression-state and phenotype-level control. The most studied class of post-transcriptional regulators with metastatic regulatory activity are small non-coding RNAs (miRNAs) (Bartel, 2009 Cell 136, 215-233; Fabian et al., 2010 Annu. Rev. Biochem, 79, 351-379; Filipowicz et al., 2008 Nat. Rev. Genet. 9, 102-114). Metastasis promoter miRNAs (Ma et al., 2007 Nature 449, 682-688; Huang et al., 2008 Nat. Cell Biol. 10, 202-210) and suppressor miRNAs (Tavazoie et al., 2008 Nature 451, 147-152) were originally discovered in breast cancer. Subsequent studies revealed many more miRNAs with regulatory roles in the tumorigenesis and metastasis of other cancer types (Hatziapostolou et al., 2011 Cell 147, 1233-1247; Hurst et al., 2009 Cancer Res. 69, 7495-7498; Olson et al., 2009 Genes Dev. 23, 2152-2165; Zhang et al., 2010 Oncogene 29, 937-948) In many cases, the expression levels of these miRNAs in human cancer samples have supported their experimental roles in metastasis. Thus, deregulated miRNA expression (Garzon et al., 2010 Nat. Rev. Drug Discov. 9, 775-789; Lujambio and Lowe, 2012 Nature 482, 347-355) and, more recently, deregulated expression of long non-coding RNAs (Calin et al., 2007 Nat. Rev. Cancer 6, 857-866; Gupta et al., 2010 Nature 464, 1071-1076; Guttman et al., 2009 Nature 458, 223-227; Huarte et al., 2010. Cell 142, 409-419; Loewer et al., 2010 Nat. Genet. 42, 1113-1117) as well as non-coding pseudogenes competing for endogenous miRNA binding (Poliseno et al., 2010 Nature 465, 1033-1038) appear to be pervasive features of human cancer. Clues regarding the robust control exerted by specific miRNAs on metastatic progression came from early work showing that concerted targeting of multiple metastasis genes by a single metastasis suppressor miRNA was responsible for the dramatic metastasis suppression effects (Tavazoie et al., 2008 Nature 451, 147-152). Such divergent gene targeting by miRNAs has appeared to be a defining feature of these regulators.

At a conceptual level, the need for divergent regulation of gene expression in cancer is readily understood. A miRNA could exert robust metastatic suppression by virtue of its ability to target multiple genes required for metastasis. The miRNA's silencing through genetic or epigenetic mechanisms would readily promote cancer progression by de-repressing multiple promoters of metastasis (Png et al., 2011 Nature 481, 190-194). A role for convergent regulation of a single gene by multiple metastasis regulatory miRNAs is more nuanced. This scenario would emerge if there existed a key gene that acted as a robust suppressor of metastatic progression. Convergent and cooperative targeting of this gene by multiple miRNAs could achieve maximal silencing of such a key metastasis suppressor gene. This scenario, as opposed to genetic deletion, may be seen in cases where complete loss of a target gene could not be tolerated by the cell, and the gene would be required at low levels to mediate metabolic actions, for example. Given this possibility, a search for cooperative metastasis promoter miRNAs may uncover novel genes that are pivotal for metastasis suppression and may provide therapeutic insights into more effective treatments for metastasis prevention.

As disclosed herein, via a systematic, in vivo selection-based approach, a set of miRNAs were identified to be deregulated in multiple independent metastatic lines derived from multiple patients with melanoma—a highly prevalent cancer with increasing incidence (Garbe and Leiter, 2009 Clin. Dermatol. 27, 3-9). As disclosed herein, miR-1908, miR-199a-3p, and miR-199a-5p act as robust endogenous promoters of melanoma metastasis through convergent targeting of the metabolic gene ApoE and the heat-shock protein DNAJA4. Through loss-of-function, gain-of-function, and epistatic analyses, a cooperative miRNA network that maximally silences ApoE signaling is delineated. Cancer cell-secreted ApoE inhibits metastatic invasion and endothelial recruitment, which is mediated through its actions on distinct receptors on melanoma and endothelial cells. These miRNAs display significant prognostic capacity in identifying patients that develop melanoma metastatic relapse, while therapeutic delivery of LNAs targeting these miRNAs significantly inhibits melanoma metastasis. The current lack of effective therapies for the prevention of melanoma metastasis after surgical resection (Garbe et al., 2011 Oncologist 16, 5-24) requires an improved molecular and mechanistic understanding of melanoma metastatic progression. To this end, the findings disclosed herein reveal a number of key novel non-coding and coding genes involved in melanoma progression and offer a novel avenue for both identifying patients at high-risk for melanoma metastasis and treating them.

The members of this network can be used as targets for treating cancer (e.g., metastatic melanoma). In addition, the members can be used a biomarkers for determining whether a subject has, or is at risk of having, cancer (e.g., metastatic melanoma) or for determining a prognosis or surveillance of patient having the disorder. Accordingly, the present invention encompasses methods of treating cancer (e.g., metastatic melanoma) by targeting one or more of the members, methods of determining the efficacy of therapeutic regimens for inhibiting the cancer, and methods of identifying anti-cancer agent. Also provided are methods of diagnosing whether a subject has, or is at risk for having, cancer (e.g., metastatic melanoma), and methods of screening subjects who are thought to be at risk for developing the disorder. The invention also encompasses various kits suitable for carrying out the above mentioned methods.

### LXR Agonists

LXR agonists include any compound described herein such as a compound of any one of Formula I-XXVI and/or any one of compounds 1-107, or pharmaceutically acceptable salts thereof.

LXRα and LXRβ, initially discovered by multiple groups at roughly the same time (Apfel et al., 1994; Willy et al., 1995; Song et al., 1994; Shinar et al., 1994; Teboul et al., 1995), belong to a family of nuclear hormone receptors that are endogenously activated by cholesterol and its oxidized derivatives to mediate transcription of genes involved in maintaining glucose, cholesterol, and fatty acid metabolism (Janowski et al., 1996; Calkin and Tontonoz, 2012). Given the intricate link between lipid metabolism and cancer cell growth (Cairns et al., 2011), the ubiquitous expression of LXRβ in melanoma is unlikely to be coincidental, allowing melanoma cells to synthesize lipids and lipoprotein particles to sustain their growth. At the same time, however, such stable basal expression levels make LXRβ an ideal therapeutic target, as exemplified by the broad-ranging responsiveness of melanoma cells to LXRβ activation therapy.

Compounds have been shown to have selectivity for LXRβ or LXRα. This selectivity may allow for increased activity and/or decreased off target effects. Examples of compounds with selectivity towards LXRβ or LXRα are shown in Table 1.

TABLE 1
EC₅₀ values for selected compounds against LXRα and LXRβ

| | | EC₅₀-LXRα | EC₅₀-LXRβ |
|---|---|---|---|
| Compound | Structure | (nM) | (nM) |
| GW3965 108 | 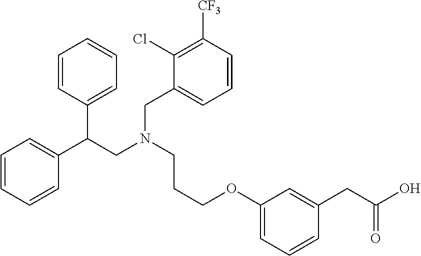 [SMILES: O=C(O)Cc1cccc(OCCCN(Cc2cccc(C(F)(F)F)c2Cl)CC(c2ccccc2)c2ccccc2)c1] | 200 | 40 |
| SB742881 109 | 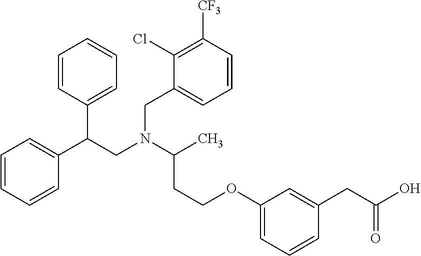 [SMILES: CC(CCOc1cccc(CC(=O)O)c1)N(Cc1cccc(C(F)(F)F)c1Cl)CC(c1ccccc1)c1ccccc1] | 74 | 25 |
| TO901317 110 | 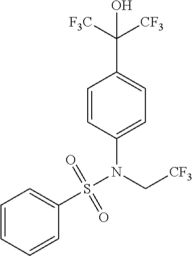 [SMILES: O=S(=O)(c1ccccc1)N(CC(F)(F)F)c1ccc(C(O)(C(F)(F)F)C(F)(F)F)cc1] | 20 | 50 |
| LXR-623 111 | 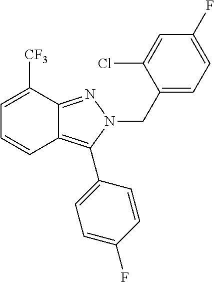 [SMILES: Fc1ccc(-c2c3cccc(C(F)(F)F)c3nn2Cc2ccc(F)cc2Cl)cc1] | 179 | 24 |
| 112 | 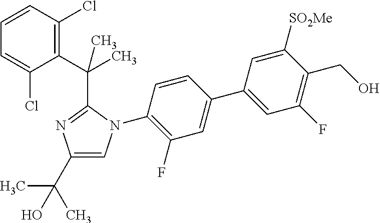 [SMILES: CC(C)(O)c1cn(-c2ccc(-c3cc(F)c(CO)c(S(C)(=O)=O)c3)cc2F)c(C(C)(C)c2c(Cl)cccc2Cl)n1] | <100 | 11 |
| 113 | 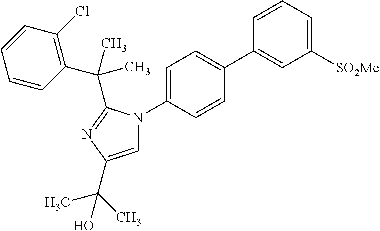 [SMILES: CC(C)(O)c1cn(-c2ccc(-c3cccc(S(C)(=O)=O)c3)cc2)c(C(C)(C)c2ccccc2Cl)n1] | 101-1000 | 630 |

As used herein, reference to the activity of an LXR agonist at LXRα and LXRβ refer to the activity as measured using the ligand sensing assay (LiSA) described in Spencer et al. Journal of Medicinal Chemistry 2001, 44, 886-897, incorporated herein by reference. In some embodiments, the LXR agonist has an EC50 of less than 1 μM in the ligand sensing assay (e.g., 0.5 nm to 500 nM, 10 nM to 100 nM). For example, the methods of the invention can be performed using an LXRβ agonist having activity for LXRβ that is at least 3-fold greater than the activity of the agonist for LXRα, or having activity for LXRβ that is at least 10-fold greater than the activity of the agonist for LXRα, or having activity for LXRβ that is at least 100-fold greater than the activity of said agonist for LXRα, or having activity for LXRβ that is at least within 3-fold of the activity of the agonist for LXRα. The term “greater activity” in the LiSA assay assay refers to a lower EC₅₀. For example, GW3965108 has approximately 6-fold greater activity for LXRβ (EC₅₀=30) compared to LXRα (EC₅₀=190).

As used herein, the term “increases the level of ApoE expression in vitro” refers to certain LXR agonists capable of increasing the level of ApoE expression 2.5-fold in the qPCR assay of Example 21 at a concentration of less than 5 μM (e.g., at a concentration of 100 nM to 2 μM, at a concentration of less than or equal to 1 μM). The LXR agonists exhibiting this in vitro effect can be highly efficacious for use in the methods of the invention.

### Treatment Methods

As disclosed herein, miR-1908, miR-199a-3p, miR-199a-5p, and CTGF were identified as endogenous metastasis promoters of metastatic invasion, endothelial recruitment, and colonization in melanoma while DNAJA4, ApoE, LRP1, LRP8, LXR, and miR7 function as metastasis suppressors or inhibitors of the same process. Cancer-secreted ApoE suppresses invasion and endothelial recruitment by activating melanoma cell LRP1 and endothelial LRP8 receptors, respectively. DNAJA4, in turn, induces ApoE expression. These miRNAs strongly predict human metastatic outcomes.

Accordingly, this invention provides methods for treating cancer via increasing in the subject the expression level or activity level of one of the metastasis suppressors by administration of an effective amount of an LXR agonist. The invention also provides methods for treating in a subject an angiogenic disorder or a disorder of angiogenesis. The terms “angiogenic disorder,” “disorder of angiogenesis,” and “angiogenesis disorder” are used interchangeably herein, and refer to a disorder characterized by pathological angiogenesis. A disorder characterized by pathological angiogenesis refers to a disorder where abnormal or aberrant angiogenesis, alone or in combination with others, contributes to causation, origination, or symptom of the disorder. Examples of this disorder include various cancers (e.g., vascularized tumors), eye disorders, inflammatory disorders, and others.

Typical vascularized tumors that can be treated with the method include solid tumors, particularly carcinomas, which require a vascular component for the provision of oxygen and nutrients. Exemplary solid tumors include, but are not limited to, carcinomas of the lung, breast, bone, ovary, stomach, pancreas, larynx, esophagus, testes, liver, parotid, biliary tract, colon, rectum, cervix, uterus, endometrium, kidney, bladder, prostate, thyroid, squamous cell carcinomas, adenocarcinomas, small cell carcinomas, melanomas, gliomas, glioblastomas, neuroblastomas, Kaposi's sarcoma, and sarcomas.

A number of disorders or conditions, other than cancer, also can be treated with the above-described method. Examples include arthritis, rheumatoid arthritis, psoriasis, atherosclerosis, diabetic retinopathy, age-related macular degeneration, Grave's disease, vascular restenosis (including restenosis following angioplasty), arteriovenous malformations (AVM), meningioma, hemangioma, neovascular glaucoma, chronic kidney disease, diabetic nephropathy, polycystic kidney disease, interstitial lung disease, pulmonary hypertension, chronic obstructive pulmonary disease (COPD), emphysema, autoimmune hepatitis, chronic inflammatory liver disease, hepatic cirrhosis, cutaneous T-cell lymphoma, rosacea, and basal cell carcinoma.

Other treatment targets include those described in, e.g., US Applications 2009004297, 20090175791, and 20070161553, such as angiofibroma, atherosclerotic plaques, corneal graft neovascularization, hemophilic joints, hypertrophic scars, Osler-Weber syndrome, pyogenic granuloma retrolental fibroplasia, scleroderma, trachoma, vascular adhesions, synovitis, dermatitis (e.g., atopic dermatitis), various other inflammatory diseases and disorders, and endometriosis.

As used herein, a “subject” refers to a human and a non-human animal. Examples of a non-human animal include all vertebrates, e.g., mammals, such as non-human mammals, non-human primates (particularly higher primates), dog, rodent (e.g., mouse or rat), guinea pig, cat, and rabbit, and non-mammals, such as birds, amphibians, reptiles, etc. In one embodiment, the subject is a human. In another embodiment, the subject is an experimental animal or animal suitable as a disease model. A subject to be treated for a disorder can be identified by standard diagnosing techniques for the disorder. Optionally, the subject can be examined for mutation, expression level, or activity level of one or more of the miR-199a-3p, miR-199a-5p, miR-1908, and CTGF mentioned above by methods known in the art or described above before treatment. If the subject has a particular mutation in the gene, or if the gene expression or activity level is, for example, greater in a sample from the subject than that in a sample from a normal person, the subject is a candidate for treatment of this invention.

To confirm the inhibition or treatment, one can evaluate and/or verify the inhibition of endothelial recruitment or resulting angiogenesis using technology known in the art before and/or after the administering step. Exemplary technologies include angiography or arteriography, a medical imaging technique used to visualize the inside, or lumen, of blood vessels and organs of the body, can generally be done by injecting a radio-opaque contrast agent into the blood vessel and imaging using X-ray based techniques such as fluoroscopy.

“Treating” or “treatment” as used herein refers to administration of a compound or agent to a subject who has a disorder with the purpose to cure, alleviate, relieve, remedy, delay the onset of, prevent, or ameliorate the disorder, the symptom of a disorder, the disease state secondary to the disorder, or the predisposition toward the disorder. An “effective amount” or “therapeutically effective amount” refers to an amount of the compound or agent that is capable of producing a medically desirable result in a treated subject. The treatment method can be performed in vivo or ex vivo, alone or in conjunction with other drugs or therapy. A therapeutically effective amount can be administered in one or more administrations, applications or dosages and is not intended to be limited to a particular formulation or administration route.

The expression “effective amount” as used herein, refers to a sufficient amount of the compound of the invention to exhibit the desired therapeutic effect. The exact amount required will vary from subject to subject, depending on the species, age, and general condition of the subject, the particular therapeutic agent and the like. The compounds of the invention are preferably formulated in dosage unit form for ease of administration and uniformity of dosage. The expression “dosage unit form” as used herein refers to a physically discrete unit of therapeutic agent appropriate for the patient to be treated. It will be understood, however, that the total daily usage of the compounds and compositions of the present invention will be decided by the attending physician within the scope of sound medical judgment. The specific therapeutically effective dose level for any particular patient or organism will depend upon a variety of factors including the disorder being treated and the severity of the disorder; the anticancer activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

A therapeutic agent can be administered in vivo or ex vivo, alone or co-administered in conjunction with other drugs or therapy, i.e., a cocktail therapy. As used herein, the term “co-administration” or “co-administered” refers to the administration of at least two agent(s) or therapies to a subject. For example, in the treatment of tumors, particularly vascularized, malignant tumors, the agents can be used alone or in combination with, e.g., chemotherapeutic, radiotherapeutic, apoptopic, anti-angiogenic agents and/or immunotoxins or coaguligands. In some embodiments, the co-administration of two or more agents/therapies is concurrent. In other embodiments, a first agent/therapy is administered prior to a second agent/therapy. Those of skill in the art understand that the formulations and/or routes of administration of the various agents/therapies used may vary.

In an in vivo approach, a compound or agent is administered to a subject. Generally, the compound is suspended in a pharmaceutically-acceptable carrier (such as, for example, but not limited to, physiological saline) and administered orally or by intravenous infusion, or injected or implanted subcutaneously, intramuscularly, intrathecally, intraperitoneally, intrarectally, intravaginally, intranasally, intragastrically, intratracheally, or intrapulmonarily.

The dosage required depends on the choice of the route of administration; the nature of the formulation; the nature of the patient's illness; the subject's size, weight, surface area, age, and sex; other drugs being administered; and the judgment of the attending physician. Suitable dosages are in the range of 0.01-100 mg/kg. Variations in the needed dosage are to be expected in view of the variety of compounds available and the different efficiencies of various routes of administration. For example, oral administration would be expected to require higher dosages than administration by i.v. injection. Variations in these dosage levels can be adjusted using standard empirical routines for optimization as is well understood in the art. Encapsulation of the compound in a suitable delivery vehicle (e.g., polymeric microparticles or implantable devices) can increase the efficiency of delivery, particularly for oral delivery.

### Compositions

Within the scope of this invention is a composition that contains a suitable carrier and one or more of the therapeutic agents described above. The composition can be a pharmaceutical composition that contains a pharmaceutically acceptable carrier, a dietary composition that contains a dietarily acceptable suitable carrier, or a cosmetic composition that contains a cosmetically acceptable carrier.

The term “pharmaceutical composition” refers to the combination of an active agent with a carrier, inert or active, making the composition especially suitable for diagnostic or therapeutic use in vivo or ex vivo. A “pharmaceutically acceptable carrier,” after administered to or upon a subject, does not cause undesirable physiological effects. The carrier in the pharmaceutical composition must be “acceptable” also in the sense that it is compatible with the active ingredient and can be capable of stabilizing it. One or more solubilizing agents can be utilized as pharmaceutical carriers for delivery of an active compound. Examples of a pharmaceutically acceptable carrier include, but are not limited to, biocompatible vehicles, adjuvants, additives, and diluents to achieve a composition usable as a dosage form. Examples of other carriers include colloidal silicon oxide, magnesium stearate, cellulose, sodium lauryl sulfate, and D&C Yellow #10.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts of amines, carboxylic acids, and other types of compounds, are well known in the art. For example, S. M. Berge, et al. describe pharmaceutically acceptable salts in detail in J. Pharmaceutical Sciences, 66:1-19 (1977), incorporated herein by reference. The salts can be prepared in situ during the final isolation and purification of the compounds of the invention, or separately by reacting a free base or free acid function with a suitable reagent, as described generally below. For example, a free base function can be reacted with a suitable acid. Furthermore, where the compounds of the invention carry an acidic moiety, suitable pharmaceutically acceptable salts thereof may, include metal salts such as alkali metal salts, e.g. sodium or potassium salts; and alkaline earth metal salts, e.g. calcium or magnesium salts. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts, include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

As described above, the pharmaceutical compositions of the present invention additionally comprise a pharmaceutically acceptable carrier, which, as used herein, includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutical compositions and known techniques for the preparation thereof. Except insofar as any conventional carrier medium is incompatible with the compounds of the invention, such as by producing any undesirable biological effect or otherwise interacting in a deleterious manner with any other component(s) of the pharmaceutical composition, its use is contemplated to be within the scope of this invention. Some examples of materials which can serve as pharmaceutically acceptable carriers include, but are not limited to, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatine; talc; excipients such as cocoa butter and suppository waxes; oils such as peanut oil, cottonseed oil; safflower oil, sesame oil; olive oil; corn oil and soybean oil; glycols; such as propylene glycol; esters such as ethyl oleate and ethyl laurate; agar; natural and synthetic phospholipids, such as soybean and egg yolk phosphatides, lecithin, hydrogenated soy lecithin, dimyristoyl lecithin, dipalmitoyl lecithin, distearoyl lecithin, dioleoyl lecithin, hydroxylated lecithin, lysophosphatidylcholine, cardiolipin, sphingomyelin, phosphatidylcholine, phosphatidyl ethanolamine, diastearoyl phosphatidylethanolamine (DSPE) and its pegylated esters, such as DSPE-PEG750 and, DSPE-PEG2000, phosphatidic acid, phosphatidyl glycerol and phosphatidyl serine. Commercial grades of lecithin which are preferred include those which are available under the trade name Phosal® or Phospholipon® and include Phosal 53 MCT, Phosal 50 PG, Phosal 75 SA, Phospholipon 90H, Phospholipon 90G and Phospholipon 90 NG; soy-phosphatidylcholine (SoyPC) and DSPE-PEG2000 are particularly preferred; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The above-described composition, in any of the forms described above, can be used for treating melanoma, or any other disease or condition described herein. An effective amount refers to the amount of an active compound/agent that is required to confer a therapeutic effect on a treated subject. Effective doses will vary, as recognized by those skilled in the art, depending on the types of diseases treated, route of administration, excipient usage, and the possibility of co-usage with other therapeutic treatment.

A pharmaceutical composition of this invention can be administered parenterally, orally, nasally, rectally, topically, or buccally. The term “parenteral” as used herein refers to subcutaneous, intracutaneous, intravenous, intrmuscular, intraarticular, intraarterial, intrasynovial, intrasternal, intrathecal, intralesional, or intracranial injection, as well as any suitable infusion technique.

A sterile injectable composition can be a solution or suspension in a non-toxic parenterally acceptable diluent or solvent. Such solutions include, but are not limited to, 1,3-butanediol, mannitol, water, Ringer's solution, and isotonic sodium chloride solution. In addition, fixed oils are conventionally employed as a solvent or suspending medium (e.g., synthetic mono- or diglycerides). Fatty acid, such as, but not limited to, oleic acid and its glyceride derivatives, are useful in the preparation of injectables, as are natural pharmaceutically acceptable oils, such as, but not limited to, olive oil or castor oil, polyoxyethylated versions thereof. These oil solutions or suspensions also can contain a long chain alcohol diluent or dispersant such as, but not limited to, carboxymethyl cellulose, or similar dispersing agents. Other commonly used surfactants, such as, but not limited to, Tweens or Spans or other similar emulsifying agents or bioavailability enhancers, which are commonly used in the manufacture of pharmaceutically acceptable solid, liquid, or other dosage forms also can be used for the purpose of formulation.

A composition for oral administration can be any orally acceptable dosage form including capsules, tablets, emulsions and aqueous suspensions, dispersions, and solutions. In the case of tablets, commonly used carriers include, but are not limited to, lactose and corn starch. Lubricating agents, such as, but not limited to, magnesium stearate, also are typically added. For oral administration in a capsule form, useful diluents include, but are not limited to, lactose and dried corn starch. When aqueous suspensions or emulsions are administered orally, the active ingredient can be suspended or dissolved in an oily phase combined with emulsifying or suspending agents. If desired, certain sweetening, flavoring, or coloring agents can be added.

Pharmaceutical compositions for topical administration according to the described invention can be formulated as solutions, ointments, creams, suspensions, lotions, powders, pastes, gels, sprays, aerosols, or oils. Alternatively, topical formulations can be in the form of patches or dressings impregnated with active ingredient(s), which can optionally comprise one or more excipients or diluents. In some preferred embodiments, the topical formulations include a material that would enhance absorption or penetration of the active agent(s) through the skin or other affected areas.

A topical composition contains a safe and effective amount of a dermatologically acceptable carrier suitable for application to the skin. A “cosmetically acceptable” or “dermatologically-acceptable” composition or component refers a composition or component that is suitable for use in contact with human skin without undue toxicity, incompatibility, instability, allergic response, and the like. The carrier enables an active agent and optional component to be delivered to the skin at an appropriate concentration(s). The carrier thus can act as a diluent, dispersant, solvent, or the like to ensure that the active materials are applied to and distributed evenly over the selected target at an appropriate concentration. The carrier can be solid, semi-solid, or liquid. The carrier can be in the form of a lotion, a cream, or a gel, in particular one that has a sufficient thickness or yield point to prevent the active materials from sedimenting. The carrier can be inert or possess dermatological benefits. It also should be physically and chemically compatible with the active components described herein, and should not unduly impair stability, efficacy, or other use benefits associated with the composition.

### Combination Therapies

In some embodiments, the pharmaceutical composition may further comprise an additional compound having antiproliferative activity. The additional compound having antiproliferative activity can be selected from a group of antiproliferative agents including those shown in Table 2.

It will also be appreciated that the compounds and pharmaceutical compositions of the present invention can be formulated and employed in combination therapies, that is, the compounds and pharmaceutical compositions can be formulated with or administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder, or they may achieve different effects (e.g., control of any adverse effects).

By “antiproliferative agent” is meant any antiproliferative agent, including those antiproliferative agents listed in Table 2, any of which can be used in combination with an LXR agonist to treat the medical conditions recited herein. Antiproliferative agents also include organo-platine derivatives, naphtoquinone and benzoquinone derivatives, chrysophanic acid and anthroquinone derivatives thereof.

TABLE 2

| | | |
|---|---|---|
| Alkylating agents | Busulfan | Chlorambucil |
| | dacarbazine | procarbazine |
| | ifosfamide | altretamine |
| | hexamethylmelamine | estramustine phosphate |
| | thiotepa | mechlorethamine |
| | lomustine | streptozocin |
| | cyclophosphamide | temozolomide |
| | | Semustine |
| Platinum agents | spiroplatin | lobaplatin (Aeterna) |
| | tetraplatin | satraplatin (Johnson Matthey) |
| | ormaplatin | BBR-3464 (Hoffmann-La Roche) |
| | iproplatin | SM-11355 (Sumitomo) |
| | ZD-0473 (AnorMED) | AP-5280 (Access) |
| | oxaliplatin | cisplatin |
| | carboplatin | |
| Antimetabolites | azacytidine | trimetrexate |
| | Floxuridine | deoxycoformycin |
| | 2-chlorodeoxyadenosine | pentostatin |
| | 6-mercaptopurine | hydroxyurea |
| | 6-thioguanine | decitabine (SuperGen) |
| | cytarabine | clofarabine (Bioenvision) |
| | 2-fluorodeoxy cytidine | irofulven (MGI Pharma) |
| | methotrexate | DMDC (Hoffmann-La Roche) |
| | tomudex | ethynylcytidine (Taiho) |
| | fludarabine | gemcitabine |
| | raltitrexed | capecitabine |
| Topoisomerase | amsacrine | exatecan mesylate (Daiichi) |
| inhibitors | epirubicin | quinamed (ChemGenex) |
| | etoposide | gimatecan (Sigma-Tau) |
| | teniposide or mitoxantrone | diflomotecan (Beaufour-Ipsen) |
| | 7-ethyl-10-hydroxy-camptothecin | TAS-103 (Taiho) |
| | dexrazoxanet (TopoTarget) | elsamitrucin (Spectrum) |
| | pixantrone (Novuspharma) | J-107088 (Merck & Co) |
| | rebeccamycin analogue (Exelixis) | BNP-1350 (BioNumerik) |
| | BBR-3576 (Novuspharma) | CKD-602 (Chong Kun Dang) |
| | rubitecan (SuperGen) | KW-2170 (Kyowa Hakko) |
| | irinotecan (CPT-11) | hydroxycamptothecin (SN-38) |
| | topotecan | |
| Antitumor antibiotics | valrubicin | azonafide |
| | therarubicin | anthrapyrazole |
| | idarubicin | oxantrazole |
| | rubidazone | losoxantrone |
| | plicamycin | MEN-10755 (Menarini) |
| | porfiromycin | GPX-100 (Gem Pharmaceuticals) |
| | mitoxantrone (novantrone) | Epirubicin |
| | amonafide | mitoxantrone |
| | | doxorubicin |
| Antimitotic | colchicine | E7010 (Abbott) |
| agents | vinblastine | PG-TXL (Cell Therapeutics) |
| | vindesine | IDN 5109 (Bayer) |
| | dolastatin 10 (NCI) | A 105972 (Abbott) |
| | rhizoxin (Fujisawa) | A 204197 (Abbott) |
| | mivobulin (Warner-Lambert) | LU 223651 (BASF) |
| | cemadotin (BASF) | D 24851 (ASTAMedica) |
| | RPR 109881A (Aventis) | ER-86526 (Eisai) |
| | TXD 258 (Aventis) | combretastatin A4 (BMS) |
| | epothilone B (Novartis) | isohomohalichondrin-B (PharmaMar) |
| | T 900607 (Tularik) | ZD 6126 (AstraZeneca) |
| | T 138067 (Tularik) | AZ10992 (Asahi) |
| | cryptophycin 52 (Eli Lilly) | IDN-5109 (Indena) |
| | vinflunine (Fabre) | AVLB (Prescient NeuroPharma) |
| | auristatin PE (Teikoku Hormone) | azaepothilone B (BMS) |
| | BMS 247550 (BMS) | BNP-7787 (BioNumerik) |
| | BMS 184476 (BMS) | CA-4 prodrug (OXiGENE) |
| | BMS 188797 (BMS) | dolastatin-10 (NIH) |
| | taxoprexin (Protarga) | CA-4 (OXiGENE) |
| | SB 408075 (GlaxoSmithKline) | docetaxel |
| | Vinorelbine | vincristine |
| | Trichostatin A | paclitaxel |
| Aromatase inhibitors | aminoglutethimide | YM-511 (Yamanouchi) |
| | atamestane (BioMedicines) | formestane |
| | letrozole | exemestane |
| | anastrazole | |
| Thymidylate | pemetrexed (Eli Lilly) | nolatrexed (Eximias) |
| synthase inhibitors | ZD-9331 (BTG) | CoFactor ™ (BioKeys) |
| DNA antagonists | trabectedin (PharmaMar) | edotreotide (Novartis) |
| | glufosfamide (Baxter International) | mafosfamide (Baxter International) |
| | albumin + 32P (Isotope Solutions) | apaziquone (Spectrum |
| | thymectacin (NewBiotics) | Pharmaceuticals) |
| | | O6 benzyl guanine (Paligent) |
| Farnesyltransferase | arglabin (NuOncology Labs) | tipifarnib (Johnson & Johnson) |
| inhibitors | lonafarnib (Schering-Plough) | perillyl alcohol (DOR BioPharma) |
| | BAY-43-9006 (Bayer) | |
| Pump inhibitors | CBT-1 (CBA Pharma) | zosuquidar trihydrochloride (Eli Lilly) |
| | tariquidar (Xenova) | biricodar dicitrate (Vertex) |
| | MS-209 (Schering AG) | |
| Histone | tacedinaline (Pfizer) | pivaloyloxymethyl butyrate (Titan) |
| acetyltransferase | SAHA (Aton Pharma) | depsipeptide (Fujisawa) |
| inhibitors | MS-275 (Schering AG) | |
| Metalloproteinase | Neovastat (Aeterna Laboratories) | CMT-3 (CollaGenex) |
| inhibitors | marimastat (British Biotech) | BMS-275291 (Celltech) |
| Ribonucleoside | gallium maltolate (Titan) | tezacitabine (Aventis) |
| reductase inhibitors | triapine (Vion) | didox (Molecules for Health) |
| TNF alpha | virulizin (Lorus Therapeutics) | revimid (Celgene) |
| agonists/antagonists | CDC-394 (Celgene) | |
| Endothelin A | atrasentan (Abbott) | YM-598 (Yamanouchi) |
| receptor antagonist | ZD-4054 (AstraZeneca) | |
| Retinoic acid | fenretinide (Johnson & Johnson) | alitretinoin (Ligand) |
| receptor agonists | LGD-1550 (Ligand) | |
| Immuno-modulators | interferon | dexosome therapy (Anosys) |
| | oncophage (Antigenics) | pentrix (Australian Cancer |
| | GMK (Progenics) | Technology) |
| | adenocarcinoma vaccine (Biomira) | ISF-154 (Tragen) |
| | CTP-37 (AVI BioPharma) | cancer vaccine (Intercell) |
| | IRX-2 (Immuno-Rx) | norelin (Biostar) |
| | PEP-005 (Peplin Biotech) | BLP-25 (Biomira) |
| | synchrovax vaccines (CTL Immuno) | MGV (Progenics) |
| | melanoma vaccine (CTL Immuno) | ß-alethine (Dovetail) |
| | p21 RAS vaccine (GemVax) | CLL therapy (Vasogen) |
| | MAGE-A3 (GSK) | Ipilimumab (BMS), |
| | nivolumab (BMS) | CM-10 (cCam Biotherapeutics) |
| | abatacept (BMS) | MPDL3280A (Genentech) |
| | pembrolizumab (Merck) | |
| Hormonal and | estrogens | dexamethasone |
| antihormonal agents | conjugated estrogens | prednisone |
| | ethinyl estradiol | methylprednisolone |
| | chlortrianisen | prednisolone |
| | idenestrol | aminoglutethimide |
| | hydroxyprogesterone caproate | leuprolide |
| | medroxyprogesterone | octreotide |
| | testosterone | mitotane |
| | testosterone propionate; | P-04 (Novogen) |
| | fluoxymesterone | 2-methoxyestradiol (EntreMed) |
| | methyltestosterone | arzoxifene (Eli Lilly) |
| | diethylstilbestrol | tamoxifen |
| | megestrol | toremofine |
| | bicalutamide | goserelin |
| | flutamide | Leuporelin |
| | nilutamide | bicalutamide |
| Photodynamic | talaporfin (Light Sciences) | Pd-bacteriopheophorbide (Yeda) |
| agents | Theralux (Theratechnologies) | lutetium texaphyrin (Pharmacyclics) |
| | motexafin gadolinium | hypericin |
| | (Pharmacyclics) | |
| Kinase Inhibitors | imatinib (Novartis) | EKB-569 (Wyeth) |
| | leflunomide (Sugen/Pharmacia) | kahalide F (PharmaMar) |
| | ZD1839 (AstraZeneca) | CEP-701 (Cephalon) |
| | erlotinib (Oncogene Science) | CEP-751 (Cephalon) |
| | canertinib (Pfizer) | MLN518 (Millenium) |
| | squalamine (Genaera) | PKC412 (Novartis) |
| | SU5416 (Pharmacia) | Phenoxodiol (Novogen) |
| | SU6668 (Pharmacia) | C225 (ImClone) |
| | ZD4190 (AstraZeneca) | rhu-Mab (Genentech) |
| | ZD6474 (AstraZeneca) | MDX-H210 (Medarex) |
| | vatalanib (Novartis) | 2C4 (Genentech) |
| | PKI166 (Novartis) | MDX-447 (Medarex) |
| | GW2016 (GlaxoSmithKline) | ABX-EGF (Abgenix) |
| | EKB-509 (Wyeth) | IMC-1C11 (ImClone) |
| | trastuzumab (Genentech) | Tyrphostins |
| | OSI-774 (Tarceva ™) | Gefitinib (Iressa) |
| | CI-1033 (Pfizer) | PTK787 (Novartis) |
| | SU11248 (Pharmacia) | EMD 72000 (Merck) |
| | RH3 (York Medical) | Emodin |
| | Genistein | Radicinol |
| | Radicinol | Vemurafenib (B-Raf enzyme |
| | Met-MAb (Roche) | inhibitor, Daiichi Sankyo) |
| | SR-27897 (CCK A inhibitor, Sanofi-Synthelabo) | ceflatonin (apoptosis promotor, ChemGenex) |
| | tocladesine (cyclic AMP agonist, Ribapharm) | BCX-1777 (PNP inhibitor, BioCryst) |
| | alvocidib (CDK inhibitor, Aventis) | ranpirnase (ribonuclease stimulant, Alfacell) |
| | CV-247 (COX-2 inhibitor, Ivy Medical) | galarubicin (RNA synthesis inhibitor, Dong-A) |
| | P54 (COX-2 inhibitor, Phytopharm) | tirapazamine (reducing agent, SRI |
| | CapCell ™ (CYP450 stimulant, Bavarian Nordic) | International) |
| | GCS-100 (gal3 antagonist, GlycoGenesys) | N-acetylcysteine (reducing agent, Zambon) |
| | G17DT immunogen (gastrin inhibitor, Aphton) | R-flurbiprofen (NF-kappaB inhibitor, Encore) |
| | efaproxiral (oxygenator, Allos Therapeutics) | 3CPA (NF-kappaB inhibitor, Active Biotech) |
| | PI-88 (heparanase inhibitor, Progen) | seocalcitol (vitamin D receptor agonist, Leo) |
| | tesmilifene (histamine antagonist, YM | 131-I-TM-601 (DNA antagonist, |
| | BioSciences) | TransMolecular) |
| | histamine (histamine H2 receptor agonist, Maxim) | eflornithine (ODC inhibitor, ILEX Oncology) |
| | tiazofurin (IMPDH inhibitor, Ribapharm) | minodronic acid (osteoclast inhibitor, |
| | cilengitide (integrin antagonist, Merck KGaA) | Yamanouchi) |
| | SR-31747 (IL-1 antagonist, Sanofi-Synthelabo) | indisulam (p53 stimulant, Eisai) |
| | CCI-779 (mTOR kinase inhibitor, Wyeth) | aplidine (PPT inhibitor, PharmaMar) |
| | exisulind (PDE V inhibitor, Cell Pathways) | gemtuzumab (CD33 antibody, Wyeth Ayerst) |
| | CP-461 (PDE V inhibitor, Cell Pathways) | PG2 (hematopoiesis enhancer, |
| | AG-2037 (GART inhibitor, Pfizer) | Pharmagenesis) |
| | WX-UK1 (plasminogen activator inhibitor, Wilex) | Immunol ™ (triclosan oral rinse, Endo) |
| | PBI-1402 (PMN stimulant, ProMetic LifeSciences) | triacetyluridine (uridine prodrug, Wellstat) |
| | bortezomib (proteasome inhibitor, Millennium) | SN-4071 (sarcoma agent, Signature |
| | SRL-172 (T cell stimulant, SR Pharma) | BioScience) |
| | TLK-286 (glutathione S transferase inhibitor, | TransMID-107 ™ (immunotoxin, KS Biomedix) |
| | Telik) | PCK-3145 (apoptosis promotor, Procyon) |
| | PT-100 (growth factor agonist, Point | doranidazole (apoptosis promotor, Pola) |
| | Therapeutics) | CHS-828 (cytotoxic agent, Leo) |
| | midostaurin (PKC inhibitor, Novartis) | trans-retinoic acid (differentiator, NIH) |
| | bryostatin-1 (PKC stimulant, GPC Biotech) | MX6 (apoptosis promotor, MAXIA) |
| | CDA-II (apoptosis promotor, Everlife) | apomine (apoptosis promotor, ILEX Oncology) |
| | SDX-101 (apoptosis promotor, Salmedix) | urocidin (apoptosis promotor, Bioniche) |
| | rituximab (CD20 antibody, Genentech | Ro-31-7453 (apoptosis promotor, La Roche) |
| | carmustine | brostallicin (apoptosis promotor, Pharmacia) |
| | Mitoxantrone | β-lapachone |
| | Bleomycin | gelonin |
| | Absinthin | cafestol |
| | Chrysophanic acid | kahweol |
| | Cesium oxides | caffeic acid |
| | BRAF inhibitors, | Tyrphostin AG |
| | PDL1 inhibitors | PD-1 inhibitors |
| | MEK inhibitors | CTLA-4 inhibitors |
| | bevacizumab | sorafenib |
| | angiogenesis inhibitors | BRAF inhibitors |
| | dabrafenib | rindopepimut |
| | ramucirumab | vedotin |
| | glembatumumab | ANG4043 |
| | ANG1005 | |

### EXAMPLES

### Example 1 Materials and Methos

Methods useful for determining the activity of LXR agonists are described in International Patent Publication No. WO2014/028461, the methods of which are incorporated herein by reference.

This example describes materials and methos used in EXAMPLES 2-11 below.

### Compounds

TABLE 3
Compound Names

| Compound # | Compound Name |
|---|---|
| 108 | GW3965 |
| 109 | SB742881 |
| 110 | T0901317 |
| 111 | LXR-623 |
| 112 | WO-2010-0138598 Ex. 9 or |
| | WO-201000138598 |
| 113 | WO-2007-002563 Ex. 19 or |
| | WO-2007-002563 |

### Animal Studies

All mouse experiments were conducted in agreement with a protocol approved by the Institutional Animal Care and Use Committee (IACUC) at The Rockefeller University. 6-8-week old age-matched and sex-matched mice were used for primary tumor growth and metastasis assays as previously described (Minn et al., 2005; Tavazoie et al., 2008). See Extended Experimental Procedures.

### Cell Culture

All cancer cell lines were cultured as previously described (Tavazoie et al., 2008). 293T and human umbilical vein endothelial cells (HUVEC's) were maintained in standard conditions. miRNA and gene knock-down/over-expression studies in cell lines and in vitro functional assays are detailed in Extended Experimental Procedures.

### Microarray Hybridization

In order to identify miRNAs deregulated across highly metastatic derivatives, small RNAs were enriched from total RNA derived from MeWo and A375 cell lines and profiled by LC sciences. In order to identify potential gene targets of miR-199a-3p, miR-199a-5p, and miR-1908, total RNA from MeWo cell lines was labeled and hybridized onto Illumina HT-12 v3 Expression BeadChip arrays by The Rockefeller University genomics core facility. See Extended Experimental Procedures for thresholds and criteria used to arrive at miRNA and mRNA targets.

Analysis of miRNA Expression in Human Melanoma Skin Lesions

All human clinical samples used in this study were obtained, processed, and analyzed in accordance with IRB guidelines. Total RNA was extracted from paraffin-embedded cross-sections of primary melanoma skin lesions previously resected from patients at MSKCC, and specific miRNA expression levels were analyzed in a blinded fashion using TaqMan miRNA Assays (Applied Biosystems). Kaplan-Meier curves representing each patient's metastasis-free-survival data as a function of primary tumor miRNA expression values were generated using the GraphPad Prism software package.

### In Vivo LNA Therapy

Following tail-vein injection of 4×10⁴ MeWo-LM2 cells, NOD-SCID mice were treated intravenously twice a week for four weeks with in vivo-optimized LNAs (Exiqon) antisense to miR-199a-3p, miR-199a-5p, and miR-1908 at a combinatorial dose of 12.5 mg/kg delivered in 0.1 mL of PBS.

### Histology

For gross macroscopic metastatic nodule visualization, 5-μm-thick lung tissue sections were H&E stained. For in vivo endothelial content analyses, lung sections were double-stained with antibodies against MECA-32 (Developmental Studies Hybridoma Bank, The University of Iowa, IA), which labels mouse endothelial cells, and human vimentin (Vector Laboratories), which labels human melanoma cells. See Extended Experimental Procedures.

### Data Analysis

All data are represented as mean±SEM. The Kolmogorov-Smirnov test was used to determine significance of differences in metastatic blood vessel density cumulative distributions. The prognostic power of the miRNAs to predict metastatic outcomes was tested for significance using the Mantel-Cox log-rank test. The one-way Mann-Whitney t-test was used to determine significance values for non-Gaussian bioluminescence measurements. For all other comparisons, the one-sided student's t-test was used. P values<0.05 were deemed to be statistically significant.

### In Vivo Selection, Experimental Metastasis, and Primary Tumor Growth Assays

All mouse experiments were conducted in agreement with a protocol approved by the Institutional Animal Care and Use Committee (IACUC) at The Rockefeller University. To generate multiple metastatic derivatives from two independent human melanoma cell lines, in vivo selection was performed as previously described (Minn et al., 2005 Nature 436, 518-524; Pollack and Fidler, 1982 J. Natl. Cancer Inst. 69, 137-141). In brief, 1×10⁶ pigmented MeWo or non-pigmented A375 melanoma parental cells were resuspended in 0.1 mL of PBS and intravenously injected into 6-8-week old immunocompromised NOD-SCID mice. Following lung metastases formation, nodules were dissociated and cells were propagated in vitro, giving rise to first generation of lung metastatic derivatives (LM1). The LM1 cells were then subjected to another round of in vivo selection by injecting 2×10⁵ cells via the tail-vein into NOD-SCID mice, giving rise to metastatic nodules, whose subsequent dissociation yielded second generation of lung metastatic derivatives (LM2). For the A375 cell line, a third round of in vivo selection was performed, yielding the highly metastatic A375-LM3 derivatives.

In order to monitor metastasis in vivo through bioluminescence imaging, A375 and MeWo parental cells and their metastatic derivatives were transduced with a retroviral construct expressing a luciferase reporter (Ponomarev et al., 2004 Eur J Nucl Med Mol Imaging 31, 740-751). For all metastasis experiments, lung or systemic colonization was monitored over time and quantified through non-invasive bioluminescence imaging as previously described (Minn et al., 2005). To determine whether in vivo selection had been achieved, 4×10⁴ MeWo parental or MeWo-LM2 cells and 1×10⁵A375 parental or A375-LM3 cells were resuspended in 0.1 mL of PBS and injected via the lateral tail vein into 6-8-week old NOD-SCID mice. For experimental metastasis assays testing the effects of putative promoter miRNAs on lung colonization, 4×10⁴ MeWo parental cells over-expressing miR-199a, miR-1908, miR-214, or a control hairpin, 4×10⁴ MeWo-LM2 cells with silenced expression of miR-199a-3p, miR-199a-5p, miR-1908, or a control sequence, and 2×10⁵ A375-LM3 cells inhibited for miR-199a-3p, miR-199a-5p, miR-1908, or a control sequence were resuspended in 0.1 mL of PBS and tail-vein injected into 6-8-week old NOD-SCID mice. For epistasis experiments, 1×10⁵ MeWo-LM2 cells expressing an shRNA targeting ApoE, DNAJA4, or a control sequence or siRNA inhibiting LRP1 or a control sequence in the setting of miRNA inhibition were intravenously injected into 6-8-week old NOD-SCID mice. For ApoE pre-treatment experiments, MeWo-LM2 cells were incubated in the presence of ApoE or BSA at 100 μg/mL at 37° C. After 24 hours, 4×10⁴ cells were injected via the tail-vein into 7-week old NOD-SCID mice. To determine the effect of pre-treating highly metastatic melanoma cells with LNAs targeting miR-199a-3p, miR-199a-5p, and miR-1908 on metastasis, MeWo-LM2 cells were transfected with each LNA individually, a cocktail of LNAs targeting all three miRNAs, or a control LNA. After 48 hours, 1×10⁵ cells, resuspended in 0.1 mL of PBS, were administered intravenously into 7-week old NOD-SCID mice for lung metastatic colonization studies or through intracardiac injection into 7-week old athymic nude mice for systemic metastasis assays. To determine the effect of genetic deletion of ApoE on metastasis, 8-week old C57BL/6-WT or C57BL/6-ApoE−/− mice were intravenously injected with 5×10⁴ B16F10 mouse melanoma cells. For primary tumor growth studies, 1×10⁶ parental MeWo cells over-expressing miR-199a, miR-1908, or a control hairpin were mixed 1:1 with matrigel and subcutaneously injected into the lower right flank of 6-week old immunodeficient NOD-SCID mice. Animals were palpated weekly for tumor formation, after which sizeable tumors were measured twice a week. Tumor volume was calculated as (small diameter)²×(large diameter)/2.

Lentiviral miRNA Inhibition and Gene Knock-Down

293T cells were seeded in a 10-cm plate and allowed to reach 60% confluency. Prior to transfection, the cell media was replaced with fresh antibiotic-free DMEM media supplemented with 10% FBS. 6 μg of vector A, 12 μg of vector K, and 12 μg of the appropriate miR-Zip (System Biosciences, Mountain View, Calif.) or shRNA plasmid construct (MSKCC HTS Core Facility, New York, N.Y.) were co-transfected using 60 μL of TransIT-293 transfection reagent (MIR 2700, Mirus Bio LLC, Madison, Wis.). The cells were incubated at 37° C. for 48 hours, and the virus was harvested by spinning the cell media for 10 minutes at 2000 g followed by virus filtration through a 0.45 μm filter. 1×10⁵ cancer cells were transduced with 2 mL of the appropriate virus in the presence of 10 μg/mL of polybrene (TR-1003-G, Millipore, Billerica, Mass.) for 6 hrs. After 48 hours, 2 μg/mL of puromycin (P8833, Sigma-Aldrich, St Louis, Mo.) was added to the cell media for lentiviral selection. The cells were kept in puromycin selection for 72 hours. The following miR-Zip sequences were used:

miR-Zip-199a-3p:
5′-GATCCGACAGTAGCCTGCACATTAGTCACTTCCTGTCAGTAACCAA
TGTGCAGACTACTGTTTTTTGAATT-3′
miR-Zip-199a-5p:
5′-GATCCGCCCAGTGCTCAGACTACCCGTGCCTTCCTGTCAGGAACAG
GTAGTCTGAACACTGGGTTTTTGAATT-3′
miR-Zip-1908
5′-GATCCGCGGCGGGAACGGCGATCGGCCCTTCCTGTCAGGACCAAT
CGCCGTCCCCGCCGTTTTTGAATT-3′

The following shRNA sequences were used:

shAPOE¹:
5′CCGGGAAGGAGTTGAAGGCCTACAACTCGAGTTGTAGGCCTTCAACTC
CTTCTTTTT3′
shAPOE²:
5′CCGGGCAGACACTGTCTGAGCAGGTCTCGAGACCTGCTCAGACAGTGT
CTGCTTTTT3′
shDNAJA4¹:
5′CCGGGCGAGAAGTTTAAACTCATATCTCGAGATATGAGTTTAAACTT
CTCGCTTTTT3′
shDNAJA4²:
5′CCGGCCTCGACAGAAAGTGAGGATTCTCGAGAATCCTCACTTTCTGTC
GAGGTTTTT3′

Retroviral miRNA and Gene Over-Expression

6 μg of vector VSVG, 12 μg of vector Gag-Pol, and 12 μg of pBabe plasmid containing the coding sequences of human ApoE, DNAJA4, or an empty vector or miR-Vec containing the precursor sequence of miR-199a, miR-214, miR-1908, or a control hairpin were co-transfected into 60%-confluent 293T cells using 60 μL of TransIT-293 transfection reagent. The cells were incubated at 37° C. for 48 hours, after which the virus was harvested and transduced into cancer cells in the presence of 10 μg/mL of polybrene for 6 hours. After 48 hours, 2 μg/mL of puromycin or 10 μg/mL of blasticidin (15205, Sigma-Aldrich, St Louis, Mo.) were added to the cell media for retroviral selection. The cells were kept in puromycin selection for 72 hours or in blasticidin selection for 7 days. The following cloning primers were used for over-expression of the coding sequences of ApoE and DNAJA4:

ApoE_CDS_Fwd:
5′-TCATGAGGATCCATGAAGGTTCTGTGGGCT-3′
ApoE_CDS_Rev:
5′-TAGCAGAATTCTCAGTGATTGTCGCTGGG-3′
DNAJA4_CDS_Fwd:
5′-ATCCCTGGATCCATGTGGGAAAGCCTGACCC-3′
DNAJA4_CDS_Rev:
5′-TACCATGTCGACTCATGCCGTCTGGCACTGC-3′

LNA-Based miRNA Knock-Down

LNAs complimentary to mature miR-199a-3p, miR-199a-5p, miR-1908, or a control sequence (426917-00, 426918-00, 426878-00, and 1990050, respectively; Exiqon, Vedbaek, Denmark) were transfected at a final concentration of 50 nM into 50% confluent MeWo-LM2 cancer cells cultured in antibiotics-free media using Lipofectamine™ 2000 transfection reagent (11668-09, Invitrogen, Carlsbad, Calif.). After 8 hours, the transfection media was replaced with fresh media. After 48 hours, 1×10⁵ cells were injected intravenously into NOD-SCID mice to assess lung metastatic colonization or through intracardiac injection into athymic nude mice to assess systemic metastasis. For cell invasion and endothelial recruitment in vitro assays, the cells were used 96 hours post-transfection.

siRNA-Based mRNA Knock-Down

siRNAs targeting LRP1, LRP8, VLDLR, LDLR, or a control sequence were transfected into cancer cells or HUVEC's at a final concentration of 100 nM using Lipofectamine™ 2000 transfection reagent. After 5 hours, the transfection media was replaced with fresh media. The cells were subjected to matrigel invasion and endothelial recruitment assays 96 hours post-transfection. Cells transduced with siRNAs targeting LRP1 or a control sequence in the setting of miRNA inhibition were tail-vein injected for lung colonization assays 72 hours post-transfecton. Control non-targeting siRNAs were obtained from Dharmacon. The following LRP1 and LRP8 target sequences were used:

siLRP1¹:
5′-CGAGGACGAUGACUGCUUA-3′;
siLRP1²:
5′-GCUAUGAGUUUAAGAAGUU-3′;
siLRP8¹:
5′-CGAGGACGAUGACUGCUUA-3′;
siLRP8²:
5′-GAACUAUUCACGCCUCAUC-3′.

### Cell Proliferation Assay

To determine the effects of miR-199a or miR-1908 over-expression and combinatorial LNA-induced miRNA inhibition on cell proliferation, 2.5×10⁴ cells were seeded in triplicate in 6-well plates, and viable cells were counted after 5 days. To assess the effects of recombinant ApoE addition on melanoma cell or endothelial cell proliferation, 3×10⁴ melanoma MeWo-LM2 cells or endothelial cells were incubated in the presence of ApoE (100 μM) or BSA (100 μM). Viable cells were counted after 8, 24, 48, 72, and 120 hours.

### Matrigel Invasion Assay

Cancer cells were serum-starved in 0.2% FBS DMEM-based media for 12 hours. Trans-well invasion chambers (354480, BD Biosciences, Bedford, Mass.) were pre-equilibrated prior to beginning the assay by adding 0.5 mL of starvation media to the top and bottom chambers. After 30 minutes, the media in the top chamber was removed, and 0.5 mL of media containing 1×10⁵ cancer cells was added into each matrigel-coated trans-well insert and incubated at 37% C for 24 hours. For neutralization antibody and/or recombinant protein experiments, antibody/recombinant protein was added to each well at the start of the assay at the following concentrations as indicated in the figures: 5-40 μg/mL anti-ApoE 1 D7 (Heart Institute, University of Ottawa), 5-40 μg/mL anti-IgG (AB-108-C, R&D Systems, Minneapolis, Minn.), 100 μM recombinant human ApoE3 (4696, BioVision, Mountain View, Calif.), and 100 μM BSA (A2153, Sigma-Aldrich). Upon completion of the assay, matrigel-coated inserts were washed with PBS, the cells at the top side of each insert were scraped off, and the inserts were fixed in 4% paraformaldehyde for 15 minutes. The inserts were then cut out and mounted onto slides using VectaShield mounting medium containing DAPI (H-1000, Vector Laboratories, Burlingame, Calif.). The basal side of each insert was imaged using an inverted fluorescence microscope (Zeiss Axiovert 40 CFL) at 5× magnification, taking three representative images for each insert. The number of invaded cells was quantified using ImageJ (NIH).

### Endothelial Recruitment Assay

5×10⁴ cancer cells were seeded into 24-well plates approximately 24 hours prior to the start of the assay. HUVEC's were grown to 80% confluency and serum starved in EGM-2 media supplemented with 0.2% FBS for 16 hours. HUVEC's were then pulsed with Cell Tracker Red CMTPX dye (C34552, Invitrogen) for 45 minutes. Meanwhile, cancer cells were washed with PBS, 0.5 mL of 0.2% FBS EGM-2 media was added to each well, and a 3.0 μm HTS Fluoroblock insert (351151, BD Falcon, San Jose, Calif.) was placed into each well. 1×10⁵ HUVEC's, resuspended in 0.5 mL of starvation media, were seeded into each trans-well insert, and the recruitment assay was allowed to proceed for 16-18 hours at 37° C. For neutralization antibody and/or recombinant protein experiments, antibody/protein was then added to each well at the appropriate concentration as indicated in the figures: 40 μg/mL anti-ApoE 1 D7, 40 μg/mL anti-IgG, 100 μM rhApoE3, and 100 μM BSA. Upon completion of the assay, the inserts were processed and analyzed as described for the matrigel invasion assay above (See Matrigel Invasion Assay).

### Endothelial Migration Assay

Serum-starved HUVEC's were pulsed with Cell Tracker Red CMTPX dye for 45 minutes and seeded into HTS Fluoroblock trans-well inserts at a concentration of 1×10⁵ HUVEC's in 0.5 mL starvation media per each insert. The assay was allowed to proceed for 16-18 hours at 37⁴C, and the inserts were processed and analyzed as described above (See Matrigel Invasion Assay).

### Chemotaxis Assay

HUVEC's were serum-starved in 0.2% FBS EGM-2 media for 16 hours and labeled with Cell Tracker Red CMTPX dye for 45 minutes. Meanwhile, the indicated amounts (1-5 μg) of recombinant human ApoE3 or BSA were mixed with 250 μL of matrigel (356231, BD Biosciences) and allowed to solidify at the bottom of a 24-well plate for 30 min. 250 μL of HUVEC EGM-2 media containing 0.2% FBS was then added to each matrigel-coated well, and 3.0 μM HTS Fluoroblock inserts were fitted into each well. 1×10⁵ HUVEC's, resuspended in 0.5 mL of starvation media, were seeded into each insert and allowed to migrate along the matrigel gradient for 16-18 hours at 37° C. Upon completion of the assay, the inserts were mounted on slides and analyzed as described above (See Matrigel Invasion Assay).

### Endothelial Adhesion Assay

HUVEC's were seeded in 6-well plates and allowed to form monolayers. Cancer cells were serum starved in 0.2% FBS DMEM-based media for 30 minutes and pulsed with Cell Tracker Green CMFDA dye (C7025, Invitrogen) for 45 minutes. 2×10⁵ cancer cells, resuspended in 0.5 mL starvation media, were seeded onto each endothelial monolayer. The cancer cells were allowed to adhere to the HUVEC monolayers for 30 minutes at 37° C. The endothelial monolayers were then washed gently with PBS and fixed with 4% paraformaldehyde for 15 minutes. Each well was then coated with PBS, and 8 images were taken for each endothelial monolayer using an inverted Fluorescence microscope (Zeiss Axiovert 40 CFL) at 10× magnification. The number of cancer cells adhering to HUVEC's was quantified using ImageJ.

### Anoikis Assay

1×10⁶ MeWo cells over-expressing miR-199a, miR-1908, or a control hairpin were seeded in low adherent plates containing cell media supplemented with 0.2% methylcellulose. Following 48 hours in suspension, the numbers of dead and viable cells were counted using trypan blue.

### Serum Starvation Assay

To determine the effects of miR-199a and miR-1908 on melanoma cell serum starvation capacity, 1×10⁵ MeWo parental cells over-expressing miR-199a, miR-1908, or a control hairpin were seeded in quadruplicate into 6-well plates and incubated in 0.2% FBS starvation DMEM-based media for 48 hours, after which the number of viable cells was counted using trypan blue. To determine the effect of recombinant ApoE3 addition on the survival of melanoma cells or endothelial cells in serum starvation conditions, 3×10⁴ MeWo-LM2 cells or endothelial cells were incubated in the presence of ApoE3 (100 μM) or BSA (100 μM) in low serum conditions (0.2% FBS). The number of viable cells was counter after 8, 16, and 24 hours.

### Colony Formation Assay

Fifty MeWo parental cells over-expressing miR-199a, miR-1908, or a control hairpin were seeded in quadruplicate into 6-cm plates. After two weeks, the cells were washed with PBS, fixed with 6% glutaraldehyde, and stained with 0.5% crystal violet. The number of positive-staining colonies was counted.

miRNA Microarray Hybridization

For identification of miRNAs showing deregulated expression across highly metastatic melanoma cell line derivatives, total RNA from multiple independent metastatic derivatives and their respective parental MeWo and A375 cell populations was used to enrich for small RNAs which were then labelled and hybridized onto microfluidic custom microarray platforms by LC sciences. The arrays were designed to detect 894 mature miRNAs corresponding to the miRNA transcripts listed in Sanger miRBase Release 13.0. Out of all the probes analyzed, those corresponding to 169 miRNAs yielded signal above a background threshold across the multiple cell lines analyzed. The raw signal intensities, corresponding to probe hybridization, were median-normalized for each cell line. A threshold of 2-fold or higher upregulation of median-normalized expression values were used in order to identify miRNAs commonly induced in multiple metastatic derivatives for two independent human melanoma cell lines.

Microarray-Based Gene Target Prediction for miR-199a and miR-1908

In order to identify potential genes targeted by miR-199a-3p, miR-199a-5p, and miR-1908, total RNA was extracted from MeWo cell lines with loss- or gain-of-function of each miRNA and submitted to the genomics core facility at The Rockefeller University for hybridization onto Illumina HT-12 v3 Expression BeadChip microarrays. The raw signal intensities, corresponding to probe hybridization, were then median-normalized for each cell line sample. Three sets of microarray profile comparisons were generated: (1) MeWo control cells relative to MeWo cells over-expressing miR-199a or miR-1908, (2) MeWo-LM2 control cells relative to MeWo-LM2 cells expressing a short hairpin (miR-Zip) targeting miR-199a-3p, miR-199a-5p, or miR-1908, and (3) MeWo parental cells relative to MeWo-LM2 cells. Based on the median-normalized expression values from these arrays, the following criteria were used to arrive at possible target genes common to miR-199a and miR-1908: (1) Genes down-regulated by more than 1.5 fold upon individual over-expression of each miR-199a and miR-1908, (2) Genes up-regulated by more than 1.5 fold upon inhibition of either both miR-199a-3p and miR-1908 or both miR-199a-5p and miR-1908, and (3) genes down-regulated by more than 1.5 fold in LM2 cells, which express physiologically higher levels of the three miRNAs, relative to MeWo parental cells.

Analysis of miRNA and mRNA Expression in Cell Lines

Total RNA was extracted from various cell lines using the miRvana kit (AM1560, Applied Biosystems, Austin, Tex.). The expression levels of mature miRNAs were quantified using the Taqman miRNA expression assay (4427975-0002228, Applied Biosystems). RNU44 was used as an endogenous control for normalization. For mRNA expression analyses, 600 ng of total RNA was reverse transcribed using the cDNA First-Strand Synthesis Kit (18080-051, Invitrogen), and roughly 200 ng of the resulting cDNA was then mixed with SYBR green PCR Master Mix (4309155, Applied Biosystems) and the appropriate primers. Each reaction was performed in quadruplicate, and mRNA expression was quantified by performing real-time PCR amplification using an ABI Prism 7900HT Real-Time PCR System (Applied Biosystems). GAPDH was used as an endogenous control for normalization. The following primers were used:

ApoE_Fwd:
5′-TGGGTCGCTTTTGGGATTAC-3′
ApoE_Rev:
5′-TTCAACTCCTTCATGGTCTCG-3′
DNAJA4_Fwd:
5′-CCAGCTTCTCTTCACCCATG-3′
DNAJA4_Rev:
5′-GCCAATTTCTTCGTGACTCC-3′
GAPDH_Fwd:
5′-AGCCACATCGCTCAGACAC-3′
GAPDH_Rev:
5′-GCCCAATACGACCAAATCC-3′
LRP1_Fwd:
5′-TTTAACAGCACCGAGTACCAG-3′
LRP1_Rev:
5′CAGGCAGATGTCAGAGCAG-3′
LRP8_Fwd:
5′-GCTACCCTGGCTACGAGATG-3′
LRP8_Rev:
5′-GATTAGGGATGGGCTCTTGC-3′

### ELISA

Conditioned cancer cell media was prepared by incubating cells in 0.2% FBS serum starvation DMEM-based media for 24 hours. ApoE levels in conditioned media were determined using the APOE ELISA kit (IRAPKT031, Innovative Research, Novi, Mich.).

### Luciferase Reporter Assays

Heterologous luciferase reporter assays were performed as previously described (Tavazoie et al., 2008). In brief, full-length 3′UTRs and CDS's of ApoE and DNAJA4 were cloned downstream of a renilla luciferase reporter into the psiCheck2 dual luciferase reporter vector (C8021, Promega, Madison, Wis.). 5×10⁴ parental MeWo cells, MeWo-LM2 cells, MeWo cells over-expressing miR-199a, miR-1908, or a control hairpin, and MeWo-LM2 cells expressing a miR-Zip hairpin targeting miR-199a-3p, miR-199a-5p, miR-1908, or a control sequence were transfected with 100 ng of the respective specific reporter constructs using TransiT-293 transfection reagent. Twenty-four hours post-transfection, the cells were lysed, and the ratio of renilla to firefly luciferase expression was determined using the dual luciferase assay (E1910, Promega). Putative miRNA binding sites in each target construct were identified by alignment to the complimentary miRNA seed sequences (miR-199a-3p: 5′-CAGUAGUC-3′; miR-199a-5p: 5′-CCAGUGUU-3′; miR-1908: 5′-GGCGGGGA-3′). The miRNA complimentary sites on each target construct were mutated using the QuickChange Multi Site-Directed Mutagenesis Kit (200514, Agilent Technologies, Santa Clara, Calif.). Based on miRNA seed sequence complimentarity analysis, the CDS of ApoE was mutated at position 141 (CTG to ACT), the 3′UTR of ApoE was mutated at positions 83 (GCC to ATA) and 98 (CTG to ACA), the CDS of DNAJA4 was mutated at positions 373 (CGC to TAT) and 917 (CTG to AGA), and the 3′UTR of DNAJA4 was mutated at positions 576 (CTG to ACA), 1096 (CTG to TCT), 1396 (CGC to TGT), and 1596 (CTG to TGT). The following primers were used to clone the 3′UTR's and CDS's of ApoE and DNAJA4:

ApoE_CDS_Fwd:
5′-AGTACCTCGAGGGGATCCTTGAGTCCTACTC-3′
APOE_CDS_Rev:
5′-TAATTGCGGCCGCTCAGACAGTGTCTGCACCCAG-3′
DNAJA4_CDS_Fwd:
5′-TAATATCTCGAGATGTGGGAAAGCCTGACCC-3′
DNAJA4_CDS_Rev:
5′-CAATTGCGGCCGCTCATGCCGTCTGGCACTGC-3′
APOE_3′UTR_Fwd:
5′-TTAGCCTCGAGACGCCGAAGCCTGCAGCCA-3′
APOE_3′UTR_Rev:
5′-TTACTGCGGCCGCTGCGTGAAACTTGGTGAATCTT-3′
DNAJA4_3′UTR_Fwd:
5′-TAATATCTCGAGCGTGGTGCGGGGCAGCGT-3′
DNAJA4_3′UTR_Rev:
5′-CAATTGCGGCCGCTTATCTCTCATACCAGCTCAAT-3′

The following primers were used to mutagenize the miRNA binding sites on each target:

APOE_CDS_mut:
5′-GCCAGCGCTGGGAACTGGCAACTGGTCGCTTTTGGGATTACCT-3′
APOE_3′UTR_mut1:
5′-CAGCGGGAGACCCTGTCCCCATACCAGCCGTCCTCCTGGGGTG-3′
APOE_3′UTR_mut2:
5′-TCCCCGCCCCAGCCGTCCTCACAGGGTGGACCCTAGTTTAATA-3′
DNAJA4_CDS_mut1:
5′-GGGATCGGTGGAGAAGTGCCTATTGTGCAAGGGGCGGGGGATG-3′
DNAJA4_CDS_mut2:
5′-GTAGGGGGCGGGGAACGTGTTATCCGTGAAGAGGTGGCTAGGG-3′
DNAJA4_3′UTR_mut1:
5′-CAGGGCCAACTTAGTTCCTAACATTCTGTGCCCTTCAGTGGAT-3′
DNAJA4_3′UTR_mut2:
5′-ACAGTTTGTATGGACTACTATCTTAAATTATAGCTTGTTTGGA-3′
DNAJA4_3′UTR_mut3:
5′-TAATTATTGCTAAAGAACTATGTTTTAGTTGGTAATGGTGTAA-3′
DNAJA4_3′UTR_mut4:
5′-CAGCTGCACGGACCAGGTTCCATAAAAACATTGCCAGCTAGTGAG-
3′

Analysis of miRNA Expression in Human Melanoma Skin Lesions

All human clinical samples used in this study were obtained, processed, and analyzed in accordance with institutional IRB guidelines. Paraffin-embedded cross-sections of primary melanoma skin lesions from 71 human patients were obtained from MSKCC. The samples were de-paraffinized by five consecutive xylene washes (5 minutes each). Following de-paraffinization, the malignancy-containing region was identified by H&E staining, dissected, and total RNA was extracted from it using the RecoverAll Total Nucleic Acid Isolation Kit (AM1975, Applied Biosystems). The expression levels of mature miR-199a-3p, miR-199a-5p, and miR-1908 in each sample were quantified in a blinded fashion using the Taqman miRNA assay. RNU44 was used as an endogenous control for normalization. The expression levels of each miRNA were compared between primary melanomas with propensity to metastasize and primary melanomas that did not metastasize. Kaplan-Meier curves were plotted using metastasis-free survival data of patients as a function of the expression levels for each miRNA in each patient's tumor. Metastatic recurrence to such sites as lung, brain, bone, and soft tissue were previously documented and allowed for a retrospective analysis of the relationship between the expression levels of identified miRNAs and metastatic recurrence.

### Histology

Animals were perfused with PBS followed by fixation with 4% paraformaldehyde infused via intracardiac and subsequently intratracheal injection. The lungs were sectioned out, incubated in 4% paraformaldehyde at 4° C. overnight, embedded in paraffin, and sliced into 5-μm-thick increments. For gross macroscopic metastatic nodule visualization, lung sections were H&E stained. For endothelial content analysis in metastatic nodules formed by human melanoma MeWo cells in mice, representative lung sections were double-stained with primary antibodies against MECA-32 (Developmental Studies Hybridoma Bank, The University of Iowa, IA), which labels mouse endothelial cells, and human vimentin (VP-V684, Vector Laboratories), which labels human melanoma cells. Various Alexa Flour dye-conjugated secondary antibodies were used to detect primary antibodies. To determine the blood vessel density within metastatic nodules, fluorescence was measured using a Zeiss laser scanning confocal microscope (LSM 510), and the MECA-32 signal within each metastatic nodule, outlined based on co-staining with human vimentin, was quantified in a blinded fashion using ImageJ (NIH). For endothelial content analysis in metastatic nodules formed by mouse B16F10 mouse melanoma cells in wild type and ApoE genetically null mice, representative lung sections were stained for MECA-32, and the MECA-32 signal within each nodule, demarcated based on cell pigmentation, was quantified in a blinded fashion. The collective vessel area, given as the percentage area covered by blood vessels relative to the total area of each metastatic nodule, was obtained by background subtraction (rolling ball radius of 1 pixel) and use of a pre-determined threshold as a cut-off. A metastatic nodule was defined as any region of greater than 2000 μm² total area. For large nodules, minimum of four representative images were obtained, and their average blood vessel density was calculated.

### In Vivo Matrigel Plug Assay

10 μg/mL recombinant human ApoE3 (4696, BioVision), 10 μg/mL BSA (A2153, Sigma Aldrich), or 400 ng/ml VEGF were mixed with matrigel (356231, BD Biosciences) as indicated. 400 μL of matrigel containing the indicated recombinant proteins were injected subcutaneously just above the ventral flank of immunocompromised NOD-SCID mice. Plugs were extracted on day 3 post-injection and fixed in 4% paraformaldehyde for 48 hours. Plugs were then paraffin-embedded and sectioned at 5-μm-thick increments. Plug cross-sectional sections were immunohistochemically stained using a primary antibody against the mouse endothelial antigen MECA-32 (Developmental Studies Hybridoma Bank, The University of Iowa, IA), detected by peroxidase-conjugated secondary antibody, and subsequently visualized by DAB oxidization. To quantify the extent of endothelial cell invasion into each matrigel plug, the number of endothelial cells was counted in 4-5 random fields for each plug, and the average number of endothelial cells per given plug area was calculated.

### Tissue Culture

The SK-Mel-334 primary human melanoma line was established from a soft tissue metastasis of a Braf-mutant melanoma of a patient at the MSKCC. Following minimum expansion in vitro, the cells were in vivo selected (Pollack and Fidler, 1982) to generate the lung-metastatic derivatives SK-Mel-334.2. The SK-Mel-239 vemurafenib-resistant clone (C1) was a gift from Poulikos Poulikakos (Mount Sinai Medical School) and the B-Raf^{V600E/+}; Pten^{-/-}; CDKN2A^{-/-} primary murine melanoma cell line was generously provided by Marcus Rosenberg (Yale University). All other cell lines used were purchased from ATCC.

### ApoE ELISA

Extracellular ApoE levels in serum-free conditioned media from melanoma cells treated with DMSO, GW3965, or T0901317 (1 μM each) were quantified using the ApoE ELISA kit (Innovative Research) at 72 hours following treatment.

### Western Blotting

Mouse lung and brain tissue samples were homogenized on ice in RIPA buffer (Sigma-Aldrich) supplemented with protease inhibitors (Roche). Mouse adipose tissue was homogenized on ice in TNET buffer (1.5 mM Tris pH 7.5, 150 mM NaCl, 2 mM EDTA 1% triton, protease inhibitors). Total protein lysate (2 μg) was separated by SDS-PAGE, transferred to PVDF membrane, and blotted with an anti-mouse ApoE (ab20874, Abcam) and anti-tubulin a/3 (2148, Cell Signaling) antibodies.

### ApoE Expression Analysis in Melanoma Clinical Samples

All clinical sample procurement, processing, and analyses were performed in strict agreement with IRB guidelines. Primary melanoma skin lesions were previously resected from patients at the MSKCC, formalin-fixed, paraffin-embedded, and sectioned into 5-μm-thick slides. ApoE protein expression was assessed by double-blinded immunohistochemical analysis using the D6E10 anti-ApoE antibody (ab1906, Abcam).

### Histochemistry

Animals were intracardially perfused with PBS followed by 4% paraformaldehyde (PFA). Fixed lungs were embedded in paraffin and sectioned into 5-μm-thick increments. Macroscopic lung metastatic nodules were visualized by H&E staining. For analysis of tumor endothelial cell content, proliferation, and apoptosis, primary tumor paraffin-embedded sections were stained with antibodies against MECA-32 (Developmental Studies Hybridoma Bank, University of Iowa), KI-67 (ab15580, Abcam), and cleaved caspase-3 (9661, Cell Signaling), respectively.

### Tail-Vein Metastasis Assays

Melanoma cells used for in vivo metastasis assays were transduced with a stably expressed retroviral construct encoding a luciferase reporter gene (Ponomarev et al., 2004), allowing us to monitor the in vivo progression of melanoma cells by bioluminescence imaging. The following numbers of melanoma cells, resuspended in 100 μL of PBS, were injected intravenously via the tail-vein: 4×10⁴ MeWo cells, 2.5×10⁵ HT-144 cells, 2×10⁵ SK-Mel-334.2 cells, 5×10⁴ B16F10 cells, and 1×10⁵ YUMM cells. The MeWo, HT-144, and SK-Mel-334.2 cells were injected into 6-8 week-old sex-matched NOD scid mice, while the B16F10 and YUMM cells were injected into 6-8 week-old sex-matched C57BL/6 mice. In all experiments assessing at the effects of GW3965 on metastasis formation, mice were pre-treated on a control diet or a GW3965-supplemented diet (20 mg/kg) for 10 days. To assess the effect of GW3965 treatment on brain metastasis, 1×10⁵ MeWo brain-metastatic derivatives were injected intracardially into athymic nude mice. Immediately following injection, mice were randomly assigned to a control diet or GW3965-supplemented diet (100 mg/kg). To determine whether oral delivery of GW3965 can inhibit the progression of incipient metastasis, NOD Scid mice were intravenously injected with 4×10⁴ MeWo cells and the cells were allowed to colonize the lungs for 42 days, after which mice were blindedly assigned to a control diet or a GW3965-supplemented diet (100 mg/kg) treatment.

### Orthotopic Metastasis Assays

To determine the effect of GW3965 treatment on lung colonization by melanoma cells dissociated from an orthotopic site, 1×10⁶ MeWo cells expressing a luciferase reporter were subcutaneously injected into both lower flanks of NOD Scid mice. Upon the formation of tumors measuring ˜300 mm³ in volume, the tumors were excised and the mice were randomly assigned to a control diet or a GW3965-supplemented diet (100 mg/kg) treatment. One month after tumor excision, the lungs were extracted and lung colonization was measured by ex vivo bioluminescence imaging. To histologically confirm the extent of melanoma lung colonization, lungs were then fixed in 4% PFA overnight, paraffin-embedded, section into 5-μM increments and stained for human vimentin (VP-V684, Vector Laboratories).

### Generation of Dacarbazine-Resistant Melanoma Cells

Dacarbazine-resistant B16F10 mouse melanoma cells were generated by continuously culturing the cells in the presence of DTIC (D2390, Sigma-Aldrich, St. Louis, Mo.). First, the cells were treated with 500 μg/mL DTIC for one week. Following this initial DTIC treatment, the remaining (˜10%) viable cells were allowed to recover for one week, after which 750 μg/mL of DTIC was added to the cell media for 5 days. Subsequent to this high-dose treatment, the cells were allowed to recover in the presence of low-dose DTIC (100 μg/mL) for one week. The cells were then continuously cultured in cell media containing 200 μg/mL DTIC for at least one month prior to grafting the cells into mice. DTIC was added to fresh cancer cell media every 3 days. For tumor growth experiments, 5×10⁴ B16F10 parental and DTIC-resistant cells were subcutaneously injected into the lower flank of 7-week-old C57BL/6 mice. Following formation of small tumors measuring 5-10 mm³ in volume, the mice were randomly assigned to the following treatment groups: (1) control diet+vehicle, i.p.; (2) control diet+DTIC i.p. (50 mg/kg); (3) GW3965-supplemented diet (100 mg/kg)+vehicle i.p. DTIC was dissolved in the presence of citric acid (1:1 by weight) in water and administered daily by intraperitoneal injection.

The DTIC-resistant MeWo human melanoma cell line clone was generated following DTIC treatment of mice bearing MeWo tumors measuring 600-800 mm³ in volume. After initial tumor shrinkage in response to daily DTIC dosing (50 mg/kg, i.p.) during the first two weeks, the tumors eventually developed resistance and resumed growth, at which point tumor cells were dissociated and the DTIC-resistant MeWo cell line was established. The cells were expanded in vitro in the presence of DTIC (200 μg/mL) for one week, after which 5×10⁵ DTIC-resistant MeWo cells were re-injected into 8-week old Nod SCID gamma mice. Following growth of tumors to 5-10 mm³ in volume, mice were blindedly assigned to the following treatment groups: (1) control diet; (2) control diet+DTIC (50 mg/kg); (3) GW3965-supplemented diet (100 mg/kg). To determine the effect of DTIC on tumor growth by parental unselected MeWo cells, 5×10⁵ MeWo cells were subcutaneously injected into Nod SCID gamma mice, and the mice were treated with a control vehicle or DTIC (50 mg/kg) subsequent to formation of tumors measuring 5-10 mm³ in volume. DTIC was administered daily, as described above, in cycles consisting of 5 consecutive daily treatments interspersed by 2-day off-treatment intervals. Tumor growth was measured twice a week.

### Genetically-Initiated Model of Melanoma Progression

The Tyr::CreER; B-Raf^{V600E/+;} Pten^{lox/+}/Tyr::CreER; B-Raf^{V600E/+;} Pten^{lox/lox} conditional model of melanoma progression was previously established and characterized by Dankort et al. (2009). Briefly, melanoma in these mice was induced at 6 weeks of age by intraperitoneally injecting 4-HT (H6278, 70% isomer, Sigma-Aldrich, St Louis, Mo.) at 25 mg/kg administered in peanut oil on three consecutive days. The 4-HT stock solution was prepared by dissolving it in 100% EtOH at 50 mg/mL by heating at 45° C. for 5 min and mixing. Once dissolved, the stock 4-HT solution was then diluted by 10-fold in peanut oil, yielding a 5 mg/mL 4-HT working solution that was then injected into mice. After the first 4-HT injection, mice were blindedly assigned to receive either a control diet or a diet supplemented with GW3965 (100 mg/kg). Mice were examined three times a week for the presence and progression of melanoma lesions. At day 35, dorsal skin samples were harvested from control-treated and GW3965-treated mice, fixed in 4% PFA and photographed at 10×. The percentage of pigmented melanoma lesion area out of the total skin area was quantified using ImageJ. For survival analyses, mice were monitored daily for melanoma progression and euthanized according to a standard body condition score, taking into account initial signs of moribund state and discomfort associated with the progression of melanoma burden. Post-mortem, the lungs, brains, and salivary glands were harvested and examined for the presence of macroscopic melanoma lesions.

### Mouse Genotyping

All mouse genotyping was performed using standard PCR conditions, as recommended by Jackson Labs. The following genotyping primers were used for the respective PCR reactions: Tyr::CreER; B-Raf^{V600E/+;} Pten^{lox/+} and Tyr::CreER; B-Raf^{V600E/+;} Pten^{lox/lox} mice:

B-Raf Forward:
5′-TGA GTA TTT TTG TGG CAA CTG C-3′
B-Raf Reverse:
5′-CTC TGC TGG GAA AGC GGC-3′
Pten Forward:
5′-CAA GCA CTC TGC GAA CTG AG-3′
Pten Reverse:
5′-AAG TTT TTG AAG GCA AGA TGC-3′
Cre Transgene Forward:
5′-GCG GTC TGG CAG TAA AAA CTA TC-3′
Cre Transgene Reverse:
5′-GTG AAA CAG CAT TGC TGT CAC TT-3′
Internal Positive Control Forward:
5′-CTA GGC CAC AGA ATT GAA AGA TCT-3′
Internal Positive Control Reverse:
5′-GTA GGT GGA AAT TCT AGC ATC ATC C-3′

ApoE−/− mice:

Common Forward:
5′-GCC TAG CCG AGG GAG AGC CG-3′
Wild-type Reverse:
5′-TGT GAC TTG GGA GCT CTG CAG C-3′
Mutant Reverse:
5′-GCC GCC CCG ACT GCA TCT-3′

LXRα−/− mice:

Common Forward:
5′-TCA GTG GAG GGA AGG AAA TG-3′
Wild-type Reverse:
5′-TTC CTG CCC TGG ACA CTT AC-3′
Mutant Reverse:
5′-TTG TGC CCA GTC ATA GCC GAA T-3′

LXRβ−/− mice:

Common Forward:
5′-CCT TTT CTC CCT GAC ACC G-3′
Wild-type Reverse:
5′-GCA TCC ATC TGG CAG GTT C-3′
Mutant Reverse:
5′-AGG TGA GAT GAC AGG AGA TC-3′

### Cell Proliferation and Viability Assay:

To determine the effects of GW3965, T0901317, and Bexarotene on in vitro cell growth, 2.5×10⁴ melanoma cells were seeded in triplicate in 6-well plates and cultured in the presence of DMSO, GW3965, T0901317, or Bexarotene at 1 μM each. After 5 days, the number of viable and dead cells was counted using the trypan blue dye (72-57-1, Sigma-Aldrich), which selectively labels dead cells.

### Cell Invasion Assay

The cell invasion assay was performed as previously described in detail (Pencheva et al., 2012) using a trans-well matrigel invasion chamber system (354480, BD Biosciences). In brief, various melanoma cells were cultured in the presence of DMSO, GW3965, T0901317, or Bexarotene at 1 μM for 56 hours, after which melanoma cells were switched to starvation media (0.2% FBS) for 16 hours in the presence of each drug. Following starvation, cells were seeded into matrigel-coated trans-well inserts, and the invasion assay was allowed to proceed for 24 hours at 37% C. For ApoE antibody neutralization experiments, 40 μg/mL 1 D7 anti-ApoE blocking antibody (Heart Institute, University of Ottawa, Ottawa, Canada) or 40p g/mL anti-IgG control antibody (AB-108-C, R&D Systems, Minneapolis, Minn.) was added to each trans-well insert at the start of the assay.

### Endothelial Recruitment Assay

The endothelial recruitment assay was carried out as previously described (Pencheva et al., 2012; Png et al., 2012). Melanoma cells were treated with DMSO, GW3965, T0901317, or Bexarotene at 1 μM for 56 hours, after which 5×10⁴ cells were seeded in a 24-well plate in the presence of each drug and allowed to attach for 16 hours prior to starting the assay. HUVEC cells were serum-starved overnight in EGM-2 media containing 0.2% FBS. The following day, 1×10⁵ HUVEC cells were seeded into a 3.0 μm HTS Fluoroblock trans-well migration insert (351151, BD Falcon, San Jose, Calif.) fitted into each well containing cancer cells at the bottom. The HUVEC cells were allowed to migrate towards the cancer cells for 20 hours at 37% C, after which the inserts were processed as previously described (Pencheva et al., 2012). For ApoE antibody neutralization experiments, 40 μg/mL 1 D7 anti-ApoE blocking antibody (Heart Institute, University of Ottawa, Ottawa, Canada) or 40 μg/mL anti-IgG control antibody (AB-108-C, R&D Systems, Minneapolis, Minn.) was added to each trans-well insert at the start of the assay.

Lentiviral shRNA-Based Gene Knockdown

shRNAs were integrated into lentiviral particles that were prepared by transfection of 6 μg of vector A, 12 μg of vector K, and 12 μg of shRNA plasmid into HEK-293T packaging cells, as previously described (Pencheva et al., 2012; Png et al., 2012). Lentiviral shRNA transduction was performed in the presence of 10 μg/mL of polybrene (TR-1003-G, Millipore, Billerica, Mass.) for 6 hours, as described previously (Pencheva et al., 2012). The cells were expanded for 72 hours after transduction and lentiviral selection was performed by culturing the cells in the presence of 2 μg/mL of puromycin (P8833, Sigma-Aldrich) for 72 hours.

The following shRNA sequences were used:

### Human:

sh₁LXRα:
5′-CCGGCCGACTGATGTTCCCACGGATCTCGAGATCCGTGGGAACATC
AGTCGGTTTTT-3′
sh₂LXRα:
5′-CCGGGCAACTCAATGATGCCGAGTTCTCGAGAACTCGGCATCATTG
AGTTGCTTTTT-3′
sh₁LXRβ:
5′-CCGGAGAGTGTATCACCTTCTTGAACTCGAGTTCAAGAAGGTGATAC
ACTCTTTTTT-3′
sh₂LXRβ:
5′-CCGGGAAGGCATCCACTATCGAGATCTCGAGATCTCGATAGTGGAT
GCCTTCTTTTT-3′
shApoE:
5′-CCGGGCAGACACTGTCTGAGCAGGTCTCGAGACCTGCTCAGACAG
TGTCTGCTTTTT-3′

### Mouse:

sh_mLXRα:
5′-CCGGGCAACTCAATGATGCTGAGTTCTCGAGAACTCAGCATCATT
GAGTTGCTTTTT-3′
sh_mLXRβ:
5′-CCGGTGAGATCATGTTGCTAGAAACCTCGAGGTTTCTAGCAACATG
ATCTCATTTTTG-3′
sh_mApoE:
5′-CGGGAGGACACTATGACGGAAGTACTCGAGTACTTCCGTCATAG
TGTCCTCTTTTT-3′

Gene Expression Analysis by qRT-PCR:

RNA was extracted from whole cell lysates using the Total RNA Purification Kit (17200, Norgen, Thorold, Canada). 600 ng of total RNA was then reverse transcribed into cDNA using the cDNA First-Strand Synthesis Kit (18080-051, Invitrogen), and quantitative real-time PCR amplification was performed as previously described (Pencheva et al., 2012) using an ABI Prism 7900HT Real-Time PCR System (Applied Biosystems, Austin, Tex.). Each PCR reaction was carried out in quadruplicates. Gene expression was normalized to GAPDH, which was used as an endogenous control.

The following primers were used:

### Human:

ApoE Forward:
5′-TGGGTCGCTTTTGGGATTAC-3′
ApoE Reverse:
5′-TTCAACTCCTTCATGGTCTCG-3′
GAPDH Forward:
5′-AGCCACATCGCTCAGACAC-3′
GAPDH Reverse:
5′-GCCCAATACGACCAAATCC-3′
LXRα_Fwd:
5′-GTTATAACCGGGAAGACTTTGC-3′
LXRα_Rev:
5′-AAACTCGGCATCATTGAGTTG-3′
LXRβ_Fwd:
5′-TTTGAGGGTATTTGAGTAGCGG-3′
LXRβ_Rev:
5′-CTCTCGCGGAGTGAACTAC-3′

### Mouse:

ApoE Forward:
5′-GACCCTGGAGGCTAAGGACT-3′
ApoE Reverse:
5′-AGAGCCTTCATCTTCGCAAT-3′
GAPDH Forward:
5′-GCACAGTCAAGGCCGAGAAT-3′
GAPDH Reverse:
5′-GCCTTCTCCATGGTGGTGAA-3′
LXRα Forward:
5′-GCGCTCAGCTCTTGTCACT-3′
LXRα Reverse:
5′-CTCCAGCCACAAGGACATCT-3′
LXRβ Forward:
5′-GCTCTGCCTACATCGTGGTC-3′
LXRβ Reverse:
5′-CTCATGGCCCAGCATCTT-3′
ABCA1 Forward:
5′-ATGGAGCAGGGAAGACCAC-3′
ABCA1 Reverse:
5′-GTAGGCCGTGCCAGAAGTT-3′

### ApoE Promoter Activity Assay

The ApoE promoter, consisting of a sequence spanning 980 base pairs upstream and 93 base pairs downstream of the ApoEgene, was cloned into a pGL3-Basic vector (E1751, Promega Corporation, Madison, Wis.) upstream of the firefly luciferase gene using NheI and SacI restriction enzymes. Then, multi-enhancer elements 1 (ME.1) and 2 (ME.2) were cloned directly upstream of the ApoE promoter using MluI and SacI restriction enzymes. To assess ApoE promoter- and ME.1/ME.2-driven transcriptional activation by LXR agonists, 5×10⁴ MeWo cells were seeded into a 24-well plate. The following day, 100 ng of pGL3-ME.1/ME.2-ApoEpromoter construct and 2 ng of pRL-CMV renilla luciferase construct (E2261, Promega) were co-transfected into cells in the presence of DMSO, GW3965, or T0901317 at 1 μM, each condition in quadruplicate. To assess transcriptional activation by LXRα or LXRβ, 5×10⁴ MeWo cells expressing a control shRNA or shRNA targeting LXRα or LXRβ were seeded into a 24-well plate. The following day, 200 ng of pGL3-ME.1/ME.2-ApoEpromoter construct and 2 ng of pRL-CMV renilla luciferase were co-transfected into cells in the presence of DMSO, GW3965, or T0901317 at 1 μM, each condition in quadruplicate. After 24 hours, cells were lysed, and cell lysate was analyzed for firefly and renilla luciferase activity using the Dual Luciferase Assay System (E1960, Promega) and a Bio-Tek Synergy NEO Microplate Reader. Firefly luciferase signal was normalized to renilla luciferase signal and all data are expressed relative to the luciferase activity ratio measured in the DMSO-treated control cells.

The following cloning primers were used:

ApoE-promoter Forward:
5′-TCA TAG CTA GCG CAG AGC CAG GAT TCA CGC CCT
G-3′
ApoE-promoter Reverse:
5′-TGG TCC TCG AGG AAC CTT CAT CTT CCT GCC TGT
GA-3′
ME.1 Forward:
5′-TAG TTA CGC GTA GTA GCC CCC ATC TTT GCC-3′
ME.1 Reverse:
5′-AAT CAG CTA GCC CCT CAG CTG CAA AGC TC-3′
ME.2 Forward:
5′-TAG TTA CGC GTA GTA GCC CCC TCT TTG CC-3′
ME.2 Reverse:
5′-AAT CAG CTA GCC CTT CAG CTG CAA AGC TCT G-3′

### Tumor Histochemistry

Tumors were excised from mice and fixed in 4% paraformaldehyde at 4° C. for 48 hours. Then, tumors were paraffin-embedded and sectioned into 5-μm-thick increments. For endothelial cell content analysis in tumors, tumor sections were stained with a primary antibody against the mouse endothelial cell marker MECA-32 (Developmental Studies Hybridoma Bank, The University of Iowa, IA) and counterstained with DAPI nuclear stain. To determine tumor cell proliferation and apoptosis, tumor sections were stained with antibodies against the proliferative marker Ki-67 (Abcam, ab15580, Cambridge, Mass.) and the apoptotic marker cleaved caspase-3 (9661, Cell Signaling, Danvers, Mass.), respectively. Various Alexa Flour dye-conjugated secondary antibodies were used to detect primary antibodies. Fluorescence was measured using inverted fluorescence microscope (Zeiss Axiovert 40 CFL) at 5× magnification for MECA-32 and Ki-67 staining and 10× magnification for cleaved caspase-3 staining. Endothelial cell content density and tumor proliferation rate were quantified by calculating the average percentage of MECA-32 or Ki-67 positively-staining area out of the total tumor area. Tumor apoptosis was measured by counting the number of cleaved caspase-3 expressing cells per given tumor area.

### Analysis of ApoE Expression in Primary Melanoma Lesions

Human primary melanoma skin samples were resected from melanoma patients at MSKCC, formalin-fixed, embedded in paraffin, and sectioned into 5-μm-thick increments. To determine ApoE protein expression, the samples were first de-paraffinized by two consecutive xylene washes (5 minutes each), and rehydrated in a series of ethanol washes (100%, 95%, 80%, and 70% EtOH). ApoE antigen was retrieved by incubating the samples in the presence of proteinase K (5 μg/mL) for 20 minutes at room temperature. To quench endogenous peroxidase activity, the slides were incubated in 3% H₂O₂ solution. The slides were then blocked in three consecutive Avidin, Biotin, and horse serum block solutions for 15 min each at room temperature (SP-2001, Vector Laboratories, Burlingame, Calif.). ApoE was detected by staining with D6E10 anti-ApoE antibody (ab1908, Abcam), which was used at a 1:100 dilution in PBS at 4° C. overnight. The primary antibody was then recognized by incubating the slides in a peroxidase-conjugated secondary antibody (PK-4002, Vector Laboratories) and exposed by DAB (SK-4105, Vector Laboratories) oxidation reaction. The slides were imaged at 10× magnification and analysed in a double-blinded manner. ApoE expression was quantified by counting the number of DAB-positive cells and measuring the area of extracellular ApoE staining. Total ApoE staining signal was expressed as the percentage staining area per given tumor area, determined based on matched H&E-stained slides for each sample. Kaplan-Meier curves depicting patients' metastasis-free survival times were generated by plotting each patient's relapse-free survival data as a function of ApoE expression in that patient's primary melanoma lesion. Patients whose tumors had ApoE levels lower than the median ApoE expression of the population were classified as ApoE-negative, whereas patients whose melanomas expressed ApoE above the median were classified as ApoE-positive. Previously documented patients' history of metastatic recurrence to sites such as lung, brain, bone, soft and subcutaneous tissues, and skin enabled us to retrospectively determine the relationship between ApoE expression at a primary melanoma site and metastatic relapse.

### Example 2 Treatment with LXR Agonist GW3965 Elevates Melanoma Cell ApoE and DNAJA4 Levels

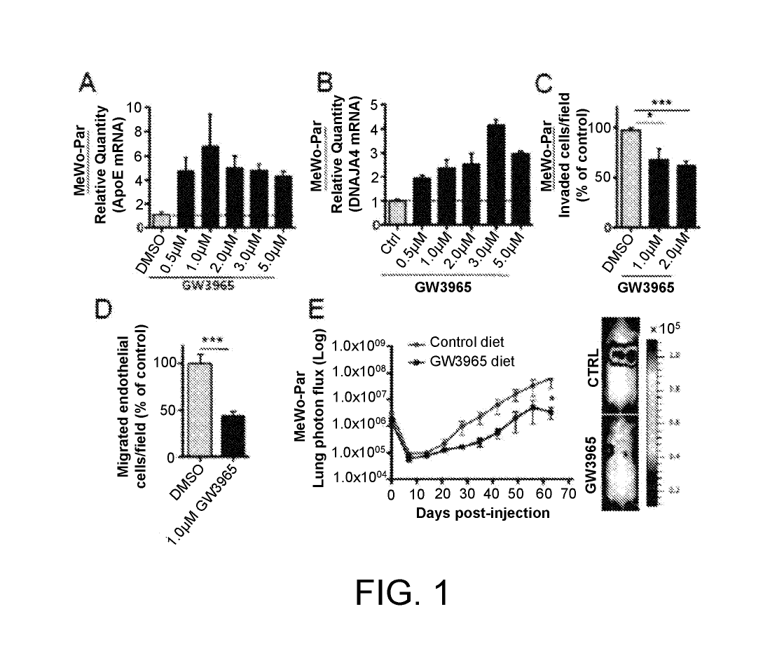

and Suppresses Cancer Cell Invasion, Endothelial Recruitment, and Metastatic Colonization Small molecule agonists of the Liver X Receptor (LXR) have previously been shown to increase Apo E levels. To investigate whether increasing Apo-E levels via LXR activation resulted in therapeutic benefit, assays were carried out to assess the effect of the LXR agonist GW3965 [chemical name: 3-[3-[N-(2-Chloro-3-trifluoromethylbenzyl)-(2,2-diphenylethyl)amino]propyloxy] phenylacetic acid hydrochloride) on Apo-E levels, tumor cell invasion, endothelial recruitment, and in vivo melanoma metastasis (FIG. 1). Incubation of parental MeWo cells in the presence of therapeutic concentrations of GW3965 increased expression of ApoE and DNAJA4 (FIGS. 1A and 1B). Pre-treatment of MeWO cells with GW3965 decreased tumor cell invasion (FIG. 1C) and endothelial recruitment (FIG. 1D). To test whether GW3965 could inhibit metastasis in vivo, mice were administered a grain-based chow diet containing GW3965 (20 mg/kg) or a control diet, and lung metastasis was assayed using bioluminescence after tail-vein injection of 4×10⁴ parental MeWo cells into the mice (FIG. 1E). Oral administration of GW3965 to the mice in this fashion resulted in a significant reduction in in vivo melanoma metastasis (FIG. 1E).

### Example 3 Identification of LXRβ Signaling as a Novel Therapeutic Target in Melanoma

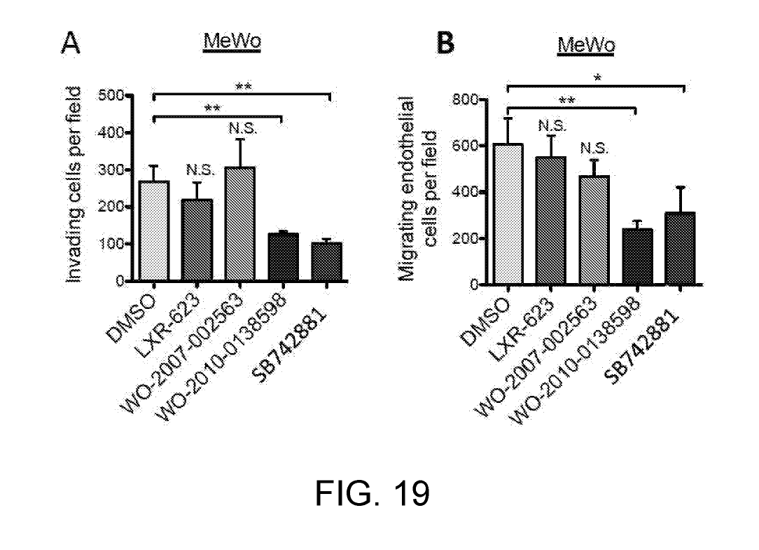

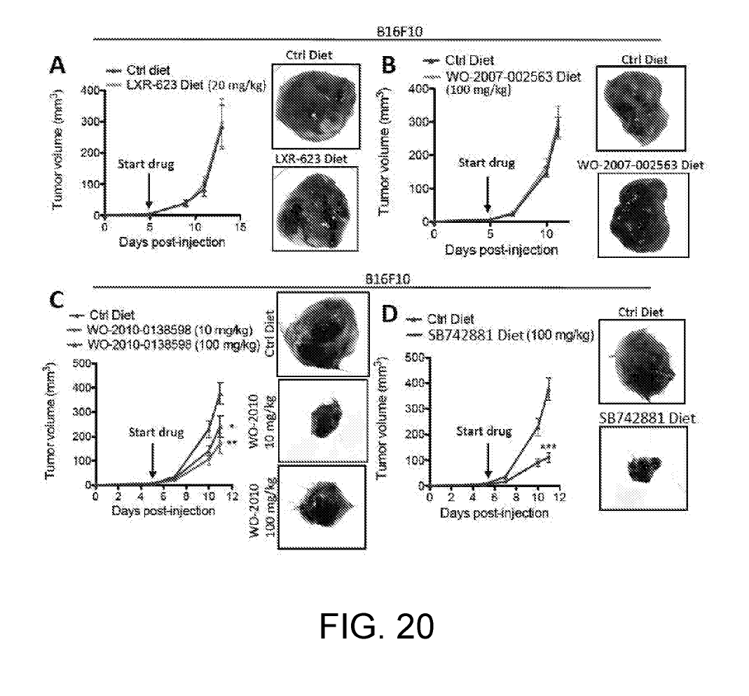

To identify nuclear hormone receptors that show broad expression in melanoma, the expression levels of all nuclear hormone receptor family members across the NCI-60 collection of human melanoma cell lines has been examined. Several receptors exhibited stable expression across multiple melanoma lines, suggesting that they could represent novel potential targets in melanoma (FIGS. 19A and 20A). Notably, out of these, liver-X receptors (LXRs) were previously shown to enhance ApoE transcription in adipocytes and macrophages (Laffitte et al., 2001), while pharmacologic activation of RXRs was found to drive ApoE expression in pre-clinical Alzheimer's models (Cramer et al., 2012).

Given the recently uncovered metastasis-suppressive role of ApoE in melanoma (Pencheva et al., 2012), the ubiquitous basal expression of LXRβ and RXRα in melanoma, and the availability of pharmacologic agents to therapeutically activate LXRs and RXRs, whether activation of LXRs or RXRs in melanoma cells might inhibit melanoma progression phenotypes has been investigated. In light of the established roles of nuclear hormone receptors such as ER and AR in regulating breast and prostate cancer cell proliferation, whether pharmacologic agonism of LXRs or RXRs in melanoma cells affects in vitro cell growth has been examined.

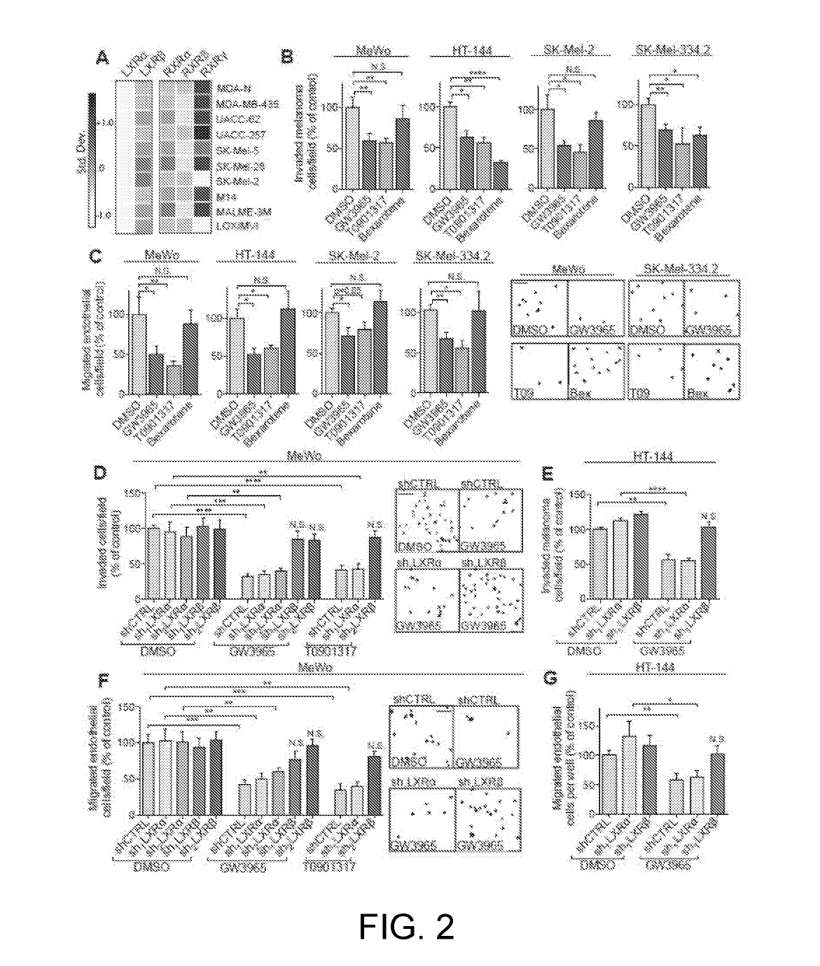

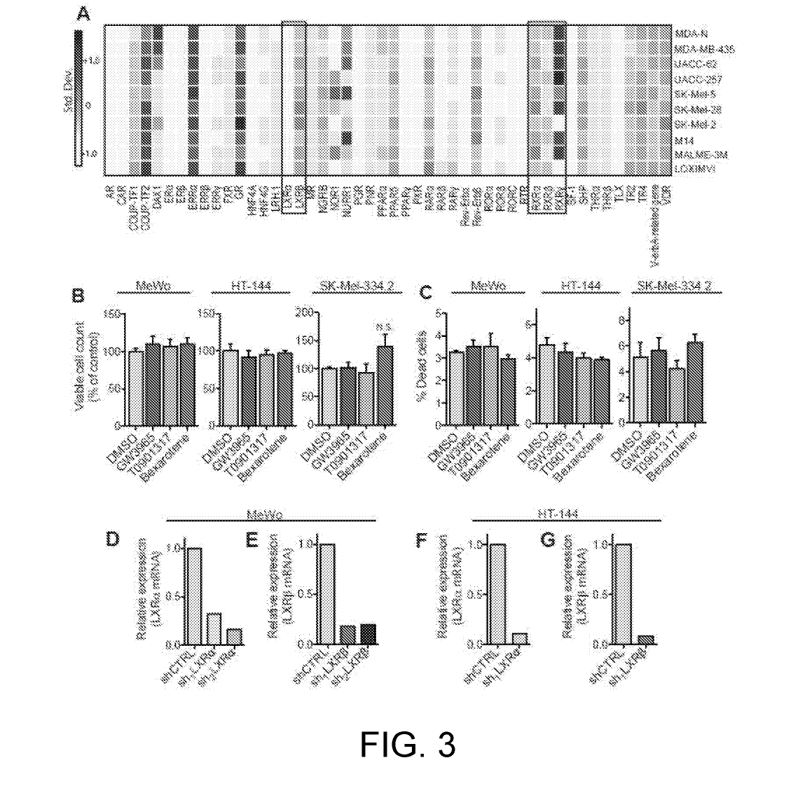

Treatment of melanoma cells with two structurally-distinct LXR agonists, GW3965 108 or T0901317 110, or the RXR agonist bexarotene did not affect cell proliferation or cell viability rates (FIG. 3 B-C). The effects of LXR or RXR activation on cell invasion and endothelial recruitment-phenotypes displayed by metastatic melanoma and metastatic breast cancer populations (Pencheva et al., 2012; Png et al., 2012) has been assessed. Treatment of the mutationally diverse MeWo (B-Raf/N-Ras wild-type), HT-144 (B-Raf mutant), and SK-Mel-2 (N-Ras mutant) human melanoma lines as well as the SK-Mel-334.2 (B-Raf mutant) primary human melanoma line with GW3965 108 or T0901 317 110 consistently suppressed the ability of melanoma cells to invade through matrigel and to recruit endothelial cells in trans-well assays (FIG. 2B-C). In comparison, treatment with bexarotene suppressed invasion only in half of the melanoma lines tested and it did not significantly affect the endothelial recruitment phenotype (FIGS. 2B-C).

Given the superiority of LXR over RXR agonism in broadly inhibiting both cell invasion and endothelial recruitment across multiple melanoma lines, the requirement for LXR signaling in mediating the suppressive effects of LXR agonists has been investigated. Knockdown of melanoma LXRβ, but not LXRα, abrogated the ability of GW3965 108 and T0901317 110 to suppress invasion and endothelial recruitment (FIG. 2D-G and FIGS. 20D-G), revealing melanoma-cell LXRβ to be the functional target of LXR agonists in eliciting the suppression of these in vitro phenotypes. The molecular findings are consistent with LXRβ being the predominant LXR isoform expressed by melanoma cells (FIG. 2A, P<0.0001).

The ubiquitous basal expression of LXRβ in melanoma is likely reflective of the general role that LXRs play in controlling lipid transport, synthesis, and catabolism (Calkin and Tontonoz, 2013). While such stable LXRβ expression would be key to maintaining melanoma cell metabolism and growth, it also makes LXR signaling an attractive candidate for broad-spectrum therapeutic targeting in melanoma.

### Example 4 Therapeutic Delivery of LXR Agonists Suppresses Melanoma Tumor Growth

LXR agonists were originally developed as oral drug candidates for the purpose of cholesterol lowering in patients with dyslipidemia and atherosclerosis (Collins et al., 2002; Joseph and Tontonoz, 2003). These compounds were abandoned clinically secondary to their inability to reduce lipid levels in large-animal pre-clinical models (Groot et al., 2005).

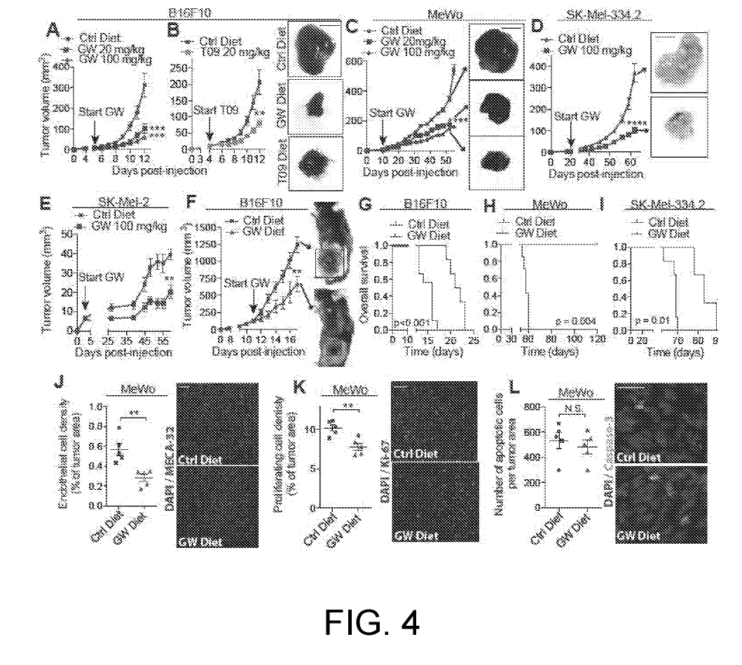

Given the robust ability of GW3965 108 and T0901317 110 to suppress in vitro melanoma progression phenotypes (FIG. 2B-C), whether therapeutic LXR activation could be utilized for the treatment of melanoma has been investigated. Indeed, oral administration of GW3965 108 or T0901317 110 at low doses (20 mg/kg), subsequent to formation of subcutaneous tumors measuring 5-10 mm° in volume, suppressed tumor growth by the aggressive B16F10 mouse melanoma cells in an immunocompetent model by 67% and 61%, respectively (FIG. 4A-B). Administration of a higher LXR agonist dose (100 mg/kg) led to an 80% reduction in tumor growth (FIG. 4A), consistent with dose-dependent suppressive effects.

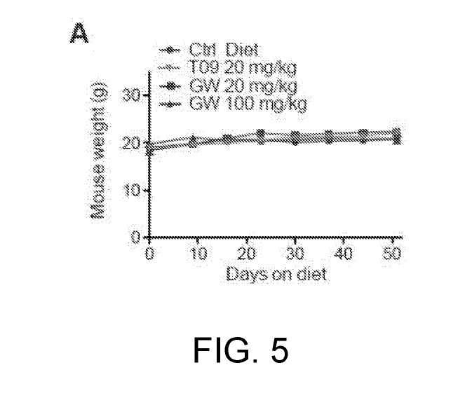

Oral administration of GW3965 108 also robustly suppressed tumor growth by the MeWo (70% inhibition) and SK-Mel-2 (49% inhibition) human melanoma cell lines, as well as the SK-Mel-334.2 primary human melanoma line (73% inhibition) (FIG. 4C-E and FIG. 5A).

Encouraged by the robust tumor-suppressive impact of LXR agonists on small tumors (5-10 mm³) (FIG. 4A-E), whether LXR activation therapy could inhibit the growth of large (˜150 mm³) tumors has been investigaged.

Treatment with GW3965 108 led to a roughly 50% reduction in the growth of established large B16F10 tumors (FIG. 4F). Importantly, therapeutic delivery of GW3965 2 subsequent to tumor establishment substantially prolonged the overall survival time of immunocompetent mice injected with mouse B16F10 cells, immunocompromised mice bearing tumor xeongrafts derived from the human MeWo established melanoma line, as well as the SK-Mel.334-2 primary human melanoma line (FIG. 4G-I). These findings are consistent with broad-spectrum responsiveness to LXR activation therapy across melanotic and amelanotic established melanoma tumors of diverse mutational subtypes: B-Raf and N-Ras wild-type (B16F10 and MeWo; FIG. 4A-C), B-Raf mutant (SK-Mel-334.2; FIG. 4D), and N-Ras mutant (SK-Mel-2; FIG. 4E).

Determination of the cell biological phenotypes regulated by LXR agonists in suppressing tumor growth has been investigated. Consistent with the inhibitory effects of GW3965 108 on endothelial recruitment by melanoma cells in vitro, GW3965 108 administration led to a roughly 2-fold reduction in the endothelial cell content of tumors (FIG. 4J). This effect was accompanied by a modest decrease (23%) in the number of actively proliferating tumor cells in vivo (FIG. 4K) without a change in the number of apoptotic cells (FIG. 4L). These results suggest that, in addition to reducing local tumor invasion, LXR activation suppresses melanoma tumor growth primarily through inhibition of tumor angiogenesis with a resulting reduction in in vivo proliferation.

### Example 5 LXR Agonism Suppresses Melanoma Metastasis to the Lung and Brain and Inhibits the Progression of Incipient Metastases

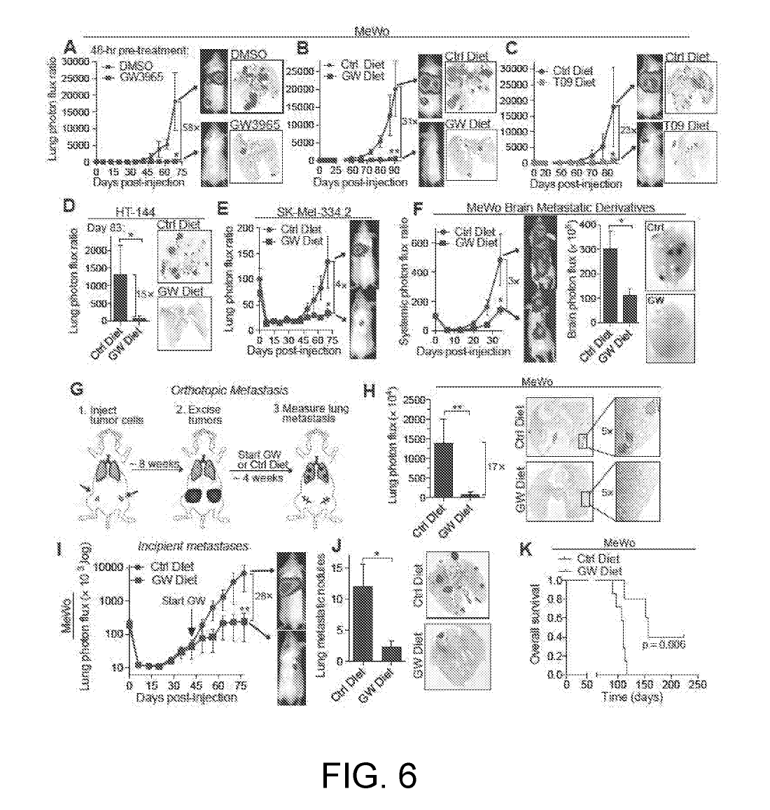

The strong suppressive effects of LXR agonists on melanoma tumor growth motivated us to examine whether LXR activation could also suppress metastatic colonization by melanoma cells. To this end, pre-treatment of human MeWo melanoma cells with GW3965 108 led to a more than 50-fold reduction in their metastatic colonization capacity (FIG. 6A). The ability of orally administered LXR agonists to suppress metastasis has been assessed. Immunocompromised mice that were orally administered GW3965 108 or T0901317 110 experienced 31-fold and 23-fold respective reductions in lung metastatic colonization by human MeWo cells (FIG. 6B-C). Treatment with GW3965 2 also suppressed metastatic colonization by the HT-144 melanoma line (FIG. 6D) as well as the SK-Mel-334.2 primary melanoma line (FIG. 6E).

GW3965 108 is a lipophilic molecule that can efficiently cross the blood brain barrier and potently activate LXR signaling in the brain. Consistent with this, oral delivery of GW3965 108 was previously shown to improve amyloid plaque pathology and memory deficits in pre-clinical models of Alzheimer's disease (Jiang et al., 2008). Notably, oral administration of GW3965 108 inhibited both systemic dissemination and brain colonization following intracardiac injection of brain-metastatic melanoma cells derived from the MeWo parental line (FIG. 6F). These results reveal robust metastasis suppression by LXR activation therapy across multiple melanoma lines and in multiple distal organ metastatic sites. Encouraged by the robust effects observed in suppressing metastasis formation (FIG. 6A-F), whether LXR activation therapy could halt the progression of melanoma cells that had already metastatically disseminated has been determined. The ability of GW3965 108 to reduce lung colonization by melanoma cells disseminating from an orthotopic site following removal of the primary tumor (FIG. 6G) has been tested. Importantly, oral administration of GW3965 108 post-tumor excision inhibited lung colonization by disseminated melanoma cells by 17-fold (FIG. 6H). Remarkably, treatment of mice with GW3965 108 also dramatically suppressed (28-fold) colonization by incipient lung metastases that had progressed 8-fold from the baseline at seeding (FIG. 6I). Consistent with LXR activation inhibiting metastatic initiation, GW3965 108 treatment decreased the number of macroscopic metastatic nodules formed (FIG. 6J). Finally, treatment of mice with GW3965 108 in this ‘adjuvant’ pre-clinical context significantly prolonged their survival times following metastatic colonization (FIG. 6K).

### Example 6 LXR Activation Reduces Melanoma Progression and Metastasis in a Genetically-Driven Mouse Model of Melanoma

Roughly 60% of human melanoma tumors are marked by activating mutations in the Braf oncogene, with one single amino acid variant, B-Raf^{V600E}, being the predominant mutation found (Davies et al., 2002). Nearly 20% of melanomas exhibit activating mutations in B-Raf with concurrent silencing of the Pten tumor-suppressor, which drives progression to a malignant melanoma state (Tsao et al., 2004; Chin et al., 2006). Recently, Tyrosinase (Tyr)-driven conditional B-Raf activation and Pten loss were shown to genetically cooperate in driving mouse melanoma progression (Dankort et al., 2009).

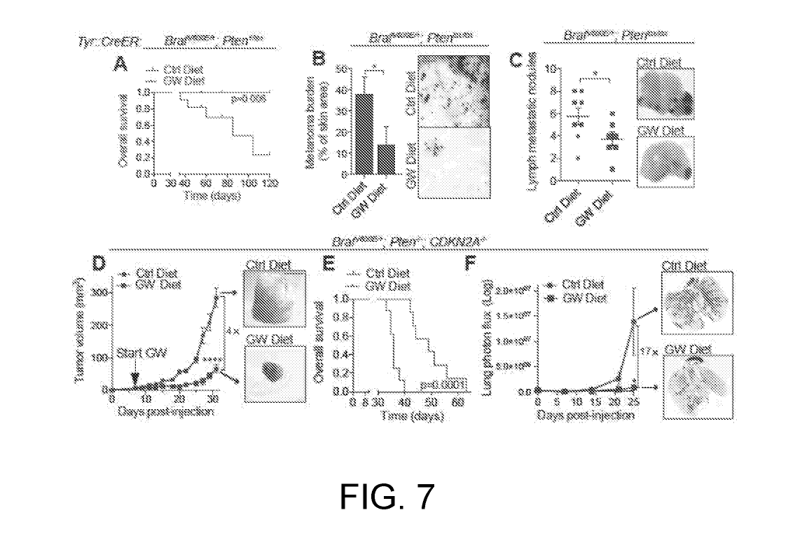

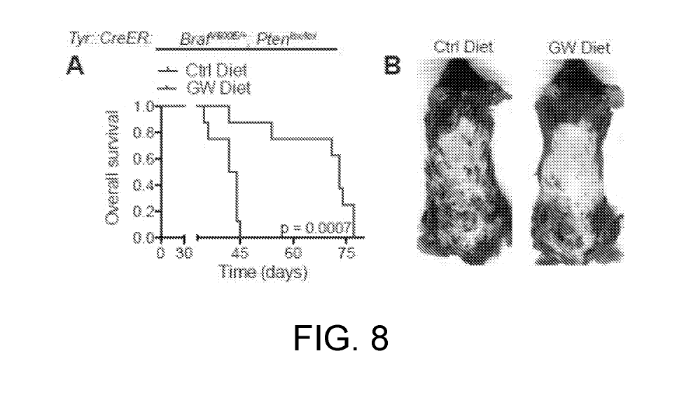

To determine whether LXR activation could suppress melanoma progression in this genetically-initiated model, melanomas in Tyr::CreER; B-Raf^{V600E/+}; Pten^{lox/+} and Tyr::CreER; B-Raf^{V600E/+}; Pten^{lox/lox} mice by intraperitoneal administration of 4-hydroxytamoxifen (4-HT) have been induced. Notably, oral administration of GW3965 108 following melanoma initiation attenuated tumor progression and significantly extended the overall survival times of both PTEN heterozygous Tyr::CreER; B-Raf^{V600E/+} Pten^{lox/lox} and PTEN homozygous Tyr::CreER; B-Raf^{V600E/E}; Pten^{lox/lox} mice (FIG. 7A-B and FIG. 8A-B). Next, the ability of GW3965 108 to suppress melanoma metastasis in this genetic context has been assessed. While macroscopic metastases in the lungs or brains of 4-HT-treated Tyr::CreER; B-Raf^{V600E/+;} Pten^{lox/lox} control mice were not detected, melanoma metastases to the salivary gland lymph nodes was consistently observed. Importantly, Tyr::CreER; B-Raf^{V600E/+;} Pten^{lox/lox} mice treated with GW3965 108 exhibited a decrease in the number of lymphatic metastases detected post-mortem (FIG. 7C). These findings indicate that LXR activation inhibits orthotopic metastasis in a genetically-driven melanoma model, in addition to its suppressive effects on primary melanoma tumor progression.

The cooperativity between B-Raf activation and Pten loss in driving melanoma progression can be further enhanced by inactivation of CDKN2A, a cell cycle regulator frequently mutated in familial melanomas (Hussussian et al., 1994; Kamb et al., 1994). The effect of LXR activation on B-Raf^{V600E/+}; Pten^{-/-}; CDKN2A^{-/-} melanomas, allowing us to test the therapeutic efficacy of LXR agonism in a more aggressive genetically-driven melanoma progression model has been examined. Importantly, therapeutic delivery of GW3965 108 robustly inhibited tumor growth and lung metastasis by B-Raf^{V600E/+}; Pten^{-/-}; CDKN2A^{-/-} primary mouse melanoma cells injected into syngeneic immunocompetent mice and extended the overall survival of mice bearing B-Raf^{V600E/+}; Pten^{-/-}; CDKN2A^{-/-} melanoma burden (FIG. 7D-F). Taken together, the robust suppression of melanoma progression across independent xenograft and genetically-induced immunocompetent melanoma mouse models that exhibit the diverse mutational profiles of human melanomas motivates the clinical testing of LXR activation therapy.

### Example 7 Pharmacologic Activation of LXRβ Suppresses Melanoma Phenotypes by Transcriptionally Inducing Melanoma-Cell ApoE Expression

Determination of the downstream molecular target of LXRβ that mediates suppression of melanoma progression has been investigated. To this end, human MeWo melanoma cells treated with the LXR agonist GW3965 108 have been transcriptomically profiled.

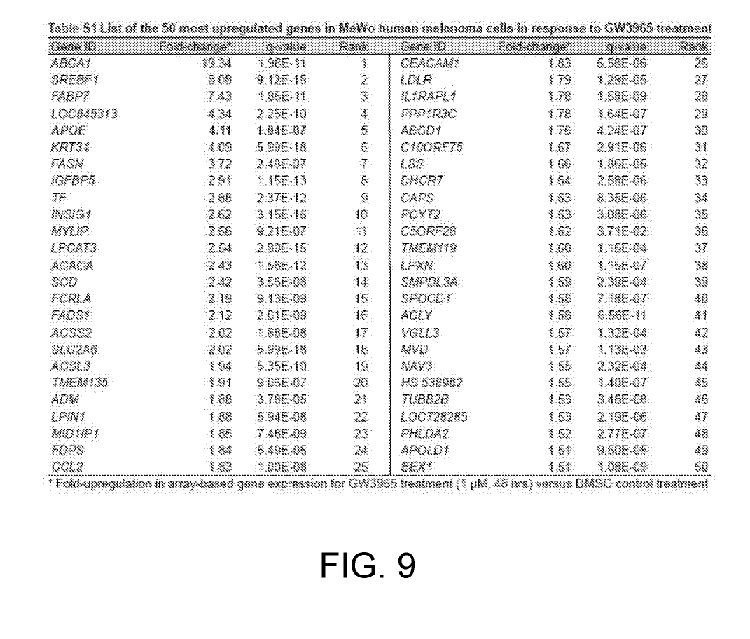

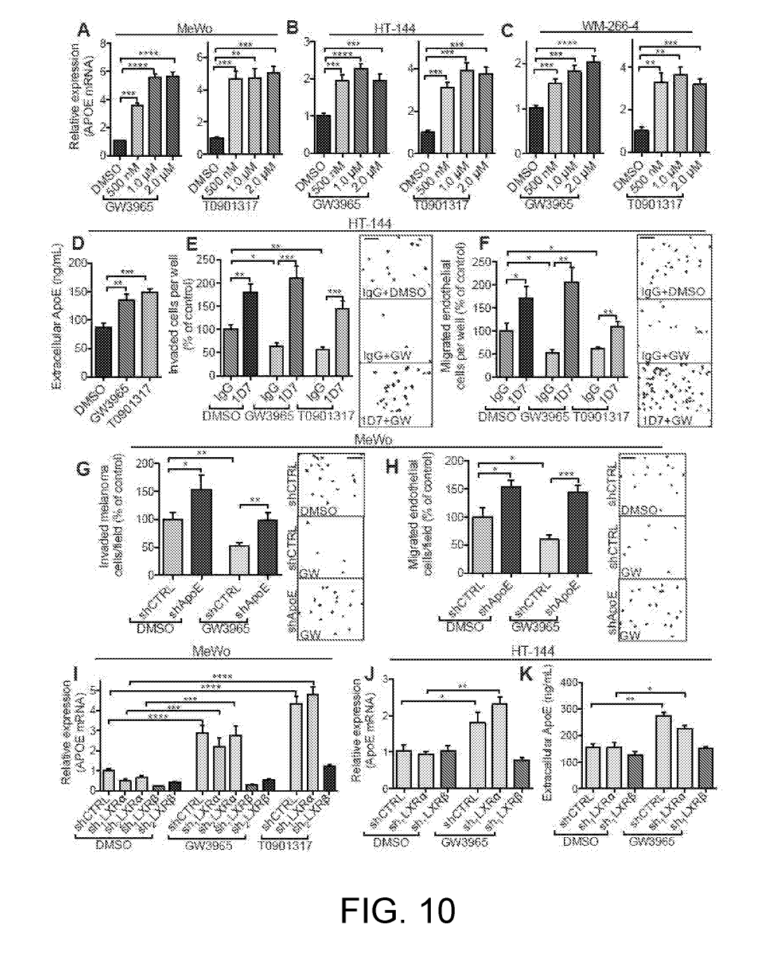

Out of the 365 genes that were significantly induced in response to LXR activation, ApoE, a previously validated transcriptional target of LXRs in macrophages and adipocytes (Laffitte et al., 2001), as the top upregulated secreted factor in melanoma cells (FIG. 9) has been identified. Quantitative real-time PCR (qRT-PCR) validation revealed robust upregulation of ApoEtranscript expression following treatment with independent LXR agonists across multiple human melanoma lines (FIG. 10A-C).

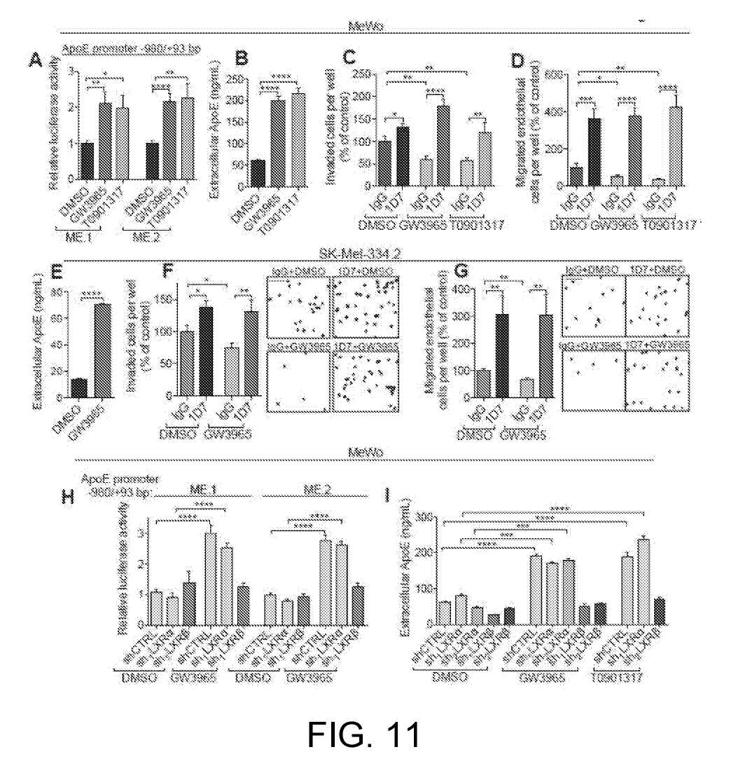

In light of the previously reported metastasis-suppressive function of ApoE in melanoma (Pencheva et al., 2012), whether LXRβ activation suppresses melanoma progression through transcriptional induction of ApoE has been investigated. Indeed, GW3965 108 and T0901317 110 were found to enhance the melanoma cell-driven activity of a luciferase reporter construct containing the ApoE promoter fused to either of two previously characterized LXR-binding multi-enhancer elements (ME.1 or ME.2) (Laffitte et al., 2001) (FIG. 11A). Importantly, this transcriptional induction resulted in elevated levels of secreted ApoE protein (FIG. 11B). Consistent with direct LXRβ targeting of ApoE in melanoma cells, neutralization of extracellular ApoE with an antibody fully blocked the LXRβ-mediated suppression of cell invasion and endothelial recruitment and further enhanced these phenotypes relative to the control IgG treatment (FIG. 11C-G and FIG. 10D-F), revealing the effects of LXR agonism to be modulated by extracellular ApoE.

Additionally, molecular knockdown of ApoE in melanoma cells also blocked the GW3965 108-mediated suppression of cell invasion and endothelial recruitment phenotypes (FIG. 10G-H). In agreement with this, melanoma cell depletion of LXRβ, but not LXRα, abrogated the ability of GW3965 108 and T0901317 110 to upregulate ApoE transcription and ultimately protein expression (FIG. 11H-I and FIG. 101-K). Collectively, these findings indicate that pharmacologic activation of LXRβ, the predominant LXR isoform expressed by melanoma cells, suppresses cell-intrinsic invasion and endothelial recruitment by melanoma cells through transcriptionally activating ApoE expression in melanoma cells.

### Example 8 Engagement of Melanoma-Derived and Systemic ApoE by LXRβ Activation Therapy

The LXRβ-induced suppression of key melanoma phenotypes by extracellular ApoE in vitro suggested that the suppressive effects of LXR agonists in vivo might be further augmented by the activation of LXRs in peripheral tissues, which could serve as robust sources of extracellular ApoE.

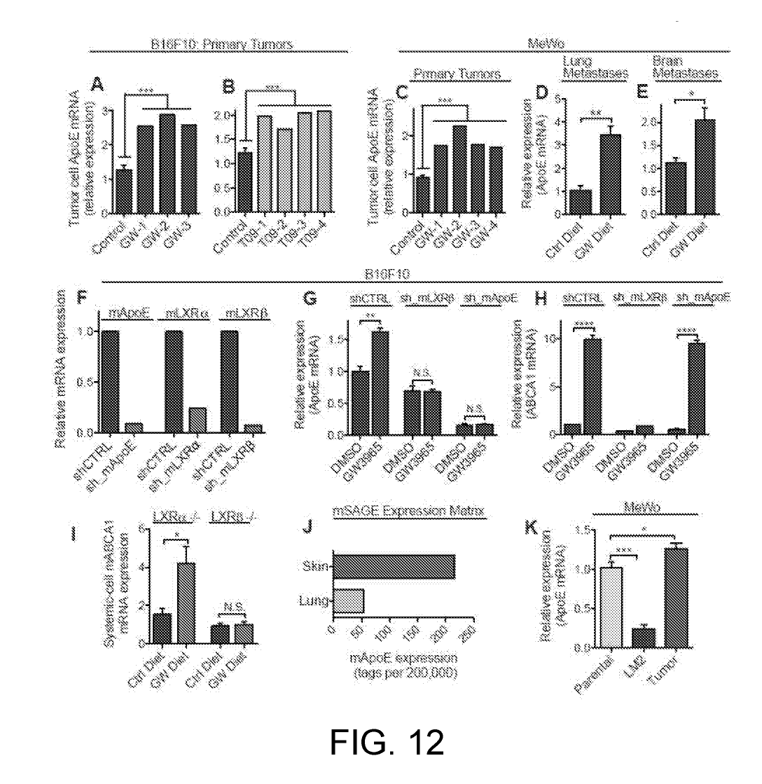

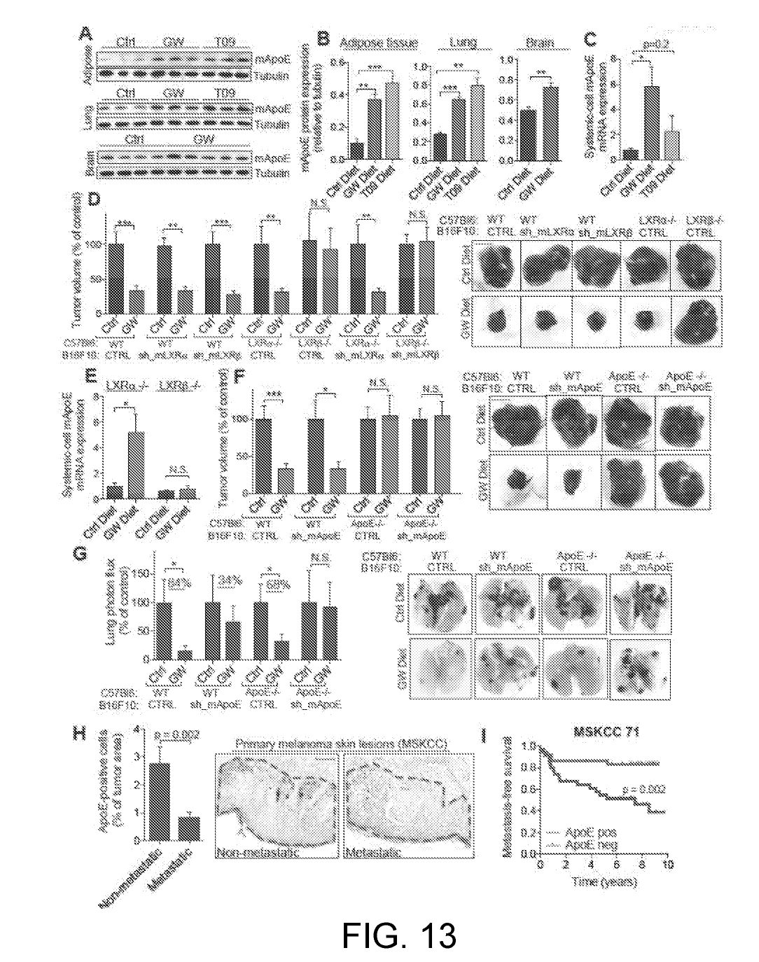

Importantly, such non-transformed tissues would be less vulnerable to developing resistance to LXR activation therapy, allowing for chronic ApoE induction in patients. Whether therapeutic LXR agonism suppresses melanoma progression by inducing ApoE derived from melanoma cells or systemic tissues has been investigated. Consistent with LXRβ agonism increasing ApoE expression in melanoma cells in vivo, ApoE transcript levels were upregulated in melanoma primary tumors as well as in melanoma lung and brain metastases dissociated from mice that were fed an LXR agonist-supplemented diet (FIG. 12A-E). Importantly, treatment of mice with either GW3965 108 or T0901317 110 significantly elevated ApoE protein expression in systemic adipose, lung, and brain tissues of mice (FIGS. 30A-B) and also upregulated ApoE transcript levels in circulating white blood cells (FIG. 13C). These results indicate that LXR activation therapy induces both melanoma-cell and systemic tissue ApoE expression in vivo.

To determine the in vivo requirement of melanoma-derived and systemic LXR activation for the tumor-suppressive effects of orally administered LXR agonists, the ability of GW3965 108 to suppress tumor growth by B16F10 mouse melanoma cells depleted of LXRβ has been tested.

Consistent with the findings in human melanoma cells, knockdown of mouse melanoma-cell LXRβ abrogated the GW3965-mediated induction of ApoE expression (FIG. 12F-H). Despite this, melanoma-cell LXRβ knockdown was unable to prevent the suppression of tumor growth by GW3965 108 (FIG. 12D), implicating a role for systemic LXR activation in tumor growth inhibition by GW3965 108. To identify the LXR isoform that mediates this non-tumor autonomous suppression of melanoma growth by LXR agonists, the effects of GW3965 108 on tumors implanted onto LXRα or LXRβ genetically null mice have been examined. Interestingly, genetic ablation of systemic LXRβ blocked the ability of GW3965 to suppress melanoma tumor growth, while LXRα inactivation had no effect on tumor growth inhibition by GW3965 (FIG. 6D). Importantly, the upregulation of systemic ApoE expression by GW3965 108, an agonist with 6-fold greater activity towards LXRβ than LXRα, was abrogated in LXRβ −/−, but not in LXRα −/− mice (FIG. 13E and FIG. 121). These results indicate that ApoE induction by GW3965 108 in peripheral tissues is predominantly driven by systemic LXRβ activation. In agreement with this, systemic LXRβ has been found to be the primary molecular target and effector of GW3965 108 in mediating melanoma tumor growth suppression.

Whether ApoE is required for the in vivo melanoma-suppressive effects of LXR agonists has been examined. Consistent with the lack of an impact for melanoma-cell LX: knockdown on the tumor-suppressive activity of GW3965 108, depletion of melanoma-cell ApoE did not prevent tumor growth inhibition by GW3965 108 neither (FIG. 12F-H and FIG. 13F). These findings suggest that the tumor suppressive effects of GW3965 108 might be primarily mediated through ApoE induction in systemic tissues.

Indeed, GW3965 108 was completely ineffective in suppressing tumor growth in mice genetically inactivated for ApoE (FIG. 13F), revealing systemic ApoE as the downstream effector of systemic LXRβ in driving melanoma tumor growth suppression. Interestingly, in contrast to primary tumor growth regulation, knockdown of melanoma-cell ApoE partially prevented the metastasis-suppressive effect of GW3965 108 (FIG. 13G). Similarly, genetic inactivation of ApoE only partially prevented the metastasis suppression elicited by GW3965 108 as well (FIG. 13G). The GW3965-driven inhibition of metastasis was completely blocked only in the context of both melanoma-cell ApoE knockdown and genetic inactivation of systemic ApoE (FIG. 13G), indicative of a requirement for both melanoma-derived and systemic ApoE engagement by LXRβ in suppressing metastasis. It has been concluded that the effects of LXRβ activation on primary tumor growth are elicited primarily through systemic ApoE induction, while the effects of LXRβ agonism on metastasis are mediated through ApoE transcriptional induction in both melanoma cells and systemic tissues.

The identification of ApoE as the sole downstream mediator of the LXRβ-induced suppression of melanoma phenotypes further highlights the importance of this gene as a suppressor of melanoma progression. To determine whether ApoE expression is clinically prognostic of melanoma metastatic outcomes, ApoE protein levels have been assessed by performing blinded immunohistochemical analysis on 71 surgically resected human primary melanoma lesions.

Patients whose melanomas had metastasized exhibited roughly 3-fold lower ApoE expression in their primary tumors relative to patients whose melanomas did not metastasize (FIG. 13H, P=0.002). Remarkably, ApoE expression levels in patients' primary melanoma lesions robustly stratified patients at high risk from those at low risk for metastatic relapse (FIG. 13I, P=0.002). These observations are consistent with previous findings that revealed significantly lower levels of ApoE in distant melanoma metastases relative to primary lesions (Pencheva et al., 2012). Collectively, this work indicates that ApoE, as a single gene, could likely act as a prognostic and predictive biomarker in primary melanomas to identify patients that i.) are at risk for melanoma metastatic relapse and as such ii.) could obtain clinical benefit from LXRβ agonist-mediated ApoE induction.

### Example 9 LXRβ Activation Therapy Suppresses the Growth of Melanomas Resistant to Dacarbazine and Vemurafenib

Encouraged by the robust ability of LXRβ activation therapy to suppress melanoma tumor growth and metastasis across a wide range of melanoma lines of diverse mutational backgrounds, whether melanomas that are resistant to two of the mainstay clinical agents used in the management of metastatic melanoma—dacarbazine and vemurafenib—could respond to LXRβ-activation therapy has been determined.

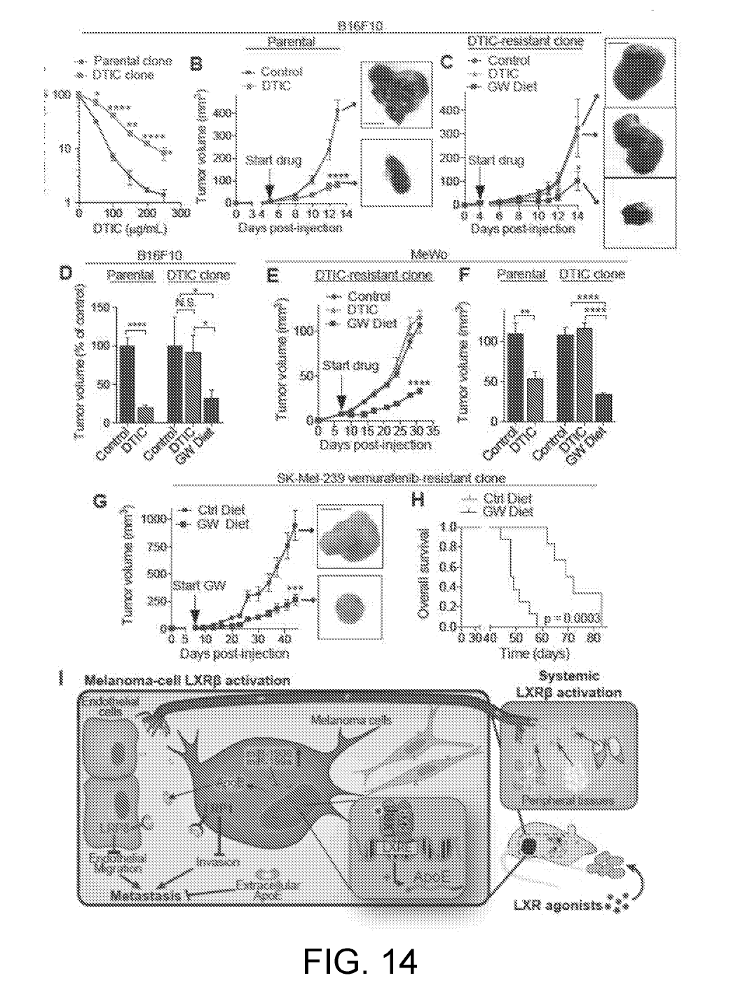

To this end, B16F10 clones resistant to dacarbazine (DTIC) by continuously culturing melanoma cells in the presence of DTIC for two months have been generated. This yielded a population of cells that exhibited a 7-fold increase in viability in response to high-dose DTIC treatment compared to the parental B16F10 cell line (FIG. 14A). To confirm that this in vitro-derived DTIC clone was also resistant to DTIC in vivo, the effects of dacarbazine treatment on tumor growth have been assessed.

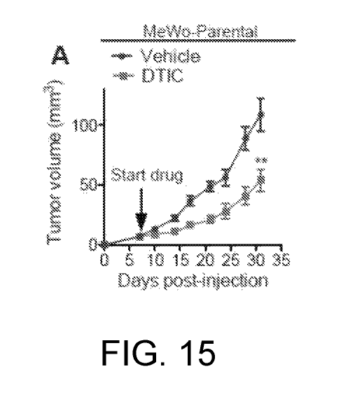

While dacarbazine significantly suppressed the growth of the DTIC-sensitive parental line (FIG. 14B), it did not affect tumor growth by B16F10 DTIC-resistant cells (FIG. 14C). GW3965 108 robustly suppressed tumor growth by the DTIC-resistant B16F10 melanoma clone by more than 70% (FIGS. 31C-D). Importantly, oral delivery of GW3965 108 also strongly inhibited the growth of in vivo-derived DTIC-resistant human melanoma tumors formed by the independent MeWo cell line (FIG. 14E-F and FIG. 15A).

These results reveal that LXRβ agonism is effective in suppressing multiple melanoma cell populations that are resistant to dacarbazine—the only FDA-approved cytotoxic chemotherapeutic in metastatic melanoma. The findings have important clinical implications for melanoma treatment since all stage IV patients who are treated with dacarbazine ultimately progress by developing tumors that are resistant to this agent.

The impact of LXRβ activation therapy on melanoma cells resistant to the recently approved B-Raf kinase inhibitor, vemurafenib—a regimen that shows activity against B-Raf-mutant melanomas (Bollag et al., 2010; Sosman et al., 2012) has been assessed. Numerous investigators have derived melanoma lines resistant to vemurafenib (Poulikakos et al., 2011; Shi et al., 2012, Das Thakur et al., 2013). GW3965 2 treatment suppressed the growth of the previously derived SK-Mel-239 vemurafenib-resistant line by 72% (FIG. 14G) and significantly prolonged the survival of mice bearing vemurafenib-resistant melanoma burden (FIG. 14H). The findings from combined pharmacologic, molecular and genetic studies in diverse pre-clinical models of melanoma establish LXRβ targeting as a novel therapeutic approach that robustly suppresses melanoma tumor growth and metastasis through the transcriptional induction of ApoE—a key suppressor of melanoma invasion and metastatic angiogenesis (Pencheva et al., 2012; FIG. 14I).

### Example 10 LXR Agonists LXR-623, WO-2007-002563 Ex. 19, WO-2010-0138598 Ex. 9, and SB742881, Induce ApoE Expression in Human Melanoma Cells

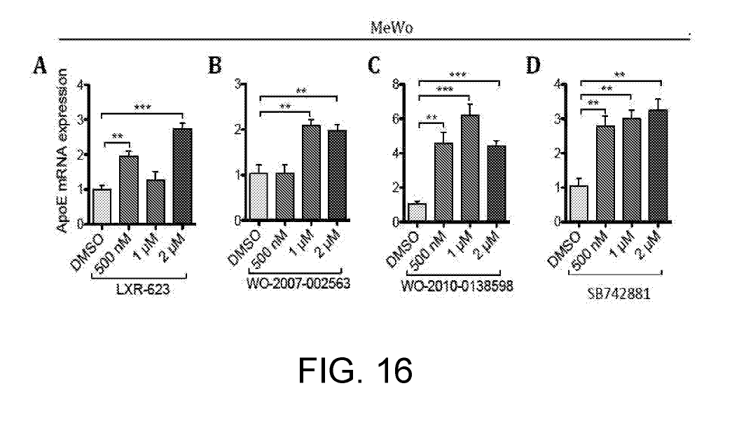

Given that ApoE activation by treatment with LXR agonists GW3965 108 and T0901317 110 resulted in therapeutic benefit for inhibiting tumor growth and metastasis, the ability of other LXR agonists to induce ApoE expression in human melanoma cell lines (FIG. 16) has been examined.

To determine the effect of the various LXR agonists (LXR-623, WO-2007-002563 Ex. 19, WO-2010-0138598 Ex. 9, and SB742881 on ApoE expression in melanoma cells, 1×10⁵ human MeWo melanoma cells were seeded in a 6-well plate. The following day, DMSO or the respective LXR agonist was added to the cell media at a concentration of 500 nM, 1 μM, or 2 μM, as indicated, and the cells were incubated in the presence of DMSO or the drug for 48 hours at 37° C. The total amount of DMSO added to the cell media was kept below 0.2%. RNA was extracted from whole cell lysates using the Total RNA Purification Kit (17200, Norgen). For every sample, 600 ng of RNA was reverse transcribed into cDNA using the cDNA First-Strand Synthesis kit (Invitrogen). Approximately 200 ng of cDNA was mixed with SYBR® green PCR Master Mix and the corresponding forward and reverse primers specific for detection of human ApoE. Each reaction was carried out in quadruplicates, and ApoE mRNA expression levels were measured by quantitative real-time PCR amplification using an ABI Prism 7900HT Real-Time PCR System (Applied Biosystems). The relative ApoE expression was determined using the ΔΔCt method. GAPDH was used as an endogenous control for normalization purposes.

Indeed, treatment with the LXR agonists LXR-623, WO-2007-002563 Ex. 19, WO-2010-0138598 Ex. 9, and SB742881 all led to varied degrees of ApoE expression induction. (FIG. 16A-C).

### Example 11 Treatment with the LXR Agonist GW3965 Inhibits In Vitro Tumor Cell Invasion of Renal Cancer, Pancreatic Cancer, and Lung Cancer

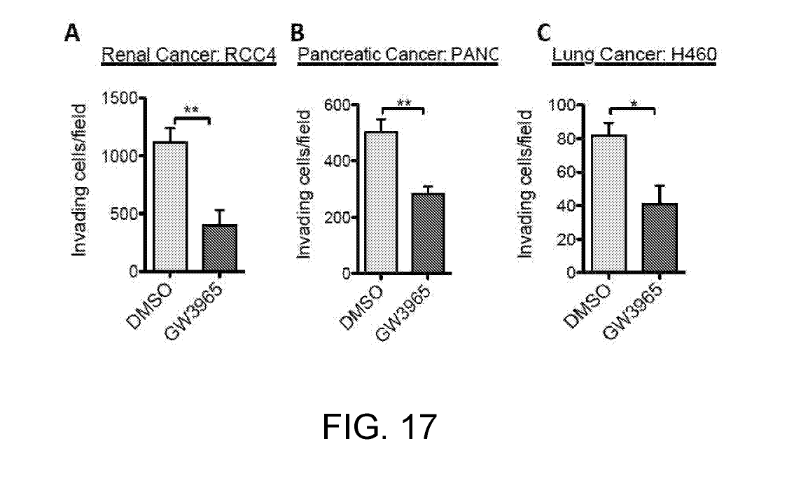

Treatment with LXR agonists resulted in inhibition of melanoma tumor cell invasion has been demonstrated. Given that this effect is mediated by activation of ApoE expression, treatment with LXR agonists would result in inhibition of in vitro tumor cell invasion in breast cancer, pancreatic cancer, and renal cancer, since these cancer types were responsive to ApoE treatment has been hypothesized. In order to test this hypothesis, in vitro tumor cell invasion assays by treating breast cancer, pancreatic cancer, and renal cell cancer cell lines with the LXR agonist GW3965 108 (FIG. 17) have been performed.

Various cell lines (5×10⁴ RCC human renal cancer cells, 5×10⁴ PANC1 human pancreatic cancer cells, and 5×10⁴H460 human lung cancer cells) were treated with DMSO or GW3965 at 1 μM for 56 hours. The cells were serum starved for 16 hours in 0.2% FBS media in the presence of DMSO or GW3965. Following serum starvation, the cells were subjected to the trans-well invasion assay using a matrigel invasion chamber system (354480, BD Biosciences). Invasion chambers were pre-equilibrated prior to the assay by adding 0.5 mL of starvation media to the top and bottom wells. Meanwhile, cancer cells were trypsinized and viable cells were counted using trypan blue. Cancer cells were then resuspended at a concentration of 1×10⁵ cells/1 mL starvation media, and 0.5 mL of cell suspension, containing 5×10⁴ cells, was seeded into each trans-well. The invasion assay was allowed to proceed for 24 hours at 37′C. Upon completion of the assay, the inserts were washed in PBS, the cells that did not invade were gently scraped off from the top side of each insert using q-tips, and the cells that invaded into the basal insert side were fixed in 4% PFA for 15 minutes at room temperature. Following fixation, the inserts were washed in PBS and then cut out and mounted onto slides using VectaShield mounting medium containing DAPI nuclear stain (H-1000, Vector Laboratories). The basal side of each insert was imaged using an inverted fluorescence microscope (Zeiss Axiovert 40 CFL) at 5× magnification, and the number of DAPI-positive cells was quantified using ImageJ.

Indeed, treatment with GW3965 108 resulted in inhibition of tumor cell invasion in all three cancer types tested (FIG. 17A-C). This further demonstrated the broad therapeutic potential of LXR agonists for treating various cancer types.

### Example 12 Treatment with the LXR Agonist GW3965 Inhibits Breast Cancer Tumor Growth In Vivo

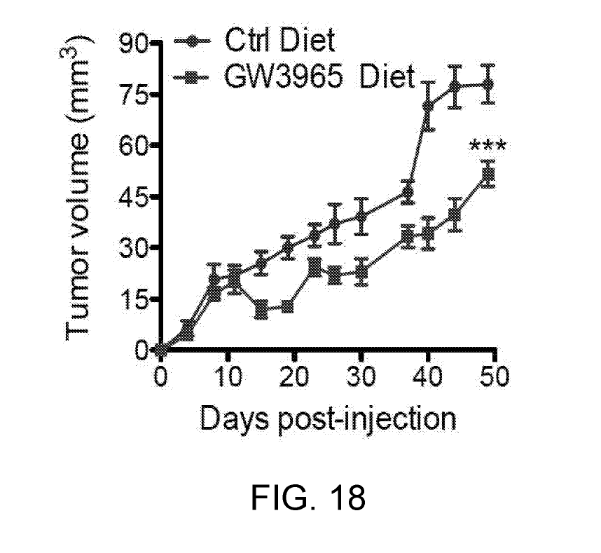

That LXR agonists inhibit in vitro cancer progression phenotypes in breast cancer, pancreatic cancer, and renal cancer has been demonstrated. To investigate if LXR agonist treatment inhibits breast cancer primary tumor growth in vivo, mice injected with MDA-468 human breast cancer cells were treated with either a control diet or a diet supplemented with LXR agonist GW3965 108 (FIG. 18).

To determine the effect of orally delivered GW3965 108 on breast cancer tumor growth, 2×10⁶ MDA-468 human breast cancer cells were resuspended in 50 μL PBS and 50 μL matrigel and the cell suspension was injected into both lower memory fat pads of 7-week-old Nod Scid gamma female mice. The mice were assigned to a control diet treatment or a GW3965-supplemented diet treatment (75 mg/kg/day) two days prior to injection of the cancer cells. The GW3965 108 drug compound was formulated in the mouse chow by Research Diets, Inc. Tumor dimensions were measured using digital calipers, and tumor volume was calculated as (small diameter)²×(large diameter)/2.

Treatment with GW3965 resulted in significant reduction in breast cancer tumor size in vivo (FIG. 18).

### Example 13 Effects of Treatment with LXR Agonists LXR-623, WO-2007-002563 Ex. 19, WO-2010-0138598 Ex. 9, and SB742881 on In Vitro Melanoma Progression Phenotypes

The ability of various LXR agonists to induce ApoE expression with varying potency in melanoma cells (FIG. 16) has been demonstrated. Since the therapeutic effect of LXR agonists on cancer is via activation of ApoE expression, that the therapeutic potency of any given LXR agonist is directly correlated with its ability to induce ApoE expression has been hypothesized. To confirm this, the effect of treatment with various LXR agonists on in vitro endothelial recruitment and tumor cell invasion of melanoma cells has been quantified. As shown in FIG. 19, the degree to which LXR agonists inhibit in vitro cancer progression phenotypes is related to the LXR agonist's ApoE induction potency.

Cell Invasion:

MeWo human melanoma cells were treated with DMSO, LXR-623, WO-2007-002563 Ex. 19, WO-2010-0138598 Ex. 9, or SB742881 at 1 μM each for 56 hours. The cells were then serum starved for 16 hours in 0.2% FBS media in the presence of each corresponding drug or DMSO. Following serum starvation, the cells were subjected to the trans-well invasion assay using a matrigel invasion chamber system (354480, BD Biosciences). Invasion chambers were pre-equilibrated prior to the assay by adding 0.5 mL of starvation media to the top and bottom wells. Meanwhile, cancer cells were trypsinized and viable cells were counted using trypan blue. Cancer cells were then resuspended at a concentration of 2×10⁵ cells/1 mL starvation media, and 0.5 mL of cell suspension, containing 1×10⁵ cells, was seeded into each trans-well. The invasion assay was allowed to proceed for 24 hours at 37° C. Upon completion of the assay, the inserts were washed in PBS, the cells that did not invade were gently scraped off from the top side of each insert using q-tips, and the cells that invaded into the basal insert side were fixed in 4% PFA for 15 minutes at room temperature. Following fixation, the inserts were washed in PBS, cut out, and mounted onto slides using VectaShield mounting medium containing DAPI nuclear stain (H-1000, Vector Laboratories). The basal side of each insert was imaged using an inverted fluorescence microscope (Zeiss Axiovert 40 CFL) at 5× magnification, and the number of DAPI-positive cells was quantified using ImageJ.

Endothelial Recruitment:

MeWo human melanoma cells were treated with DMSO, LXR-623, WO-2007-002563 Ex. 19, WO-2010-0138598 Ex. 9, or SB742881 at 1 μM each for 56 hours. Subsequently, 5×10⁴ cancer cells were seeded into 24-well plates in the presence of each drug or DMSO and allowed to attach for 16 hours prior to starting the assay. Human umbilical vein endothelial cells (HUVEC cells) were serum-starved in 0.2% FBS-containing media overnight. The following day, 1×10⁵ HUVEC cells were seeded into a 3.0 μm HTS Fluoroblock insert (351151, BD Falcon) fitted into each well containing the cancer cells at the bottom. The HUVEC cells were allowed to migrate towards the cancer cells for 20 hours, after which the inserts were washed in PBS, fixed in 4% PFA, labeled with DAPI, and mounted on slides. The basal side of each insert was imaged using an inverted fluorescence microscope (Zeiss Axiovert 40 CFL) at 5× magnification, and the number of DAPI-positive cells was quantified using ImageJ.

LXR agonists that potently induce ApoE expression (e.g. WO-2010-0138598 Ex. 9 and SB742881) are more effective at inhibiting cancer progression phenotypes (FIG. 19) than lower potency LXR agonists. This further demonstrates that the therapeutic benefit of LXR agonist treatment for cancer is a result of ApoE induction.

### Example 14 Treatment with LXR Agonists Inhibit Melanoma Tumor Growth In Vivo

That LXR agonists that induce ApoE expression inhibit in vitro tumor activity has been demonstrated. To confirm if these agonists inhibit melanoma tumor growth in vivo, mice that were injected with B16F10 melanoma cells were treated with either LXR-623, WO-2007-002563 Ex. 19, WO-2010-0138598 Ex. 9, or SB742881.

To assess the effect of orally administered LXR-623, WO-2007-002563 Ex. 19, WO-2010-0138598 Ex. 9, or SB742881 on melanoma tumor growth, 5×10⁴ B16F10 mouse melanoma cells were resuspended in 50 μL PBS and 50 μL matrigel and the cell suspension was subcutaneously injected into both lower dorsal flanks of 7-week-old C57BL/6 mice. The mice were palpated daily for tumor formation and after detection of tumors measuring 5-10 m³ in volume, the mice were assigned to a control chow or a chow containing each respective LXR agonist: LXR-623 (20 mg/kg/day), WO-2007-002563 Ex. 19 (100 mg/kg/day), WO-2010-0138598 Ex. 9 (10 mg/kg/day or 100 mg/kg/day), or SB742881 (100 mg/kg/day). The LXR drug compounds were formulated in the mouse chow by Research Diets, Inc. Tumor dimensions were measured using digital calipers, and tumor volume was calculated as (small diameter)²×(large diameter)/2.

Consistent with the in vitro data, LXR agonists that potently induce ApoE expression in vitro (WO-2010-0138598 Ex. 9, and SB742881) significantly inhibited melanoma primary tumor growth in vivo (FIG. 20). This is also consistent with the results demonstrating that other LXR agonists which potently induce ApoE expression (GW3965 108, T0901317 110) also inhibit primary tumor growth in vivo (FIG. 4).

Accordingly, the above examples focused on characterizing the molecular and cellular mechanisms by which it exerts its effects. To this end, it was found that ApoE targets two distinct, yet homologous, receptors on two diverse cell types. ApoE acting on melanoma cell LRP1 receptors inhibits melanoma invasion, while its action on endothelial cell LRP8 receptors suppresses endothelial migration. The results from loss-of-function, gain-of-function, epistasis, clinical correlation, and in vivo selection derivative expression analyses give rise to a model wherein three miRNAs convergently target a metastasis suppressor network to limit ApoE secretion, thus suppressing ApoE/LRP1 signaling on melanoma cells and ApoE/LRP8 signaling on endothelial cells (FIG. 7K). Although the above systematic analysis has identified ApoE and DNAJA4 as key targets and direct mediators of the metastatic phenotypes regulated by these miRNAs, it cannot be excluded that the three miRNAs may individually retain additional target genes whose silencing may contribute to metastatic progression. The ability of ApoE or DNAJA4 knock-down to fully rescue the metastasis suppression phenotypes seen with individual miRNA silencing, however, strongly suggests that these genes are the key mediators of the miRNA-dependent effects on metastasis.

The above results reveal combined molecular, genetic, and in vivo evidence for a required and sufficient role for ApoE in the suppression of melanoma metastatic progression. ApoE can distribute in the circulatory system both in a lipoprotein-bound and a lipid-free state (Hatters et al., 2006). While it has been shown that lipid-free recombinant ApoE is sufficient to suppress melanoma invasion and endothelial migration, it is possible that ApoE contained in lipoprotein particles could also suppress melanoma invasion and endothelial recruitment. The ability of recombinant ApoE to inhibit these pro-metastatic phenotypes, as well as the increased melanoma invasion and endothelial recruitment phenotypes seen with antibody-mediated ApoE neutralization suggests that the ApoE molecule itself is the key mediator of these phenotypes. Consistent with the findings disclosed herein, a synthetic peptide fragment of ApoE was previously found to inhibit endothelial migration through unknown mechanisms (Bhattacharjee et al., 2011). The findings disclosed herein are consistent with a role for melanoma cell-secreted and systemic endogenous ApoE in inhibiting endothelial recruitment, which is not secondary to impaired endothelial cell growth.

The above-described molecular, genetic, and in vivo studies reveal a role for endogenous cancer-derived ApoE in the modulation of endothelial migration and cancer angiogenesis through endothelial LRP8 receptor signaling. This robust non-cell-autonomous endothelial recruitment phenotype mediated by ApoE/LRP8 signaling suggests that ApoE may also modulate metastatic angiogenesis in other cancer types, and such a general role for ApoE in cancer angiogenesis biology remains to be explored. ApoE is a polymorphic molecule with well-established roles in lipid, cardiovascular, and neurodegenerative disorders. Its three major variants, ApoE2, ApoE3, and ApoE4, display varying representations in the human population, with ApoE3 being the most common variant (Hatters et al., 2006). The three isoforms differ at residues 112 and 158 in the N-terminal domain, which contains the ApoE receptor-binding domain. These structural variations are thought to give rise to distinct functional attributes among the variants. Consistent with this, the three ApoE isoforms differ in their binding affinity for members of the LDL receptor family, lipoprotein-binding preferences, and N-terminus stability. Namely, ApoE2 has 50- to 100-fold attenuated LDL receptor binding ability compared to ApoE3 and ApoE4 (Weisgraber et al., 1982), while ApoE4, unlike the other two variants, preferentially binds to large lower-density lipoproteins (Weisgraber et al., 1990) and exhibits the lowest N-terminus stability (Morrow et al., 2000). These functional differences confer pathophysiological properties to select ApoE isoforms. While ApoE3, found in 78% of the population, is considered a neutral allele, ApoE2 is associated with type III hyperlipoprotenemia (Hatters et al., 2006) and ApoE4 represents the major known genetic risk factor for Alzheimer's disease (Corder et al., 1993) and also correlates with a modest increase in the risk of developing cardiovascular disease (Luc et al., 1994). Given that the multiple human melanoma cell lines analyzed in the above study are homozygous for the ApoE3 allele, as well as the ability of recombinant ApoE3 to inhibit melanoma invasion and endothelial recruitment, the above findings are consistent with ApoE3 being sufficient and required for the suppression of melanoma metastatic progression. However, it will be of interest in the future to determine whether ApoE2 and ApoE4 can modulate these pro-metastatic phenotypes to a similar extent as ApoE3 and whether specific ApoE genotypes confer enhanced risk of melanoma metastatic progression.

Besides surgical resection of primary melanoma lesions, there are currently no effective therapies for the prevention of melanoma metastasis with interferon therapy increasing overall survival rates at 5 years by a meager 3% based on meta-analyses, while phase III trial data demonstration of significant survival benefits is still outstanding (Garbe et al., 2011). The dramatic enhancement of melanoma metastatic progression in the context of genetic ablation of systemic ApoE suggests that modulating ApoE levels may have significant therapeutic implications for melanoma—a disease that claims approximately 48,000 lives a year globally (Lucas et al., 2006). Given the robust ability of ApoE to suppress melanoma invasion, endothelial migration, metastatic angiogenesis, and metastatic colonization, therapeutic approaches aimed at pharmacological induction of endogenous ApoE levels may significantly reduce melanoma mortality rates by decreasing metastatic incidence.

The above-described unbiased in vivo selection based approach led to discovery of deregulated miRNAs that synergistically and dramatically promote metastasis by cancer cells from independent patients' melanoma cell lines representing both melanotic and amelanotic melanomas. While miR-1908 has not been previously characterized, miR-199a has been implicated in hepatocellular carcinoma (Hou et al., 2011; Shen et al., 2010) and osteosarcoma (Duan et al., 2011) that, contrary to melanoma, display down-regulation of miR-199a expression levels. These differences are consistent with the established tissue-specific expression profiles of miRNAs in various cancer types. The identification of miR-199a as a promoter of melanoma metastasis is supported by a previous clinical association study revealing that increased miR-199a levels correlate with uveal melanoma progression (Worley et al., 2008), suggesting that induced miR-199a expression may be a defining feature of metastatic melanoma regardless of site of origin. Previous studies have implicated additional miRNAs in promoting melanoma metastatic progression such as miR-182 (Segura et al., 2009), miR-214 (which was upregulated in metastatic melanoma cells, but it did not functionally perform in the above studies; Penna et al., 2011), and miR-30b/miR-30d (Gaziel-Sovran et al., 2011). Each of these miRNAs have been reported to only modestly modulate melanoma metastasis, leading to 1.5- to 2-fold increased or decreased metastasis modulation upon miRNA over-expression or knock-down, respectively. In contrast, over-expression of either miR-199a or miR-1908 enhanced metastasis by 9-fold (FIG. 1C), while combinatorial miRNA knock-down synergistically suppressed melanoma metastasis by over 70-fold (FIG. 7E). Therefore, the study disclosed herein represents the first systematic discovery of multiple miRNAs that convergently and robustly promote human melanoma metastasis, as well as the first to assign dual cell-autonomous/non-cell-autonomous roles to endogenous metastasis-regulatory miRNAs in cancer.

Previous systematic analysis of miRNAs in breast cancer revealed primarily a decrease in the expression levels of multiple microRNAs in in vivo selected metastatic breast cancer cells (Tavazoie et al., 2008). Those findings were consistent with the subsequent discovery of many additional metastasis suppressor miRNAs in breast cancer (Shi et al., 2010; Wang and Wang, 2011), the identification of a number of miRNAs as direct transcriptional targets of the p53 tumour suppressor (He et al., 2007), the downregulation of miRNAs in breast cancer relative to normal tissues (Calin and Groce, 2006; lorio et al., 2005), the downregulation of drosha and dicer in breast cancer (Yan et al., 2011) and metastatic breast cancer (Grelier et al., 2011), as well as the pro-tumorigenic and pro-metastatic effects of global miRNA silencing through dicer knock-down (Kumar et al., 2007; Kumar et al., 2009; Martello et al., 2010; Noh et al., 2011). In contrast to breast cancer, the above findings in melanoma reveal a set of miRNAs upregulated in metastatic human melanoma, raising the intriguing possibility that miRNA processing may actually act in a pro-tumorigenic or pro-metastatic manner in melanoma. Consistent with this, dicer is required for melanocytic development (Levy et al., 2010), and dicer expression was recently found to positively correlate with human melanoma progression in a clinico-pathological study (Ma et al., 2011). These findings, when integrated with the findings disclosed here, motivate future studies to investigate the functional requirement for dicer (Bernstein et al., 2001) in melanoma metastasis.

The establishment of in vivo selection models of melanotic and amelanotic melanoma metastasis has allowed one to identify the cellular phenotypes displayed by highly metastatic melanoma cells. The work reveals that, in addition to enhanced invasiveness, the capacity of melanoma cells to recruit endothelial cells is significantly enhanced in highly metastatic melanoma cells relative to poorly metastatic melanoma cells. Additionally, it was found that three major post-transcriptional regulators of metastasis strongly mediate endothelial recruitment. It was further found that the downstream signaling pathway modulated by these miRNAs also regulates endothelial recruitment. These findings reveal endothelial recruitment to be a defining feature of metastatic melanoma cells. Enhanced endothelial recruitment capacity was also recently found to be a defining feature of metastatic breast cancer, wherein suppression of metastasis by miR-1 26 was mediated through miRNA targeting of two distinct signaling pathways that promote endothelial recruitment (Png et al., 2012). In breast cancer, endothelial recruitment increased the likelihood of metastatic initiation rather than tumor growth. Similarly, the melanoma metastasis promoter miRNAs studied here dramatically enhanced metastatic colonization, without enhancing primary tumor growth, and increased the number of metastatic nodules-consistent with a role for these miRNAs and their regulatory network in metastatic initiation rather than tumor growth promotion. Taken together, these findings are consistent with endothelial recruitment into the metastatic niche acting as a promoter of metastatic initiation and colonization in these distinct epithelial cancer types. Such a non-canonical role for endothelial cells in cancer progression would contrast with the established role of endothelial cells in angiogenic enhancement of blood flow spurring enhanced tumor growth. Endothelial cells are known to play such non-canonical roles in development by supplying cues to neighboring cells during organogenesis (Lammert et al., 2001). Such cues have also been recently shown to promote organ regeneration (Ding et al., 2011; Ding et al., 2010; Kobayashi et al., 2010). Future work is needed to determine the metastasis stimulatory factors provided by endothelial cells that catalyze metastatic initiation.

The ability of miR-199a-3p, miR-199a-5p, and miR-1908 to individually predict metastasis-free survival in a cohort of melanoma patients indicates the significance of each miRNA as a clinical predictor of melanoma cancer progression. Importantly, the dramatic and highly significant capacity of the three miRNA aggregate signature (FIG. 7D) to stratify patients at high risk from those at essentially no risk for metastatic relapse reveals both the cooperativity of these miRNAs, as well as their clinical potential as melanoma biomarkers (Sawyers, 2008) for identifying the subset of patients that might benefit from miRNA inhibition therapy. Therapeutic miRNA targeting has gained momentum through the use of in vivo LNAs that have been shown to antagonize miRNAs in mice (Elmer et al., 2008(b); KrOtzfeldt et al., 2005; Obad et al., 2011) and primates (Elmer et al., 2008(a)) and are currently being tested in human clinical trials. The powerful prognostic capacity of the three miRNAs, proof-of-principle demonstration of robust synergistic metastasis prevention achieved by treating highly metastatic melanoma cells with a cocktail of LNAs targeting miR-199a-3p, miR-199a-5p, and miR-1908 (FIG. 7E), as well as the metastasis suppression effect of therapeutically delivered in vivo-optimized LNAs targeting these miRNAs (FIG. 7J) motivate future clinical studies aimed at determining the therapeutic potential of combinatorially targeting these pro-metastatic and pro-angiogenic miRNAs in patients at high risk for melanoma metastasis—an outcome currently lacking effective chemotherapeutic options.

The foregoing examples and description of the preferred embodiments should be taken as illustrating, rather than as limiting the present invention as defined by the claims. As will be readily appreciated, numerous variations and combinations of the features set forth above can be utilized without departing from the present invention as set forth in the claims. Such variations are not regarded as a departure from the scope of the invention, and all such variations are intended to be included within the scope of the following claims. All references cited herein are incorporated in their entireties.

1. A method of treating cancer comprising administering an effective amount of a compound of any one of Formula I-XXVI, any one of compounds 1-107, or pharmaceutically acceptable salts thereof to a subject in need thereof.

2. A method of treating cancer comprising contacting a cell with an effective amount of a compound of any one of Formula I-XXVI, any one of compounds 1-107, or pharmaceutically acceptable salts thereof.

3. A method of slowing the spread of a migrating cancer comprising administering an effective amount of a compound of any one of Formula I-XXVI, any one of compounds 1-107, or pharmaceutically acceptable salts thereof to a subject in need thereof.

4-6. (canceled)

7. The method of claim 1, wherein said effective amount is an amount sufficient to increase the expression level or activity level of ApoE to a level sufficient to slow the spread of metastasis of said cancer.

8. The method of claim 1, wherein said cancer is drug resistant or has failed to respond to a prior therapy.

9. The method of claim 1, wherein said cancer is metastatic.

10. The method of claim 1, wherein said cancer is breast cancer, colon cancer, renal cell cancer, non-small cell lung cancer, hepatocellular carcinoma, gastric cancer, ovarian cancer, pancreatic cancer, esophageal cancer, prostate cancer, sarcoma, glioblastoma, diffuse large B-cell lymphoma, acute myeloid leukemia, or melanoma.

11. The method of claim 1, wherein said method further comprises administration of an additional anticancer therapy.

12. The method of claim 11, wherein said additional anticancer therapy is any one of the antiproliferatives listed in Table 2.

13. The method of claim 11, wherein said additional anticancer therapy is an immunotherapy.

14. The method of claim 13, wherein said immunotherapy is a PD-1 inhibitor, a PD-L1 inhibitor, a CTLA-4 inhibitor, a CSF-1R inhibitor, an IDO inhibitor, an A1 adenosine inhibitor, an A2A adenosine inhibitor, an A2B adenosine inhibitor, an A3A adenosine inhibitor, or an HDAC inhibitor.

15. The method of claim 11, wherein said additional anticancer therapy is administered within 28 days of administration of a compound of any one of Formula I-XXVI, any one of compounds 1-107, or pharmaceutically acceptable salts thereof.
